(19) United States
(12) Patent Application Publication
(10) Pub. No.: US 2020/0093852 A1
(43) Pub. Date: Mar. 26, 2020

(54) Methods of Treating Ocular Disorders

(71) Applicants: Keats NELMS, Hawker (AU); Riccardo NATOLI, Bungendore (AU); Brett SCHWARTZ, Gungahlin (AU)

(72) Inventors: Keats NELMS, Hawker (AU); Riccardo NATOLI, Bungendore (AU); Brett SCHWARTZ, Gungahlin (AU)

(21) Appl. No.: 16/475,671

(22) PCT Filed: Dec. 13, 2017
(86) PCT No.: PCT/AU2017/051382

§ 371 Date: Jul. 2, 2019

Related U.S. Application Data
(60) Provisional application No. 62/433,639, filed on Dec. 13, 2016.
(60) Provisional application No. 62/433,652, filed on Dec. 13, 2016.

(30) Foreign Application Priority Data
Jun. 20, 2017 (AU) 2017902346

(51) Int. Cl. A61K 31/737; A61K 9/00; A61P 27/02; A61P 29/00; A61K 39/395

(52) U.S. Cl. CPC A61K 31/737; A61K 9/0051; A61K 39/3955; A61P 29/00; A61P 27/02

(57) ABSTRACT

This invention relates to the use of a heparanase inhibitor for treating, or inhibiting the progression or development of, an ocular inflammatory disorder, such as age-related macular degeneration, diabetic retinopathy, retinitis pigmentosa, retinal 5 vein occlusion, retinoblastoma, uveitis, macular edema, dry eye, ocular inflammation associated with an infection or keratoconus.

This application claims priority to Australian Provisional Application No. 2017902346 entitled “Methods of Treating Ocular Disorders” filed on 20 Jun. 2017, U.S. Provisional Application No. 62/433,652 entitled “Heparanase Inhibitors and Use Thereof” filed on 13 December 2016, and U.S. Provisional Application No. 62/433,639 entitled “Heparanase Inhibitors and Use Thereof” filed on 13 Dec. 2016, the entire contents of which are hereby incorporated herein by reference.

### FIELD OF THE INVENTION

This invention relates generally to the use of a heparanase inhibitor for treating, or inhibiting the progression or development of, an ocular inflammatory disorder, such as age-related macular degeneration or diabetic retinopathy.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Ocular inflammatory disorders, such as diabetic retinopathy, age-related macular degeneration (AMD), retinitis pigmentosa and uveitis, are leading causes of vision loss in industrialized countries. In particular, diabetic retinopathy affects approximately one third of the United States population diagnosed with diabetes, and AMD affects approximately 6.5% of the United States population aged 40 years and older (Lee et al. (2015) Eye Vis (Lond), 2: 17; and Klein et al. (2011) Arch Ophthalmol, 129(1): 75-80). Such conditions represent a significant global economic burden and, accordingly, effective therapies for these conditions are desired.

Inflammation is a complex process involving a number of immune cells. One such immune cell is a macrophage, the activation of which has been found to be of high importance in the pathogenesis of ocular inflammatory disorders. Macrophages are present in all ocular tissues and are highly specialized to support ocular homeostasis and coordinate inflammatory responses to pathogenic or injurious stimuli (Chinnery et al. (2017) Eur J Physiol, 469: 501-515). In the retina, microglia are the resident macrophage cells. Although previously considered to be phenotypically indistinguishable from retinal infiltrating macrophages, retinal microglia have recently been shown to have a unique surface marker expression profile (Koren et al. (2016) Sci Rep, 6: 20636). Macrophages are generally understood to encompass mononuclear phagocytes that include microglia, which are tissue-based retinal macrophages derived from yolk sac erythroid-myeloid progenitors, and blood-borne monocytes, which are derived from hematopoietic cells.

In a diseased eye, excessive macrophage activation and accumulation in particular tissues, such as the activation and accumulation of microglia in the sub-retinal space in degenerative disorders, can disrupt the immune privilege of the eye (Li et al. (2015) Experimental Eye Research, 136: 116-130). Activated macrophages, including microglia, produce different kinds of products including complement proteins, pro-inflammatory cytokines, reactive oxygen species, growth factors and other products, which can result in a chronic local inflammation and can typically lead to further damage (Li et al. (2015) Experimental Eye Research, 136: 116-130). For example, in the pathogenesis of AMD, microglia activated by cell death migrate to the damaged area to phagocytose cellular debris but also secrete molecules that kill neighboring photoreceptors around the area of primary degeneration (Li et al. (2015) Experimental Eye Research, 136: 116-130). Furthermore, chemokines secreted by activated microglia trigger the infiltration of macrophages from surrounding tissues (Chen and Xu (2015) J Leukoc Biol, 98(5): 713-725). It is evident that ocular macrophage, including microglial, activation is an important target for the treatment and prevention of ocular inflammatory disorders, such as diabetic retinopathy and AMD.

Diabetic retinopathy is the most frequent complication of diabetes and can lead to blindness. This condition results from damage to small blood vessels and neurons in the retina and is characterized by retinal vasculopathy with endothelial cell dysfunction, breakdown of the blood-retinal barrier, inflammation and ischemia-induced angiogenesis (El-Asrar et al. (2015) IOVS, 56(13): 8239-8247). A number of factors are involved in the pathogenesis of diabetic retinopathy, some of which include VEGF activity and, importantly, macrophage, including microglial, and complement activation (Schroder et al. (1991) Am J Pathol, 139: 81-100; and Rangasamy et al. (2014) PLOS ONE, 9: e108508; Chen and Xu (2015) J Leukoc Biol, 98(5): 713-725). Current treatments for diabetic retinopathy include laser photocoagulation and anti-angiogenic therapy, such as treatment with a VEGF inhibitor.

AMD is a progressive degenerative disorder of the macula, which results in a loss of vision in the center of the visual field. The prevalence of this condition is increased in older adults. Factors which have been implicated in the development of AMD comprise macrophage, including microglial, activation, oxidative stress, chronic inflammation and complement activation (Dithmer et al. (2014) PLOS ONE, 9(2): e89150; Rutar et al. (2010) Curr Eye Res, 35(7): 631-643; and Rutar et al. (2012) J Neuroinflammation, 9: 221). Macrophage and complement activation, in particular, are considered to play major roles in the pathogenesis of AMD (Chen and Xu (2015) J Leukoc Biol, 98(5): 713-725). These factors are implicated in both neovascularization of the macula (‘wet’ AMD) and progressive photoreceptor degeneration (‘dry’ AMD) (Knickelbein et al. (2015) Int Ophthalmol Clin, 55(3): 63-78; and Janik-Papis et al. (2009) Klin Oczna, 111(4-6): 168-173). Present treatments for exudative ‘wet’ AMD target the predominant angiogenic factor in the eye, VEGF, and slow the progression of vessel growth and vision loss (van Lookeren Campagne et al. (2014) J Pathol, 232(2): 151-164). There are currently no treatments available for the more prevalent atrophic ‘dry’ AMD.

There is a need for the development of new therapies which effectively treat or prevent an ocular inflammatory disorder, such as diabetic retinopathy and AMD.

Heparanase, an enzyme which breaks down heparan sulfate, is implicated in angiogenesis, inflammation and metastasis (Klein et al. (1985) Adv Exp Med Biol, 189: 321-335). Heparanase has also been shown to activate macrophages but, interestingly, treatment with heparanase inhibitors does not block macrophage activation (Gutter-Kapon et al. (2016) PNAS, 113(48): E7808-E7817).

Surprisingly, it has been found that most of the heparanase that is produced in ocular diseases such as AMD and diabetic retinopathy is from activated ocular macrophages and this production is the major contributing factor in exacerbation of inflammatory mediators leading to ocular inflammatory disease. It has also been found unexpectedly that heparanase inhibitors can block ocular macrophage activation and complement fixation when locally administered to an eye and that this inhibition leads to significant alleviation of inflammation and associated disease. Accordingly, the inventors have conceived that heparanase inhibitors are useful for the treatment, and inhibition of the progression or development of, an ocular inflammatory disorder when locally administered to an eye.

### SUMMARY OF THE INVENTION

The present invention is predicated in part on the discovery that heparanase inhibitors block ocular macrophage activation, including activation of microglia, and complement fixation when locally administered to an eye.

In one aspect of the invention, there is provided a method of treating, or inhibiting the progression or development of, an ocular inflammatory disorder in a subject comprising locally administering a heparanase inhibitor or a pharmaceutically acceptable salt thereof to an eye of the subject.

In another aspect of the invention, there is provided a method of treating, or inhibiting the progression or development of, an ocular inflammatory disorder in a subject comprising locally administering a heparanase inhibitor or a pharmaceutically acceptable salt thereof to an eye of the subject, wherein the heparanase inhibitor inhibits one or more activities of heparanase including heparanase catalytic activity.

The invention also provides a use of a heparanase inhibitor or a pharmaceutically acceptable salt thereof for treating, or inhibiting the progression or development of, an ocular inflammatory disorder in a subject, wherein the heparanase inhibitor is formulated for local administration to an eye of the subject.

The invention also contemplates a use of a heparanase inhibitor or a pharmaceutically acceptable salt thereof for treating, or inhibiting the progression or development of, an ocular inflammatory disorder in a subject, wherein the heparanase inhibitor is formulated for local administration to an eye of the subject and the heparanase inhibitor is an inhibitor of one or more activities of heparanase including heparanase catalytic activity.

In a further aspect, there is provided a heparanase inhibitor or a pharmaceutically acceptable salt thereof for use in treating, or inhibiting the progression or development of, an ocular inflammatory disorder in a subject, wherein the heparanase inhibitor is formulated for local administration to an eye of the subject.

In yet another aspect of the invention, there is provided a heparanase inhibitor or a pharmaceutically acceptable salt thereof for use in treating, or inhibiting the progression or development of, an ocular inflammatory disorder in a subject, wherein the heparanase inhibitor is formulated for local administration to an eye of the subject and the heparanase inhibitor is an inhibitor of one or more activities of heparanase including heparanase catalytic activity.

In still another aspect of the invention, there is provided a use of a heparanase inhibitor or a pharmaceutically acceptable salt thereof in the manufacture of a medicament for treating, or inhibiting the progression or development of, an ocular inflammatory disorder in a subject, wherein the heparanase inhibitor is formulated for local administration to an eye of the subject.

The present invention also provides a use of a heparanase inhibitor or a pharmaceutically acceptable salt thereof in the manufacture of a medicament for treating, or inhibiting the progression or development of, an ocular inflammatory disorder in a subject, wherein the heparanase inhibitor is formulated for local administration to an eye of the subject and the heparanase inhibitor is an inhibitor of one or more activities of heparanase including heparanase catalytic activity.

In any of the above aspects, the ocular inflammatory disorder preferably arises from, or is otherwise associated with, microglial activation.

Suitably, in any of the above aspects, the ocular inflammatory disorder arises from, or is otherwise associated with, retinal inflammation.

In still another aspect, there is provided a topical ocular composition for treating, or inhibiting the progression or development of, an ocular inflammatory disorder in a subject, comprising, consisting or consisting essentially of a heparanase inhibitor or a pharmaceutically acceptable salt thereof, and a pharmaceutically acceptable carrier or diluent.

The present invention further provides a pharmaceutical ocular composition formulated for local administration to an eye of a subject, comprising, consisting or consisting essentially of a heparanase inhibitor or a pharmaceutically acceptable salt thereof and a pharmaceutically acceptable carrier or diluent.

Another aspect of the present invention provides a method for treating, or inhibiting the progression or development of, an ocular inflammatory disorder associated with macrophage activation in a subject comprising locally administering a heparanase inhibitor or a pharmaceutically acceptable salt thereof to an eye of the subject. In some embodiments, the ocular inflammatory disorder is further associated with complement fixation.

In still another aspect, the present invention provides a use of a heparanase inhibitor or a pharmaceutically acceptable salt thereof for treating, or inhibiting the progression or development of, an ocular inflammatory disorder associated with macrophage activation in a subject, wherein the heparanase inhibitor is formulated for local administration to an eye of the subject. In some embodiments, the ocular inflammatory disorder is further associated with complement fixation.

The present invention provides, in a further aspect, the use of a heparanase inhibitor or a pharmaceutically acceptable salt thereof in the manufacture of a medicament for treating, or inhibiting the progression or development of, an ocular inflammatory disorder associated with macrophage activation in a subject, wherein the heparanase inhibitor is formulated for local administration to an eye of the subject. In some embodiments, the ocular inflammatory disorder is further associated with complement fixation.

In a still further aspect, there is provided a heparanase inhibitor or a pharmaceutically acceptable salt thereof for use for treating, or inhibiting the progression or development of, an ocular inflammatory disorder associated with macrophage activation in a subject, wherein the heparanase inhibitor is formulated for local administration to an eye of the subject. In some embodiments, the ocular inflammatory disorder is further associated with complement fixation.

In some embodiments of any one or more of the above aspects, the ocular inflammatory disorder is selected from the group consisting of AMD, diabetic retinopathy, retinitis pigmentosa, retinal vein occlusion, retinoblastoma, macular edema, uveitis, dry eye and keratoconus; especially AMD, diabetic retinopathy or retinitis pigmentosa; most especially AMD.

In some embodiments of any one or more of the above aspects, the ocular inflammatory disorder is selected from the group consisting of AMD, diabetic retinopathy, retinitis pigmentosa, retinal vein occlusion, retinoblastoma, macular edema, uveitis, dry eye, ketatoconus and ocular inflammation associated with an infection; especially ocular inflammation associated with an infection, AMD, diabetic retinopathy or retinitis pigmentosa; more especially AMD, diabetic retinopathy or retinitis pigmentosa; most especially AMD.

The present invention also contemplates a method for inhibiting complement fixation comprising contacting an ocular macrophage cell with a heparanase inhibitor or a pharmaceutically acceptable salt thereof.

In yet another aspect, there is provided a use of a heparanase inhibitor or a pharmaceutically acceptable salt thereof for inhibiting complement fixation, wherein an ocular macrophage cell is contacted with the heparanase inhibitor. A use of the heparanase inhibitor or a pharmaceutically salt thereof in the manufacture of a medicament for this purpose is also contemplated by the present invention.

The present invention provides, in a further aspect, a heparanase inhibitor or a pharmaceutically acceptable salt thereof for use in inhibiting complement fixation, wherein an ocular macrophage cell is contacted with the heparanase inhibitor.

In some embodiments of any one or more of the above aspects, the heparanase inhibitor is an inhibitor of one or more of the following: heparanase catalytic activity, macrophage activation, complement fixation, growth factor activity and/or oxidative damage; preferably macrophage activation and/or complement fixation.

In some embodiments of any one or more of the above aspects, the heparanase inhibitor is an inhibitor of one or more heparanase activities, including heparanase catalytic activity.

In any one or more of the above aspects, the complement fixation preferably arises from, or is otherwise associated with, microglial activation.

Suitably, in any one or more of the above aspects, the complement fixation arises from, or is otherwise associated with, retinal inflammation.

### BRIEF DESCRIPTION OF THE DRAWINGS

FIG. 1 displays representative images of C3 expression and deposition by macrophages in human eyes using in situ hybridization (ISH). FIG. 1A is a representative image of a matched control eye, FIGS. 1B-D represent an eye with early AMD and FIGS. 1E-G represent an eye with late AMD.

FIG. 2 displays representative images of C3 expression and deposition by macrophages in rodent eyes using in situ hybridization (ISH). FIG. 2A is a representative image of a control eye and FIGS. 2B-F represent an eye seven days post retinal damage. FIGS. 2D-F show double labelling with a broad macrophage marker, IBA1, using immunohistochemistry (IHC).

FIG. 3 presents retinal expression of heparanase 1 (HPA1) protein via immunohistochemical (IHC) labelling. FIG. 3A is a representation of dim-reared control animals that have no photo-oxidative damage and that demonstrate no macrophages based on F4/80⁺ IHC labelling in the outer retina, between the outer nuclear layer (ONL) and the retinal pigment epithelium (RPE). FIG. 3B presents that after 5 days of photo-oxidative damage (PD), F4/80⁺ macrophages (red stain on representative cell indicated by white arrow) were recruited into the outer retina, and these macrophages were found to express heparanase 1 (green/yellow stain on/in the F4/80⁺ cell).

FIG. 4 presents a heparanase inhibition curve of pentosan polysulfate (PPS) and PI-88.

FIG. 5 presents the efficacy of pentosan polysulfate in vivo. FIG. 5A presents the number of TUNEL positive photoreceptors, FIG. 5B presents the number of IBA1 positive cells, FIG. 5C presents the a-wave amplitude and FIG. 5D presents the b-wave amplitude following treatment with pentosan polysulfate (HI 2 μg) in comparison to control and PBS alone in normal mouse eyes. FIG. 5E displays the effect of pentosan polysulfate on the number of C3 positive IBA1 positive cells in the outer retina, FIG. 5F presents the effect of pentosan polysulfate on the a-wave amplitude and FIG. 5G displays the effect of pentosan polysulfate on the b-wave amplitude in comparison to PBS and aflibercept (Eylea 2 μg) in mouse eyes five days after photo-oxidative damage.

FIG. 6 displays the efficacy of heparanase inhibitors in vivo in a mouse model of age-related macular degeneration. FIG. 6A presents the a-wave amplitude and FIG. 6B presents the b-wave amplitude in mouse eyes following treatment with pentosan polysulfate (PPS; 2 μg) in comparison to PBS (n=11). FIG. 6C presents the a-wave amplitude and FIG. 6D presents the b-wave amplitude in mouse eyes following treatment with BT-2172 (1 μL of a 200 μM solution) in comparison to PBS (n=7-11). Data is presented as mean±SEM. *=p<0.05.

FIG. 7 displays the retinal function of mouse eyes treated with pentosan polysulfate (PPS; 2 μg) or BT-2172 (1 μL of a 200 μM solution) in comparison to PBS treatment and C3 complement factor gene knockout mice (C3 KO) in a model of age-related macular degeneration. The amplitude of the a-wave of eyes at a flash intensity of 1.9 log cd.s/m² is presented. Data is presented as mean+SEM. *=p<0.05.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “acyl” is used herein to denote a group containing the moiety C═O (and not being a carboxylic acid, ester or amide). Preferred acyl groups include C(O)—R, wherein R is hydrogen or an alkyl, alkenyl, alkynyl, aryl, heteroaryl or heterocyclyl residue, preferably a C₁₋₂₀ residue. Examples of acyl groups include formyl; straight chain or branched alkanoyl such as, acetyl, propanoyl, butanoyl, 2-methyl propanoyl, pentanoyl, 2,2-di methylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutanoyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenyl methacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and naphthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl.

The terms “administration concurrently”, “administering concurrently” or “administered concurrently” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and preferably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example, at an interval of about one day to several weeks or months. The active agents may be administered in any order. The term “sequentially” as used herein means that the agents are administered in sequence, for example, at an interval of minutes, hours, days or weeks. If appropriate, the active agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs, solvates, hydrates, and the like. When the above term is used, then it is to be understood that this includes the active agent per se as well as pharmaceutically acceptable, pharmacologically active salts, esters, amides, prodrugs, metabolites, analogs, solvates, hydrates, etc. The term “agent” is not to be construed narrowly but extends to small molecules, saccharides and derivatives thereof, proteinaceous molecules such as peptides, polypeptides and proteins as well as compositions comprising them and genetic molecules such as RNA, DNA and mimetics and chemical analogs thereof as well as cellular agents.

As used herein, the term “alkenyl” includes within its meaning monovalent (“alkenyl”) and divalent (“alkenylene”) straight-chain or branched chain unsaturated hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Unless indicated otherwise, the stereochemistry about each double bond may be independently cis or trans, or E or Z as appropriate. Examples of suitable alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, heptenyl, octenyl, nonenyl, and decenyl.

The term “alkoxy” as used herein refers to straight chain or branched alkoxy (O-alkyl) groups, wherein alkyl is as defined herein. Examples include methoxy, ethoxy, n-propoxy, i-propoxy, s-butoxy, t-butoxy, and the like.

As used herein, the term “alkyl” includes within its meaning monovalent (“alkyl”) and divalent (“alkylene”) straight chain or branched chain saturated hydrocarbon group having 1 to 10 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, s-butyl, amyl, 1,2-dimethylpropyl, 1,1-dimethylpropyl, pentyl, i-pentyl, hexyl, 1-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-methylbutyl, 3-methyl butyl, 4-methylbutyl, n-hexyl, 2-methylpentyl, 3-methylpentyl, 4-methylpentyl, 5-methylpentyl, 2-ethylbutyl, 3-ethyl butyl, heptyl, octyl, nonyl, and decyl.

The term “alkynyl” includes within its meaning monovalent (“alkynyl”) and divalent (“alkynylene”) straight chain or branched chain unsaturated hydrocarbon group, containing from 2 to 10 carbon atoms and at least one carbon to carbon triple bond. Up to three carbon-carbon triple bonds may be present. Thus, “C₂-C₆alkynyl” means an alkynyl radical having from 2 to 6 carbon atoms. Alkynyl groups include, but are not limited to, ethynyl, 1-propynyl, 1-butynyl, 2-butynyl, 1-methyl-2-butynyl, 3-methyl-1-butynyl, 1-pentynyl, 1-hexynyl, methylpentynyl and so on. The straight or branched portion of the alkynyl group may contain triple bonds and may be substituted if a substituted alkynyl group is indicated.

As used herein, the term “and/or” refers to and encompasses any and all possible combinations of one or more of the associated listed items, as well as the lack of combinations when interpreted in the alternative (or).

“Aralkyl” means alkyl as defined above which is substituted with an aryl group as defined herein, e.g., —CH₂phenyl, —(CH₂)₂phenyl, —(CH₂)₃phenyl, —CH₂CH(CH₃)CH₂phenyl, and the like and derivatives thereof.

As used herein, the term “aryl” or “aromatic” as a group or part of a group denotes (i) an optionally substituted monocyclic, or fused polycyclic, aromatic carbocycle (ring structure having ring atoms that are all carbon) that may have up to 10 atoms per ring, for example, from 6 to 10 atoms per ring denoted C₆₋₁₀aryl. Examples of aryl groups include phenyl, naphthyl, phenanthryl and the like; (ii) an optionally substituted partially saturated bicyclic aromatic carbocyclic moiety in which a phenyl and a C₅₋₇cycloalkyl or C₅₋₇cycloalkenyl group are fused together to form a cyclic structure, such as tetrahydronaphthyl, indenyl or indanyl. The group may be a terminal group or a bridging group.

In certain instances, substituents may be defined with a range of carbons that includes zero, such as (C₀-C₆)alkylene-aryl. If aryl is taken to be phenyl, this definition would include phenyl itself as well as, for example, —CH₂Ph, —CH₂CH₂Ph, CH(CH₃)CH₂CH(CH₃)Ph.

It will also be recognized that the compounds described herein may possess asymmetric centers and are therefore capable of existing in more than one stereoisomeric form. The invention thus also relates to compounds in substantially pure isomeric form at one or more asymmetric centers e.g. greater than about 90% ee, such as about 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

The term “C₁₋₃alkylenedioxy” as used herein refers to an —O(CH₂)₁₋₃O— group wherein the oxygen atoms of the alkylenedioxy group are attached to two adjacent carbon atoms of the parent molecular moiety forming a 5-, 6- or 7-membered ring. Exemplary alkylenedioxy groups are methylenedioxy and 1,2-ethylenedioxy.

The term “catalytic activity” in relation to heparanase is used herein to refer to the cleavage of heparan sulfate, specifically the hydrolysis of the β-glycosidic bond within heparan sulfate.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “cycloalkyl” means a saturated or partially saturated, monocyclic, fused or spiro polycyclic, carbocycle that may contain from 3 to 9 carbon atoms per ring, such as cyclopropyl, cyclobutyl, cyclopentyl, cyclohexyl, spiro[3.3]heptane, and the like, unless otherwise specified. It includes monocyclic systems such as cyclopropyl and cyclohexyl, bicyclic systems such as decalin, and polycyclic systems such as adamantane. The group may be a terminal group or a bridging group.

By “derivative” is meant a molecule, such as a polysaccharide, that has been derived from the basic molecule by modification, for example by conjugation or complexing with other chemical moieties as would be understood in the art.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the formulation of the composition, the assessment of the medical situation, and other relevant factors. It is expected that the amount will fall in a relatively broad range that can be determined through routine trials.

As used herein, the terms “halogen” or “halo” are synonymous and refer to fluorine, chlorine, bromine or iodine.

The term “heteroaryl” or “heteroaromatic” either alone or as part of a group means a monocyclic heteroaryl group having a 5- or 6-membered aromatic ring having one or more heteroatoms as ring atoms in the aromatic ring with the remainder of the ring atoms being carbon atoms, or a 8-10 membered bicyclic heteroaryl consisting of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The monocyclic heteroaryl and the bicyclic heteroaryl may be connected to the parent molecular moiety through any carbon atom or any nitrogen atom contained within the monocyclic heteroaryl or the bicyclic heteroaryl. Representative examples of monocyclic heteroaryl include, but are not limited to, furyl, imidazolyl, isoxazolyl, isothiazolyl, oxadiazolyl, oxazolyl (e.g. 1,3-oxazolyl, 1,2-oxazolyl), pyridinyl (e.g. 2-, 3-, 4-pyridinyl), pyridazinyl, pyrimidinyl, pyrazinyl, pyrazolyl, pyrrolyl, tetrazolyl, thiadiazolyl, thiazolyl, thienyl, triazolyl (e.g. 1,2,3-triazolyl, 1,2,4-triazolyl), and triazinyl. Representative examples of bicyclic heteroaryl include, but are not limited to, benzimidazolyl, benzofuranyl, benzothienyl, benzoxadiazolyl (e.g. 2,1,3-benzoxadiazolyl), cinnolinyl, dihydroquinolinyl, dihydroisoquinolinyl, furopyridinyl, indazolyl, indolyl (e.g. 2- or 3-indolyl), isoquinolinyl (e.g. 1-, 3-, 4-, or 5-isoquinolinyl), naphthyridinyl (e.g. 1,5-naphthyridinyl, 1,7-naphthyridinyl, 1,8-naphthyridinyl, etc), pyrrolopyridinyl (e.g. pyrrolo[2,3-b]pyridinyl), quinolinyl (e.g. 2-, 3-, 4-, 5-, or 8-quinolinyl), quinoxalinyl, tetrahydroquinolinyl, and thienopyridinyl. In some embodiments, the heteroaryl group is an N-heteroaryl group having one or more nitrogen heteroatoms, e.g. 1, 2, 3 or 4 nitrogen heteroatoms depending on the particular structure. N-heteroaryl groups may also have heteroatoms other than nitrogen, but N-heteroaryl groups are characterized by having at least one nitrogen heteroatom. Exemplary N-heteroaryl groups include imidazolyl, indolyl, (e.g. 2- or 3-indolyl), naphthyridinyl, pyrazinyl, pyridyl (e.g. 2-, 3- or 4-pyridyl), pyrrolyl, pyrimidinyl, quinolinyl (e.g. 2-, 3-, 4-, 5-, or 8-quinolinyl), isoquinolinyl, quinazolinyl, quinoxalinyl, triazinyl, among others. The heteroaryl group may be a terminal group or a bridging group and may be attached through a heteroatom or any carbon ring atom. As with the definition of heterocycle below, “heteroaryl” is also understood to include the N-oxide derivative of any nitrogen-containing heteroaryl.

As used herein, the term “heteroatom” or variants such as “hetero-” refers to 0, N, NH and S.

The term “heterocycle” or “heterocyclyl” as used herein is intended to mean a 5- to 10-membered nonaromatic heterocycle containing from 1 to 4 heteroatoms selected from the group consisting of O, N, NH and S, and includes bicyclic groups.

The term “heterocycloalkyl” as used herein refers to monovalent (heterocycloalkyl”) and divalent (“heterocycloalkylene”), saturated, monocyclic, bicyclic, fused or spiro polycyclic, hydrocarbon radicals having from 3 to 10 ring atoms, wherein from 1 to 5, 1 to 4 or from 1 to 3, typically 1 or 2 ring atoms are heteroatoms independently selected from O, N, NH, or S. The heterocycloalkyl group may be C₃₋₆ heterocycloalkyl. The heterocycloalkyl group may be C₃₋₅heterocycloalkyl. Representative examples of heterocycloalkyl groups include aziridinyl, azetidinyl, pyrrolidinyl, piperidinyl, piperazinyl, quinuclidinyl, morpholinyl, diazaspiro[3.3]heptane (e.g. 2,6-diazaspiro[3.3]heptane), tetrahydrothiophenyl, tetrahydrofuranyl, tetrahydropyranyl, and the like. In one or more embodiments, the heterocycloalkyl group is an N-heterocycloalkyl having one or more nitrogen heteroatoms, e.g. 1, 2, 3 or 4 nitrogen heteroatoms depending on the particular structure. N-heterocycloalkyl groups may also have heteroatoms other than nitrogen, but are characterized by having at least one nitrogen heteroatom. Exemplary N-heterocycloalkyl groups include aziridinyl, azetidinyl, pyrrolidinyl, piperidinyl, piperazinyl, morpholinyl, 2,6-diazaspiro[3.3]heptane among others. The heterocycloalkyl group may be a terminal group or a bridging group and may be attached through a heteroatom or any carbon ring atom.

The term “inhibitor” as used herein refers to an agent that decreases, inhibits or impairs at least one function or biological activity of a target molecule. As used herein, the term “heparanase inhibitor” refers to an agent that decreases, inhibits or impairs at least one function or biological activity of heparanase. Heparanase inhibitors may decrease, inhibit or impair heparanase catalytic activity, heparanase protein binding, heparanase-mediated modulation of gene transcription, heparanase-mediated initiation of cell signaling and/or angiogenesis. In particular embodiments, the heparanase inhibitor decreases, inhibits or impairs one or more biological activities of heparanase, including heparanase catalytic activity. In particular embodiments, the heparanase inhibitor is an inhibitor of the type 1 heparanase isoform. The heparanase inhibitor may also inhibit complement fixation, macrophage activation, oxidative damage and/or growth factor activity. In preferred embodiments, the heparanase inhibitor inhibits one or both of macrophage, preferably microglial, activation and complement fixation.

The term “ocular inflammatory disorder” as used herein refers to any condition affecting the eye which contains an inflammatory component and affects the vision of a subject. In some embodiments, the ocular inflammatory disorder is associated with a reduction in vision, such as a decrease in the visual field and/or a loss of visual acuity which is typically associated with diminishing or lessening of the acuteness or clearness of vision. A decrease in “visual acuity” typically refers to any measurable diminishing or lessening in the acuteness or clearness of form vision, which is dependent on the sharpness of the retinal focus within the eye and the sensitivity of the interpretative faculty of the brain. In certain embodiments, visual acuity refers to the Snellen acuity (e.g. 20/20).

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug, solvate, hydrate or derivative of a compound as provided herein is a salt, ester, amide, prodrug, solvate, hydrate or derivative that this not biologically or otherwise undesirable.

The terms “reduce”, “inhibit”, “suppress”, “decrease”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a subject refers to their subjective perception of disease symptoms, such as trouble with vision, satisfaction with vision and overall quality of vision, etc. In another embodiment, the reduction may be determined objectively, for example using visual acuity, contrast sensitivity, visual function index (such as VF-14), visual field test, fluorescein angiogram, optical coherence tomography, corneal tomography, corneal topography, tonometry, vascular features such as microvascular lesions, and electroretinogram. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

As used herein, the terms “salts” and “prodrugs” include any pharmaceutically acceptable salt, ester, hydrate or any other compound which, upon administration to the recipient, is capable of providing (directly or indirectly) a desired compound, or an active metabolite or residue thereof. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, boric, sulfamic, and phosphoric acid. Appropriate organic acids may be selected from aliphatic, cycloaliphatic, aromatic, heterocyclic carboxylic and sulfonic classes of organic acids, examples of which are formic, acetic, propionic, succinic, butyric, glycolic, gluconic, lactic, mucic, malic, tartaric, citric, ascorbic, glucoronic, fumaric, maleic, edetic, hydroxymaleic, pyruvic, alkyl sulfonic, arylsulfonic, aspartic, glutamic, benzoic, palmitic, oleic, lauric, valeric, benzenesulfonic, oxalic, anthranilic, mesylic, methanesulfonic, toluenesulfonic, salicylic, p-hydroxybenzoic, phenylacetic, mandelic, ambonic, pamoic, pantothenic, tannic, sulfanilic, cyclohexylaminosulfonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, suitable pharmaceutically acceptable base addition salts of the compounds of the present invention include organic salts made from N,N′-dibenzylethylenediamine, chloroprocaine, choline, diethanolamine, ethylenediamine, meglumine (N-methylglucamine), procaine, ammonium salts, alkylamonnium, quaternary salts such as tetramethylammonium salt, amino acid addition salts such as salts with glycine and arginine. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. In the case of compounds that are solids, it will be understood by those skilled in the art that the inventive compounds, agents and salts may exist in different crystalline or polymorphic forms, all of which are intended to be within the scope of the present invention and specified formulae. However, it will be appreciated that non-pharmaceutically acceptable salts also fall within the scope of the invention since these may be useful in the preparation of pharmaceutically acceptable salts. The preparation of salts and prodrugs can be carried out by methods known in the art. The salts can be prepared in situ during the final isolation and purification of the compounds of the invention, or separately by reacting the free base function with a suitable organic acid. Metal salts can be prepared by reaction of a desired compound with a metal hydroxide. An acid salt can be prepared by reacting an appropriate acid with a desired compound.

As used herein the term “selective” and “selectivity” refers to agents that inhibit heparanase without displaying substantial inhibition of one or more other endoglycosidases. Accordingly, an agent that is selective for heparanase exhibits heparanase selectivity of greater than about 2-fold, 5-fold, 10-fold, 20-fold, 50-fold, 100-fold or greater than about 500-fold with respect to inhibition of one or more other endoglycosidases.

As used herein, a “small molecule” refers to a compound that has a molecular weight of less than 3 kilodaltons (kDa), and typically less than 2 kDa, and suitably less than about 1 kDa. Small molecules may be peptides, polypeptides, peptidomimetics, carbohydrates such as oligosaccharides or polysaccharides, lipids or other organic or inorganic molecules.

The term “stereoisomer” as used herein refers to any two or more isomers that have the same molecular constitution and differ only in the three dimensional arrangement of their atomic groupings in space. Stereoisomers may be diastereoisomers or enantiomers. It will be recognized that the compounds described herein may possess asymmetric centers and are therefore capable of existing in more than one stereoisomeric form. The invention thus also relates to compounds in substantially pure isomeric form at one or more asymmetric centers e.g., greater than about 90% ee, such as about 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

The term “subject” as used herein refers to a vertebrate subject, particularly a mammalian or avian subject, for whom therapy or prophylaxis is desired. Suitable subjects include, but are not limited to, primates; avians; livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In some embodiments, the subject is a human. However, it will be understood that the aforementioned terms do not imply that symptoms are present.

The term “substituted” and variants such as “optionally substituted” as used herein, unless otherwise indicated, mean that a substituent may be further substituted by one or more additional substituents, which may be optional or otherwise. The term “optionally substituted” unless stated otherwise, denotes that the group may or may not be further substituted or fused (so as to form a polycyclic system), with one or more non-hydrogen substituent groups. Suitable chemically viable optional substituents for a particular functional group will be apparent to those skilled in the art. Typical optional substituents include C₁₋₄ alkyl, C₂₋₄ alkenyl, OH, halogen, O(C₁₋₄ alkyl), CN, NO₂, NR′R″ wherein R′ and R″ are independently selected from H and C₁-C₃ alkyl, CONR′R″, SH, S(C₁₋₃ alkyl), SO₂(C₁₋₃alkyl), CH₂—(C₁₋₃)alkoxy, C₁₋₃alkylenedioxy, C₆₋₁₀ aryl, —CH₂— phenyl, O—CH₂-phenyl, hydroxy(C₁₋₃ alkyl), halo(C₁₋₃ alkyl), CO₂H, CO₂(C₁₋₄ alkyl), among others. Preferred optional substituents include halogen, OH, NH₂, C₁₋₃ alkyl, C₁₋₃ alkoxy, —CH₂—(C₁₋₃)alkoxy, CH₂OH, halo-(C₁₋₃)alkyl, e.g. CF₃, halo-(C₁₋₃)alkoxy, e.g. OCF₃, phenyl and —CH₂-phenyl.

As used herein, the terms “treatment”, “treating”, and the like, refer to obtaining a desired pharmacologic and/or physiologic effect. The effect may be therapeutic in terms of a partial or complete cure for a disease or condition (e.g. an ocular inflammatory disorder) and/or adverse affect attributable to the disease or condition. These terms also cover any treatment of a condition or disease in a subject, and include: (a) inhibiting the disease or condition, i.e., arresting its development; or (b) relieving the disease or condition, i.e., causing regression of the disease or condition.

Each embodiment described herein is to be applied mutatis mutandis to each and every embodiment unless specifically stated otherwise.

### 2. Heparanase Inhibitors

The present invention is based, in part, on the identification that heparanase inhibitors block ocular macrophage activation, including microglial activation, and complement fixation, which stimulates the progression and/or development of ocular inflammatory disorders. Based on these findings, the present inventors tested the efficacy of locally administered heparanase inhibitors in an animal model of age-related macular degeneration and found that they were effective at improving retinal function. Based on these findings and due to their inhibition of heparanase, macrophage activation and complement fixation, the present inventors conceived that heparanase inhibitors would be useful for treating, or inhibiting the development or progression of, an ocular inflammatory disorder, such as AMD or diabetic retinopathy.

The heparanase inhibitor includes and encompasses any active agent that decreases, inhibits or impairs at least one function or biological activity of heparanase, such as heparanase catalytic activity, heparanase protein binding, heparanase-mediated modulation of gene transcription, heparanase-mediated initiation of cell signaling and/or angiogenesis. In particular embodiments, the heparanase inhibitor decreases, inhibits or impairs one or more biological activities of heparanase, including heparanase catalytic activity. In some embodiments, the heparanase inhibitor is an inhibitor of heparanase catalytic activity. In some embodiments, the heparanase inhibitor is other than an inhibitor of heparanase expression.

In some embodiments, the heparanase inhibitor further inhibits one or more of the following: complement fixation, macrophage activation, growth factor activity, such as VEGF activity, and oxidative damage. In some embodiments, the heparanase inhibitor further inhibits macrophage activation. In some embodiments, the heparanase inhibitor further inhibits macrophage activation and complement fixation. In some embodiments, the heparanase inhibitor further inhibits macrophage activation, complement fixation and growth factor activity, especially VEGF activity. In some embodiments, the heparanase inhibitor further inhibits macrophage activation, complement fixation and oxidative damage. In some embodiments, the heparanase inhibitor further inhibits macrophage activation, complement fixation, growth factor activity, especially VEGF activity, and oxidative damage. In any one or more of the above embodiments, the heparanase inhibitor is an inhibitor of heparanase catalytic activity.

The heparanase inhibitor may be a selective or non-selective inhibitor. In some embodiments, the heparanase inhibitor is a selective heparanase inhibitor.

Such inhibitors include, but are not limited to, small molecules and macromolecules including peptides; polypeptides; proteins; peptidomimetics; carbohydrates such as oligosaccharides and polysaccharides; oligosaccharide-aglycone conjugates; antibodies; lipopolysaccharides; lipids; polymers; or other organic or inorganic molecules. In some embodiments, the heparanase inhibitor is selected from a small molecule, polysaccharide, oligosaccharide, oligosaccharide-aglycone conjugate, antibody, protein and polymer.

The heparanase inhibitor may be in the form of a derivative, such as a pharmaceutically acceptable salt and/or solvate thereof, or prodrug thereof. In some embodiments, the heparanase inhibitor is in the form of a hydrate.

2.1 Small Molecules

The present invention contemplates small molecule agents that decreases, inhibits or impairs at least one function or biological activity of heparanase. Accordingly, in some embodiments, the heparanase inhibitor is a small molecule.

In some embodiments, the small molecule is an anionic compound, suitable examples of which include sulfated, phosphorylated or carboxylated compounds; especially a sulfated compound. In preferred embodiments, the small molecule is a polyanionic compound.

Suitable small molecules include, but are not limited to, trachyspic acid; reduced trachyspic acid derivatives described in Shiozawa et al. (1995) J Antibiot (Tokyo), 48(5): 357-362; trachyspic acid stereoisomers described in Zammit et al. (2007) Org Biomol Chem, 5: 2826-2834; suramin; naphthalenetrisulfonic acid derivatives of suramin including NF 127, NF 145 and NF 171 described in Gagliardi et al. (1998) Cancer Chemother Pharmacol, 41: 117-124, and Marchetti et al. (2003) Int J Cancer, 104(2): 167-174; 3-alkanoyl-5-hydroxymethyl tetronic acid derviatives including (R)-3-hexadecanoyl-5-hydroxymethyltetronic acid (RK-682) and 4-benzyl-RK-682 described in Ishida et al. (2004) J Antibiot (Tokyo), 57: 136-142, and Ishida et al. (2004) Mol Cancer Ther, 3(9): 1069-1077; indole derivatives described in WO 02/060373 A2; diphenyl ether derivatives described in WO 02/060375 A2; carbazole or fluorene derivatives described in WO 02/060867 A2; benz-1,3-azole derivatives described in WO 02/060374 A2; 2,3-dihydro-1,3-dioxo-1H-isoindole-5-carboxylic acid; 2,3-dihydro-1,3-dioxo-1H-isoindole-5-carboxylic acid derivatives described in Courtney et al. (2004) Bioorg Med Chem Lett, 14(12): 3269-3273, including 2-[4-propylamino-5-[5-(4-chloro)phenyl-benzoxazol-2-yl]phenyl]-2,3-dihydro-1,3-dioxo-1H-isoindole-5-carboxylic acid and OGT2492; furanyl-1,3-thiazol-2-yl and benzaoxazol-5-yl acetic acid derivatives described in Courtney et al. (2005) Bioorg Med Chem Lett, 15(9): 2295-2299, including OGT2115; N-(4-{[4-(1H-benzoimidazol-2-yl)-arylamino]-methyl}-phenyl)-benzamide derivatives described in Xu et al. (2006) Bioorg Med Chem Lett, 16(2): 404-408, including N-(4-{[4-(1H-benzoimidazol-2-yl)-phenylamino]-methyl}-phenyl)-3-bromo-4-methoxy-benzamide and N-(4-{[5-(1H-benzoimidazol-2-yl)-pyridin-2-ylamino]-methyl}-phenyl)-3-bromo-4-methoxy-benzamide; 1-[4-(1H-benzoimidazol-2-yl)-phenyl]-3-[4-(1H-benzoimidazol-2-yl)-phenyl]-urea derivatives described in Pan et al. (2006) Bioorg Med Chem Lett, 16(2): 409-412, including 1,3-bis-[4-(1H-benzoimidazol-2-yl)-phenyl]-urea and 1,3-bis-[4-(5,6-dimethyl-1H-benzoimidazol-2-yl)-phenyl]-urea; 4-(1H-benzoimidazol-2-yl)-phenylamine described in US 2011/020477 A1; amodiaquine; amodiaquine derivatives described in Gozalbes et al. (2013) Bioorg Med Chem, 21(7): 1944-1951; 2-(2,6-difluorophenyl)-5-(4-methoxyphenyl)-1-oxa-3-azaspiro[5.5]undecane (DMBO) described in Basappa et al. (2010) Cancer Letters, 297: 231-243; iminosugars described in Kawase et al. (1996) J Antibiot (Tokyo), 49(1): 61-64, including A-72363 C; uronic acid-type gem-diamine 1-N-iminosugars described in Nishimura et al. (2000) J Org Chem, 65(1): 2-11; the pseudodisaccharide described in Takahashi et al. (2001) Tetrahedron, 57(32): 6915-6926; CRM-646-A and CRM-646-B as described in Ko et al. (2000) J Antibiot (Tokyo), 53(2): 211-214; a sulfated linked cyclitol, including a sulfated pentameric cyclitol as described in Freeman et al. (2005) J Biol Chem, 280(10): 8842-8849, and WO 2003/004454 A1; inositol hexasulfate; KI-105 described in Ishida et al. (2004) Chem Biol, 11(3): 367-377; the glycoamino acid oligomers described in Suhara et al. (2002) Bioorg Med Chem, 10(6): 1999-2013, Suhara et al. (1996) Tetrahedron Letters, 37(15): 2549-2552, and Suhara et al. (1996) Tetrahedron Letters, 37(10): 1575-1578; the bridged saccharide compounds described in WO 1995/005182 A1; pyrithione described in Guo et al. (2017) Veterinary Microbiology, 201: 231-239; azasugar derivatives described in US 2007/0270354 A1; 4-alkylresorcinol described in EP 2484349 A1; benzoxazole, benzthiazole or benzimidazole acid derivatives described in WO 2004/046122 A1; cinnamic acid derivatives described in JP 2011074027 A including (E)-N-(5-methylisoxazol-3-yl)-3-(3,4,5-trimethoxyphenyl)acrylamide and (E)-3-(2-chlorophenyl)-N-(pyridin-3-ylmethyl)acrylamide; a cyclic carboxamide derivative described in EP 2484359 A1; a tetrazole derivative described in JP 2011074024 A, including 1-[1-(2-ethyl-6-methylphenyl)tetrazol-5-yl]cyclopentanamine; a naphthalene derivative described in JP 2011074024 A, including 1-(1-piperidylmethyl)naphthalene-2-ol; a cycloalkanone derivative described in JP 2011074024 A, including 2,6-bis[(4-hydroxyphenyl)methylene]cyclohexanone; sulfated lactobionic acid amide; a sulfated bis-lactobionic acid amide described in Klauser et al. (1991) Semin Thromb Hemost, 17(Suppl 1): 118-125, including LW 10082 (aprosulfate); a sulfated bis-aldonic acid amide; a 1,2,4-triazolo-1,3,4-thiadiazole bearing compound described in Baburajeev et al. (2017) BMC Cancer, 17: 235, including 2,4-diiodo-6-(3-phenyl-[1,2,4]triazolo[3,4-b][1,3,4]thiadiazol-6-yl)phenol; a quinazoline compound of Formula 1 described in U.S. Provisional Patent Application No. 62/433,652, especially BT-2172, BT-2229, BT-2162, BT-2169, BT-2185 or BT-2173; a tetrahydroquinazoline or dihydroquinazoline compound of Formula 2, 3 or 4 described in U.S. Provisional Patent Application No. 62/433,639; and pharmaceutically acceptable salts thereof. The entire contents of the publications listed above is herein incorporated by reference.

In some embodiments, the heparanase inhibitor is selected from the group consisting of trachyspic acid, suramin, 2,3-dihydro-1,3-dioxo-1H-isoindole-5-carboxylic acid, RK-682, 4-benzyl-RK-682, 2-[4-propylamino-5-[5-(4-chloro)phenyl-benzoxazol-2-yl]phenyl]-2,3-dihydro-1,3-dioxo-1H-isoindole-5-carboxylic acid, OGT2492, OGT2115, N-(4-{[4-(1H-benzoimidazol-2-yl)-phenylamino]-methyl}-phenyl)-3-bromo-4-methoxy-benzamide, N-(4-{[5-(1H-benzoimidazol-2-yl)-pyridin-2-ylamino]-methyl}-phenyl)-3-bromo-4-methoxy-benzamide, 1,3-bis-[4-(1H-benzoimidazol-2-yl)-phenyl]-urea, 1,3-bis-[4-(5,6-dimethyl-1H-benzoimidazol-2-yl)-phenyl]-urea, 4-(1H-benzoimidazol-2-yl)-phenylamine, amodiaquine, 2-(2,6-difluorophenyl)-5-(4-methoxyphenyl)-1-oxa-3-azaspiro[5.5]undecane, A-72363 C, CRM-646-A, CRM-646-B, inositol hexasulfate, KI-105, 4-alkylresorcinol, (E)-N-(5-methylisoxazol-3-yl)-3-(3,4,5-trimethoxyphenyl)acrylamide, (E)-3-(2-chlorophenyl)-N-(pyridin-3-ylmethyl)acrylamide, 1-[1-(2-ethyl-6-methylphenyl)tetrazol-5-yl]cyclopentanamine, 1-(1-piperidylmethyl)naphthalene-2-ol, 2,6-bis[(4-hydroxyphenyl)methylene]cyclohexanone, sulfated lactobionic acid amide, aprosulfate, a quinazoline compound of Formula 1, a tetrahydroquinazoline or dihydroquinazoline compound of Formula 2, 3 or 4, and a pharmaceutically acceptable salt and combinations thereof; especially trachyspic acid, suramin, 2,3-dihydro-1,3-dioxo-1H-isoindole-5-carboxylic acid, 2-[4-propylamino-5-[5-(4-chloro)phenyl-benzoxazol-2-yl]phenyl]-2,3-dihydro-1,3-dioxo-1H-isoindole-5-carboxylic acid, N-(4-{[4-(1H-benzoimidazol-2-yl)-phenylamino]-methyl}-phenyl)-3-bromo-4-methoxy-benzamide, N-(4-{[5-(1H-benzoimidazol-2-yl)-pyridin-2-ylamino]-methyl}-phenyl)-3-bromo-4-methoxy-benzamide, 1,3-bis-[4-(1H-benzoimidazol-2-yl)-phenyl]-urea, 1,3-bis-[4-(5,6-dimethyl-1H-benzoimidazol-2-yl)-phenyl]-urea, 4-(1H-benzoimidazol-2-yl)-phenylamine, amodiaquine, 2-(2,6-difluorophenyl)-5-(4-methoxyphenyl)-1-oxa-3-azaspiro[5.5]undecane, inositol hexasulfate, 4-alkylresorcinol, (E)-N-(5-methylisoxazol-3-yl)-3-(3,4,5-trimethoxyphenyl)acrylamide, (E)-3-(2-chlorophenyl)-N-(pyridin-3-ylmethyl)acrylamide, 1-[1-(2-ethyl-6-methyl phenyl)tetrazol-5-yl]cyclopentanamine, 1-(1-piperidylmethyl)naphthalene-2-ol, 2,6-bis[(4-hydroxyphenyl)methylene]cyclohexanone, sulfated bis-aldonic acid amide, sulfated lactobionic acid amide, aprosulfate, 2,4-diiodo-6-(3-phenyl-[1,2,4]triazolo[3,4-b][1,3,4]thiadiazol-6-yl)phenol, BT-2172, BT-2229, BT-2162, BT-2169, BT-2185, BT-2173, and a pharmaceutically acceptable salt and combinations thereof.

In some embodiments, the heparanase inhibitor is a quinazoline compound of Formula 1:

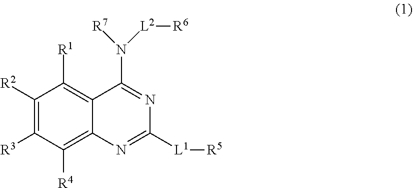

or a salt, hydrate, solvate, tautomer or stereoisomer thereof,
wherein:
R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
or R¹ and R², or R² and R³, or R³ and R⁴ together form C₁₋₃alkylenedioxy;
L¹ is selected from C₆₋₁₀aryl, NH, NHC₁₋₄alkyl, NHC₁₋₄alkyl-NHC(O)—, NHC₁₋₄alkyl-NHSO₂—, azetidinyl-NHC(O)—, azetidinyl-NHSO₂—, N(C₁₋₄alkyl)₂ wherein each alkyl is the same or different and is optionally substituted with a halo or hydroxyl group, or 12 is absent;
R⁵ is selected from H, halo, hydroxyl, C₁₋₆alkyl, C₁₋₆alkenyl, C₁₋₆alkynyl, C₃₋₆cycloalkyl, C₆₋₁₀aryl optionally substituted with 1 or 2 R^{X} groups, C₂₋₉heteroaryl optionally substituted with 1 or 2 R^{X} groups, heterocycloalkyl optionally substituted with 1 or 2 R^{X} groups, alkylheterocycloalkyl optionally substituted with 1 or 2 R^{X} groups, C(O)-heterocycloalkyl optionally substituted with 1 or 2 R^{X} groups, NHC(NH)NR′R″ wherein R′ and R″ are independently selected from H and C₁₋₃alkyl, NHC(O)NR′R″ wherein R′ and R″ are independently selected from H and C₁₋₃alkyl, or R⁵ is absent;
L² is selected from C₁₋₄alkyl, azetidinyl-C(O)—, C₁₋₄alkyl-NHC(O)—, C₁₋₄alkyl-NHSO₂—, —C(O)—, —SO₂—; or L² is absent;
R⁶ is selected from H, C₁₋₆ alkyl, guanidinyl, NHC(NH)NH(C₁₋₃alkyl), ureido, NHC(O)NH(C₁₋₃alkyl), C₆₋₁₀ aryl optionally substituted with 1 or 2 R^{X} groups, C₁₋₉ heteroaryl optionally substituted with 1 or 2 R^{X} groups, C₂₋₅ heterocycloalkyl optionally substituted with 1 or 2 R^{X} groups, C₃₋₆cycloalkyl optionally substituted with 1 or 2 R^{X} groups;
R⁷ is H or C₁₋₆alkyl;
or when L² is absent R⁶ and R⁷ together with the nitrogen to which they are attached form a heterocycloalkyl ring optionally substituted with 1 or 2 R^{X} groups;
each R^{X} is independently selected from hydroxyl, halo, nitro, NR′R″ wherein R′ and R″ are independently selected from H and C₁₋₃alkyl, C₁₋₄ alkyl, C₃₋₆cycloalkyl, C₁₋₄alkoxy, C(O)C₁₋₃alkyl, C(O)OC₁₋₄alkyl, C(O)NHR^{Y}, C₆₋₁₀aryl optionally substituted with 1 or 2 R^{Y} groups, C₂₋₉heteroaryl optionally substituted with 1 or 2 R^{Y} groups, C₁₋₄alkyl-(C₂₋₉heteroaryl), C₂₋₅heterocycloalkyl optionally substituted with 1 or 2 C₁₋₄alkyl groups, C₁₋₄alkyl-(C₂₋₅heterocycloalkyl) optionally substituted with 1 or 2 C₁₋₄alkyl groups, C(O)—C₂₋₉heteroaryl optionally substituted with 1 or 2 C₁₋₄ alkyl groups, SO₂—C₂₋₉heteroaryl optionally substituted with 1 or 2 C₁₋₄ alkyl groups, or haloC₁₋₄ alkyl groups; or two adjacent R^{X} groups together form C₁₋₃alkylenedioxy; and
R^{Y} is selected from H, hydroxyl, halo, C₁₋₄alkyl, C₁₋₄alkoxy.

In preferred embodiments, each heteroaryl and each heterocycloalkyl group has at least one nitrogen heteroatom. In some embodiments, each heteroaryl is independently selected from indolyl (e.g. N-indolyl, 2-indolyl, 3-indolyl, 5-indolyl), pyridyl (e.g. 2-pyridyl, 3-pyridyl, 4-pyridyl), triazolyl, oxazolyl (e.g. 1,3-oxazolyl, 1,2-oxazolyl), oxadiazolyl, quinolinyl, isoquinolinyl, pyrrolyl, or pyrazolyl, each of which may be optionally substituted with 1 or 2 R^{X} groups. In some embodiments, each heterocycloalkyl is independently selected from aziridinyl, morpholinyl, piperidinyl, piperazinyl, each of which may be optionally substituted with 1 or 2 R^{X} groups.

In some embodiments, R¹ and R⁴ are H. In some embodiments, R² and R³ are independently H, halo, C₁₋₃alkoxy. In some embodiments, R² and R³ are not both H. In some embodiments, R² and R³ are both C₁₋₃alkoxy e.g. methoxy, ethoxy. In some embodiments, R² and R³ together are methylenedioxy. In some embodiments, R¹ and R⁴ are H and R² and R³ are C₁₋₃alkoxy, preferably methoxy. In some embodiments, R² is C₁₋₃alkoxy and R¹, R³ and R⁴ are H.

In some embodiments, L¹ is NH, or NHC₁₋₂alkyl. In some embodiments, L¹ is phenyl. In some embodiments, L¹ is NHC₁₋₂alkyl-NHC(O)—, azetidinyl-NHC(O)—, NHC₁₋₄alkyl-NHSO₂—, or azetidinyl-NHSO₂—. In some embodiments, L¹ is absent.

In some embodiments, R⁵ is halo, guanidinyl, ureido, or a group selected from C₃₋₆cycloalkyl, phenyl, naphthyl, indolyl (e.g. 2-indolyl or 3-indolyl), pyridyl (e.g. 2-pyridyl, 3-pyridyl or 4-pyridyl), quinolinyl, isoquinolinyl, morpholinyl, piperidinyl, piperazinyl, triazolyl (e.g. 4-triazolyl), pyrazolyl (e.g. N(1)-pyrazolyl, 3-pyrazolyl), oxazolyl (e.g. 1,3-oxazolyl, 1,2-oxazolyl), oxadiazolyl, benzodiazolyl, pyrrolopyridinyl (e.g. pyrrolo[2,3-b]pyridinyl), wherein each group is optionally substituted with 1 or 2 R^{X} groups.

In some embodiments, L¹ is absent and R⁵ is selected from C₁₋₆ alkyl, C₁₋₆ alkenyl and C₁₋₆ alkynyl. In some embodiments, L¹ is absent and R⁵ is C₆₋₁₀aryl (e.g. phenyl, naphthyl) optionally substituted with 1 or 2 R^{X} groups (e.g. CF₃, methoxy, methylenedioxy, 1,2-ethylenedioxy, morpholinyl, CH₂-morpholinyl). In some embodiments, L¹ is absent and R⁵ is C(O)piperazinyl (e.g. C(O)(N(1)-piperazinyl) optionally substituted with 1 or 2 C₁₋₃alkyl groups. In some embodiments, L¹ is absent and R⁵ is C₂₋₉heteroaryl (e.g. indolyl, quinolinyl) optionally substituted with 1 or 2 R^{X} groups.

In some embodiments, L¹ is phenyl and R⁵ is C(O)piperazinyl (e.g. C(O)(N(1)-piperazinyl) optionally substituted with 1 or 2 C₁₋₃alkyl groups.

In some embodiments, L¹ is NHC₁₋₂alkyl and R⁵ is guanidinyl, ureido, or C₂₋₉heteroaryl (e.g. indolyl, quinolinyl) optionally substituted with 1 or 2 R^{X} groups.

In some embodiments, L¹ is NHC₁₋₂alkyl-NHC(O)—, azetidinyl-NHC(O)— or azetidinyl-NHSO₂—, and R⁵ is C₂₋₉heteroaryl (e.g. 1,3-oxazolyl, 1,2-oxazolyl, oxadiazolyl) optionally substituted with 1 or 2 groups selected from C₆₋₁₀aryl (e.g. phenyl), or C₂₋₉heteroaryl (e.g. indolyl).

In some embodiments, L¹ is absent and R⁵ is C₆₋₁₀aryl (e.g. phenyl, naphthyl) optionally substituted with 1 or 2 groups selected from C₁₋₃ alkyl, C₁₋₄alkoxy, or two adjacent groups which together form methylenedioxy or 1,2-ethylenedioxy. In some embodiments, L¹ is absent and R⁵ is C₂₋₉heteroaryl (e.g. indolyl, quinolinyl, pyridinyl) optionally substituted with 1 or 2 groups selected from C₁₋₃alkyl, or C₁₋₃alkoxy.

In some embodiments, R⁵ is piperazinyl (e.g. N(1)-piperazinyl) optionally substituted with 1 or 2 groups selected from C₁₋₃alkyl, C₂₋₉heteroaryl (e.g. indolyl, pyridyl) optionally substituted with 1 or 2 C₁₋₄alkyl or haloC₁₋₄ alkyl groups, or SO₂—C₂₋₉heteroaryl (e.g. indolyl, pyridyl) optionally substituted with 1 or 2 C₁₋₄ alkyl or haloC₁₋₄ alkyl groups.

In some embodiments, L¹ is absent; L₂ is absent; R⁵ is N-piperazinyl optionally substituted with 1 or 2 groups selected from C₁₋₃alkyl, C₂₋₉heteroaryl (e.g. indolyl, pyridyl) optionally substituted with 1 or 2 C₁₋₄alkyl or haloC₁₋₄ alkyl groups, or SO₂—C₂₋₉heteroaryl (e.g., indolyl, pyridyl) optionally substituted with 1 or 2 C₁₋₄ alkyl or haloC₁₋₄ alkyl groups; R⁶ is H and R⁷ is H.

In some embodiments, L² is C₁₋₂alkyl, C₁₋₂alkyl-NHC(O)—, C₁₋₂alkyl-NHSO₂, or azetidinyl-NHC(O)—.

In some embodiments, R⁷ is H, methyl or ethyl.

In some embodiments, L² is C₁₋₂alkyl and R⁶ is indolyl (e.g. 2-indolyl or 3-indolyl) optionally substituted with 1 or 2 R^{X} groups.

In some embodiments, R⁷ is H, L² is C(O), and R⁶ is C₁₋₄alkyl or C₃₋₆cycloalkyl.

In some embodiments, L² is azetidinyl-C(O)— and R⁶ is indolyl (e.g. 2-indolyl or 3-indolyl) optionally substituted with 1 or 2 R^{X} groups.

In some embodiments, L² is absent; and R⁶ and R⁷ together with the nitrogen to which they are attached form a piperazinyl ring optionally substituted with 1 or 2 R^{X} groups.

In some embodiments, L² is absent, R⁶ is H and R⁷ is H.

In some embodiments, L¹ is absent, R⁶ is C₆₋₁₀ aryl (e.g. phenyl) optionally substituted with 1 or 2 R^{X} groups, L² is absent, R⁶ is H and R⁷ is H.

In some embodiments, L¹ is absent, R⁵ is quinolinyl optionally substituted with 1 or 2 R^{X} groups, L² is C₁₋₂alkyl, R⁶ is C₂₋₉ heteroaryl (e.g. pyridinyl, indolyl) or C₆₋₁₀ aryl (e.g. phenyl) and R⁷ is H.

In some embodiments, each R^{X} is independently selected from hydroxyl, halo, C₁₋₃alkyl, C₁₋₃alkoxy, C(O)C₁₋₃alkyl, C(O)OC₁₋₃alkyl, NR′R″ wherein R′ and R″ are independently selected from H and C₁₋₃alkyl, phenyl optionally substituted with 1 or 2 R^{Y} groups, morpholinyl optionally substituted with 1 or 2 R^{Y} groups, piperazinyl optionally substituted with 1 or 2 R^{Y} groups, C(O)piperazinyl optionally substituted with 1 or 2 R^{Y} groups, C(O)morpholinyl optionally substituted with 1 or 2 R^{Y} groups, pyridyl (e.g. 2-, 3- or 4-pyridyl) optionally substituted with 1 or 2 R^{Y} groups, indolyl (e.g. 2-, 3- or 5-indolyl) optionally substituted with 1 or 2 R^{Y} groups, or SO₂-indolyl (e.g. 2-, 3- or 5-indolyl) optionally substituted with 1 or 2 R^{Y} groups, or two adjacent R^{X} groups together form methylenedioxy.

In some embodiments, R^{Y} is selected from hydroxyl, halo, C₁₋₃alkyl, haloC₁₋₃alkyl, C₁₋₃alkoxy.

In some embodiments, the compound of Formula 1 is a compound of Formula 1A, 1B, 1C or 1D:

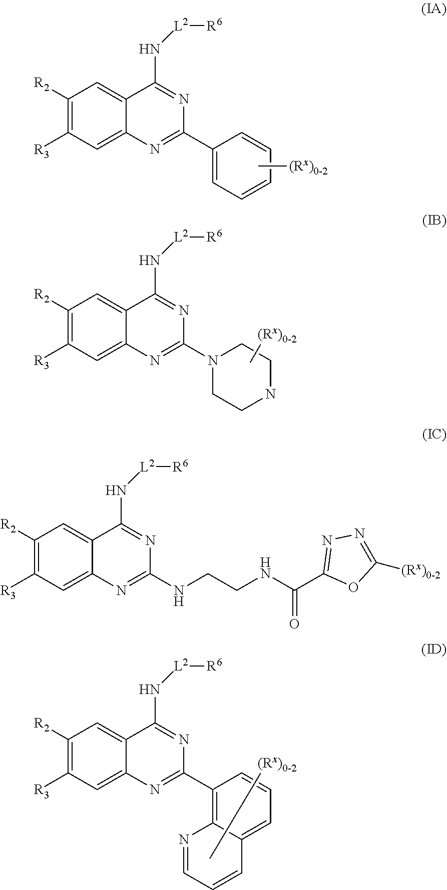

or a salt, hydrate, solvate, tautomer or stereoisomer thereof, wherein R², R³, R⁶, R^{X} and L², are as defined for Formula 1, including each of the preferred embodiments.

In some embodiments, the compound of Formula 1 is a compound of Formula 1E:

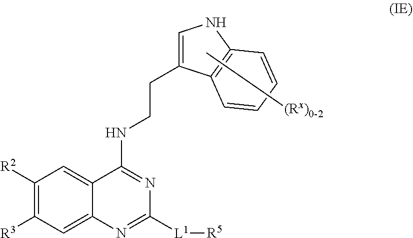

or a salt, hydrate, solvate, tautomer or stereoisomer thereof, wherein R², R³, R⁵, L¹ and R^{X} are independently defined as for Formula 1, including each of the preferred embodiments.

In particular embodiments, the compound of Formula 1 is selected from:

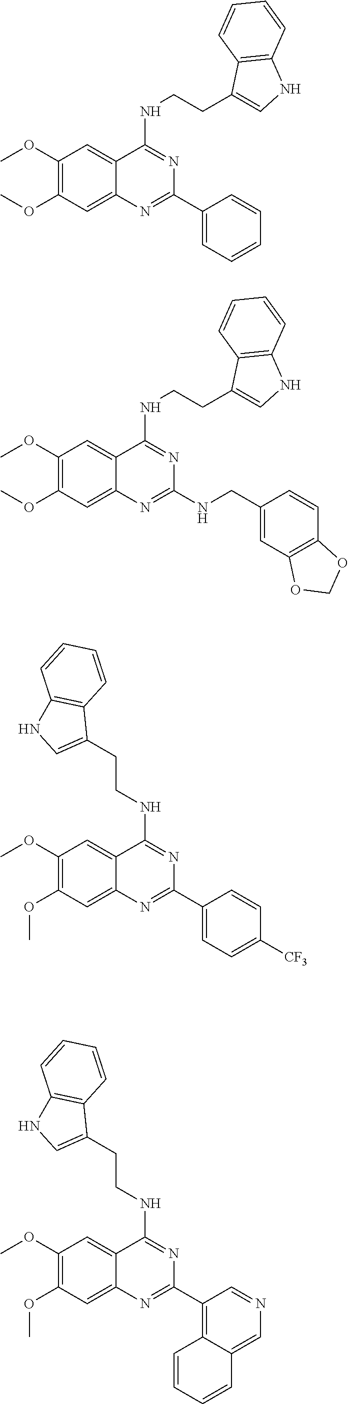

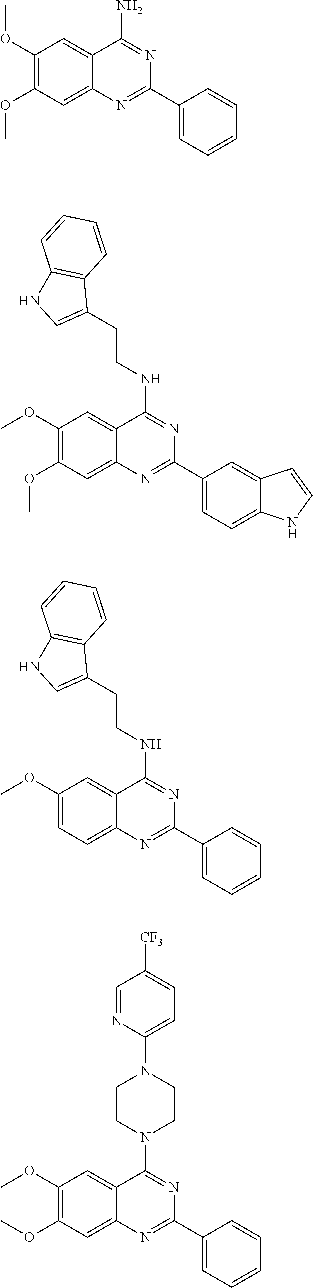 [SMILES: COc1cc2nc(-c3ccccc3)nc(N)c2cc1OC ; COc1cc2nc(-c3ccc4[nH]ccc4c3)nc(NCCc3c[nH]c4ccccc34)c2cc1OC ; COc1ccc2nc(-c3ccccc3)nc(NCCc3c[nH]c4ccccc34)c2c1 ; COc1cc2nc(-c3ccccc3)nc(N3CCN(c4ccc(C(F)(F)F)cn4)CC3)c2cc1OC]

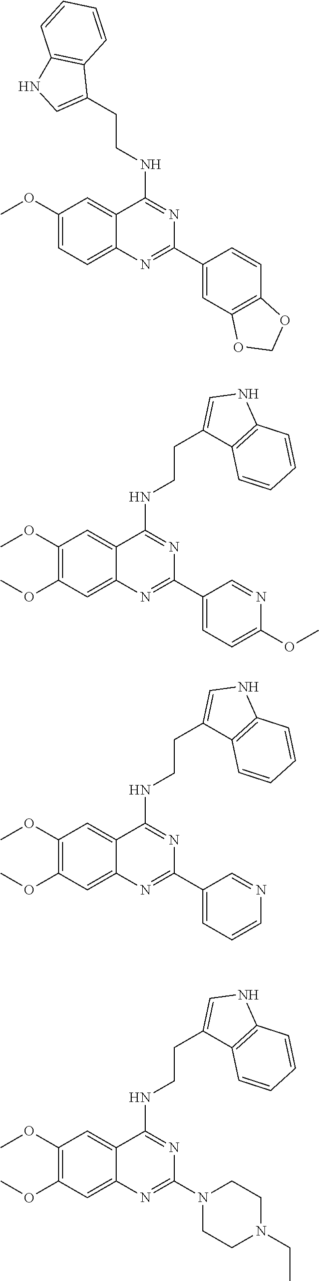

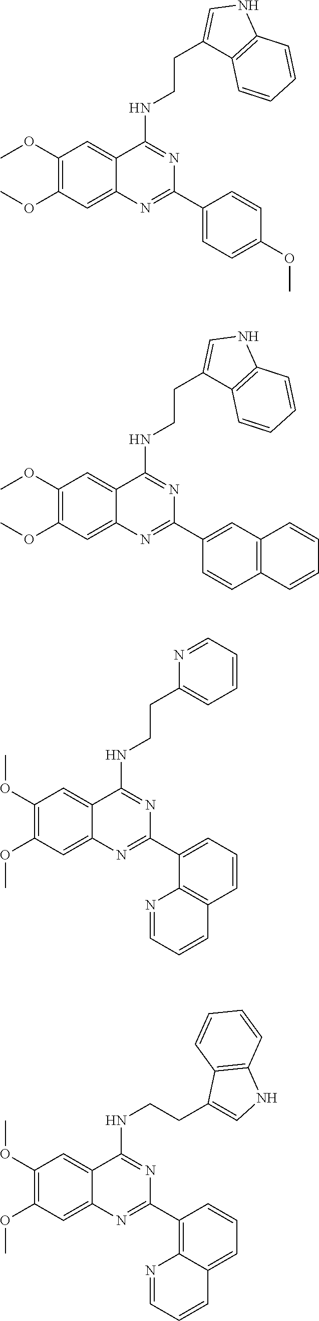 [SMILES: COc1ccc(-c2nc(NCCc3c[nH]c4ccccc34)c3cc(OC)c(OC)cc3n2)cc1 ; COc1cc2nc(-c3ccc4ccccc4c3)nc(NCCc3c[nH]c4ccccc34)c2cc1OC ; COc1cc2nc(-c3cccc4cccnc34)nc(NCCc3ccccn3)c2cc1OC ; COc1cc2nc(-c3cccc4cccnc34)nc(NCCc3c[nH]c4ccccc34)c2cc1OC]

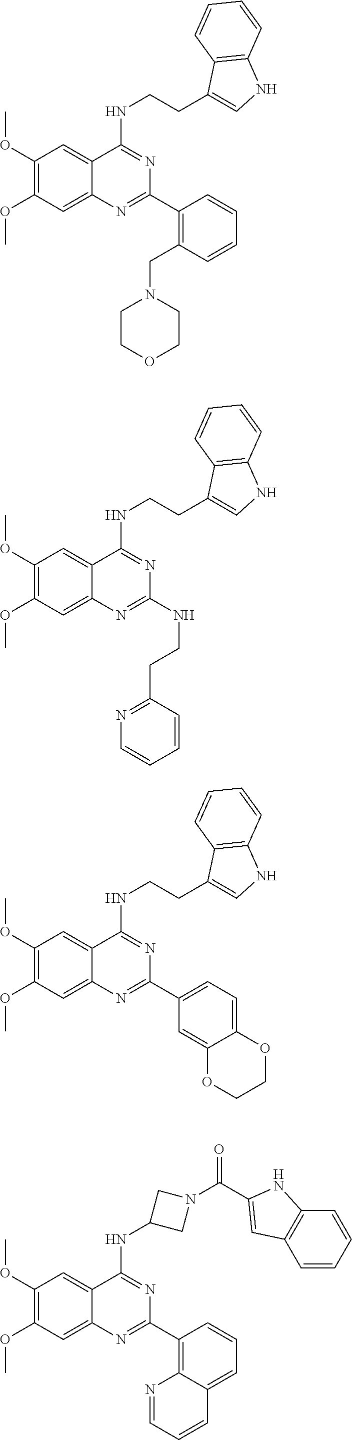 [SMILES: COc1cc2nc(-c3ccccc3CN3CCOCC3)nc(NCCc3c[nH]c4ccccc34)c2cc1OC ; COc1cc2nc(NCCc3ccccn3)nc(NCCc3c[nH]c4ccccc34)c2cc1OC ; COc1cc2nc(-c3ccc4c(c3)OCCO4)nc(NCCc3c[nH]c4ccccc34)c2cc1OC ; COc1cc2nc(-c3cccc4cccnc34)nc(NC3CN(C(=O)c4cc5ccccc5[nH]4)C3)c2cc1OC]

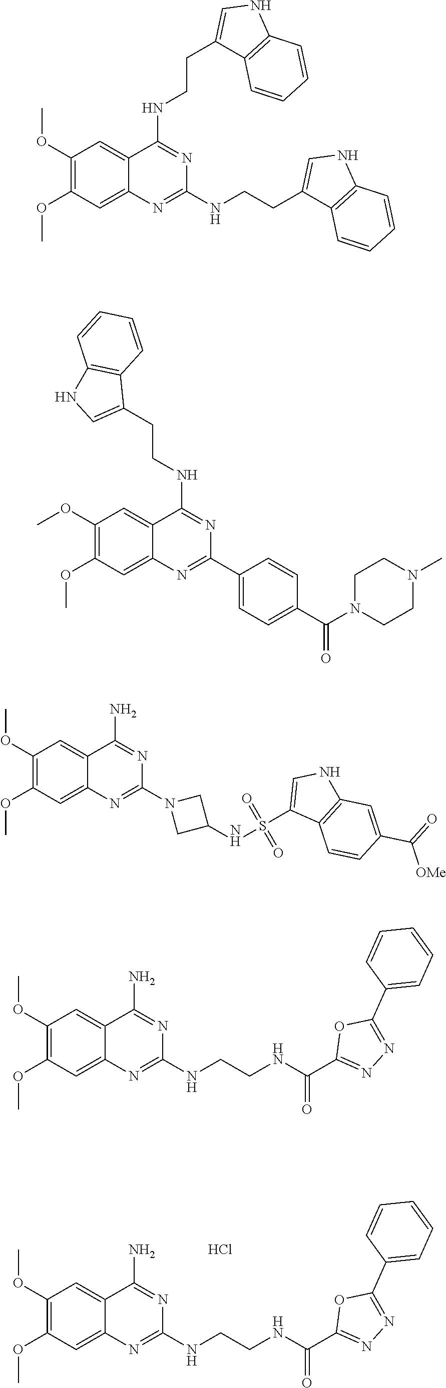

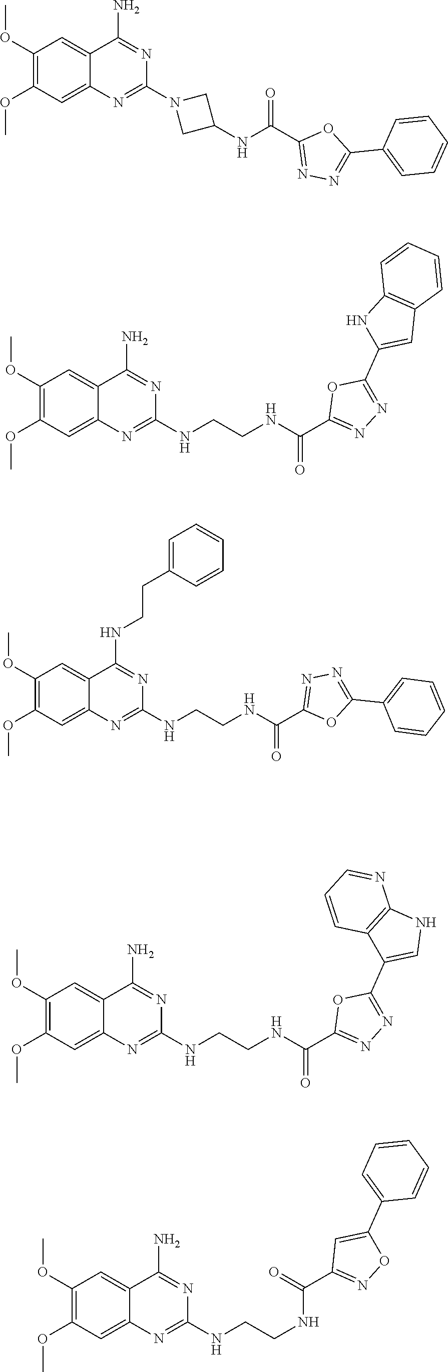 [SMILES: COc1cc2nc(N3CC(NC(=O)c4nnc(-c5ccccc5)o4)C3)nc(N)c2cc1OC ; COc1cc2nc(NCCNC(=O)c3nnc(-c4cc5ccccc5[nH]4)o3)nc(N)c2cc1OC ; COc1cc2nc(NCCNC(=O)c3nnc(-c4ccccc4)o3)nc(NCCc3ccccc3)c2cc1OC ; COc1cc2nc(NCCNC(=O)c3nnc(-c4c[nH]c5ncccc45)o3)nc(N)c2cc1OC ; COc1cc2nc(NCCNC(=O)c3cc(-c4ccccc4)on3)nc(N)c2cc1OC]

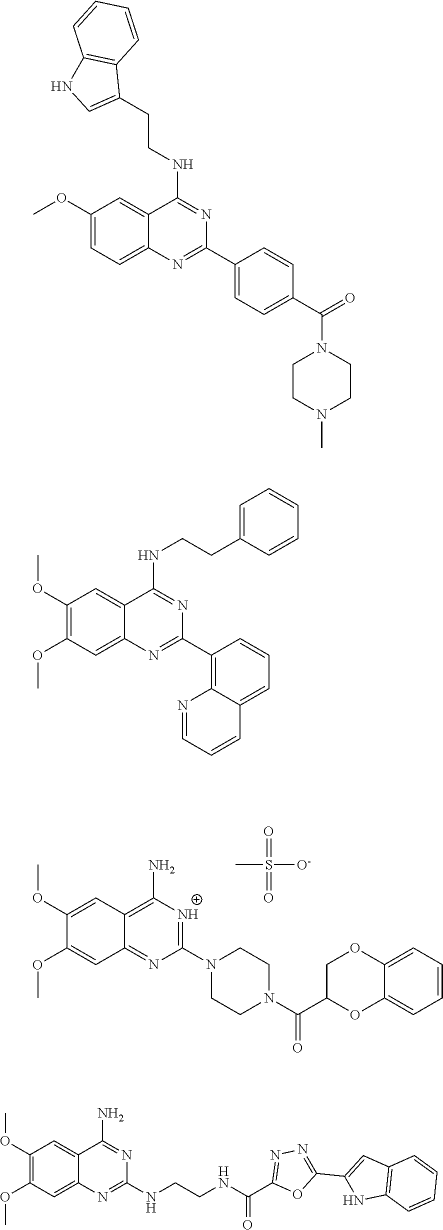

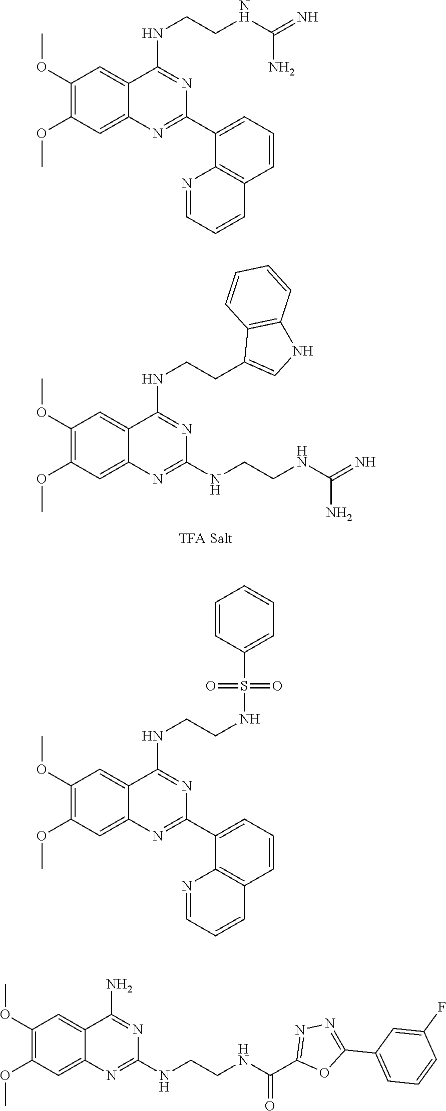

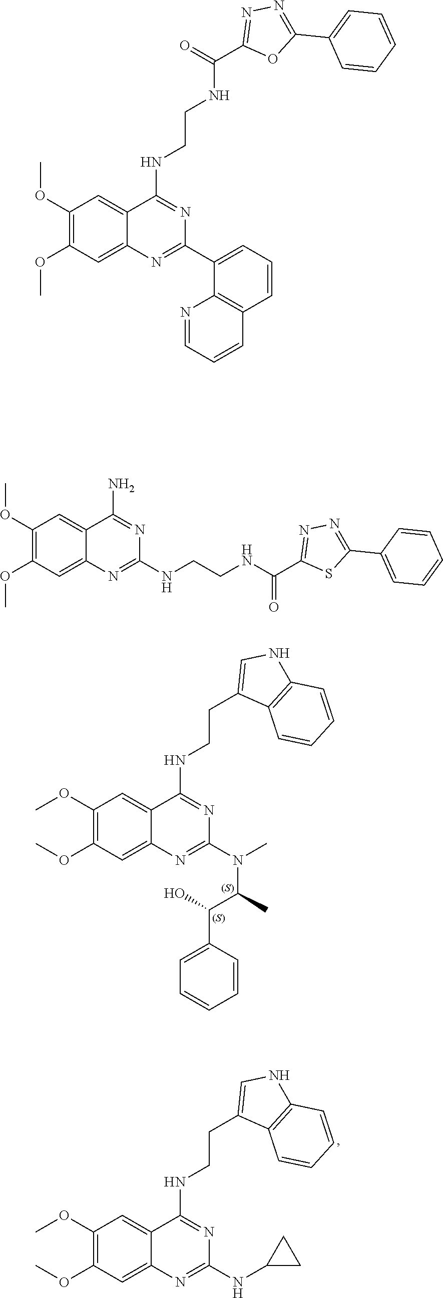

and a salt, hydrate, solvate, tautomer or stereoisomer thereof. In some embodiments, the heparanase inhibitor is

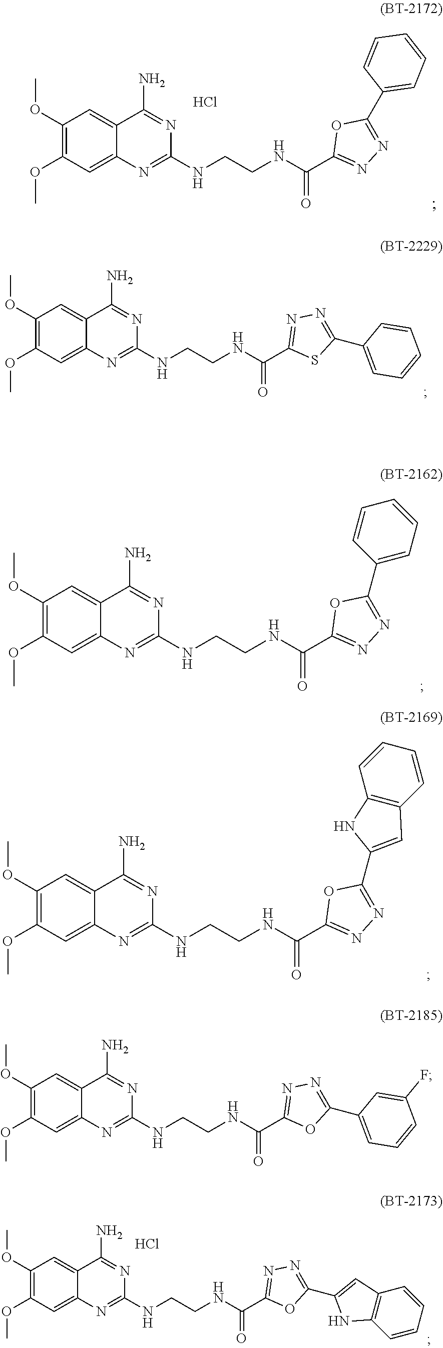

especially BT-2172.

A skilled person will be well aware of suitable reagents and reaction conditions for synthesizing compounds of Formula 1 from, for example the literature and text books, including for example March, Advanced Organic Chemistry, 4th Ed (John Wiley & Sons, New York, 1992) and Vogel's Textbook of Practical Organic Chemistry, 5th Ed (John Wiley & Sons, New York, 1989). Suitable reagents and reaction conditions may also be ascertained from SciFinder Scholar or other reaction databases.

The reaction schemes presented below are illustrative of general methods that may be employed to prepare the compounds of Formula 1. Alternative methods, including routine modifications of the methods disclosed herein, will be apparent to those skilled in the art.

A general synthesis for the preparation of compounds of Formula 1 is illustrated in Scheme 1.

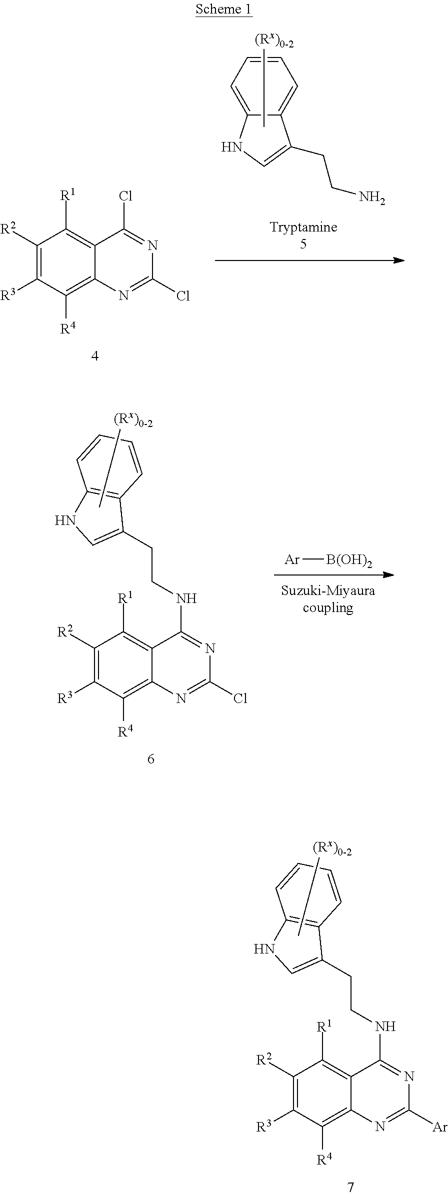

The synthesis shown in Scheme 1 commences with the condensation of a 2,4-dichloroquinazoline compound (4) with an amine compound represented by an optionally substituted tryptamine compound (5) to afford the amine-substituted quinazoline compound (6). This is followed by a Suzuki-Miyaura cross-coupling reaction with an optionally substituted aryl boronate to provide compounds of Formula 1 represented by structure 7.

Typically, in the first reaction depicted in Scheme 1, an optionally substituted amine (5) (represented by tryptamine) is dissolved or suspended in a suitable solvent, such as tetrahydrofuran (THF), then treated with the 2,4-dichloroquinazoline compound (4), followed by addition (typically dropwise addition) of a base (e.g. triethylamine), after which the reaction is stirred for a period of time sufficient for the reaction to proceed substantially to completion. The precise period of time will depend on the scale of the reaction, however those skilled in the art will readily be able to determine suitable time and temperature conditions using standard techniques, such as Thin Layer Chromatography (TLC), 1H NMR, etc. In a typical reaction, the reagents are stirred at a temperature between 15° C.-40° C., typically room temperature, for a period of about 4-24 hours, e.g., about 12 hours, or about 18 hours. The product may be isolated and purified using standard techniques known to those skilled in the art, e.g., solvent extraction (e.g. using an organic solvent such as ethyl acetate, chloroform, or the like, and washing with water and/or aqueous solution (e.g. sodium carbonate, sodium hydrogen carbonate, brine), followed by column chromatography and/or recrystallization.

The second step is a Suzuki-Miyaura cross-coupling reaction, which involves reacting compound (6) with an aryl boronate compound to produce compound (7). In a typical reaction, a mixture of phenylboronic acid, compound (6) and a base (e.g. potassium carbonate), is treated with a de-gassed solvent mixture (e.g. a mixture of dimethoxyethane, water, ethanol). Bis(triphenylphosphine)palladium(II) dichloride catalyst is then added and the resultant mixture is sealed then irradiated with microwave radiation under nitrogen at a temperature and period of time until the reaction is judged to be substantially complete (typically 120° C./0.33 h, ramp time 1 minute, maximum power 200 W). Those skilled in the art know how to monitor the progress of a reaction using standard techniques, such as TLC, ¹H NMR, etc. The product may be isolated and purified using standard techniques known to those skilled in the art, such as solvent extraction, e.g., using an organic solvent such as ethyl acetate, chloroform, or the like, and washing with water and/or aqueous solution (e.g. sodium carbonate, sodium hydrogen carbonate, brine), as well as other well-known conventional techniques such as column chromatography and/or recrystallization.

An alternative synthetic sequence is illustrated in Scheme 2 in which the indole-substituted quinazoline compound (6) is condensed with an amine compound (8) to afford compounds of Formula 1, represented by structure (9).

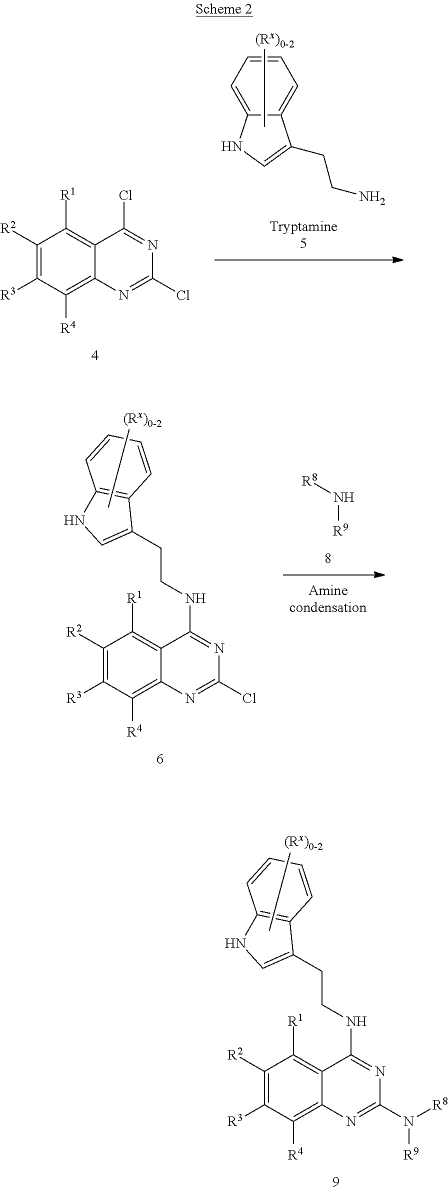

The amine condensation reaction depicted in Scheme 2 is typically conducted under an inert gaseous atmosphere (e.g. nitrogen, argon) and involves reacting an amine (8), compound (6) in the presence of a non-nucleophilic base (e.g. N,N-diisopropylethylamine, 2,6-dimethylpyridine, DABCO, N-methylmorpholine, triethylamine, etc), in a suitable protic solvent (e.g. an alcohol such as propanol, butanol, e.g. n-butanol) in a sealed vessel, which is then subjected to microwave irradiation sufficient for the reaction to be substantially complete. Those skilled in the art know how to monitor the progress of a reaction using standard techniques, such as TLC, 1H NMR, etc. (exemplary microwave irradiation is 160° C./0.5 h, ramp time 2 minutes, maximum power 200 W). The product may be isolated and purified using standard techniques known to those skilled in the art, including e.g. solvent extraction, column chromatography, recrystallization, etc.

An alternative approach to preparing compounds of Formula 1 is illustrated in Scheme 3, which is based on a reaction sequence described by Seijas et al. (2000) Tetrahedron Lett, 41: 2215-2217, the content of which is incorporated by reference in its entirety.

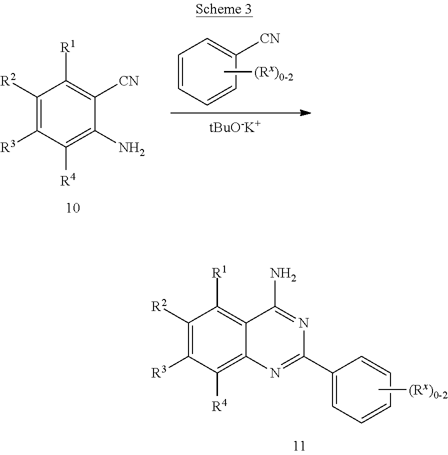

The reaction involves reacting a 2-aminobenzonitrile compound (10) with an arylnitrile (such as benzonitrile) in the presence of a strong base (e.g. potassium t-butoxide) under an inert (e.g. nitrogen or argon) atmosphere. The reaction mixture is subjected to microwave irradiation to produce a compound of Formula 1 represented by structure (11) (exemplary microwave irradiation conditions are 180° C./1 min., ramp time 1 min., maximum power 200 W). The product may be isolated and purified using standard techniques known to those skilled in the art, e.g., solvent extraction, column chromatography, recrystallization, and the like.

Where appropriate or necessary, protecting groups may be employed at any stage in the synthesis of compounds of Formula 1. Similarly, a person skilled in the art will also appreciate that the compounds can also be prepared with certain protecting groups that are useful for purification or storage that can be removed before administration to a subject. Suitable protecting groups and their use are well known to those skilled in the art and include, for example, protecting groups described in Wuts and Greene (2007) Greene's Protective Groups in Organic Synthesis, 4th Edition. (John Wiley & Sons, Inc.). The protection and deprotection of functional groups is also described in Protective Groups in Organic Chemistry (1973) edited by J. W. F. McOmie, Plenum Press.

A skilled person will recognize the versatility of the reactions illustrated in the above Schemes, which can provide access to a wide range of substituted quinazoline compounds of Formula 1.

Compounds of Formula 1 may be isolated or purified using standard techniques known to those skilled in the art. Such techniques include precipitation, crystallization, recrystallization, column chromatography (including flash column chromatography), HPLC, formation of salts, lyophilization, among others. Suitable solvents for use in these techniques will be known or can be readily ascertained by those skilled in the art using routine practices.

Salts, including pharmaceutically acceptable salts, of compounds of Formula 1 may be prepared by methods known to those skilled in the art, including for example:

(i) by reacting the compound of Formula 1 with the desired acid or base;
(ii) by removing an acid- or base-labile protecting group from a suitable precursor of the compound of Formula 1 or by ring-opening a suitable cyclic precursor, for example, a lactone or lactam, using the desired acid or base; or
(iii) by converting one salt of the compound of Formula 1 into another salt by reaction with an appropriate acid or base, or by means of a suitable ion exchange column.

The above reactions are typically carried out in solution. Suitable solvent systems (including mixed solvent systems) are well known to those skilled in the art and those skilled in the art can readily select or determine a suitable solvent system using routine methods taking into consideration the nature of the compound of Formula 1, the particular salt being formed, and the amount of the compound of Formula 1. Exemplary solvent systems include methanol, ethanol, water, acetone, tetrahydrofuran, dichloromethane, pentane, hexane, diethyl ether, ethyl acetate, and any mixture of two or more such solvents. The resulting salt may precipitate out and be collected by filtration or may be recovered by evaporation of the solvent. The degree of ionization in the resulting salt may vary from completely ionized to almost non-ionized.

In some embodiments, the heparanase inhibitor is a tetrahydroquinazoline or dihydroquinazoline compound of Formula 2 or 3:

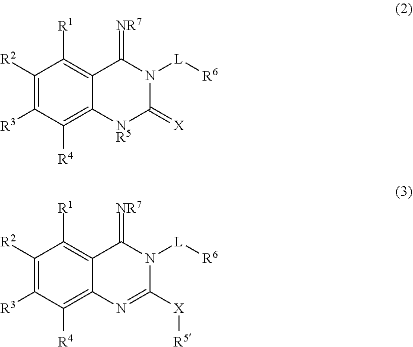

or a salt, hydrate, solvate, tautomer or stereoisomer thereof,
wherein:

### X is S or O;

R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
or R¹ and R², or R² and R³, or R³ and R⁴ together form C₁₋₃alkylenedioxy;
R⁵ and R^{5′} are independently selected from H, C₁₋₆ alkyl, C₁₋₃alkylC(O)OC₁₋₄alkyl and C₁₋₃alkylC₆₋₁₀aryl optionally substituted with 1 or 2 groups independently selected from haloC₁₋₃alkyl and haloC₁₋₃alkoxy;
L is selected from C₁₋₆ alkyl, azetidinyl, C₁₋₆ alkyl-indolyl, NH, C₁₋₆alkyl-NHC(O)O, azetidinyl-C(O)—, C₁₋₆alkyl-NHC(O)-indolyl, C₁₋₆alkyl-NHSO₂—, or is absent;
R⁶ is selected from H, halo, hydroxyl, C₁₋₆alkyl, C₁₋₆alkenyl, C₆₋₁₀aryl optionally substituted with 1 or 2 R^{X} groups, C₁₋₉heteroaryl optionally substituted with 1 or 2 R^{X} groups, C₂₋₅heterocycloalkyl optionally substituted with 1 or 2 R^{X} groups, C(O)—(C₁₋₉heteroaryl) optionally substituted with 1 or 2 R^{X} groups, C(O)(C₂₋₅heterocycloalkyl) optionally substituted with 1 or 2 R^{X} groups, C(O)NHR^{Y}, or is absent;
each R^{X} is independently selected from hydroxyl, halo, nitro, NR′R″, C₃₋₆cycloalkyl, 4alkyl, C₁₋₄alkoxy, C₆₋₁₀aryl optionally substituted with 1 or 2 R^{Y} groups, C₁₋₉heteroaryl, C₁₋₄alkyl-(C₁₋₉heteroaryl), C(O)OC₁₋₄alkyl, C(O)NHR^{Y}, C₂₋₅heterocycloalkyl optionally substituted with 1 or 2 C₁₋₄alkyl groups, C(O)-(heterocycloalkyl) optionally substituted with 1 or 2 C₁₋₄alkyl groups, or two adjacent R^{X} groups together form C₁₋₃alkylenedioxy;
R^{Y} is selected from H, hydroxyl, halo, C₁₋₄ alkyl, haloC₁₋₄ alkyl, C₁₋₄ alkoxy, C₁₋₄alkylheterocycloalkyl, C(O)—(C₁₋₄alkylheterocycloalkyl), C₁₋₄alkylNR′R″;
R′ and R″ are independently selected from H, C₁₋₄alkyl, C₁₋₄alkylheterocycloalkyl;
R⁷ is selected from H, C₁₋₆alkylC₁₋₉heteroaryl.

In preferred embodiments, each heteroaryl and each heterocycloalkyl group comprises at least one nitrogen heteroatom.

In preferred embodiments, X is S.

In some embodiments, each heteroaryl is independently selected from indolyl (e.g. N-indolyl, 2-indolyl, 3-indolyl), pyridyl (e.g. 2-pyridyl, 3-pyridyl, 4-pyridyl), pyrazolyl, pyrrolyl, benzoxadiazolyl, triazolyl, oxazolyl, oxadiazolyl, each of which may be optionally substituted with 1 or 2 R^{X} groups.

In some embodiments, each heterocycloalkyl is independently selected from aziridinyl, morpholinyl, piperidinyl, piperazinyl, each of which may be optionally substituted with 1 or 2 R^{X} groups.

In some embodiments, R¹ and R⁴ are H. In some embodiments, R² and R³ are independently H, halo, or C₁₋₄alkoxy. In some embodiments, R² and R³ are not both H. In some embodiments, R² and R³ are both C₁₋₄alkoxy e.g. methoxy, ethoxy. In some embodiments, R² and R³ together are methylenedioxy.

In some embodiments, R⁵ or R^{5′} is independently H. In some embodiments, R⁵ or R^{5′} is independently a C₁₋₆ alkyl group, e.g. methyl, ethyl. In some embodiments, R⁵ or R^{5′} is independently C₁₋₃alkylC(O)OC₁₋₃alkyl. In some embodiments, R⁵ or R^{5′} is independently a benzyl group optionally substituted with 1 or 2 groups selected from CF₃ and OCF₃.

In some embodiments, L is C₁₋₃ alkyl, C₁₋₃ alkylNHC(O)—, or azetidinyl. In some embodiments, L is C₁₋₆ alkyl-NHC(O)-indolyl.

In some embodiments, R⁶ is a group selected from indolyl (e.g. 2-indolyl or 3-indolyl), phenyl, pyridyl (e.g. 2-pyridyl, 3-pyridyl or 4-pyridyl), N-morpholinyl, N-piperidinyl, N-piperazinyl, pyrrolyl, diazolyl, triazolyl (e.g. 4-triazolyl), pyrazolyl (e.g. N(1)-pyrazolyl); oxazolyl, ozadiazolyl, benzodiazolyl and pyrrolopyridinyl (e.g. pyrrolo[2,3-b]pyridinyl), wherein each group is optionally substituted with 1 or 2 R^{X} groups.

In some embodiments, R⁷ is H or C₁₋₄ alkyl. In some embodiments, R⁷ is C₁₋₆ alkylC₁₋₉heteroaryl. In some embodiments, R⁷ is C₁₋₂ alkyl-(2-indolyl) or C₁₋₂ alkyl-(3-indolyl).

In some embodiments, L is absent and R⁶ is selected from C₁₋₆alkyl, C₁₋₆alkenyl. C₁₋₆alkynyl, and C₂₋₅heterocycloalkyl (e.g. piperidinyl, piperazinyl or morpholinyl) optionally substituted with 1 or 2 R^{X} groups.

In some embodiments, L is NH and R⁶ is H or phenyl.

In some embodiments, L is C₁₋₂alkyl and R⁶ is indolyl (e.g. 2-indolyl or 3-indolyl) optionally substituted with 1 or 2 R^{X} groups. In some embodiments, L is C₁₋₂ alkyl-NHC(O)— and R⁶ is C₁₋₉ heteroaryl (e.g. indolyl, pyrrolyl, pyridinyl, oxazolyl, oxadiazolyl) optionally substituted with 1 or 2 R^{X} groups, R⁵ or R^{5′} is independently H and R⁷ is H.

In some embodiments, L is C₁₋₂ alkyl-NHC(O)— and R⁶ is C₁₋₉ heteroaryl (e.g. indolyl, pyrrolyl, pyridinyl, oxazolyl, oxadiazolyl) optionally substituted with 1 or 2 R^{X} groups. In some embodiments, L is C₁₋₃ alkylNHC(O)— and R⁶ is indolyl (e.g. 2-indolyl or 3-indolyl) optionally substituted with 1 or 2 R^{X} groups or 3-indolyl optionally substituted with 1 or 2 R^{X} groups. In some embodiments, L is C₁₋₆ alkyl-NHC(O)— and R⁶ is C₁₋₉ heteroaryl (e.g. pyrrolyl, pyridinyl, oxazolyl, oxadiazolyl) optionally substituted with 1 or 2 R^{X} groups. In some embodiments, L is C₁₋₃ alkylNHC(O)— and R⁶ is indolyl (e.g. 2-indolyl or 3-indolyl) optionally substituted with a pyridinyl group (e.g. 2-, 3- or 4-pyridinyl). In some embodiments, L is C₁₋₆ alkyl-NHC(O)— and R⁶ is C₁₋₉ heteroaryl (e.g. pyrrolyl, pyridinyl, oxazolyl, oxadiazolyl) optionally substituted with 1 or 2 R^{X} groups, R⁵ or R^{5′} is independently selected from C₁₋₃ alkyl, C₁₋₃alkylC(O)OC₁₋₄alkyl and C₁₋₃alkylC₈₋₁₀aryl optionally substituted with 1 or 2 groups independently selected from haloC₁₋₃alkyl (e.g. CF₃) and haloC₁₋₃alkoxy (e.g. OCF₃), and R⁷ is H.

In some embodiments, L is C₁₋₃ alkyl-NHC(O)-indolyl and R⁶ is selected from phenyl optionally substituted with 1 or 2 R^{X} groups, C₃₋₈ heteroaryl optionally substituted with 1 or R^{X} groups, C(O)(C₃₋₈heteroaryl) optionally substituted with 1 or 2 R^{X} groups, C(O)(C₂₋₅heterocycloalkyl) optionally substituted with 1 or 2 R^{X} groups, C(O)NHR^{Y}, or is absent. In some embodiments, L is C₁₋₃ alkyl-NHC(O)-indolyl and R⁶ is selected from phenyl optionally substituted with C(O)NHR^{Y}, C(O)-(heterocycloalkyl) optionally substituted with 1 or 2 C₁₋₄ alkyl groups (e.g. methyl, ethyl).

In some embodiments, L is C₁₋₂ alkyl-NHC(O)-indolyl, R⁶ is pyridyl, and R⁵ or R^{5′} is independently selected from H, C₁₋₆ alkyl, C₁₋₃alkylC(O)OC₁₋₄alkyl and C₁₋₃alkylC₆₋₁₀aryl optionally substituted with 1 or 2 groups independently selected from haloC₁₋₃alkyl or haloC₁₋₃alkoxy.

In some embodiments, L is azetidinylC(O)— and R⁶ is indolyl (e.g. 2-indolyl or 3-indolyl) optionally substituted with 1 or 2 R^{X} groups. In some embodiments, L is azetidinylC(O)- and R⁶ is 2-indolyl optionally substituted with a C₁₋₉ heteroaryl group. In some embodiments, L is azetidinylC(O)— and R⁶ is 2-indolyl optionally substituted with a pyridinyl group (e.g. 2-, 3- or 4-pyridinyl). In some embodiments, L is azetidinylC(O)— and R⁶ is 2-indolyl optionally substituted with a C₁₋₉ heteroaryl group, R⁵ or R^{5′} is independently H and R⁷ is H. In some embodiments, L is azetidinylC(O)— and R⁶ is 2-indolyl optionally substituted with a C₁₋₉ heteroaryl group, R⁵ or R^{5′} is independently selected from C₁₋₃ alkyl, C₁₋₃alkylC(O)OC₁₋₄alkyl and C₁₋₃alkylC₆₋₁₀aryl optionally substituted with 1 or 2 groups independently selected from haloC₁₋₃alkyl and haloC₁₋₃alkoxy, and R⁷ is H.

In some embodiments, R⁵ or R^{5′} is independently H and R⁷ is H.

In some embodiments, R⁵ or R^{5′} is independently H and R⁷ is C₁₋₂alkyl-(3-indolyl).

In some embodiments, each R^{X} is independently selected from hydroxyl, halo, C₁₋₃ alkyl, C₁₋₄alkoxy, C(O)OC₁₋₄ alkyl, phenyl, NR′R″ wherein R′ and R″ are independently selected from H and C₁₋₃ alkyl, morpholinyl optionally substituted with 1 or 2 C₁₋₃ alkyl groups, piperazinyl optionally substituted with 1 or 2 C₁₋₃ alkyl groups, C(O)morpholinyl optionally substituted with 1 or 2 C₁₋₃alkyl groups, C₆₋₁₀aryl optionally substituted with 1 or 2 C(O)—(C₁₋₄alkylheterocycloalkyl) groups, C(O)piperazinyl optionally substituted with 1 or 2 C₁₋₃ alkyl groups, or two adjacent R^{X} groups together form methylenedioxy or 1,2-ethylenedioxy. In one or more embodiments, R^{X} is pyridyl (e.g. 2-, 3- or 4-pyridyl).

In some embodiments, the compound of Formula 2 is a compound of Formula 2A, 2B or 2C, and/or the compound of Formula 3 is a compound of Formula 3A or 3B:

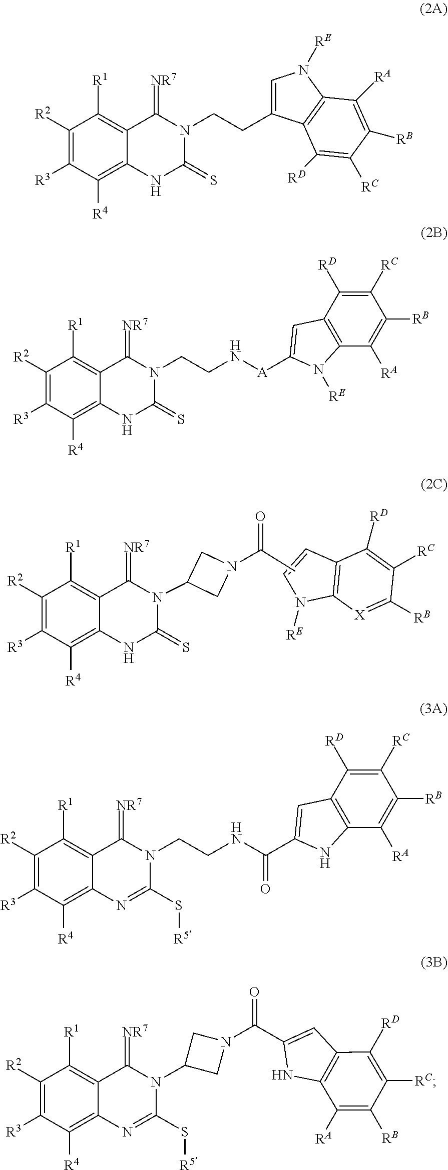

or a salt, hydrate, solvate, tautomer or stereoisomer thereof;
wherein:
R¹ and R⁴ are both H;
R² is H, halo, C₁₋₃alkoxy, O—CH₂phenyl, O-phenyl;
R³ is H, halo, C₁₋₃alkoxy, O—CH₂phenyl, O-phenyl;
R⁷ is H or C₁₋₃ alkyl;

### A is C=O or SO₂;

### X is C or N;

R^{E} is H, C₁₋₃alkyl, or C(O)-heterocycloalkyl (e.g. C(O)—(N-morpholino));
R^{5′} is H, C₁₋₆ alkyl, C₁₋₃alkylC(O)OC₁₋₃alkyl or C₁₋₃ alkylC₆₋₁₀ aryl optionally substituted with 1 or 2 groups independently selected from haloC₁₋₃alkyl or haloC₁₋₃alkoxy;
R^{A}, R^{B}, R^{C} and R^{D} are independently selected from H, OH, C₁₋₃ alkyl, OC₁₋₃ alkyl, C(O)—(N-heterocycloalkyl) (e.g. C(O)morpholinyl), C(O)piperazinyl) optionally substituted with 1 or 2 C₁₋₃ alkyl groups; N-heteroaryl (e.g. 3-pyridyl, 4-pyridyl, 2,1,3-benzoxadiazolyl, pyrazolyl) optionally substituted with 1 or 2 groups selected from OH, halo, C₁₋₃ alkyl, OC₁₋₃ alkyl; phenyl optionally substituted with 1 or 2 groups selected from OH, halo, C₁₋₃ alkyl, OC₁₋₃alkyl, C(O)NHC₁₋₃alkyl-[N(C₁₋₃alkyl)₂], C(O)(heterocycloalkyl) (e.g. morpholinyl, piperazinyl, piperidinyl) optionally substituted with 1 or 2 C₁₋₃ alkyl groups.

In some embodiments, R^{A} and R^{D} are H. In some embodiments, R^{A}, R^{C} and R^{D} are H. In some embodiments, R^{A}, R^{B}, R^{C} and R^{D} are H.

In some embodiments, R^{E} is H. In one or more embodiments, R^{E} is C₁₋₃alkyl.

In some embodiments, A is C═O.

Those skilled in the art will recognise that when the linker group L comprises an amide or cyclic moiety (e.g. a heterocycloalkyl, cycloalkyl moiety), rotation of the groups attached to the linker will be restricted. In one or more embodiments of the invention, such compounds may have increased activity.

In some embodiments, the heparanase inhibitor is a dihydroquinazoline compound of Formula 4:

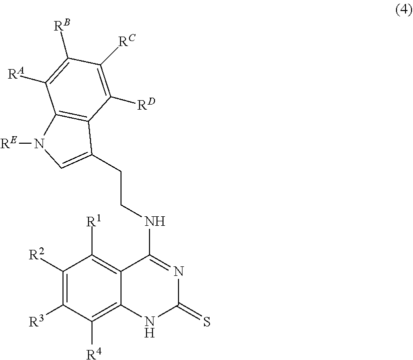

wherein:
R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
R^{E} is H, C(O)-heterocycloalkyl (e.g. C(O)—(N-morpholinyl));
R^{A}, R^{B}, R^{C} and R^{D} are independently selected from H, OH, C₁₋₃alkyl, C(O)—(N-heterocycloalkyl) optionally substituted with 1 or 2 C₁₋₃alkyl groups; N-heteroaryl optionally substituted with 1 or 2 groups selected from OH, halo, C₁₋₃alkyl, C₁₋₃alkoxy; phenyl optionally substituted with 1 or 2 groups selected from OH, halo, C₁₋₃alkyl, C₁₋₃alkoxy, C(O)NHC₁₋₃alkyl-[N(C₁₋₃alkyl)₂], C(O)-heterocycloalkyl optionally substituted with 1 or 2 C₁₋₃alkyl groups.

Preferred embodiments, for R¹, R², R³, R⁴, R^{A}, R^{B}, R^{C}, R^{D} and R^{E} are as described above for compounds of Formula 2, 3, 2A-2C and 3A-3B.

In particular embodiments, the compound of Formula 2, 3 or 4 is selected from:

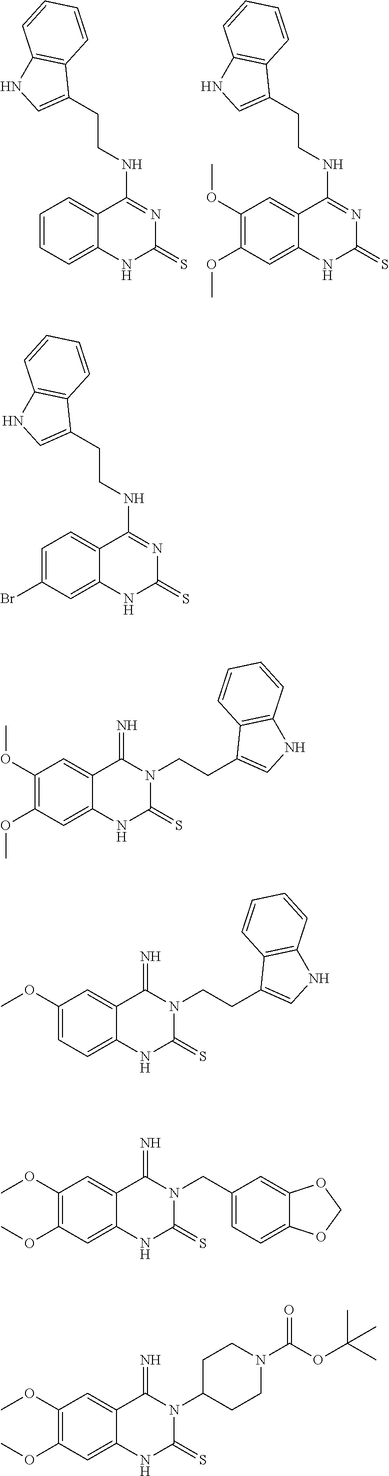

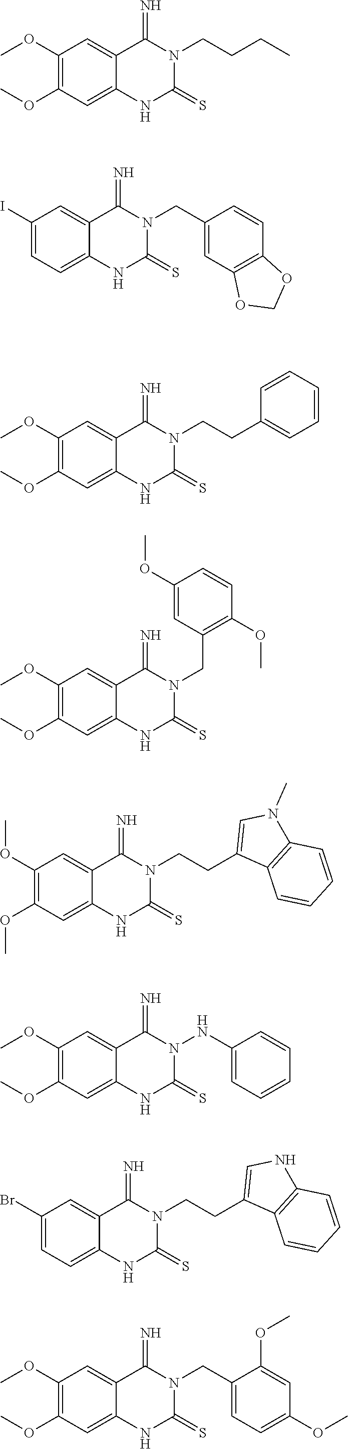

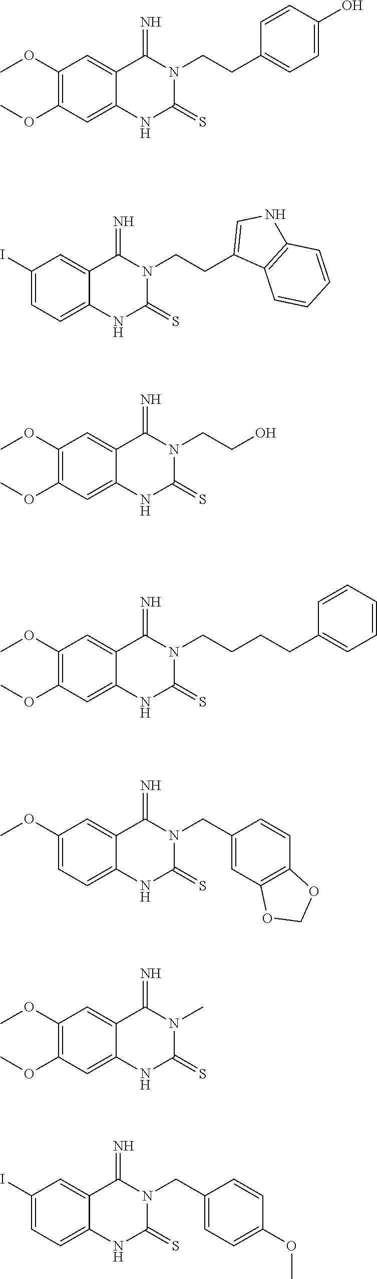

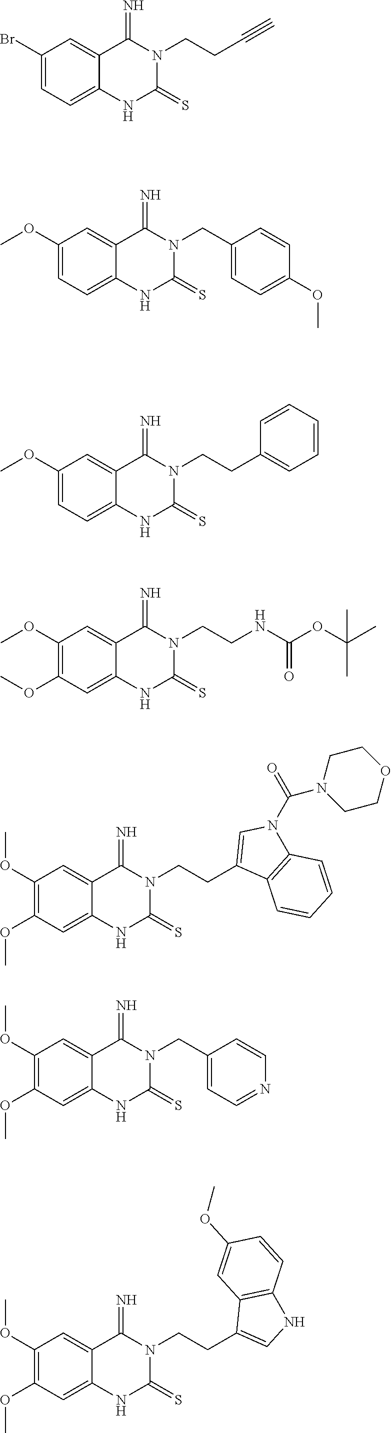

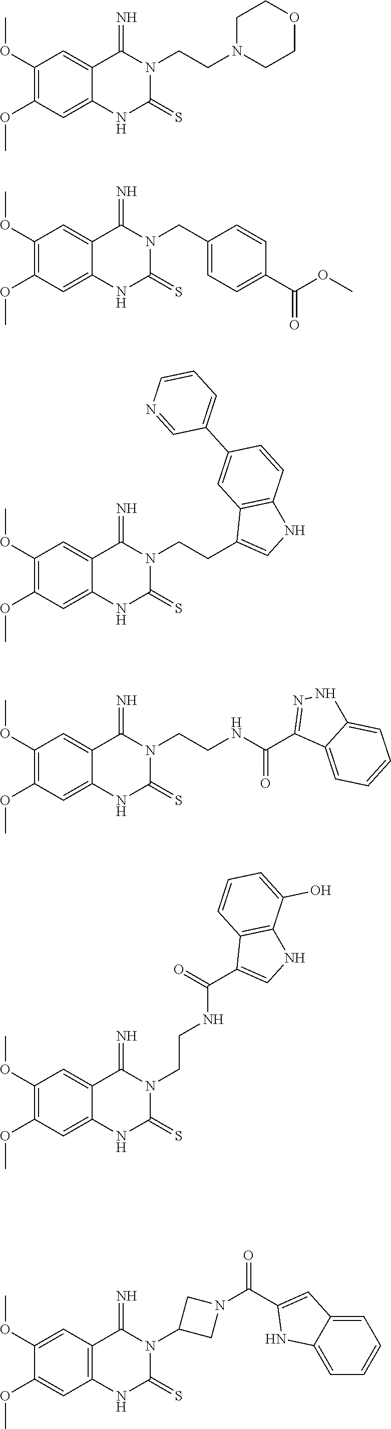

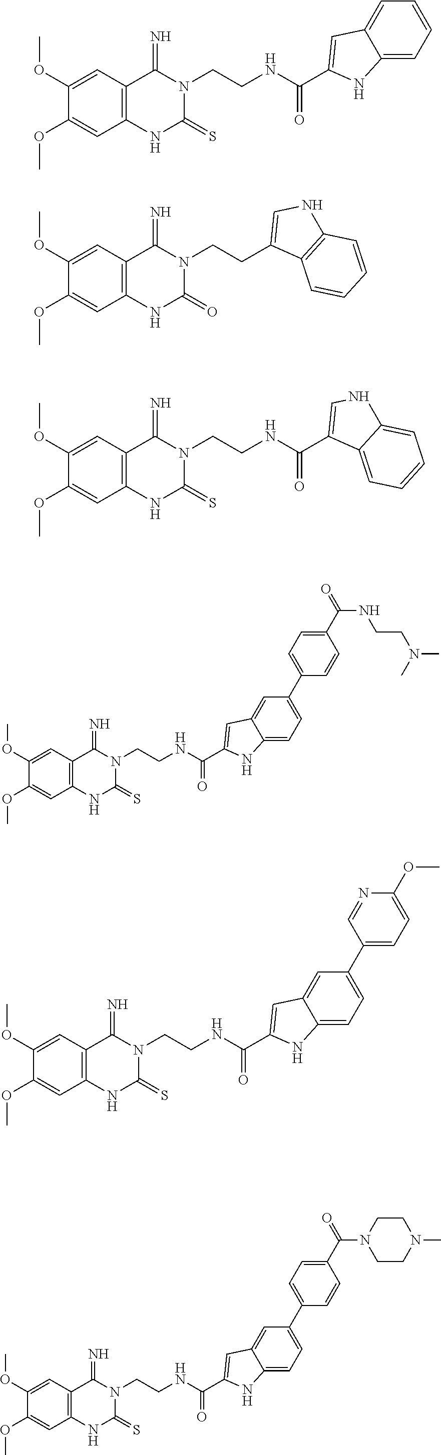

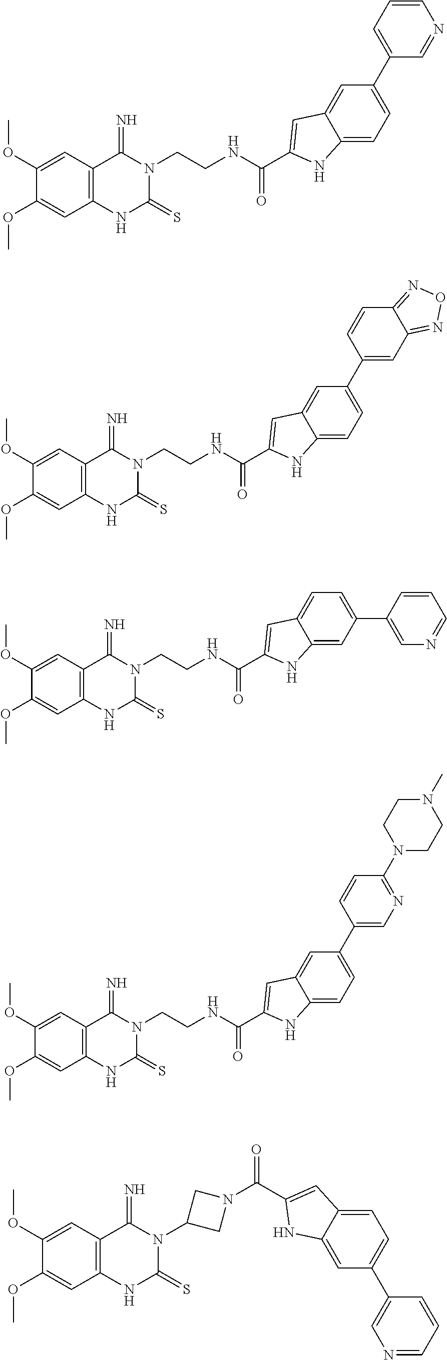 [SMILES: COc1cc2[nH]c(=S)n(CCNC(=O)c3cc4cc(-c5cccnc5)ccc4[nH]3)c(=N)c2cc1OC ; COc1cc2[nH]c(=S)n(CCNC(=O)c3cc4cc(-c5ccc6nonc6c5)ccc4[nH]3)c(=N)c2cc1OC ; COc1cc2[nH]c(=S)n(CCNC(=O)c3cc4ccc(-c5cccnc5)cc4[nH]3)c(=N)c2cc1OC ; COc1cc2[nH]c(=S)n(CCNC(=O)c3cc4cc(-c5ccc(N6CCN(C)CC6)nc5)ccc4[nH]3)c(=N)c2cc1OC ; COc1cc2[nH]c(=S)n(C3CN(C(=O)c4cc5ccc(-c6cccnc6)cc5[nH]4)C3)c(=N)c2cc1OC]

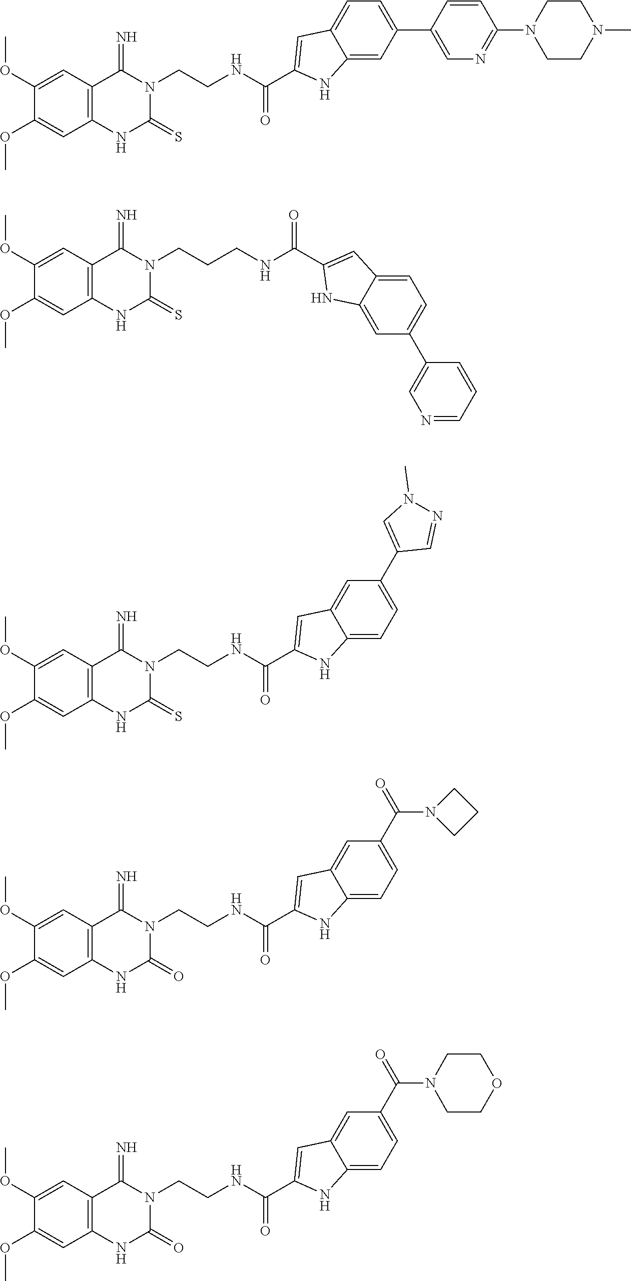

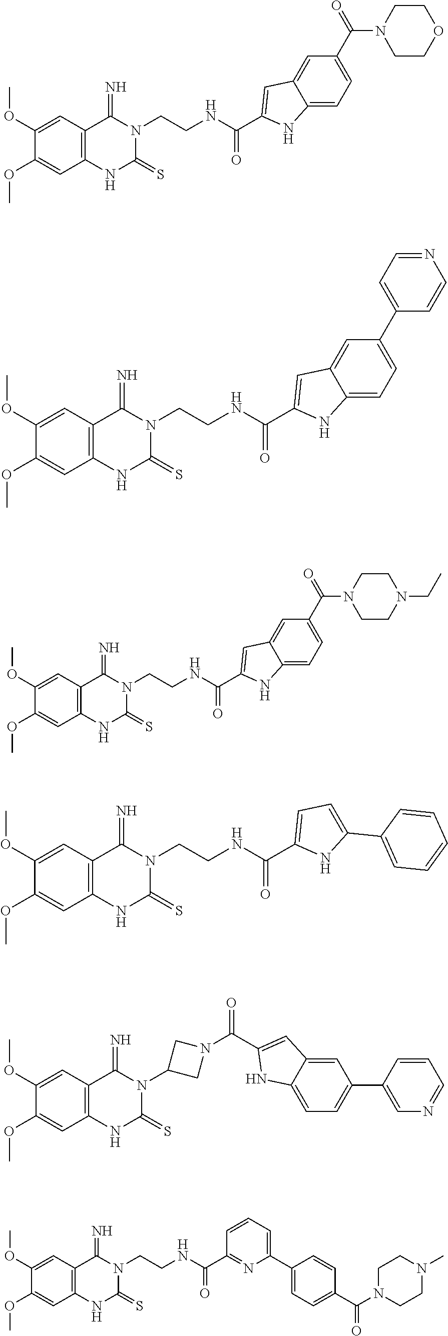 [SMILES: COc1cc2[nH]c(=S)n(CCNC(=O)c3cc4cc(C(=O)N5CCOCC5)ccc4[nH]3)c(=N)c2cc1OC ; COc1cc2[nH]c(=S)n(CCNC(=O)c3cc4cc(-c5ccncc5)ccc4[nH]3)c(=N)c2cc1OC ; CCN1CCN(C(=O)c2ccc3[nH]c(C(=O)NCCn4c(=S)[nH]c5cc(OC)c(OC)cc5c4=N)cc3c2)CC1 ; COc1cc2[nH]c(=S)n(CCNC(=O)c3ccc(-c4ccccc4)[nH]3)c(=N)c2cc1OC ; COc1cc2[nH]c(=S)n(C3CN(C(=O)c4cc5cc(-c6cccnc6)ccc5[nH]4)C3)c(=N)c2cc1OC ; COc1cc2[nH]c(=S)n(CCNC(=O)c3cccc(-c4ccc(C(=O)N5CCN(C)CC5)cc4)n3)c(=N)c2cc1OC]

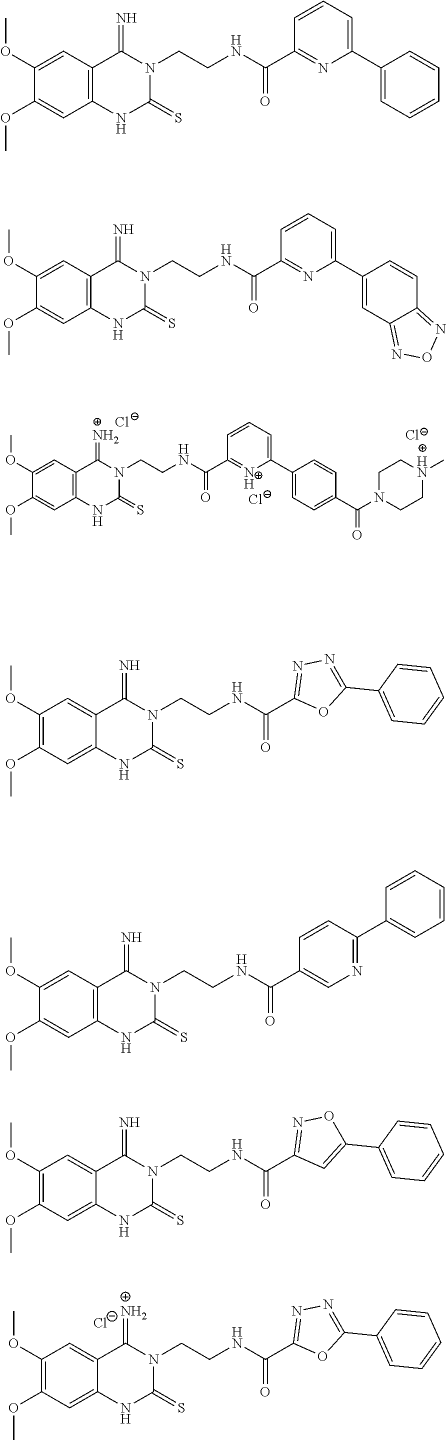

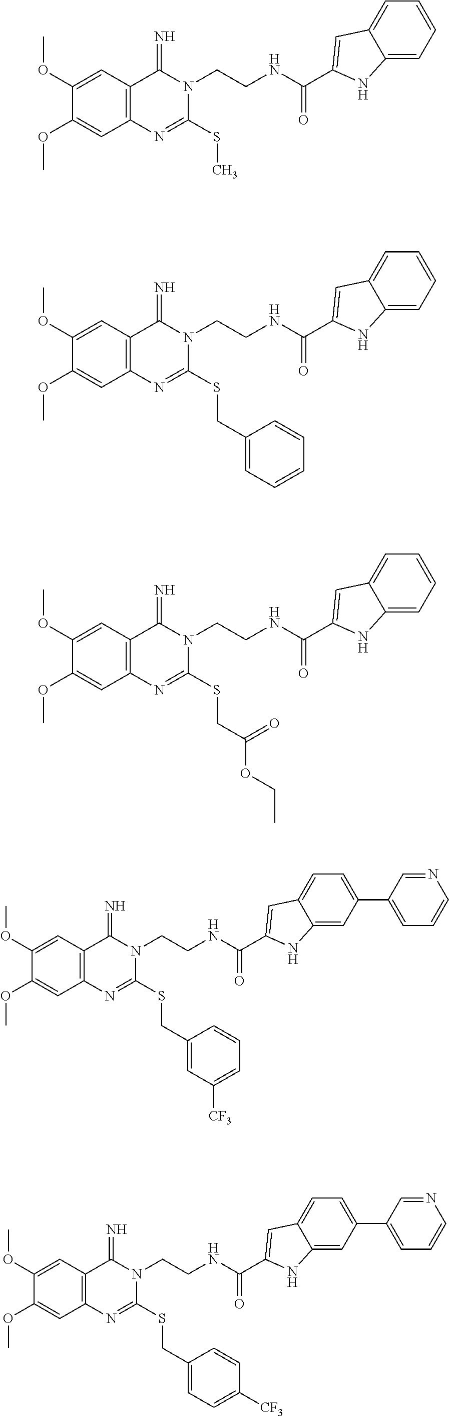 [SMILES: COc1cc2nc(SC)n(CCNC(=O)c3cc4ccccc4[nH]3)c(=N)c2cc1OC ; COc1cc2nc(SCc3ccccc3)n(CCNC(=O)c3cc4ccccc4[nH]3)c(=N)c2cc1OC ; CCOC(=O)CSc1nc2cc(OC)c(OC)cc2c(=N)n1CCNC(=O)c1cc2ccccc2[nH]1 ; COc1cc2nc(SCc3cccc(C(F)(F)F)c3)n(CCNC(=O)c3cc4ccc(-c5cccnc5)cc4[nH]3)c(=N)c2cc1OC ; COc1cc2nc(SCc3ccc(C(F)(F)F)cc3)n(CCNC(=O)c3cc4ccc(-c5cccnc5)cc4[nH]3)c(=N)c2cc1OC]

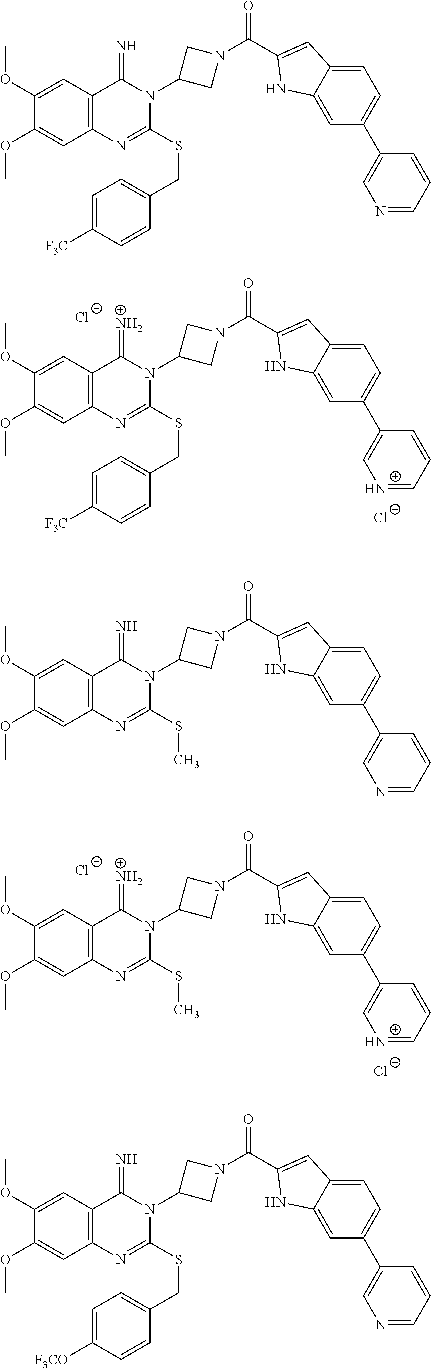

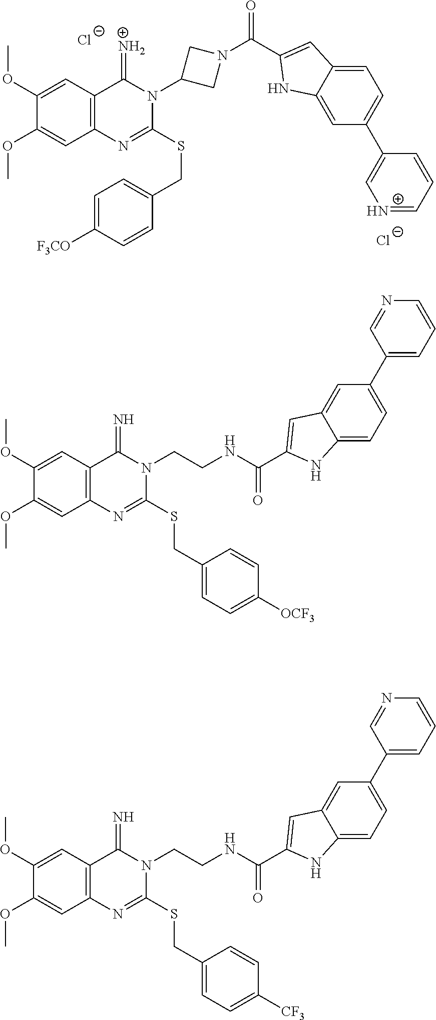 [SMILES: COc1cc2nc(SCc3ccc(OC(F)(F)F)cc3)n(C3CN(C(=O)c4cc5ccc(-c6ccc[nH+]c6)cc5[nH]4)C3)c(=[NH2+])c2cc1OC ; [Cl-] ; [Cl-] ; COc1cc2nc(SCc3ccc(OC(F)(F)F)cc3)n(CCNC(=O)c3cc4cc(-c5cccnc5)ccc4[nH]3)c(=N)c2cc1OC ; COc1cc2nc(SCc3ccc(C(F)(F)F)cc3)n(CCNC(=O)c3cc4cc(-c5cccnc5)ccc4[nH]3)c(=N)c2cc1OC]

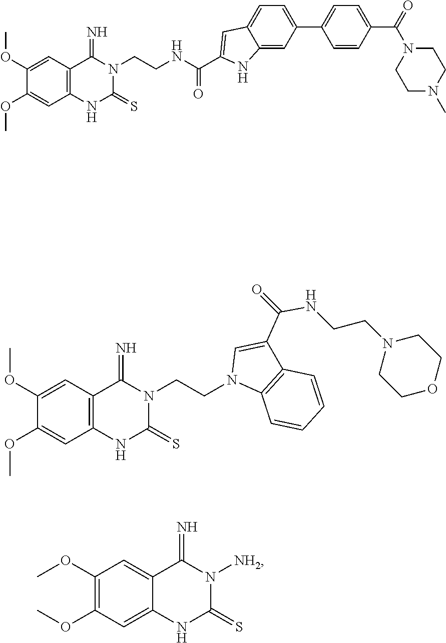

and a salt, hydrate, solvate, tautomer or stereoisomer thereof.

In some embodiments, the compound of Formula 2, 3 or 4 is selected from:

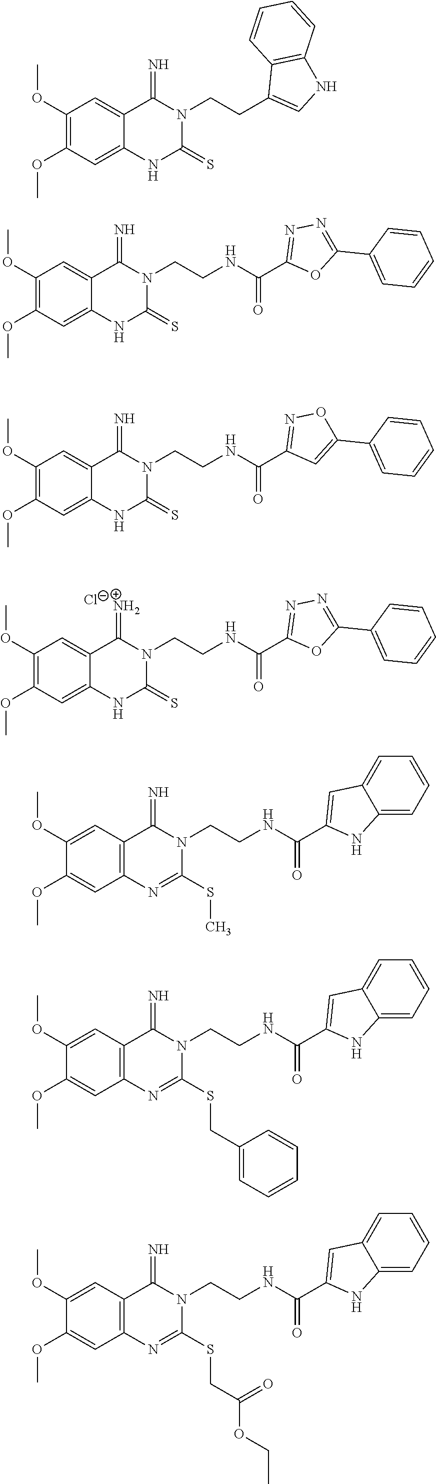

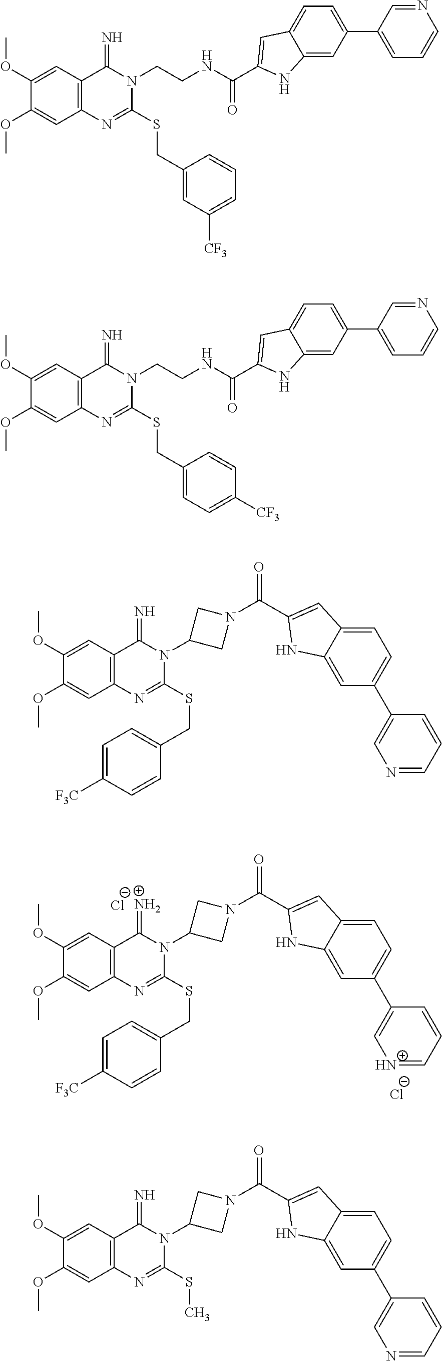

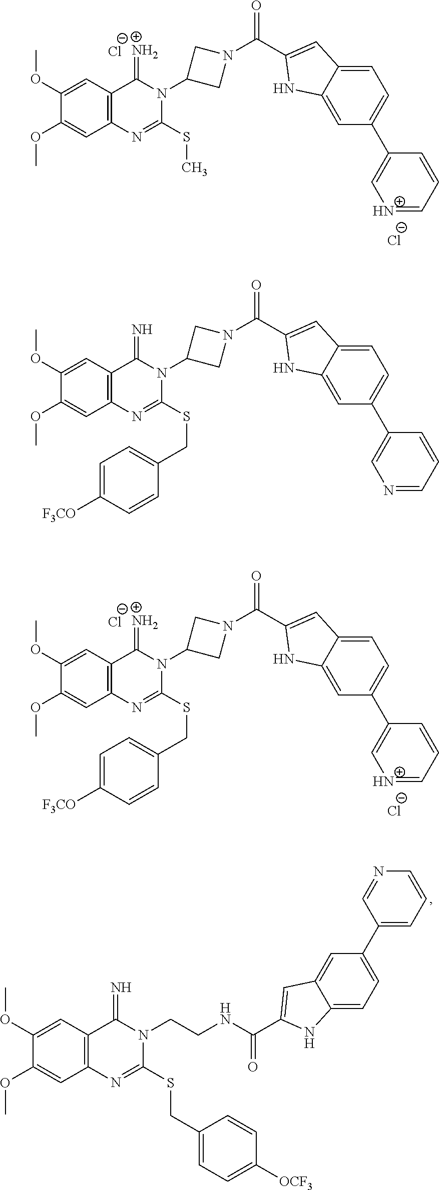

and a salt, hydrate, solvate, tautomer or stereoisomer thereof.

Compounds of Formula 2 may be prepared via the condensation reaction described in Pazdera et al. (1989) Chem Papers, 43: 771 (the content of which is incorporated by reference in its entirety), which involves the condensation of a 2-isothiocyanatobenzonitrile 1 (X=S) or 2-isocyanatobenzonitrile 1 (X=O) with a primary amine 2 as illustrated in Scheme 4:

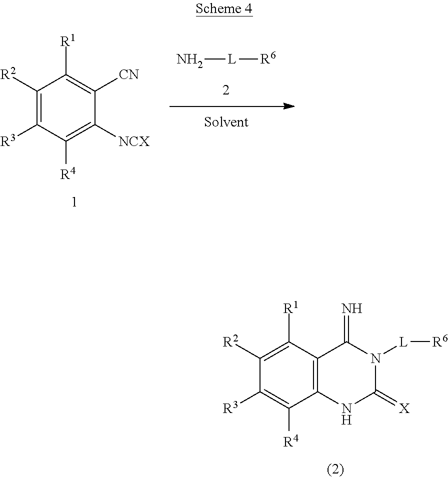

The reaction scheme depicted in Scheme 4 is versatile and allows ready access to the 4-imino-3,4-dihydroquinazoline-2(1H)-thione (X=S) or 4-imino-3,4-dihydroquinazoline-2(1H)-one (X=O) scaffold, with substituent groups R¹-R⁴ in Formula 2 derived from the benzonitrile reactant 1. Advantageously, such reactions may be performed in one-pot.

Typical solvents include, but are not limited to, alcohols (e.g. ethanol, propanol), tetrahydrofuran, petroleum spirit/dichloromethane mixtures.

The reaction is typically performed at a temperature in the range of about 20° C. to about 100° C., e.g., about 20° C. to about 80° C., or about 25° C. to about 80° C.

An exemplary reaction scheme for preparing compounds of Formula 2 is shown in Scheme 4A in which linker L is an alkylene and R⁶ is an optionally substituted 3-indole.

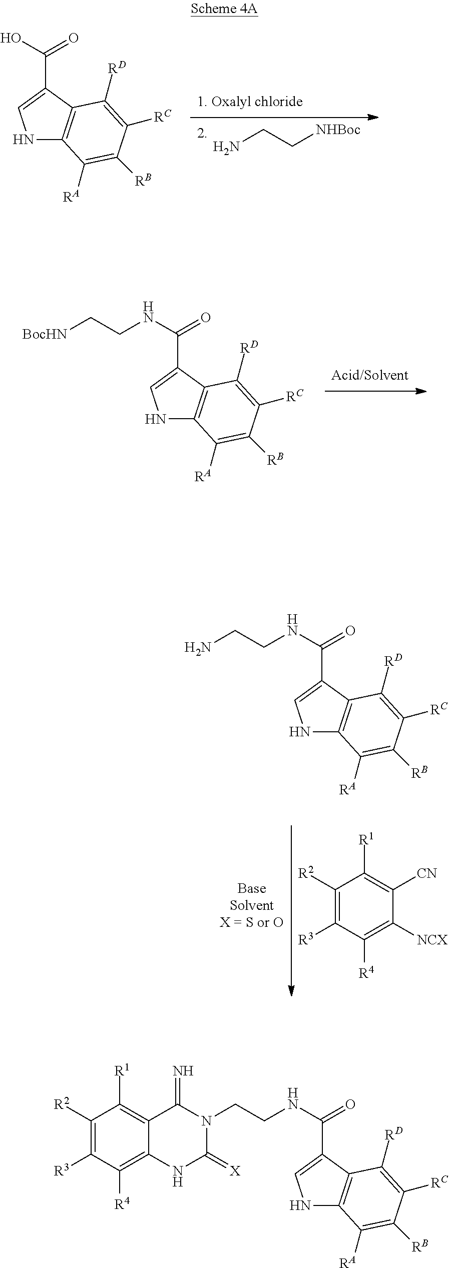

Suitable acids for removal of the t-butoxycarbonyl (Boc) protecting group from the amine in step 2 of Scheme 4A include but are not limited to an inorganic acid such as hydrochloric acid or trifluoracetic acid, and suitable solvents include alcohols such as methanol or ethanol, dioxane, tetrahydrofuran or acetonitrile. Suitable bases for the condensation reaction shown in the third step include but are not limited to triethylamine, diisopropylethylamine, 2,6-dimethylpyridine, and suitable solvents include alcohols e.g. methanol, ethanol, propanol, tetrahydrofuran, and petroleum spirit/dichloromethane mixtures. The reactions are typically carried out at temperatures below 60° C., typically at room temperature.

Another exemplary reaction scheme for preparing compounds of Formula 2 is depicted in Scheme 4B:

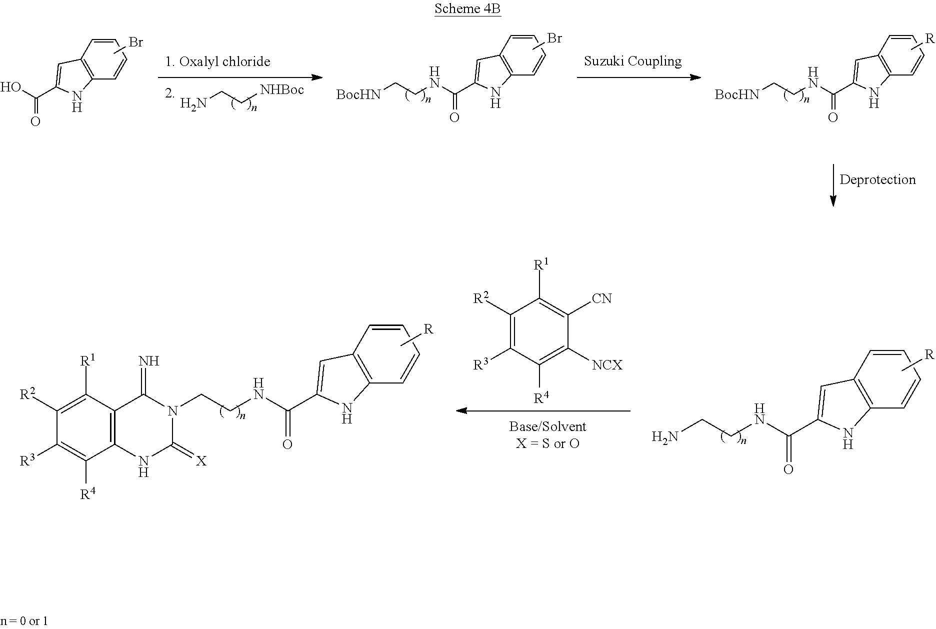

The Suzuki coupling step to introduce functionality onto the indole ring is well known to those skilled in the art and can be performed, for example, by reacting aryl bromide with a boronic acid or ester in base, e.g, potassium carbonate, sodium carbonate in a degassed mixture of dimethoxyethane, water and ethanol, followed by addition of bis(triphenylphosphine)palladium(II) dichloride. The reaction mixture is typically heated to a temperature above 100° C. The deprotection step to remove the Boc protecting group from the amine is also well known in the art and suitable conditions include trifluoroacetic acid/CH₂Cl₂, or hydrochloric acid/dioxane. The final step typically may use a base such as triethylamine, diisopropylethylamine, 2,6-dimethylpyridine, among others, and suitable solvents include alcohols e.g. methanol, ethanol, propanol, tetrahydrofuran, and petroleum spirit/dichloromethane mixtures. The reactions are typically carried out at temperatures below 60° C., typically at room temperature.

Another exemplary reaction scheme useful in the preparation of compounds of Formula 2 is depicted in Scheme 4C below:

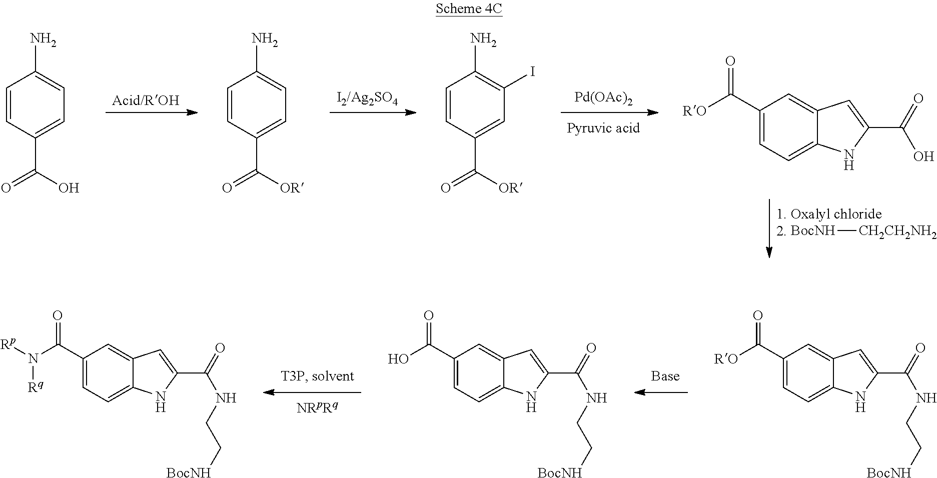

Scheme 4C illustrates methods for introducing amide functionality at the 5-position of the indole ring. In various embodiments, the groups RP and Rq together with the nitrogen atom to which they are attached may form a morpholinyl, piperazinyl or piperidinyl ring. The first step shown in Scheme 4C involves standard methodology to protect the carboxylic acid as an ester. Suitable alcohols (R′OH) include methanol, ethanol, butanol (e.g. n-butanol), etc. A suitable base for use in the fifth step to convert the ester into a carboxylic acid without removing the Boc protecting group includes, but is not limited to, lithium hydroxide. A suitable exemplary solvent for the sixth step which involves formation of the amide bond, is N,N-dimethylformamide. The amide product shown in Scheme 4C may then be deprotected to remove the Boc protecting group and reacted with a 2-isothiocyanatobenzonitrile (X=S) or 2-isocyanatobenzonitrile (X=O) following the general procedures known in the art, including those described above for Scheme 4, Scheme 4A and Scheme 4B.

Compounds of Formula 3 may be prepared from compounds of general Formula 2 (when R⁵=H). For example, as illustrated in general Scheme 5, a compound of Formula 2 represented by quinazoline compound 3 is first treated with base to form an intermediate thioamide enolate (X=S) or an amide enolate (X=O), followed by a nucleophilic substitution reaction with a halide compound 4 (R^{5′}—X′) (where X′=Cl, Br, I) to afford the compound of Formula 3.

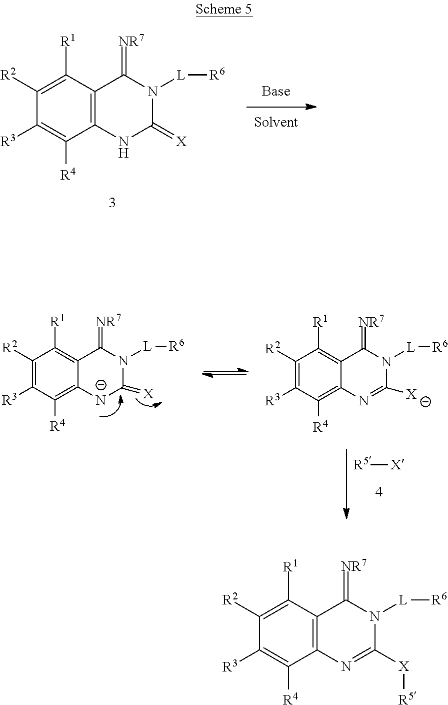

An exemplary reaction scheme for preparing compounds of Formula 3 is shown in Scheme 5A in which linker L is an alkyl amide, R⁶ is an optionally substituted 2-indole and R^{5′}—X′ is methyl iodide.

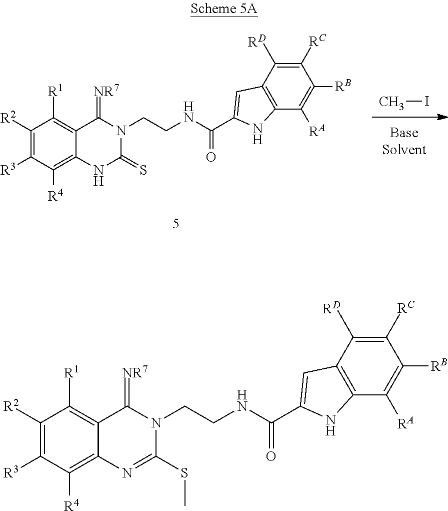

As illustrated in Scheme 5A, a quinazoline compound 5 of Formula 2 is reacted with a base (e.g. potassium carbonate) in a suitable solvent such as acetone. The reaction mixture is stirred, typically at room temperature, until substantially complete. The reaction will typically require at least 2-4 hours, more typically about 12-48 hours, although longer periods may be required for large scale reactions. If necessary, routine methods of purification such as flash column chromatography can be used to isolate compounds of Formula 3.

Compounds of Formula 4 may be prepared by performing a nucleophilic reaction at the 4-position of the dihydroquinazoline 6 (X=S or O) according to the method described in Moreno et al. (2012) Eur J Med Chem, 47: 283, and Lee et al. (1995) J Med Chem, 38(18): 3547-3557 (the contents of which is incorporated by reference in its entirety), as illustrated in general Scheme 6:

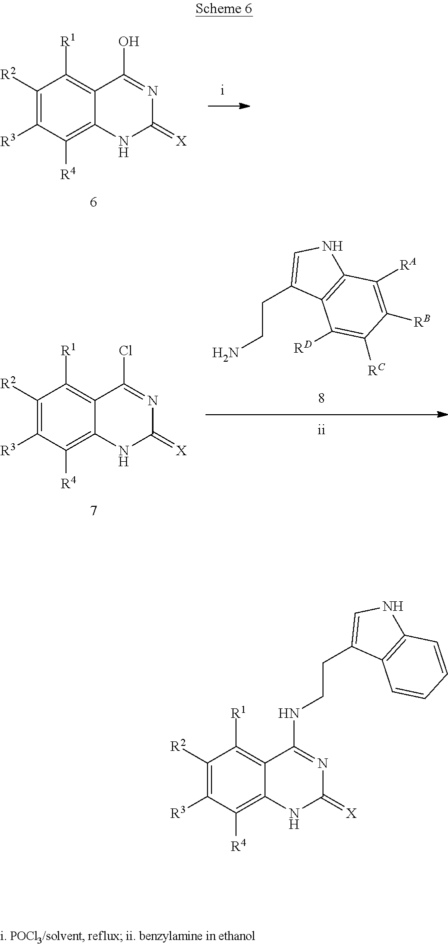

Suitable solvents for step i include, for example, N,N-dimethylformamide (DMF), and THF. Typically the reaction mixture is heated at reflux until substantially complete. Step ii is typically performed by heating the reaction mixture at temperatures above 60° C., typically about 75° C. until the reaction is judged to be substantially complete. Typical reaction times are about 2-24 hours, e.g., about 12 hours or 15 hours, depending on the scale of the reaction.

In an alternative procedure, compounds of Formula 4 may be prepared by thionation of the corresponding 2-chloroquinazoline 9 as illustrated in Scheme 7:

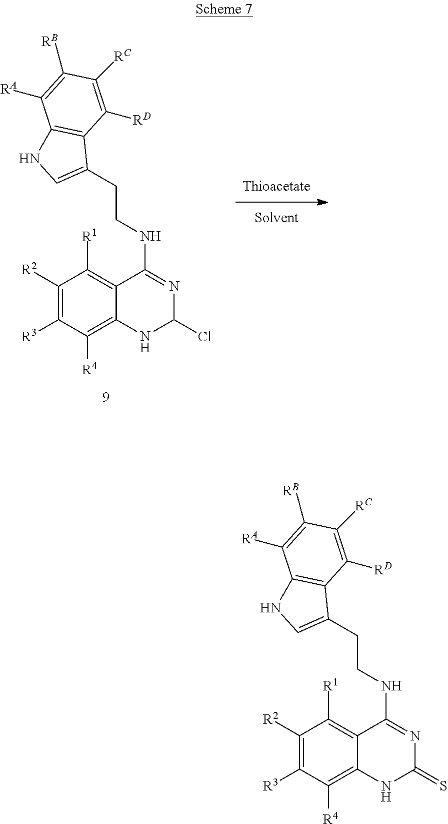

As illustrated in Scheme 7, a 2-chloro-4-amino substituted dihydro-quinazoline 9 is reacted with a salt of thioacetate (e.g. potassium thioacetate) in a suitable solvent such as dioxane or THF. The reaction mixture is typically heated to a temperature above 60° C., typically above 100° C., more typically about 120° C. and the reaction is allowed to proceed for a period of time sufficient for the reaction to proceed substantially to completion. Those skilled in the art know how to monitor the progress of a chemical reaction using standard techniques, such as Thin Layer Chromatography (TLC), ¹H NMR, etc. The reaction will typically require at least 1 hour, more typically about 3 hours, although longer periods may be required for large scale reactions. When the reaction is judged to be sufficiently complete, the reaction mixture is typically cooled, and the reaction solvent removed (e.g. under vacuum), after which the residue is treated with ammonia in a protic solvent such as methanol. The compound of Formula 4 may then be isolated using routine methods such as flash column chromatography.

Where appropriate or necessary, protecting groups may be employed at any stage in the synthesis of the compounds. Similarly, those skilled in the art will also appreciate that the compounds can also be prepared with certain protecting groups that are useful for purification or storage that can be removed before administration to a subject. Suitable protecting groups and their use are well known to those skilled in the art and include, for example, protecting groups described in Wuts and Greene (2007) Greene's Protective Groups in Organic Synthesis, 4th Edition. (John Wiley & Sons, Inc.). The protection and deprotection of functional groups is also described in Protective Groups in Organic Chemistry, edited by J. W. F. McOmie, Plenum Press (1973).

A skilled person will recognize the versatility of the reactions illustrated in the above Schemes, which can provide access to a wide range of substituted dihydro and tetrahydro quinazoline compounds of Formulae 2, 3 and 4.

Compounds of Formula 2, 3 or 4 may be isolated or purified using standard techniques known to those skilled in the art. Such techniques include precipitation, crystallization, recrystallization, column chromatography (including flash column chromatography), HPLC, formation of salts, lyophilization, among others. Suitable solvents for use in these techniques will be known or can be readily ascertained by those skilled in the art using routine practices.

Salts, including pharmaceutically acceptable salts of compounds of Formula 2, 3 or 4 may be prepared by methods known to those skilled in the art, including for example:

(i) by reacting the compound of Formula 2, 3 or 4 with the desired acid or base;
(ii) by removing an acid- or base-labile protecting group from a suitable precursor of the compound of Formula 2, 3 or 4 or by ring-opening a suitable cyclic precursor, for example, a lactone or lactam, using the desired acid or base; or
(iii) by converting one salt of the compound of Formula 2, 3 or 4 into another salt by reaction with an appropriate acid or base, or by means of a suitable ion exchange column.

The above reactions are typically carried out in solution. Suitable solvent systems (including mixed solvent systems) are well known to those skilled in the art and those skilled in the art can readily select or determine a suitable solvent system using routine methods taking into consideration the nature of the compound, the particular salt being formed, and the amount of the compound. Exemplary solvent systems include methanol, ethanol, water, acetone, tetrahydrofuran, dichloromethane, pentane, hexane, diethyl ether, ethyl acetate, and any mixture of two or more such solvents. The resulting salt may precipitate out and be collected by filtration or may be recovered by evaporation of the solvent. The degree of ionization in the resulting salt may vary from completely ionized to almost non-ionized.

In some embodiments, the heparanase inhibitor is a sulfated linked cyclitol as described in WO 2003/004454 A1, the content of which is herein incorporated by reference in its entirety. Accordingly, in some embodiments, the heparanase inhibitor is a compound of Formula 5:

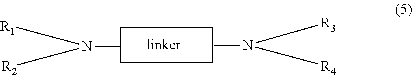

wherein:
R₁, R₂, R₃ and R₄ are independently a substituted or unsubstituted cyclitol with a ring comprising six carbon atoms, or hydrogen, substituted or unsubstituted alkyl, cycloalkyl, aryl, acyl, alkyloxycarbonyl, or alkylaminocarbonyl, with the proviso that at least two of R₁, R₂, R₃ and R₄ comprise said substituted or unsubstituted cyclitol; or
R₁ and R₃ are independently a substituted or unsubstituted cyclitol carbamide with a ring comprising six carbon atoms with the linker bond at the carbamide nitrogen, and R₂ and R₄ are independently hydrogen, substituted or unsubstituted alkyl, cycloalkyl or aryl; and the linker is selected from the group consisting of —(CH₂)_{w}—, —(CH₂)_{X}—C₆H₄—(CH₂)_{X}—, —(CH₂)_{y}—NR₅—(CH₂)_{y}—, and —(CH₂)_{Z}—HCR₆—(CH₂)_{Z}—; wherein: w, x, y and z are independently an integer having a value of 0-10; R₅ is a substituted or unsubstituted cyclitol with a ring comprising six carbon atoms; and, R₆ is —OH, —OSO₃Na, —OSO₃Na substituted with alkyl, cycloalkyl or aryl, or substituted or unsubstituted alkyl, cycloalkyl or aryl; and wherein each substituent on said substituted cyclitol is independently selected from:
(a) phosphoryl groups such as phosphate, thiophosphate —O—P(S)(OH)₂; phosphate esters —O—P(O)(OR)₂; thiophosphate esters —O—P(S)(OR)₂; phosphonate —O—P(O)OHR; thiophosphonate —O—P(S)OHR; substituted phosphonate —O—P(O)OR₁R₂; substituted thiophosphonate —O—P(S)OR₁R₂; —O—P(S)(OH)(SH); and cyclic phosphate;
(b) other phosphorus containing compounds such as phosphoramidite —O—P(OR)—NR₁R₂; and phosphoramidate —O—P(O)(OR)—NR₁R₂;
(c) sulfur groups such as —O—S(O)(OH), —O—S(O)₂(OH), RO—S(O)₃⁻, —SH, —SR, —S(→O)—R, S(O)₂R, RO—S(O)₂, —O—SO₂NH₂, —O—SO₂R₁R₂ or sulfamide —NHSO₂NH₂;
(d) amino groups such as —NHR, —NR₁R₂, —NHAc, —NHCOR, —NH—O—COR, HSO₃, —NHSO₂R, —N(SO₂R)₂, and/or amidino groups such as —NH—C(═NH)NH₂ and/or ureido groups such as —NH—CO—NR₁R₂ or thiouriedo groups such as —H—C(S)—NH₂;
(e) substituted hydroxy groups such as —OR₃, where R₃ is C₁₋₁₀ unsubstituted or substituted alkyl, alkoxyalkyl, aryloxyalkyl, cycloalkyl, alkenyl (unsubstituted alkyl), alkylene (C₃₋₇ cycloalkyl), —OCOR, aryl, heteroaryl, acetal, or where two hydroxyl groups are joined as a ketal;
(f) a halogen;
(g) a cyclitol or substituted cyclitol as defined above; or
(h) a physiologically acceptable salt of any of the above;
   wherein in (a) to (g) above R, R₁ and R₂ are independently hydrogen or C₁₋₁₀ unsubstituted or substituted alkyl or aryl.

In particular embodiments, the compound of Formula 5 is selected from:

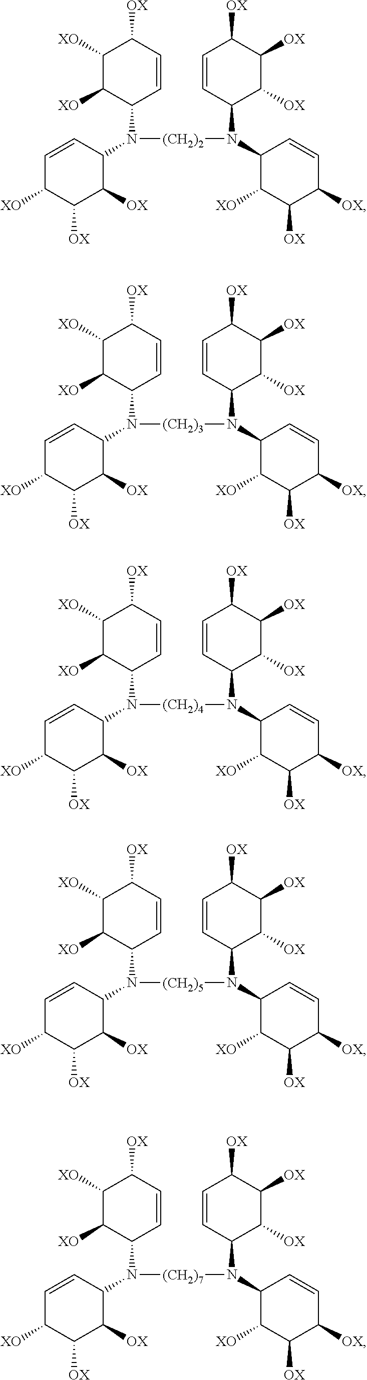

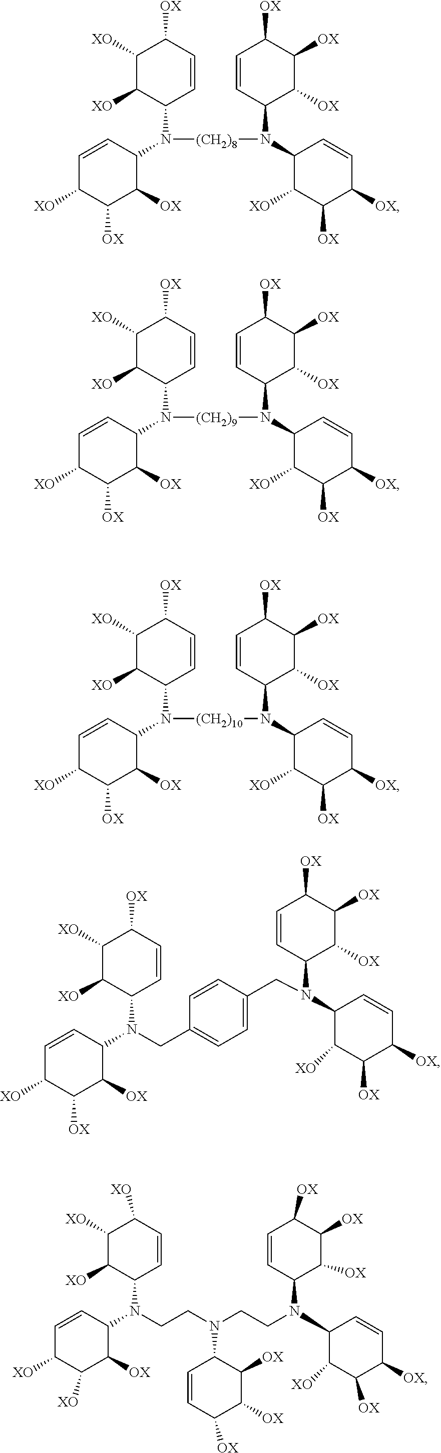

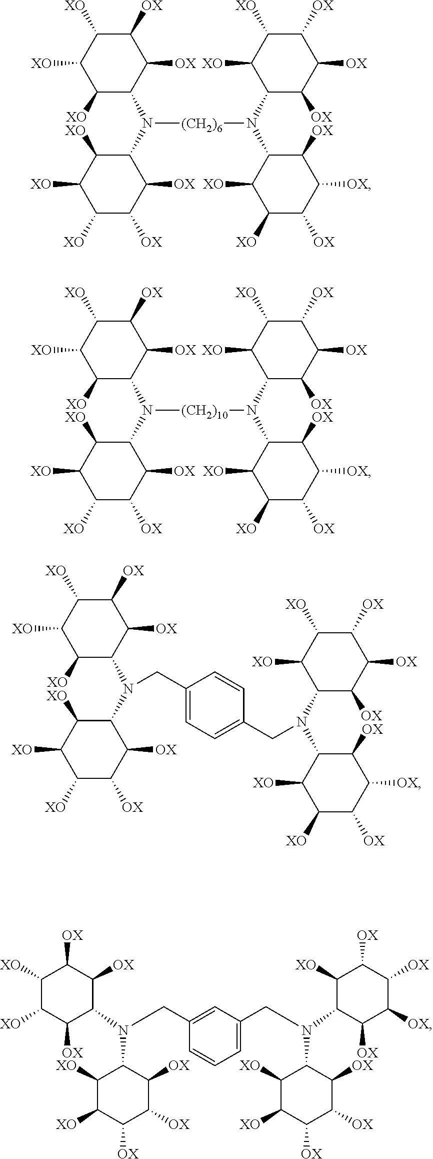

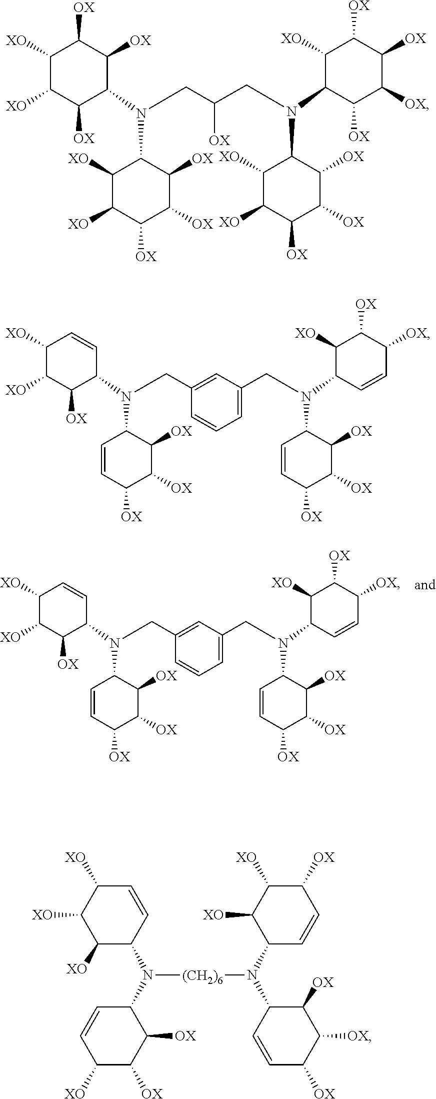

wherein X=SO₃Na.

In some embodiments, the heparanase inhibitor is a diphenyl ether derivative described in WO 02/060375 A2, the content of which is incorporated by reference in its entirety. Accordingly, in some embodiments, the heparanase inhibitor is a diphenyl ether of Formula 6:

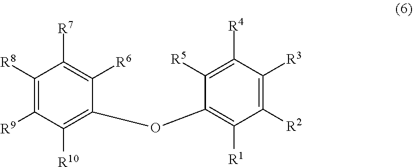

wherein
R1, R5, R6 and R7 each independently represents hydrogen or halogen;
R2, R3, R4 and R8 each independently represents hydrogen, halogen, nitro, —OR′, —SW, —NR11R12, —COOR′, —CONR11R12, —SO₃H, —SO₂NR11R12, C1-C6 alkyl, C1-C6 alkoxy, C2-C6 alkenyl, C6-C14 aryl or heteroaryl;
R9 and R10 each independently is hydrogen or halogen, or R9 and R10 together with the carbon atoms to which they are attached form a condensed benzene ring;
R11 and R12, each independently represents hydrogen, C1-C6 alkyl, C2-C6 alkenyl, C6-C14 aryl or heteroaryl;
or R11 is H and R12 is C2-C7 alkanoyl or C7-C15 aroyl, or R11 and R12 together with the N atom to which they are attached form a saturated 5-7 membered heterocyclic ring containing one to three heteroatoms selected from N, O and/or S;
R′ is hydrogen, C1-C6 alkyl, C2-C6 alkenyl, C6-C14 aryl or heteroaryl;
“heteroaryl” in radicals R2, R3, R4, R8, R11, R12 and R′ is a radical derived from a mono- or poly-cyclic heteroaromatic ring containing one to three heteroatoms selected from N, O and/or S;
any “C1-C6 alkyl”, “C2-C7 alkanoyl” and C2-C6 alkenyl in radicals R2, R3, R4, R8, R11, R12 and R′ may be substituted by at least one group selected from halogen, —OR′, —SW, —NR11R12, —COOR′, —CONR11R12, nitro, —SO₃H, —SO₂NR11R12, C6-C14 aryl, and heteroaryl;
any “C6-C14 aryl”, “C7-C15 aroyl” and “heteroaryl” in radicals R2, R3, R4, R8, R11, R12 and R′ may be substituted by at least one group selected from halogen, —OR′, —SW, —NR11R12, —COOR′, —CONR11R12, nitro, —SO₃H, —SO₂NR11R12, C1-C6 alkyl, C1-C6 alkoxy, C2-C6 alkenyl, and C5-C6 cycloalkyl;
and pharmaceutically acceptable salts thereof.

In particular embodiments, the diphenyl ether of Formula 6 is selected from:

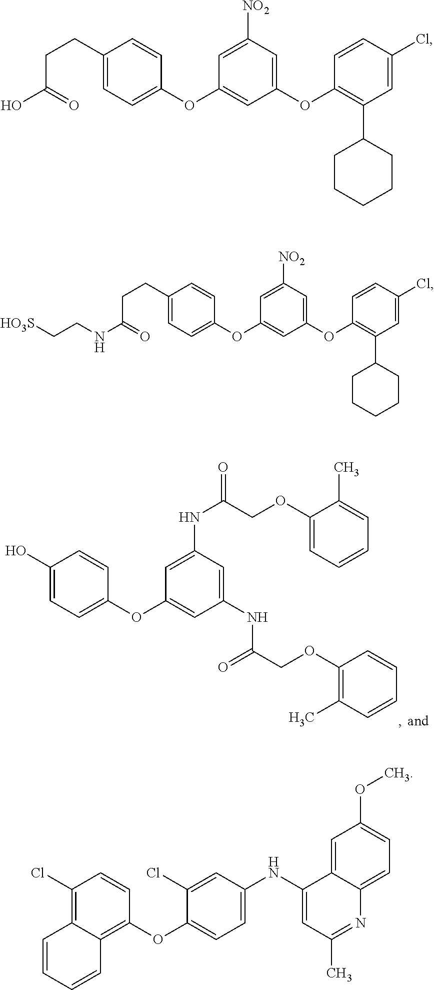 [SMILES: O=C(O)CCc1ccc(Oc2cc(Oc3ccc(Cl)cc3C3CCCCC3)cc([N+](=O)[O-])c2)cc1 ; O=C(CCc1ccc(Oc2cc(Oc3ccc(Cl)cc3C3CCCCC3)cc([N+](=O)[O-])c2)cc1)NCCS(=O)(=O)O ; Cc1ccccc1OCC(=O)Nc1cc(NC(=O)COc2ccccc2C)cc(Oc2ccc(O)cc2)c1 ; COc1ccc2nc(C)cc(Nc3ccc(Oc4ccc(Cl)c5ccccc45)c(Cl)c3)c2c1]

In some embodiments, the heparanase inhibitor is an indole derivative described in WO 02/060373 A2, the entire content of which is herein incorporated by reference. Accordingly, in some embodiments, the heparanase inhibitor is an indole derivative of Formula 7 or Formula 8:

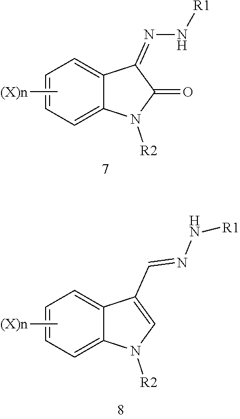

wherein
R1 is C7-C15 aroyl optionally substituted by at least one radical selected from halogen, hydroxy, nitro, —NR3R4, —SO₃H, C1-C6 alkyl, C2-C6 alkenyl, or C1-C6 alkoxy; or heteroaryl derived from a mono- or poly-cyclic heteroaromatic ring containing one to three heteroatoms selected from N, O and/or S, and being optionally substituted by at least one radical selected from halogen, hydroxy, nitro, —NR3R4, —SO₃H, C1-C6 alkyl, C2-C6 alkenyl, or C1-C6 alkoxy;
R2 is hydrogen; C1-C6 alkyl optionally substituted by halogen, hydroxy, nitro, —NR3R4, —COOR3, —CONR3R4, —SO₃H or C6-C14 aryl; C2-C6 alkenyl; C6-C14 aryl; or heteroaryl derived from a mono- or poly-cyclic heteroaronnatic ring containing one to three heteroatoms selected from N, O and/or S; said C6-C14 aryl or heteroaryl being optionally substituted by at least one radical selected from halogen, hydroxy, nitro, —NR3R4, —SO₃H, C1-C6 alkyl, C2-C6 alkenyl, or C1-C6 alkoxy;
R3 and R4 each independently represents hydrogen, C1-C6 alkyl, C2-C6 alkenyl, or C6-C14 aryl optionally substituted by halogen, hydroxy, nitro, —NH₂, —SO₃H, —COOR2, C1-C6 alkyl, or C2-C6 alkenyl;
or R3 is H and R4 is a C7-C15 aroyl optionally substituted by halogen, hydroxy, nitro, —NH₂, —SO₃H, —COOR2, C1-C6 alkyl, or C2-C6 alkenyl;
X represents halogen, nitro, —OR3, —SR3, —NR3R4, —SO₃H, —COOR3, C1-C6 alkyl, C2-C6 alkenyl, or C6-C14 aryl optionally substituted by at least one radical selected from halogen, hydroxy, nitro, —NR3R4, —SO₃H, C1-C6 alkyl, C2-C6 alkenyl, or C1-C6 alkoxy;
n is an integer from 0 to 4;
and pharmaceutically acceptable salts thereof.

In some embodiments, the indole derivative of Formula 7 is selected from:

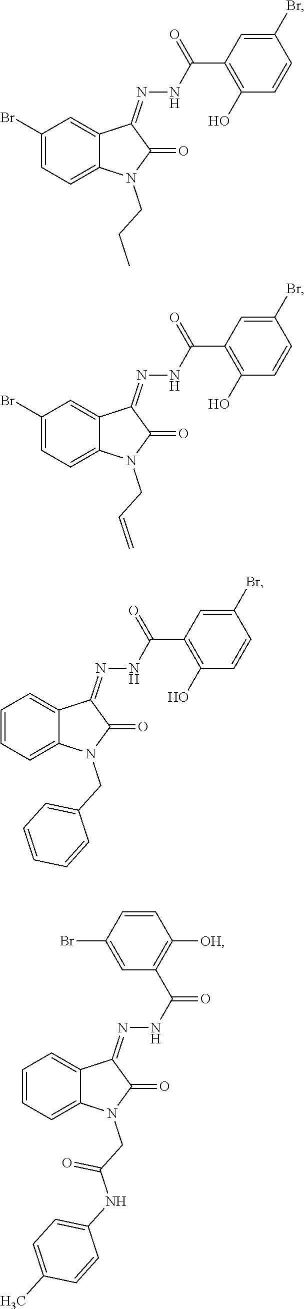 [SMILES: CCCN1C(=O)/C(=N\NC(=O)c2cc(Br)ccc2O)c2cc(Br)ccc21 ; C=CCN1C(=O)/C(=N\NC(=O)c2cc(Br)ccc2O)c2cc(Br)ccc21 ; O=C(N/N=C1\C(=O)N(Cc2ccccc2)c2ccccc21)c1cc(Br)ccc1O ; Cc1ccc(NC(=O)CN2C(=O)/C(=N\NC(=O)c3cc(Br)ccc3O)c3ccccc32)cc1]

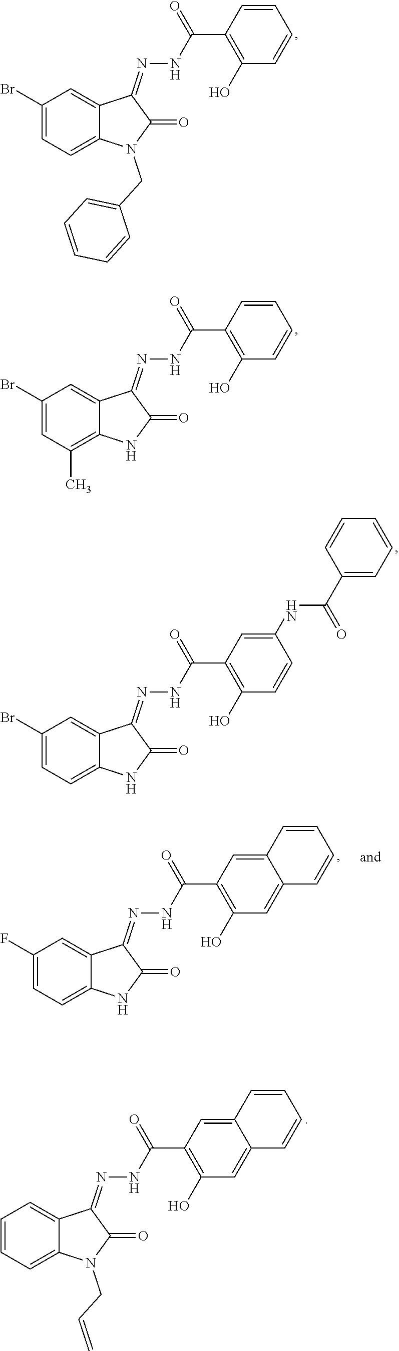 [SMILES: O=C(N/N=C1\C(=O)N(Cc2ccccc2)c2ccc(Br)cc21)c1ccccc1O ; Cc1cc(Br)cc2c1NC(=O)/C2=N\NC(=O)c1ccccc1O ; O=C1Nc2ccc(Br)cc2/C1=N/NC(=O)c1cc(NC(=O)c2ccccc2)ccc1O ; O=C1Nc2ccc(F)cc2/C1=N/NC(=O)c1cc2ccccc2cc1O ; C=CCN1C(=O)/C(=N\NC(=O)c2cc3ccccc3cc2O)c2ccccc21]

In some embodiments, the indole derivative of Formula 8 is:

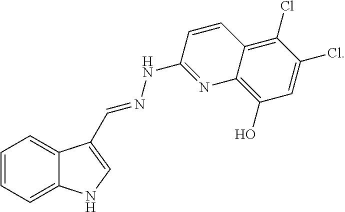 [SMILES: Oc1cc(Cl)c(Cl)c2ccc(N/N=C/c3c[nH]c4ccccc34)nc12]

In some embodiments, the heparanase inhibitor is a carbazole or fluorine derivative described in WO 02/060867 A2, the content of which is incorporated by reference in its entirety. Accordingly, in some embodiments, the heparanase inhibitor is a carbazole or fluorene derivative of Formula 9:

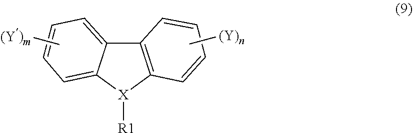

wherein
either X is N and R1 is a 3-amino-2-hydroxy-propyl radical of the formula:

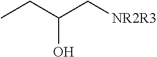

or X is C and R1 is a radical of the formula:

### ═N—NH—R4

and
Y and Y′ each independently represents hydrogen, halogen, C1-C6 alkyl, C2-C6 alkenyl, C6-C14 aryl, nitro, —OR, —SR, —CONRR′, —NRCONRR′, —NRR′, —SO₃H, or —SO₂NRR′;
R2 and R3 each independently represents hydrogen, C1-C6 alkyl, C2-C6 alkenyl, C6-C14 aryl, or heteroaryl;
or R2 is hydrogen, C1-C6 alkyl, C2-C6 alkenyl, C6-C14 aryl, or heteroaryl and R3 is —CONRR′, —CSNRR′, or —CONHNRR′;
or R2 and R3 together with the N atom to which they are attached form a saturated 5-7 membered heterocyclic ring optionally containing at least one further heteroatom selected from N, O, and/or S, said at least one further N atom being optionally substituted by R5;

### R4 is —CONRR′, —CSNRR′ or —CONHNRR′;

R5 is C1-C6 alkyl substituted by carbazolyl at the terminal carbon atom and by a further group selected from halogen, —OH, —SH, —NH₂, C1-C6 alkoxy or C1-C6 alkylthio; or heteroaryl derived from a bicyclic heteroaromatic ring containing one to three heteroatoms selected from N, O and/or S;
R and R′ each independently represents (i) hydrogen; (ii) C1-C6 alkyl optionally substituted by halogen, —OH, —SH, —NH₂, C1-C6 alkoxy, C1-C6 alkylthio, C6-C14 aryl, and/or heteroaryl; (iii) C2-C6 alkenyl optionally substituted by halogen, —OH, —SH, —NH₂, C1-C6 alkoxy, C1-C6 alkylthio, C6-C14 aryl and/or heteroaryl; (iv) C6-C14 aryl optionally substituted by halogen, —OH, —SH, —NH₂, —SO₃H, C1-C6 alkyl, C2-C6 alkenyl, C1-C6 alkoxy, and/or C1-C6 alkylthio; or (v) heteroaryl optionally substituted by halogen, —OH, —SH, —NH₂, —SO₃H, C1-C6 alkyl, C2-C6 alkenyl, C1-C6 alkoxy, and/or C1-C6 alkylthio;
“heteroaryl” in radicals R, R′, R2, and R3 is a radical derived from a mono- or poly-cyclic heteroaromatic ring containing one to three heteroatoms selected from N, O and/or S;
any “C1-C6 alkyl” or “C2-C6 alkenyl” in radicals R2 and R3 may be substituted by at least one radical selected from halogen, —OH, —SH, C1-C6 alkoxy, C1-C6 alkylthio, C6-C14 aryl, heteroaryl, —NRR′, —COOR, —CONRR′, —SO₃H, or —SO₂NRR′;
any “C6-C14 aryl” and “heteroaryl” in radicals R2, R3 and R5 may be substituted by at least one radical selected from halogen, —OH, —SH, C1-C6 alkyl, C2-C6 alkenyl, C1-C6 alkoxy, C1-C6 alkylthio, —NRR′, —COOR, —CONRR′, —SO₃H, or —SO₂NRR′;
m and n, the same or different, are integers from 0 to 4;
and pharmaceutically acceptable salts thereof.

In some embodiments, the carbazole or fluorene derivative of Formula 9 is selected from:

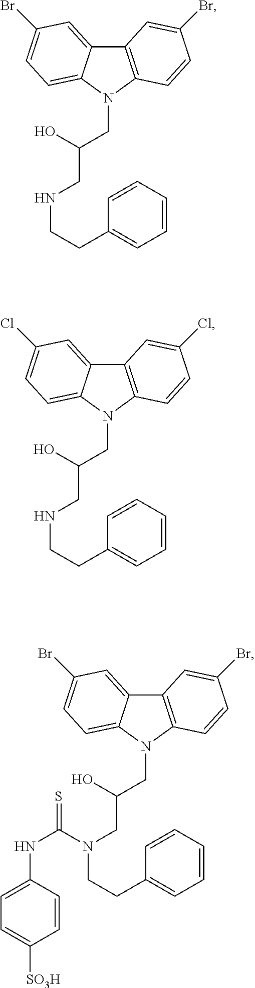 [SMILES: OC(CNCCc1ccccc1)Cn1c2ccc(Br)cc2c2cc(Br)ccc21 ; OC(CNCCc1ccccc1)Cn1c2ccc(Cl)cc2c2cc(Cl)ccc21 ; O=S(=O)(O)c1ccc(NC(=S)N(CCc2ccccc2)CC(O)Cn2c3ccc(Br)cc3c3cc(Br)ccc32)cc1]

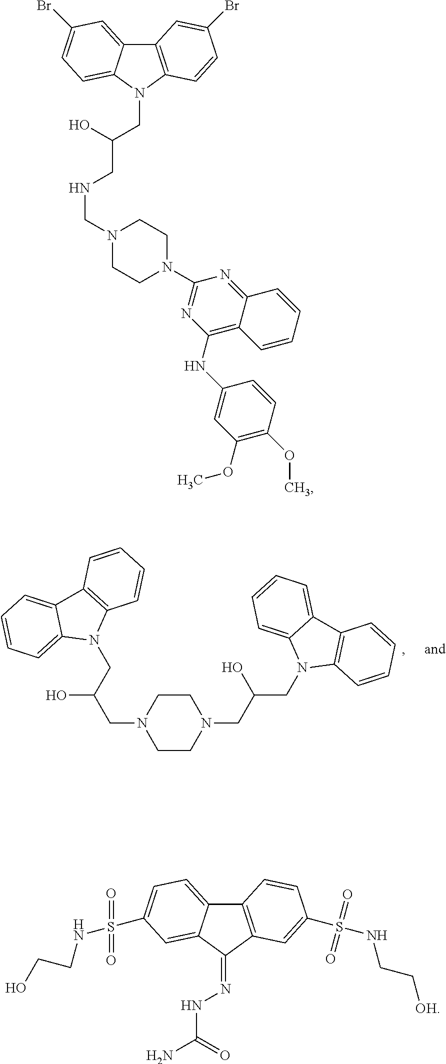 [SMILES: COc1ccc(Nc2nc(N3CCN(CNCC(O)Cn4c5ccc(Br)cc5c5cc(Br)ccc54)CC3)nc3ccccc23)cc1OC ; OC(CN1CCN(CC(O)Cn2c3ccccc3c3ccccc32)CC1)Cn1c2ccccc2c2ccccc21 ; NC(=O)NN=C1c2cc(S(=O)(=O)NCCO)ccc2-c2ccc(S(=O)(=O)NCCO)cc21]

In some embodiments, the heparanase inhibitor is a benz-1,3-azole derivative described in WO 02/060374 A2, the content of which is incorporated by reference in its entirety. Accordingly, in some embodiments, the heparanase inhibitor is a benz-1,3-azole derivative of Formula 10:

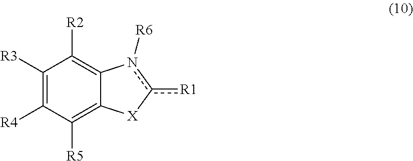

wherein
R1 is a radical selected from radicals (a)-(i):

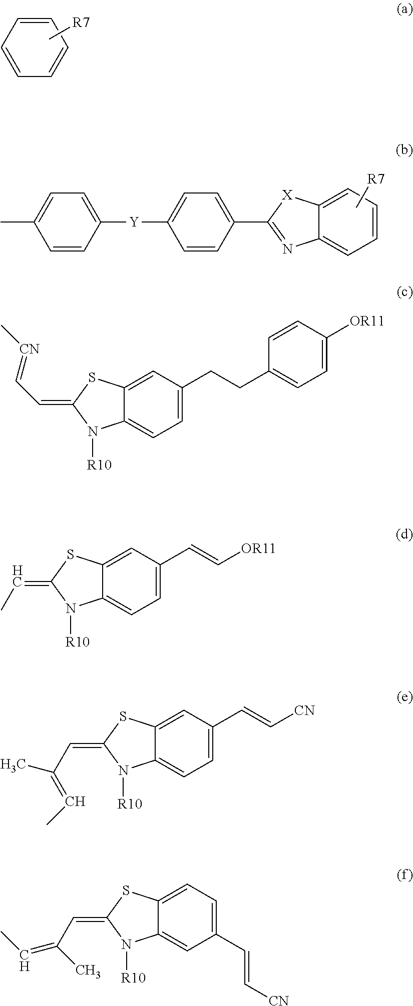

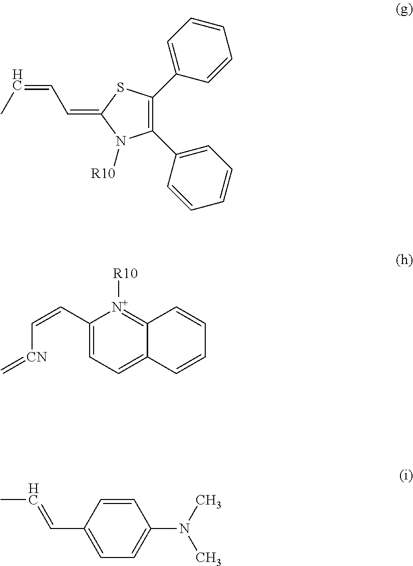

and wherein
R2 and R5 each independently represents hydrogen; halogen; —SO₃H; C1-C6 alkoxy optionally substituted by halogen or —SO₃H; C2-C6 alkenyl; C2-C7 alkanoyl; C1-C6 alkyl optionally substituted by halogen or C1-C6 alkoxy; C1-C6 alkylthio; or C6-C14 aryl; R3 and R4 each independently represents hydrogen, methyl, ethyl, methoxy, ethoxy, nitro, —CH═CH—CN, or —NR8R9;
or R2 and R3 are both H and R4 and R5 together with the carbon atoms to which they are attached form a condensed benzene ring; or R4 and R5 are both H and R2 and R3 together with the carbon atoms to which they are attached form a condensed benzene ring; or R3 is H and R4 is a radical of the formula (j):

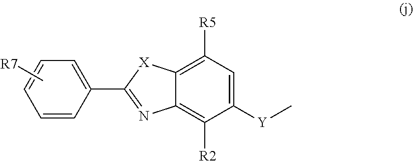

and wherein in all formulas above:

### X is NH, O or S;

Y is a direct bond, —CH₂—, —O—, —CO—, —SO—, —SO₂— or —NR′ where R′ is C1-C6 alkyl optionally substituted with halogen, preferably fluoro; C2-C6 alkenyl or C6-C14 aryl;
R6 is absent or is C1-C6 alkyl or C2-C6 alkenyl, wherein said C1-C6 alkyl may optionally be substituted at the terminal carbon atom by —NR8R9 or —COOR, where R is H, C1-C6 alkyl, C2-C6 alkenyl or C6-C14 aryl;
R7 is hydrogen or at least one group selected from (i) halogen; (ii) nitro; (iii) —NR8R9; (iv) —SO₃H; (v) —OR12; (vi) —SR12; (vii) C1-C6 alkyl optionally substituted by halogen or C1-C6 alkoxy; (viii) C2-C6 alkenyl; (ix) C2-C7 alkanoyl; (x) C6-C14 aryl; (xi) —N═N—R″ where R″ is heteroaryl derived from a mono- or poly-cyclic heteroaromatic ring containing one to three heteroatoms selected from N, O and/or S and being optionally substituted by at least one radical selected from —OH, —COOH or —SO₃H; (xii) benzimidazol-2-yl; (xiii) benzthiazol-2-yl; or (xiv) benzoxazol-2-yl, said radicals (xii), (xiii) and (xiv) being optionally substituted by at least one radical selected from halogen, —NR8R9, —SO₃H, C1-C6 alkyl, C2-C6 alkenyl, C2-C7 alkanoyl, or C1-C6 alkoxy;
R8 and R9 each independently represents hydrogen or C1-C6 alkyl, or R8 is H and R9 is C2-C7 alkanoyl or C7-C15 aroyl optionally substituted by oxo, —SO₃H, —COOH, and/or —NH₂; or the radicals R8 and R9, together with the N atom to which they are attached, form a saturated 5-7 membered heterocyclic ring optionally containing at least one further heteroatom selected from 0, S and/or N, said further N atom being optionally substituted by C1-C6 alkyl;
R10 is hydrogen; C1-C6 alkyl optionally substituted at the terminal carbon atom by —COOR wherein R is H, C1-C6 alkyl, C2-C6 alkenyl or aryl; or C2-C6 alkenyl;
R11 is C1-C6 alkyl optionally substituted by fluoro; C1-C6 alkoxy; C1-C6 alkylthio; or —COOR wherein R is H, C1-C6 alkyl, C2-C6 alkenyl, or aryl;
R12 is C1-C6 alkyl or C2-C6 alkenyl;
and wherein the dotted lines indicate either a double bond stretching from the carbon atom at the 2 position of the benz-1,3-azole ring to the N atom at the ring in which case said N atom is positively charged when R6 is present, or the dotted line represents a double bond stretching from the carbon atom at the 2 position of the benz-1,3-azole ring to the first carbon atom of R1; and
pharmaceutically acceptable salts thereof.

In some embodiments, the benz-1,3-azole derivative of Formula 10 is selected from:

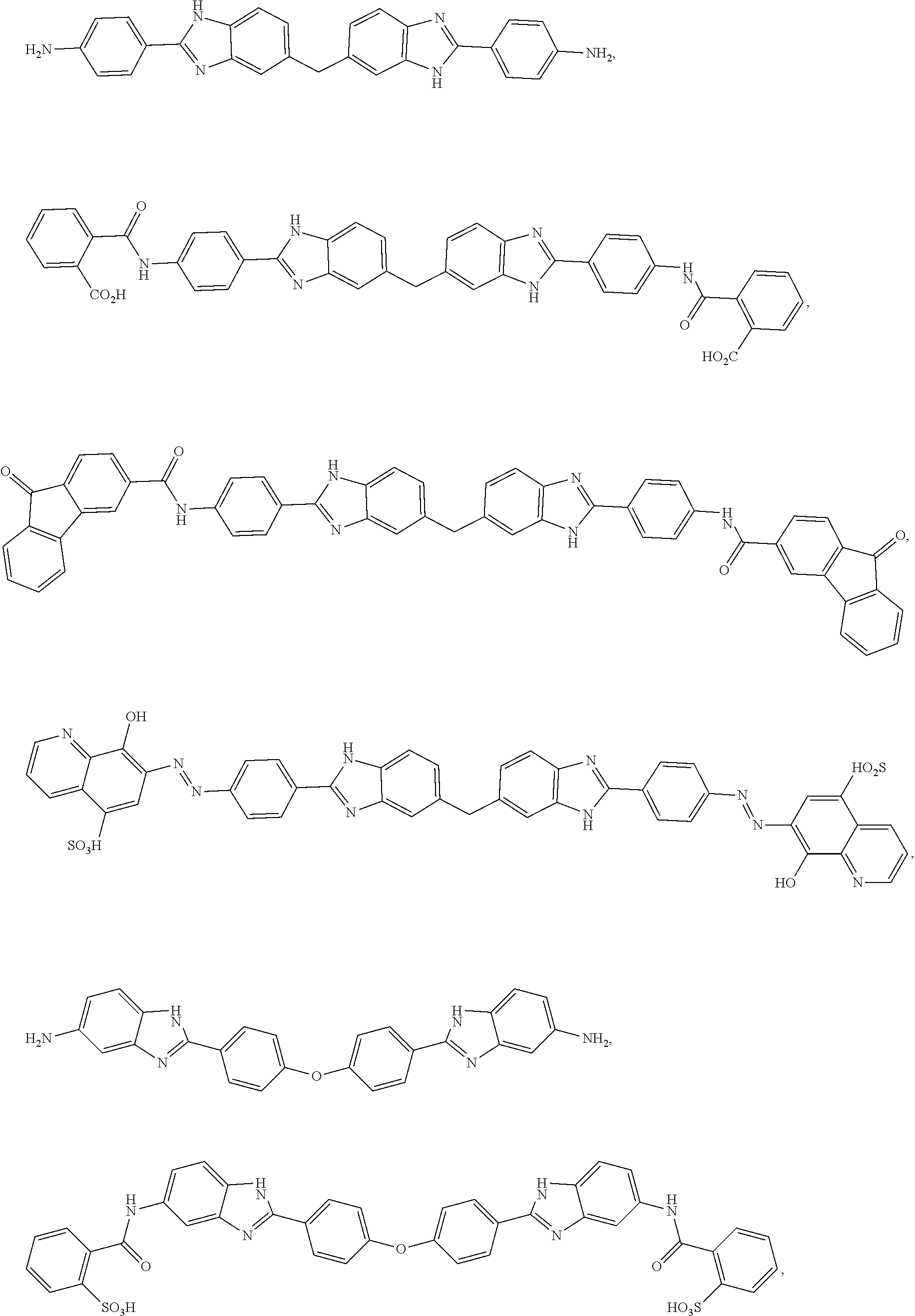

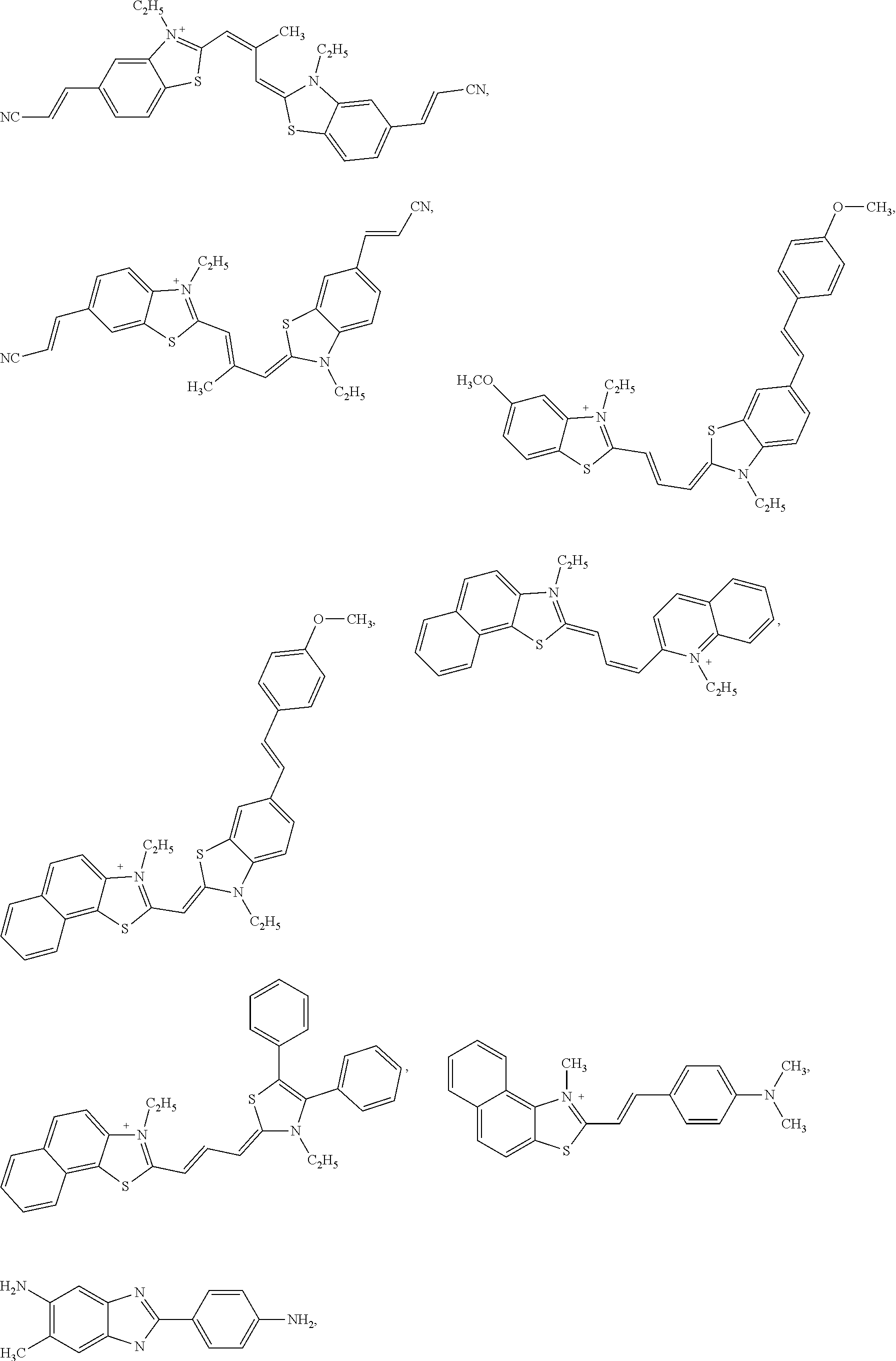

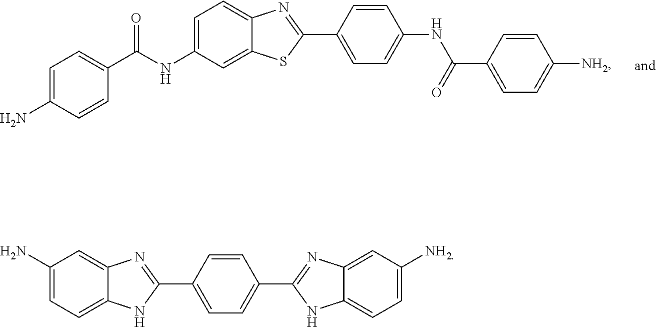 [SMILES: Nc1ccc(C(=O)Nc2ccc(-c3nc4ccc(NC(=O)c5ccc(N)cc5)cc4s3)cc2)cc1 ; Nc1ccc2[nH]c(-c3ccc(-c4nc5cc(N)ccc5[nH]4)cc3)nc2c1]

In some embodiments, the heparanase inhibitor is a bridged saccharide compound described in WO 1995/005182 A1, the entire content of which is herein incorporated by reference. Accordingly, in some embodiments, the heparanase inhibitor is a bridged saccharide compound, comprising the structure:

R¹—[X¹—R²]ₙ—X²—R³

wherein R¹, R² and R³ are independently one or more saccharide(s);
X¹ and X² are independently difunctional or polyfunctional alkyl, aryl or aralkyl compounds capable of covalently joining together said saccharides; and
n is an integer of zero to ten.

In some embodiments, the bridged saccharide compound is selected from ethane-1,2-diyl bis(β-D-glucopyranoside), ethane-1,2-diyl bis(β-D-glucopyranoside) sulfate, 3,6,9,12,15-pentaoxa-heptadecane-1,17-diyl bis(β-D-glucopyranoside), 3,6,9,12,15-pentaoxa-heptadecane-1,17-diyl bis(β-D-glucopyranoside) sulfate, ethane-1,2-diyl bis(O-α-D-glucopyranosyl-(1→4)-β-D-glucopyranoside), ethane-1,2-diyl bis(O-α-D-glucopyranosyl-(1→4)-β-D-glucopyranoside) sulfate, 3,6,9,12,15-pentaoxa-heptadecane-1,17-diyl bis(O-α-D-glucopyranosyl-(1→4)-β-D-glucopyranoside), 3,6,9,12,15-pentaoxa-heptadecane-1,17-diyl bis(O-α-D-glucopyranosyl-(1→4)-β-D-glucopyranoside) sulfate, ethane-1,2-diyl bis(O-α-D-glucopyranosyl-(1→4)-O-α-D-glucopyranosyl-(1→4)-β-D-glucopyranoside), ethane-1,2-diyl bis(O-α-D-glucopyranosyl-(1→4)-O-α-D-glucopyranosyl-(1→4)-β-D-glucopyranoside) sulfate, 3,6,9,12,15-pentaoxa-heptadecane-1,17-diyl bis(O-α-D-glucopyranosyl-(1→4)-O-α-D-glucopyranosyl-(1→4)-β-D-glucopyranoside), 3,6,9,12,15-pentaoxa-heptadecane-1,17-diyl bis(O-α-D-glucopyranosyl-(1→4)-O-α-D-glucopyranosyl-(1→4)-β-D-glucopyranoside) sulfate, ethane-1,2-diyl bis(O-α-D-glucopyranosyl-(1→4)-O-α-D-glucopyranosyl-(1→4)-β-D-glucopyranosyl-(1→4)-O-α-D-glucopyranosyl-(1→4)-O-β-D-glucopyranoside), ethane-1,2-diyl bis(O-α-D-glucopyranosyl-(1→4)-O-α-D-glucopyranosyl-(1→4)-β-D-glucopyranosylucopyranosyl D-glucopyranoside) sulfate, methyl 4-O-{4-O-[4-O-(α-D-glucopyranosyl)-(1→4)-β-D-glucopyranosyloxyethyl}-α-D-glucopyranosyl]-(1→4)-β-D-glucopyranoside, methyl 4-O-{4-O-[4-O-(α-D-glucopyranosyl)-(1→4)-β-D-glucopyranosyloxyethyl]-α-D-glucopyranosyl}-(1→4)-β-D-glucopyranoside sulfate, 2,7-naphthyl bis(O-β-D-galactopyranosyl-(1→4)-β-D-glucopyranoside), 2,7-naphthyl bis(O-β-D-galactopyranosyl-(1→4)-β-D-glucopyranoside) sulfate, 2,7-naphthyl bis(O-β-D-galactopyranosyl-(1→3)-α-D-arabinopyranoside), 2,7-naphthyl bis(O-β-D-galactopyranosyl-(1→3)-α-D-arabinopyranoside) sulfate, 1,5-naphthyl bis(O-β-D-galactopyranosyl)-(1→3)-α-D-arabinopyranoside), 1,5-naphthyl bis(O-β-D-galactopyranosyl)-(1→3)-α-D-arabinopyranoside) sulfate, bis(4-O-β-D-galactopyranosyl-(1→4)-β-D-glucopyranosyl)-1,3-dithio-benzene, and bis(4-O-β-D-galactopyranosyl-(1→4)-β-D-glucopyranosyl)-1,3-dithio-benzene sulfate.

In some embodiments, the heparanase inhibitor is an azasugar derivative described in US 2007/0270354 A1, the entire content of which is incorporated by reference. Accordingly, in some embodiments, the heparanase inhibitor is a compound of Formula 11:

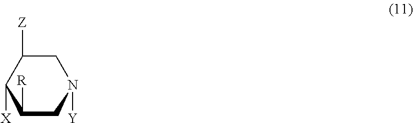

in which:
R represents a hydrogen atom, a hydroxyl radical, an 13 OSO₃⁻ radical, an —O—(C₁-C₅) alkyl radical or an —O-aralkyl radical;
Z represents a COO⁻ radical or a hydroxyl radical;
X represents a hydroxyl radical or a saccharide unit of formula A:

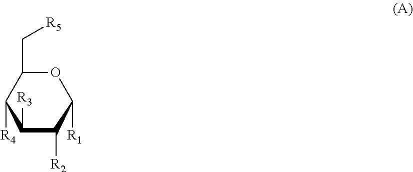

in which:
R₁ represents an oxygen atom, allowing A to bind to the azasugar unit or to another saccharide unit,
R₂ represents an —NH₂ radical, an —NHCO(C₁-C₅)-alkyl radical, an —NHCOaryl radical, an —NHSO₃⁻ radical, a hydroxyl radical, an —O—(C₁-C₅)alkyl radical, an —O-aralkyl radical or an —OSO₃⁻ radical,
R₃ represents a hydroxyl radical, an —OSO₃⁻ radical, an —O—(C₁-C₅)alkyl radical or an —O-aralkyl radical,
R₄ represents a hydroxyl radical, an —OSO₃⁻ radical, an —O—(C₁-C₅) alkyl radical, an —O-aralkyl radical or a saccharide unit of formula B:

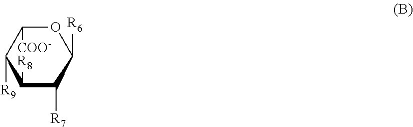

in which:
R₆ represents an oxygen atom, allowing B to bind to another saccharide unit of formula A,
R₇ and R₈ have the same definition as R₃ as defined above,
R₉ represents a hydroxyl group, an —OSO₃⁻ radical, an —O—(C₁-C₅)alkyl radical, an —O-aralkyl radical or a saccharide unit of formula A as defined above,
R₅ has the same definition as R₃ as defined above;
Y represents a hydrogen atom, a (C₁-C₅)alkyl radical or a saccharide unit of formula D

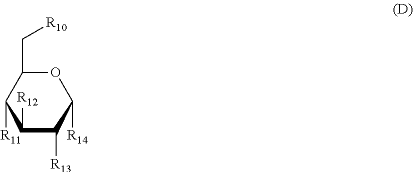

in which:
R₁₀, R₁₂ and R₁₃ have the same definitions as R₅, R₃ and R₂ respectively as defined above,
R₁₁ represents:
a (C₁-C₃)alkylene radical allowing D to attach to the azasugar unit, or
an oxygen atom allowing D to attach to another saccharide unit,
R₁₄ represents an —O—(C₁-C₅)alkyl radical or a radical of formula —O-E in which E represents a radical of formula E:

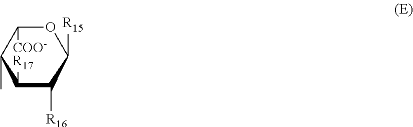

in which:
R₁₅ represents an —O—(C₁-C₅)alkyl radical, an —O-aralkyl radical or a saccharide unit of formula D in which R₁₁ represents an oxygen atom,
R₁₆ and R₁₇ have the same definition as R₃ as defined above,
provided, however, that when X and R each represent a hydroxyl radical, Y does not represent a hydrogen atom, and it being understood that the number of saccharide units of which the compound of Formula 11 is composed is between 1 and 10,
in free form or in the form of salts formed with a pharmaceutically acceptable base or acid, and in the form of solvates or hydrates.

In preferred embodiments, the compound of Formula 11 is selected from:

(2,4-di-O-sodium sulfonato-α-L-idopyranosyluronate of sodium)-(1-4)-(2-acetamido-2-deoxy-6-O-sodium sulfonato-α-D-glucopyranosyl)-(1-4)-(2-O-sodium sulfonato-α-L-idopyranosyluronate of sodium)-(1-4)-(2-acetamido-2-deoxy-6-O-sodium sulfonato-α-D-glucopyranosyl)-(1-4)-(5-(hydroxy)-4-oxypiperidine-3-carboxylate of sodium (3S,4R,5R));
(2,4-di-O-sodium sulfonato-α-L-idopyranosyluronate of sodium)-(1-4)-(2-N-sodium sulfonato-2-deoxy-6-O-sodium sulfonato-α-D-glucopyranosyl)-(1-4)-(2-O-sodium sulfonato-α-L-idopyranosyluronate of sodium)-(1-4)-(2-N-sodium sulfonato-2-deoxy-6-O-sodium sulfonato-α-D-glucopyranosyl)-(1-4)-(5-(hydroxy)-4-oxy-piperidine-3-carboxylate of sodium (3S,4R,5R));
(3-O-methyl-2,4-di-O-sodium sulfonato-α-L-idopyranosyluronate of sodium)-(1-4)-(3-O-methyl-2,6-di-O-sodium sulfonato-α-D-glucopyranosyl)-(1-4)-(3-O-methyl-2-O-sodium sulfonato-α-L-idopyranosyluronate of sodium)-(1-4)-(3-O-methyl-2,6-di-O-sodium sulfonato-α-D-glucopyranosyl)-(1-4)-(5-(hydroxy)-4-oxy-piperidine-3-carboxylate of sodium (3S,4R,5R));
(2,4-di-O-sodium sulfonato-α-L-idopyranosyluronate of sodium)-(1-4)-(2,6)-di-O-sodium sulfonato-α-D-glucopyranosyl)-(1-4)-(2-O-sodium sulfonato-α-L-idopyranosyluronate of sodium)-(1-4)-(2,6-di-O-sodium sulfonato-α-D-glucopyranosyl)-(1-4)-(5-(hydroxy)-4-oxypiperidine-3-carboxylate of sodium (3S,4R,5R));
(4-O-propyl-2-O-sodium sulfonato-α-L-idopyranosyluronate of sodium)-(1-4)-(2,6-di-O-sodium sulfonato-α-D-glucopyransyl-(1-4)-(2-O-sodium sulfonato-α-L-idopyranosyluronate of sodium)-(1-4)-(2,6-di-O-sodium sulfonato-α-D-glucopyranosyl)-(1-4)-(5-(hydroxy)-4-oxypiperidine-3-carboxylate of sodium (3S,4R,5R));
(2,4-di-O-sodium sulfonato-α-L-idopyranosyluronate of sodium)-(1-4)-(2-acetamido-2-deoxy-6-O-sodium sulfonato-α-D-glucopyranosyl)-(1-4)-(2-O-sodium sulfonato-α-L-idopyranosyluronate of sodium)-(1-4)-(2-acetamido-2-deoxy-6-O-sodium sulfonato-α-D-glucopyranosyl)-(1-4)-(3-(hydroxy)-5-hydroxymethyl-4-oxypiperidine (3R,4R,5R)); and
(4-O-phenylpropyl-2-O-sodium sulfonato-α-L-idopyranosyluronate of sodium)-(1-4)-(2-acetamido-2-deoxy-6-O-sodium sulfonato-α-D-glucopyranosyl)-(1-4)-(2-O-sodium sulfonato-α-L-idopyranosyluronate of sodium)-(1-4)-(2-acetamido-2-deoxy-6-O-sodium sulfonato-α-D-glucopyranosyl)-(1-4)-(5-(hydroxy)-4-oxy-piperidine-3-carboxylate of sodium (3S,4R,5R)).

In some embodiments, the heparanase inhibitor is a 4-alkylresorcinol described in EP 2484349 A1, the entire content of which is incorporated herein by reference. Accordingly, in some embodiments, the heparanase inhibitor is a compound of Formula 12:

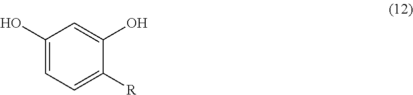

wherein R represents a C1-6 linear or branched alkyl group.

In particular embodiments, the compound of Formula 12 is 4-isobutylresorcinol.

In some embodiments, the heparanase inhibitor is a benzoxazole, benzthiazole or benzimidazole acid derivative described in WO 2004/046122 A1, the entire content of which is herein incorporated by reference. Accordingly, in some embodiments, the heparanase inhibitor is a compound of Formula 13 or a pharmaceutically acceptable salt or prodrug thereof:

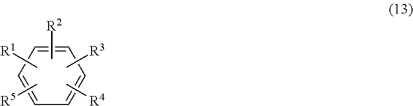

wherein
R¹, R² and R³ are independently, hydrogen, halogen, CF₃, OR⁶, NR⁷R⁸, NR⁸COR¹⁰, NR⁸SO₂R¹⁰ or C₁₋₆ alkyl optionally substituted by hydroxy, C₁₋₆ alkoxy or NR⁷R⁸;
R⁴ is NR⁸CONR⁸R⁹, NR⁸COR⁹, NR⁸SO₂R⁹, or W—CONR⁸R⁹, where W is a bond, C₁₋₆ alkylene, C₂₋₆ alkenylene or C₂₋₆ alkynylene;

R⁵ is

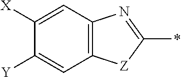

wherein one of X and Y is CO₂H or tetrazole, or C₁₋₆ alkyl or C₂₋₆ alkenyl wherein one of the —CH₂— groups may be replaced with O and wherein the alkyl or alkenyl is substituted with one or more CO₂H or tetrazole groups, and the other is hydrogen; and Z is NR⁸, O or S; R⁶ is hydrogen or C₁₋₆ alkyl, C₃₋₆ alkenyl or C₃₋₆ alkynyl any of which is optionally substituted by hydroxy, C₁₋₆ alkoxy or NR⁷R⁸;
R⁷ is hydrogen or C₁₋₆ alkyl or C₃₋₆ alkenyl either of which is optionally substituted by C₁₋₆ alkoxy or a 5- or 6-membered heterocyclic ring containing up to three heteroatoms selected from NR⁸, S and O;
R⁸ is hydrogen or C₁₋₆ alkyl;
or the groups R⁷ and R⁸ may together with the nitrogen to which they are attached form a 5- or 6-membered ring which optionally contains up to two further heteroatoms selected from NR⁸, S and O;
R⁹ is a group —W—Ar, wherein W is a bond, C₁₋₆ alkylene, C₂₋₆ alkenylene or C₂₋₆ alkynylene and Ar is a 5- to 10-membered carbocyclic group or heterocyclic group which contains up to three heteroatoms selected from O, NR¹¹ and S; the Ar group being optionally substituted by one or more substituents selected from C₁₋₆ alkyl, C₂₋₆ alkenyl, C₃₋₆ alkynyl, halogen, OR⁶, CN, CF₃, OCF₃, NR⁷R⁸, SO₂R¹⁰, COR¹⁰, R¹⁰, methylenedioxo, an oxo group and a 5- to 6-membered heteroaryl group which contains up to two heteroatoms selected from S, O and NR⁸ and which is optionally substituted by one or more substituents selected from halogen, C₁₋₆ alkyl and OR⁶;
R¹⁰ is C₁₋₆ alkyl, C₂₋₆ alkenyl, C₃₋₆ alkynyl or phenyl optionally substituted by one or more substituents selected from halogen, C₁₋₆ alkyl, C₂₋₆ alkenyl, C₃₋₆ alkynyl, CF₃, OCF₃, OR⁶, CN, and methylenedioxo; and
R¹¹ is hydrogen or C₁₋₆ alkyl optionally substituted by phenyl, wherein the phenyl is optionally substituted by one or more substituents selected from halogen, C₁₋₆ alkyl, C₂₋₆ alkenyl, C₃₋₆ alkynyl, CF₃, OCF₃, OR⁶, CN, and methylenedioxo.

In particular embodiments, the compound of Formula 13 is selected from 2-[2-chloro-4-[(4-bromo)phenylacryloylamino]phenyl]-5-benzoxazoleacetic acid, trans 2-[4-[3-(4-bromophenyl)-2-propenamido]-3-fluorophenyl]benzoxazol-5-ylacetic acid, 2-[4-(6-chloro-2H-1-benzopyran-3-carbonylamino)-3-fluorophenyl]benzoxazol-5-ylacetic acid, 2-[4-[3-(4-chlorophenyl)-5-isoxazolecarbonylamino]-3-fluorophenyl]benzoxazol-5-ylacetic acid, trans 2-[4-[3-(2-fluoro-4-trifluoromethylphenyl)-2-propenamido]-3-fluorophenyl]benzoxazol-5-ylacetic acid, and trans 2-[4-[3-(4-bromophenylamino)-3-oxo-1-propenyl]phenyl]benzoxazol-5-yl acetic acid.

In some embodiments, the heparanase inhibitor is a cyclic carboxamide derivative described in EP 2484359 A1, the content of which is incorporated by reference herein in its entirety. Accordingly, in some embodiments, the heparanase inhibitor is a compound of Formula 14:

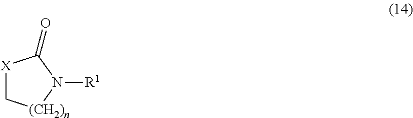

wherein
n is an integer of 1 to 3,
R¹ is hydrogen or a C₁₋₆ hydrocarbon group optionally substituted with hydroxyl,
X is —CH₂— or a group represented by —N(R²)—, where R² is hydrogen or a C₁₋₆ hydrocarbon group optionally substituted with hydroxyl,
or a salt thereof.

In particular embodiments, the compound of Formula 14 is selected from 2-imidazolidinone, 1-(2-hydroxyethyl)-2-imidazolidinone and 1-(2-hydroxyethyl)-2-pyrrolidone.

2.2 Polysaccharides and Derivatives Thereof

In some embodiments, the heparanase inhibitor is a polysaccharide or a derivative thereof. In particular embodiments, the polysaccharide is an anionic polysaccharide, suitable examples of which include a sulfated, phosphorylated or carboxylated polysaccharide; especially a sulfated polysaccharide. In preferred embodiments, the polysaccharide is a polyanionic polysaccharide. The term “polyanionic” is used herein to refer to compounds with more than one anion group. Similarly, the term “polysulfated” is used herein to refer to compounds with more than one sulfate group and, thus, encompasses partially and fully sulfated compounds. The term “sulfated” is used herein to refer to compounds with at least one sulfate group and encompases monosulfated and polysulfated compounds.

In particular embodiments, the polysaccharide is a sulfated polysaccharide, especially a polysulfated polysaccharide.

Suitable polysaccharides include, but are not limited to, pentosan polysulfate; roneparstat (SST0001); fucoidan; heparin; a naturally occurring high molecular weight heparin; a low molecular weight heparin, such as fondaparinux, dalteparin, tinzaparin and enoxaparin; a heparan sulfate; a glycol split heparin, such as those described in Lapierre et al. (1996) Glycobiology, 6(3): 355-366, and Naggi et al. (2005) J Biol Chem, 280(13): 12103-12113, including (periodate-oxidized, borohydride-reduced) heparin, and N-acetylated, glycol split heparin; an N-acetylated heparin, such as those described in Naggi et al. (2005) J Biol Chem, 280(13): 12103-12113; a desulfated heparin, such as those described in Naggi et al. (2005) J Biol Chem, 280(13): 12103-12113, and Lapierre et al. (1996) Glycobiology, 6(3): 355-366, including 2,3-O desulfated heparin; chondroitin polysulfate; chitosan polysulfate; dermatan polysulfate; sulodexide; dextran sulfate; laminarin sulfate; polysulfated inulin; sulfated carboxymethyl chitin III (SCM-chitin III), for example, as described in Saiki et al. (1990) Cancer Res, 50: 3631-3637; calcium spirulan; sulfated beta-cyclodextrin; sulfated gamma-cyclodextrin; necuparanib (M402); a carrageenan, including λ-carrageenan and κ-carrageenan; a glycosaminoglycan derivative described in WO 01/55221 A1 or WO 03/022291 A1; a compound of Formula 15; a compound of Formula 16; a compound of Formula 17; and pharmaceutically acceptable salts and combinations thereof. The entire contents of the publications listed above are herein incorporated by reference.

In some embodiments, the polysaccharide is selected from the group consisting of a low molecular weight heparin, such as fondaparinux, dalteparin, tinzaparin and enoxaparin, pentosan polysulfate, roneparstat, fucoidan, chondroitin polysulfate, chitosan polysulfate, dermatan polysulfate, sulodexide, dextran sulfate, laminarin sulfate, polysulfated inulin, SCM-chitin III, calcium spirulan, sulfated beta-cyclodextrin, sulfated gamma-cyclodextrin, necuparanib, λ-carrageenan, a compound of Formula 15, a compound of Formula 16, a compound of Formula 17, and pharmaceutically acceptable salts and combinations thereof; especially pentosan polysulfate, roneparstat, fucoidan, necuparabib and pharmaceutically acceptable salts and combinations thereof; more especially pentosan polysulfate, roneparstat and pharmaceutically acceptable salts and combinations thereof; most especially pentosan polysulfate or a pharmaceutically acceptable salt thereof.

In particular embodiments, the polysulfated polysaccharide is the sodium salt of pentosan polysulfate, the magnesium salt of pentosan polysulfate, the potassium salt of pentosan polysulfate or the calcium salt of pentosan polysulfate; especially the sodium salt of pentosan polysulfate.

In some embodiments, the heparanase inhibitor is a polysulfated xylan. The polysulfated xylan may be a synthetic, semi-synthetic or naturally occurring polysaccharide or oligosaccharide and may be a purified compound or fraction, or may be a heterogenous mixture. In preferred embodiments, the polysulfated xylan is a linear xylose polymer. Suitable polysulfated xylans include, but are not limited to, xylobiose, xylotriose, xylotetraose, xylohexaose, xyloheptaose, xylooctaose, xylononaose, xylodecaose, pentosan polysulfate and pharmaceutically acceptable salts, derivatives and combinations thereof; especially pentosan polysulfate or a pharmaceutically acceptable salt thereof. In particular embodiments, the polysulfated xylan is pentosan polysulfate.

Pentosan polysulfate, derived form the exhaustive sulfation of xylan, is comprised of a complex mixture of sulfated xylooligosaccharides and polysaccharides, such as linear polymers of β-1→4-linked xylose (Formula 15), and linear polymers where a xylose residue is occasionally substituted at the 2-position with a sulfated 4-O-methyl-α-D-glucuronic acid (Formula 16). In those cases where a 4-O-methyl-α-D-glucuronic acid is present, the xylose residue bearing the glucuronic acid unit may be substituted with an acetyl group on the oxygen of

C-3 (=OAc). The exhaustive sulfation process of pentosan and related oligosaccharides and polysaccharides may not necessarily go to completion, resulting in a partially sulfated oligosaccharide or polysaccharide (i.e. R₁, R₂ and R₄=H) which are contemplated by the invention. Furthermore, as a result of the sulfation process with pyridine sulfur trioxide complex (generated in situ from chlorosulfonic acid and pyridine), pyridine may be incorporated into the oligomer or polymer, for example compounds of Formula 17, and also the reducing ends of the compounds of Formula 16 as described in WO 2014/114723 A1, the content of which is incorporated herein in its entirety. Fractions of pentosan polysulfate with various molecular weight ranges may be obtained by precipitation with various solvents such as ethanol or methanol as described in WO 2012/101544 A1, the entire content of which is herein incorporated by reference.

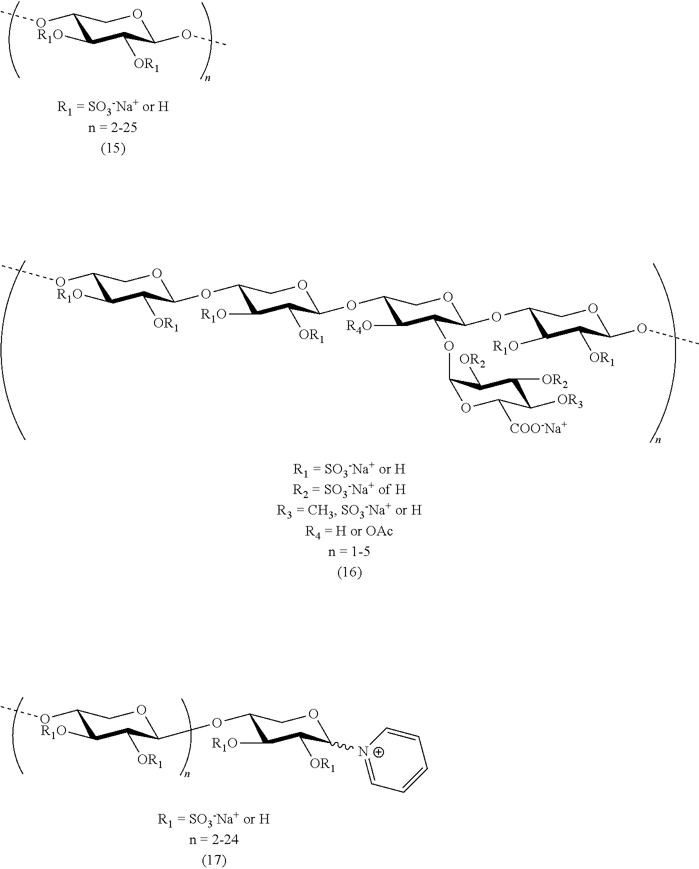

In addition to being a heparanase inhibitor, pentosan polysulfate has been shown to reduce growth factor activity in vivo (Barthlein et al. (2003) J Pediatric Surgery, 38(9): 1296-1304; the entire content of which is incorporated by reference). Reactive oxygen species modulation has also been demonstrated through the protective effect of pentosan polysulfate on pancreas islet beta cells that are highly sensitive to oxidative damage in vitro (US 2013/0143840 A1; and Ziolkowski et al. (2012) J Clin Invest, 122(1): 132-141; the contents of which are hereby incorporated by reference in their entirety).

In some embodiments, the heparanase inhibitor is a glycosaminoglycan derivative described in WO 01/55221 A1, the entire content of which is herein incorporated by reference. Accordingly, in some embodiments, the heparanase inhibitor is a glycosaminoglycan derivative, particularly a desulfated heparin, with a desulfation degree not greater than 60% of the total uronic units.

In particular embodiments, the glycosaminoglycan derivative is a compound of Formula 18:

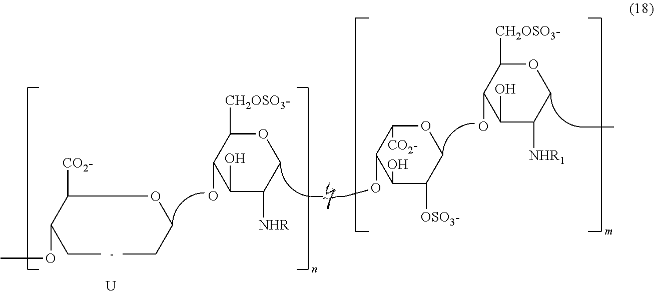

where the U ring can have the following meanings:

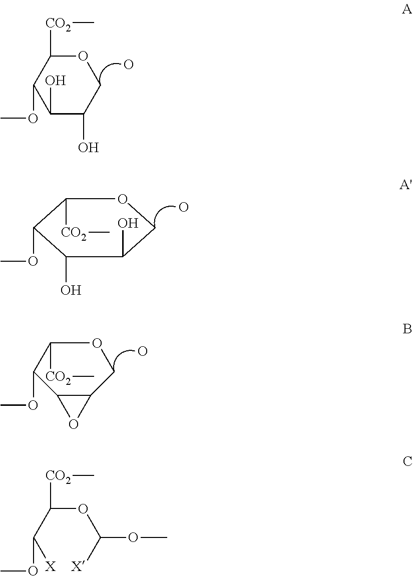

X and X′, which can be the same or different, are an aldehyde group or the —CH₂-D group, where D is hydroxy or an amino acid, a peptide or a residue of a carbohydrate or oligosaccharide;
R and R₁, which can be the same or different, are an SO₃ or acetyl residue;
n and m, which can be the same or different, may vary from 1 to 40; the sum of n+m ranges from 6 to 40; the nn:n ratio ranges from 10:2 to 1:1, the symbol [symbol] indicates that the units marked m and n are statistically distributed along the polysaccharide chain and are not necessarily in sequence.

In particular embodiments, the heparanase inhibitor of Formula 18 is selected from:

heparin partially 2-O-desulfated with a molecular weight (MW) of 12900 Da, a polydispersion index D of 1.5, a desulfation degree of 2.05 (expressed as the SO₃⁻:COO⁻ molar ratio), percentage of modified uronic acids compared to total uronic acids: 5% epoxide groups, 29% oxidated and reduced uronic residues;
heparin partially 2-O-desulfated with a molecular weight (MW) of 11000 Da, a polydispersion index D of 1.5, a desulfation degree of 2.05 (expressed as the SO₃⁻:COO⁻ molar ratio), percentage of modified uronic acids compared to total uronic acids: 5% epoxide groups, 29% uronic residues oxidated and reduced uronic residues;
heparin partially 2-O-desulfated with a molecular weight (MW) of 9200 Da, a polydispersion index D of 1.5, percentage of modified uronic acids compared to total uronic acids: 11% epoxide groups, 27.5% oxidated and reduced uronic residues; and
heparin partially 2-O-desulfated with a molecular weight (MW) of 11200 Da, a polydispersion index D of 1.3, a desulfation degree of 1.99 (expressed as the SO₃⁻:COO⁻ molar ratio), percentage of modified uronic acids compared to total uronic acids approximately 50%.

In some embodiments, the heparanase inhibitor is a glycosaminoglycan derivative described in WO 03/022291 A1, the entire content of which is herein incorporated by reference. Accordingly, in some embodiments, the heparanase inhibitor is a compound of Formula 19:

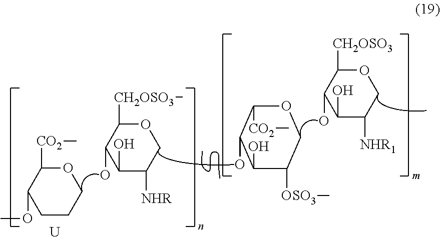

where the U ring can have the following meanings:

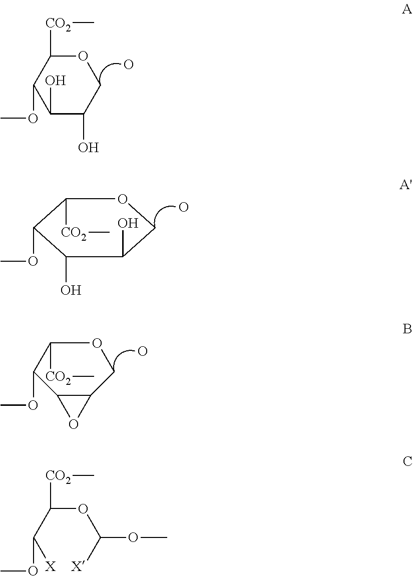

X and X′, which can be the same or different, are an aldehyde group or the —CH₂-D group, where D is hydroxy or an amino acid, a peptide or a residue of a carbohydrate or oligosaccharide;
R and R₁, which can be the same or different, are an SO₃, a C₁-C₈ acyl residue, optionally bearing at least a further carboxy group;
n and m, which can be the same or different, may vary from 1 to 40; the sum of n+m ranges from 6 to 40; the m:n ratio ranges from 10:2 to 1:1, the symbol [symbol] indicates that the units marked m and n are statistically distributed along the polysaccharide chain and are not necessarily in sequence.

In particular embodiments, the compound of Formula 19 is selected from:

partially 2-O-desulfated heparin with a molecular weight (MW) of 11200 Da, a polydispersion index D of 1.3, a desulfation degree of 1.99 (expressed as the SO₃⁻:COO⁻ molar ratio), percentage of modified uronic acids compared to total uronic acids approximately 50%, m:n=1:1 and the units marked m and n are distributed along the polysaccharide chain in a regular, alternating manner;
LMW heparin partially 2-O-desulfated with a molecular weight (MW) of 3050 Da, a polydispersion index of 2.2, a desulfation degree of 1.99 (expressed as the SO₃⁻:COO⁻ molar ratio), a percentage of modified uronic acids compared to total uronic acids of approximately 50%, nn:n=1:1 and the units marked m and n are distributed along the polysaccharide chain in a regular, alternating manner;
LMW heparin partially 2-O-desulfated with a molecular weight of Mn=5800, Mw=7520, a polydispersion index of 1.294, a percentage of modified uronic acids compared to total uronic acids of approximately 50%, nn:n=1:1 and the units marked m and n are distributed along the polysaccharide chain in a regular, alternating manner;
partially 2-O-desulfated heparin with a molecular weight (MW) of 12900 Da, a polydispersion index D of 1.5, a desulfation degree of 1.9 (expressed as the SO₃⁻:COO⁻ molar ratio), percentage of modified uronic acids compared to total uronic acids: 5% epoxide groups, 29% oxidated and reduced uronic residues nn:n=1:1 and the units marked m and n are distributed along the polysaccharide chain in a regular, alternating manner;
partially 2-O-desulfated heparin with a molecular weight (MW) of 9200 Da, a polydispersion index D of 1.5, percentage of modified uronic acids compared to total uronic acids: 11% epoxide groups, 27.5% oxidated and reduced uronic residues, nn:n=1:1 and the units marked m and n are distributed along the polysaccharide chain in a regular, alternating manner; and
2-O-desulfated heparin with a molecular weight (MW) of 11000 Da, a polydispersion index D of 1.5, a desulfation degree of 1.93 (expressed as the SO₃⁻:COO⁻ molar ratio), a percentage of modified uronic acids compared to total uronic acids: 5% epoxide groups, 29% oxidated and reduced uronic residues.

2.3 Oligosaccharides and Derivatives Thereof

In some embodiments, the heparanase inhibitor is an oligosaccharide or a derivative thereof. In particular embodiments, the oligosaccharide is an anionic oligosaccharide, suitable examples of which include a sulfated, phosphorylated or carboxylated oligosaccharide; especially a sulfated oligosaccharide. In preferred embodiments, the oligosaccharide is a polyanionic oligosaccharide.

In particular embodiments, the oligosaccharide is a sulfated oligosaccharide, especially a polysulfated oligosaccharide.

Suitable oligosaccharides or derivatives thereof include, but are not limited to, sucrose octasulfate; muparfostat (PI-88); a PI-88 derivative such as those described in WO 2005/085264 A1, and Karoli et al. (2005) J Med Chem, 48(26): 8229-8236; a sulfated hexasaccharide with a (L-IdoA-D-GlcN)₃ backbone such as hexasaccharide 1, hexasaccharide 2 or hexasaccharide 3 described in Roy et al. (2014) J Med Chem, 57(11): 4511-4520; a sulfated oligosaccharide described in Parish et al. (1999) Cancer Res, 59: 3433-3441; sulfated maltotetraose; sulfated maltoheptaose; sulfated maltopentaose; sulfated maltohexaose; GM 1474 (sulfated maltoheptaoside) described in Tressler et al. (1996) In Molecular, Cellular, and Clinical Aspects of Angiogenesis, Plenum Press New York, page 199; λ-carrageenan oligosaccharides described in Niu et al. (2015) Carbohydrate Polymers, 125: 76-84, including λ-carraheptaose, and Chen et al. (2007) J Agric Food Chem, 55: 6910-6917; a low molecular weight oligosaccharide fraction derived from heparin or heparan sulfate described in WO 90/12580; a trisaccharide or glycol-split trisaccharide described in Ni et al. (2016) Molecules, 21(11): 1602; a sulfated oligosaccharide mimetic described in WO 2010/006982 A1; a sulfated maltooligosaccharide described in WO 1996/009828 A1 and WO 1995/009637 A1, including sulfated maltotetraose, sulfated maltopentaose, sulfated maltohexaose and sulfated maltoheptaose; a sulfated xylan such as a xylobiose, xylotriose, xylotetraose, xylohexaose, xyloheptaose, xylooctaose, xylononaose and xylodecaose; and pharmaceutically acceptable salts and combinations thereof. The entire contents of the publications listed above are herein incorporated by reference.

In some embodiments, the oligosaccharide is selected from the group consisting of muparfostat, sucrose octasulfate, sulfated maltotetraose, sulfated maltoheptaose, sulfated maltopentaose, sulfated maltohexaose, GM 1474, λ-carraheptaose, a sulfated xylan, and pharmaceutically acceptable salts and combinations thereof; especially muparfostat, sulodexide or a pharmaceutically acceptable salt or combination thereof; most especially muparfostat or a pharmaceutically acceptable salt thereof.

In some embodiments, the heparanase inhibitor is a sulfated maltooligosaccharide described in WO 1996/009828 A1 and WO 1995/009637 A1, which are hereby incorporated by reference in their entirety. Accordingly, in some embodiments, the heparanase inhibitor is compound of Formula 20:

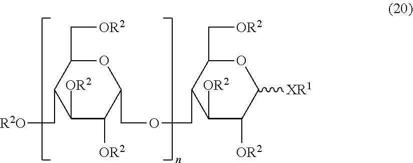

wherein X represents O or S;
each R¹ independently represents an alkyl, aryl, or aralkyl group, a reduced or oxidized glucose unit, SO₃M, or H;
R² represents a SO₃M group or H;
M represents a biologically acceptable cation; and n represents an integer from 1 to 9; with the proviso that at least 50% of R² groups are sulfates.

In particular embodiments, the compound of Formula 20 is selected from a sulfated maltotetraose, sulfated maltopentaose, sulfated maltohexaose and sulfated maltoheptaose.

In some embodiments, the heparanase inhibitor is a low molecular weight oligosaccharide fraction derived from heparin or heparan sulfate described in WO 90/12580, which is hereby incorporated by reference in its entirety. Accordingly, in some embodiments, the heparanase inhibitor is a fraction derived from heparin or heparan sulfate, said fraction comprising low molecular weight oligosaccharides having a molecular weight between 1,000 and 2,000, characterized in that the majority of the low molecular weight oligosaccharides are low sulfate oligosaccharides having a sulfur content of less than 9 weight percent.

In some embodiments, the heparanase inhibitor is a PI-88 derivative described in WO 2005/085264 A1, which is hereby incorporated by reference in its entirety. Accordingly, in some embodiments, the heparanase inhibitor is a compound of Formula 21:

X—[Y]ₙ—Z—UR¹ (21)

wherein:
X, Y and Z are each a monosaccharide unit with a group UR bonded via a single or multiple bond to each non-linking carbon of X, Y and Z, except carbon-1 of monosaccharide Z which bears UR¹ bonded via a single or multiple bond;
n is an integer having a value of 0-6;
each U is independently C, N, S or O or their higher oxidation states, including CO, COO, NO, NO₂, S(O), S(O)O;
each R is independently SO₃M or H, where M is any pharmaceutically acceptable cation or is any alkyl, aryl, acyl, aroyl, alkyl sulfonyl, aryl sulfonyl, PEG, a PEG derivative, H or the group

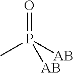

where independently in each AB group, A is O or NH, and B is H, or M where M is as defined above, or an alkyl, aryl or any other suitable group;
R¹ is SO₃M, H, alkyl, aryl, acyl, aroyl, alkyl sulfonyl, aryl sulfonyl, PEG or a PEG derivative, or R¹ together with U is N₃ or a substituted triazole or derivative, or a substituted tetrazole or derivative, or a substituted aryl or derivative, or a substituted heteroaryl or derivative;
with the proviso that when all UR¹ and UR groups are OSO₃M or OH (excluding the exocyclic methylene group of monosaccharide X), the exocyclic methylene group of monosaccharide X cannot be a OPO₃M₂ group.

Optional substituents, unless defined otherwise, include halo (e.g. bromo, fluoro, chloro or iodo), hydroxy, C₁₋₆alkyl (e.g. methyl, ethyl or propyl (n- and i-isomers)), C₁₋₆alkoxy (e.g. methoxy, ethoxy, propoxy (n- and i-isomers), butoxy (n-, sec- and t-isomers)), nitro, amino, C₁₋₆alkylamino (e.g. methyl amino, ethyl amino, propyl (n- and i-isomers) amino), C₁₋₆dialkylamino (e.g. dimethylamino, diethylamino or diisopropylamino), halomethyl (e.g. trifluoromethyl, tribromomethyl or trichloromethyl), halomethoxy (e.g. trifluoromethoxy, tribromomethoxy or trichloromethoxy) and acetyl.

In particular embodiments, the compound of Formula 21 is selected from:

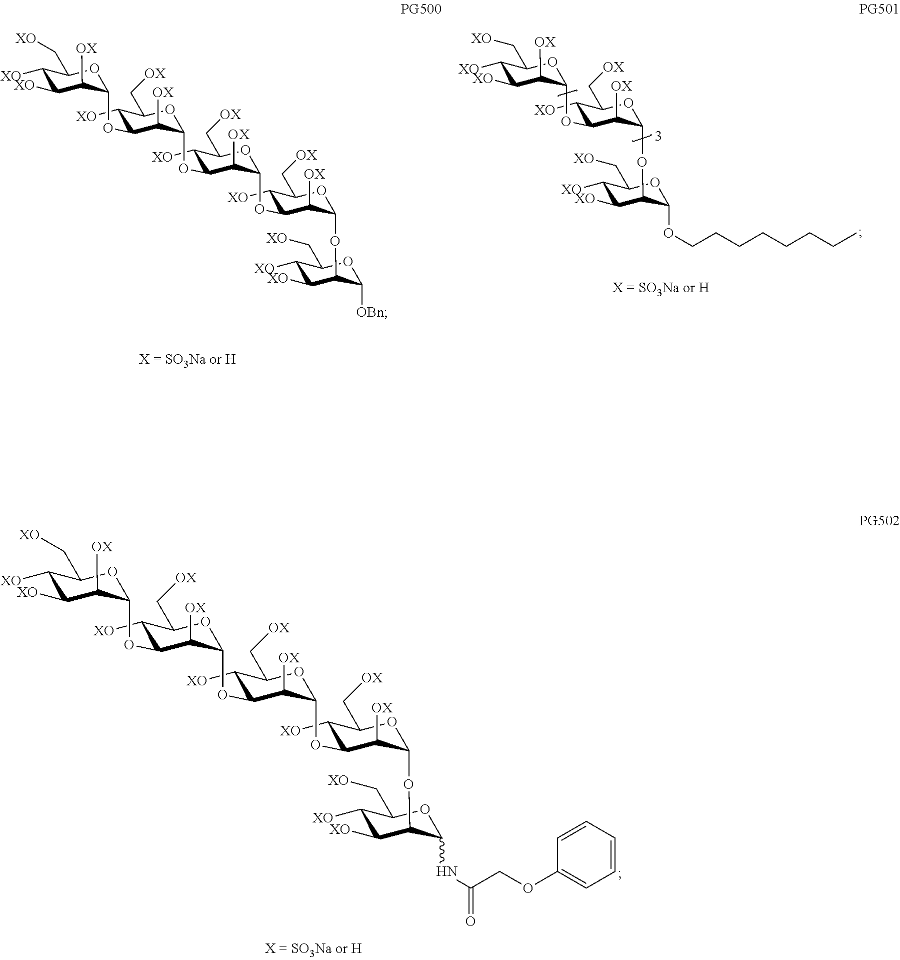

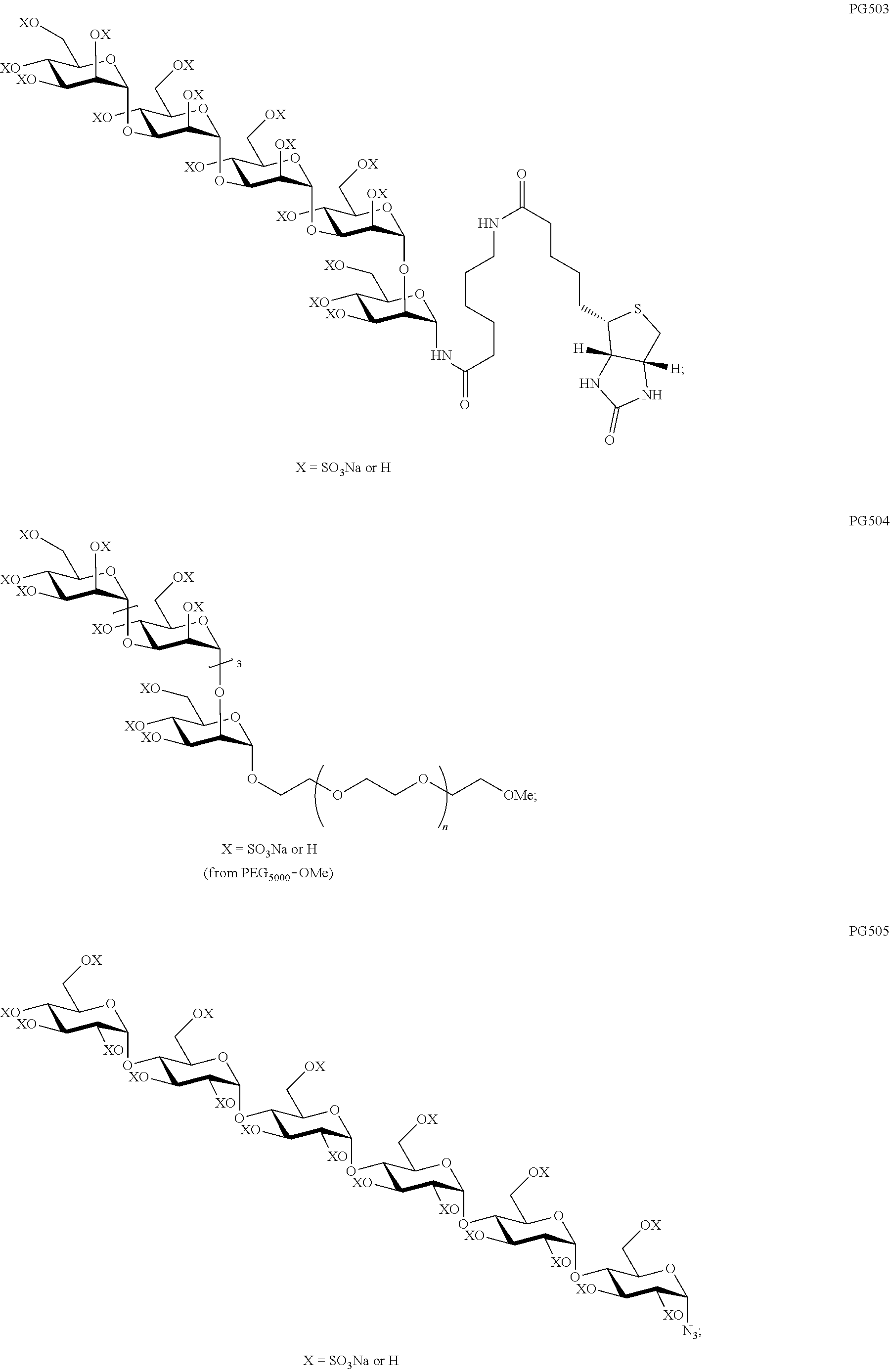

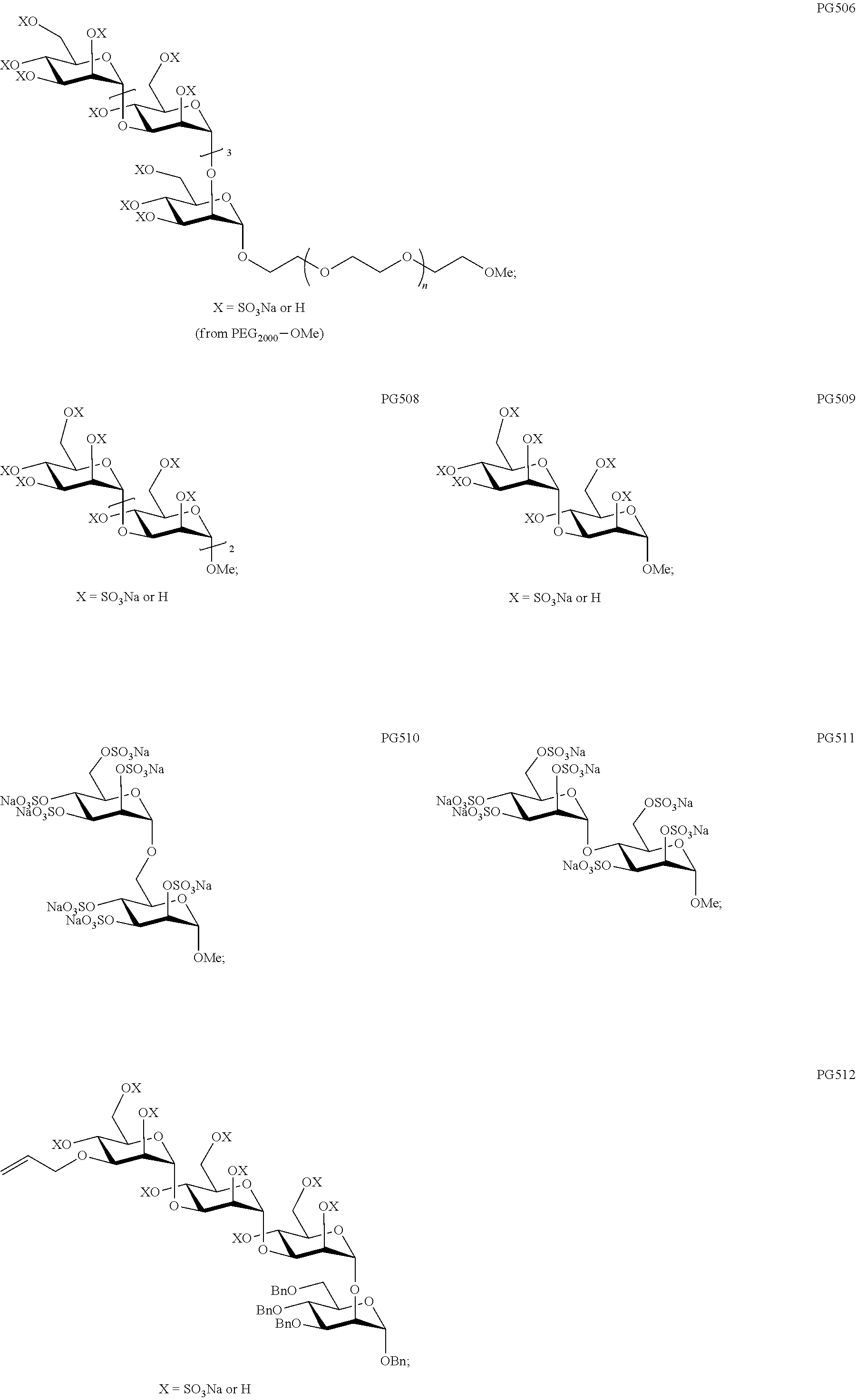

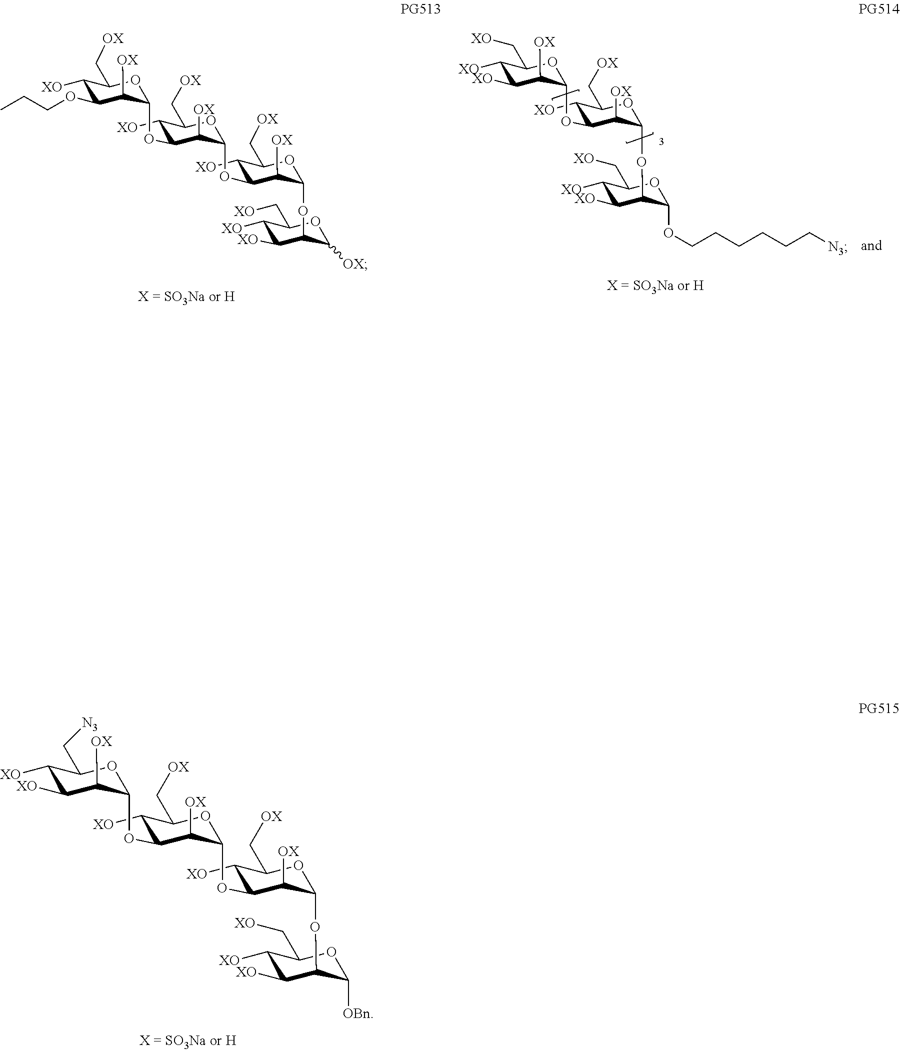

In some embodiments, the heparanase inhibitor is a sulfated oligosaccharide mimetic described in WO 2010/006982 A1, the entire content of which is incorporated herein. Accordingly, in some embodiments, the heparanase inhibitor is a sulfated oligosaccharide wherein a glycosidic bond between two saccharide units is substituted by a C—C bond, and wherein the sulfation degree, expressed as a percentage of OH groups substituted by an OSO₃⁻ group, is comprised between 50 and 100%. In particular embodiments, the sulfated oligosaccharide comprises 4, 5 or 6 saccharide units, preferably 6 saccharide units. In some embodiments, the C—C bond is between positions 1-1, 2-2, 5-5 or 6-6. In preferred embodiments, the sulfated oligosaccharide comprises a C—C bond between the third and fourth saccharide unit.

In some embodiments, the heparanase inhibitor is a glycomimetic saccharopeptide compound described in WO 1996/035700 A1, the entire content of which is herein incorporated by reference. Accordingly, in some embodiments, the heparanase inhibitor is a compound of Formula 22:

W—(X)ₙ—Y—[(X)ₙ—W—(X)ₙ—Y]ₘ—(X)ₙ—W (22)

wherein
W is independently selected from the group consisting of:
a) saccharides;
b) aryl, aralkyl;
c) alkyl of 1 to 8 carbon atoms, optionally substituted with 1-2 substituents selected from the group consisting of lower aryl, lower alkyl, ═O, —OR, —NR′₂, —SR, —SO₄R, —SO₃R, —COOR, -alk-COOR; and
d) cyclic-alkyl or 5-7 carbon atoms, heterocyclic alkyl of 5-7 ring atoms and 1-2 heteroatoms selected from the group consisting of N, O, and S, all optionally substituted with 1-5 substituents selected from the group consisting of ═O, —OH, —OR, —NR′₂, —SR, —SO₄R, —SO₃R, —COOR, and -alk-COOR;
Y is independently selected from the group consisting of —NR³—C(O)— and —C(O)—NR³—;
X is a difunctional or polyfunctional group selected from the group consisting of:
a) aryl, aralkyl;
b) alkyl of 1-8 carbon atoms, optionally substituted with 1-3 substituents in the alkyl backbone or exo to the backbone selected from the group consisting of lower aryl, lower alkyl, —O—, —NR′—, —S—, ═O, —OH, —OR, —NR′₂, —SH, —SR, —SO₄R, —SO₃R, —COOR, and -alk- COOR;
each n is independently 0 or 1;
each m is independently 0 or an integer from 1 to 99 with the proviso that the total number of W groups is 2-100;
R is —H, or lower alkyl, lower aryl, and lower aralkyl;
R′ is independently selected from the group consisting —H, lower alkyl of 1-4 carbon atoms, aralkyl of 2 to 19 carbon atoms, and —C(O)R″;
R″ is lower alkyl of 1 to 4 carbon atoms; and
R³ is selected from the group consisting of —H, alkyl of 1-8 carbon atoms, and aralkyl of 5-8 carbon atoms;
and pharmaceutically acceptable salts thereof;
with the following provisos:
a) when the total number of W groups is 2, then both W groups may not be 2-amino hexoses;
b) at least one W group is a saccharide; and
c) if the terminal W is a N-acetylglucosamine, it may not be linked through —NHCO— at the anomeric carbon to a natural amino acid.

In some embodiments, the heparanase inhibitor is a heparin or heparin mimetic. In this regard, the heparanase inhibitor may be a polysaccharide, oligosaccharide or derivative thereof. In some embodiments, the heparanase inhibitor is selected from the group consisting of pentosan polysulfate; muparfostat; a PI-88 derivative such as those described in WO 2005/085264 A1, and Karoli et al. (2005) J Med Chem, 48(26): 8229-8236; a sulfated hexasaccharide with a (L-IdoA-D-GlcN)₃ backbone such as hexasaccharide 1, hexasaccharide 2 or hexasaccharide 3 described in Roy et al. (2014) J Med Chem, 57(11): 4511-4520; a sulfated oligosaccharide described in Parish et al. (1999) Cancer Res, 59: 3433-3441; sulfated maltotetraose; sulfated maltoheptaose; sulfated maltopentaose; sulfated maltohexaose; GM 1474 (sulfated maltoheptaoside) described in Tressler et al. (1996) In Molecular, Cellular, and Clinical Aspects of Angiogenesis, Plenum Press New York, 199-211; λ-carrageenan oligosaccharides described in Niu et al. (2015) Carbohydrate Polymers, 125: 76-84, including λ-carraheptaose, and Chen et al. (2007) J Agric Food Chem, 55: 6910-6917; a low molecular weight oligosaccharide fraction derived from heparin or heparan sulfate described in WO 90/12580; a sulfated oligosaccharide mimetic described in WO 2010/006982 A1; a sulfated maltooligosaccharide described in WO 1996/009828 A1 and WO 1995/009637 A1, including sulfated maltotetraose, sulfated maltopentaose, sulfated maltohexaose and sulfated maltoheptaose; a sulfated xylan such as a xylotetraose, xylohexaose, xyloheptaose, xylooctaose, xylononaose and xylodecaose; roneparstat (SST0001); fucoidan; heparin; a naturally occurring high molecular weight heparin; a low molecular weight heparin; a heparan sulfate; a glycol split heparin, such as those described in Lapierre et al. (1996) Glycobiology, 6(3): 355-366, and Naggi et al. (2005) J Biol Chem, 280(13): 12103-12113, including (periodate-oxidized, borohydride-reduced) heparin, and N-acetylated, glycol split heparin; an N-acetylated heparin, such as those described in Naggi et al. (2005) J Biol Chem, 280(13): 12103-12113; a desulfated heparin, such as those described in Naggi et al. (2005) J Biol Chem, 280(13): 12103-12113, and Lapierre et al. (1996) Glycobiology, 6(3): 355-366, including 2,3-O desulfated heparin; chondroitin polysulfate; chitosan polysulfate; dermatan polysulfate; sulodexide; dextran sulfate; laminarin sulfate; sulfated carboxymethyl chitin III (SCM-chitin III), for example, as described in Saiki et al. (1990) Cancer Res, 50(12): 3631-3637; calcium spirulan; necuparanib (M402); a carrageenan, including λ-carrageenan and κ-carrageenan; a glycosaminoglycan derivative described in WO 01/55221 A1 or WO 03/022291 A1; a compound of Formula 15; a compound of Formula 16; a compound of Formula 17; and pharmaceutically acceptable salts and combinations thereof; especially pentosan polysulfate, muparfostat or a pharmaceutically acceptable salt thereof; most especially pentosan polysulfate or a pharmaceutically salt thereof. The entire contents of the publications listed above are herein incorporated by reference.

2.4 Oligosaccharide-Aglycone Conjugates

In some embodiments, the heparanase inhibitor is an oligosaccharide-aglycone conjugate. Suitable oligosaccharide-aglycone conjugates include, but are not limited to, 3β-cholestanyl 2,3,4,6-tetra-O-sulfo-α-D-glucopyranosyl-(1→4)-2,3,6-tri-O-sulfo-α-D-glucopyranosyl-(1→4)-2,3,6-tri-O-sulfo-α-D-glucopyranosyl-(1→4)-2,3,6-tri-O-sulfo-β-D-glucopyranoside, tridecasodium salt (PG545); the trisaccharide analogue of PG545 (compound 1), tetrasaccharide analogue of PG545 sulfated at the site occupied by the cholestenol aglycone of PG545 (compound 2) and trisaccharide analogue of PG545 sulfated at the site occupied by the cholestenol aglycone of PG545 (compound 3) described in Hammond et al. (2013) FEBS Open Bio, 3: 346-351; the compounds described in WO 2009/049370 A1; the compounds described in Ferro et al. (2012) J Med Chem, 55(8): 3804-3813; and pharmaceutically acceptable salts and combinations thereof.

The entire contents of the publications listed above is herein incorporated by reference.

In some embodiments, the oligosaccharide-aglycone conjugate is PG545.

In some embodiments, the oligosaccharide-aglycone conjugate is a compound described in WO 2009/049370 A1, the content of which is incorporated by reference in its entirety. Accordingly, in some embodiments, the heparanase inhibitor is a compound of Formula 23:

[X]ₙ—Y—ZR¹R² (23)

wherein:
X and Y are each a monosaccharide unit wherein each hydroxyl group not involved in a glycosidic linkage is substituted independently by a group 50₃M or H, where M is any pharmaceutically acceptable cation;
X and Y are any D- or L-hexose or pentose;
Y is in a cyclic or ring opened form;
Z is O, N, S or C or their higher oxidation states, or a bond, and is linked to the anomeric carbon when Y is a reducing monosaccharide;
R¹ is a linker selected from the group including alkyl, alkenyl, alkynyl, aryl, heteroalkyl, heteroaryl, acyl, aroyl, alkylamido, alkylthioamido, triazolyl, or is a bond;
R² is a lipophilic moiety selected from the group including cholesteryl, cholestanyl, cholate, deoxycholate, straight chain alkyl, branched alkyl, substituted alkyl, straight chain acyl, branched acyl, substituted acyl;
n is an integer from 0-6; and
the level of sulfation of each compound is between 70 and 100% of the total hydroxyl groups.

Optional substituents, unless defined otherwise, include halo (e.g. bromo, fluoro, chloro or iodo), hydroxy, C₁₋₆alkyl (e.g. methyl, ethyl or propyl (n- and i-isomers)), C₁₋₆alkoxy (e.g. methoxy, ethoxy, propoxy (n- and i-isomers), butoxy (n-, sec- and t-isomers)), nitro, amino, C₁₋₆alkylamino (e.g. methyl amino, ethyl amino, propyl (n- and i-isomers) amino), C₁₋₆dialkylamino (e.g. dimethylamino, diethylamino or diisopropylamino), halomethyl (e.g. trifluoromethyl, tribromomethyl or trichloromethyl), halomethoxy (e.g. trifluoromethoxy, tribromomethoxy or trichloromethoxy) and acetyl.

In particular embodiments, the compound of Formula 23 is selected from 3β-cholestanyl 2,3,4,6-tetra-O-sodium sulfonato-α-D-glucopyranosyl-(1→4)-2,3,6-tri-O-sodium sulfonato-α-D-glucopyranosyl-(1→4)-2,3,6-tri-O-sodium sulfonato-α-D-glucopyranosyl-(1→4)-2,3,6-tri-O-sodium sulfonato-β-D-glucopyranoside; 4-(cholestan-3-yl-oxymethyl)[1,2,3]triazol-1-yl 2,3,4,6-tetra-O-sodium sulfonato-α-D-glucopyranosyl-(1→4)-2,3,6-tri-O-sodium sulfonato-α-D-glucopyranosyl-(1→4)-1-deoxy-2,3,6-tri-O-sodium sulfonato-β-D-glucopyranoside; 4-(cholestan-3(3-yl-oxymethyl)[1,2,3]triazol-1-yl 2,3,4,6-tetra-O-sodium sulfonato-α-D-glucopyranosyl-(1→4)-2,3,6-tri-O-sodium sulfonato-α-D-glucopyranosyl-(1→4)-1-deoxy-2,3,6-tri-O-sodium sulfonato-β-D-glucopyranoside; 3β-cholestanyl 2,3,4,6-tetra-O-sodium sulfonato-α-D-glucopyranosyl-(1→4)-2,3,6-tri-O-sodium sulfonato-α-D-glucopyranosyl-(1→4)-2,3,6-tri-O-sodium sulfonato-β-D-glucopyranoside; 3-stearamidopropyl 2,3,4,6-tetra-O-sulfo-α-D-glucopyranosyl-(1→4)-2,3,6-tri-O-sulfo-α-D-glucopyranosyl-(1→4)-2,3,6-tri-O-sulfo-β-D-glucopyranoside, decasodium salt; and 3-stearamidopropyl 2,3,4,6-tetra-O-sulfo-α-D-glucopyranosyl-(1→4)-2,3,6-tri-O-sulfo-α-D-glucopyranosyl-(1→4)-2,3,6-tri-O-sulfo-α-D-glucopyranosyl-(1→4)-2,3,6-tri-O-sulfo-β-D-glucopyranoside, tridecasodium salt.

2.5 Antibodies

The present invention also contemplates the use of antibodies which inhibit heparanase. Accordingly, in some embodiments, the heparanase inhibitor is an antibody. The antibody may be any antibody which inhibits at least one activity of heparanase. In particular embodiments, the antibody is an anti-heparanase antibody, preferably a heparanase neutralizing antibody. While the invention contemplates antibodies which are selective or non-selective heparanase inhibitors, the antibody is preferably a selective heparanase inhibitor. In some embodiments, the antibody inhibits heparanase catalytic activity.

The antibody may be raised against one or more heparanase epitopes, including, but not limited to, a heparan sulfate binding site flanking region, a catalytic proton donor site, a catalytic nucleophilic site, an active site and binding site linking region or a C-terminal sequence of heparanase, such as a C-terminal sequence of the heparanase P8 subunit (described in WO 2004/108065 A1).

Suitable antibodies include, but are not limited to, a rabbit IgG antibody raised against recombinant heparanase described in He et al. (2004) Cancer Res, 64(11): 3928-3933; a rabbit polyclonal antibody raised against the peptide sequence Arg 382 to Phe 398 of human heparanase (UniProt Accession No. Q9Y251) described in Levidiotus et al. (2004) J Am Soc Nephrol, 15(1): 68-78; a rabbit IgG antibody raised against the peptide sequence corresponding to the active site of platelet-derived heparanase (Gly 215 to Asp 234) described in Myler et al. (2006) J Biochem, 139(3): 339-345; a rabbit polyclonal antibody raised against an eight branched peptide consisting of residues 1-15 of the 50 kDa subunit of human heparanase as described in Yang et al. (2009) Cancer Immunol Immunother, 58(9): 1387-1396; a rabbit polyclonal antibody raised against an eight branched peptide consisting of residues 279-293 of the 50 kDa subunit of human heparanase as described in Yang et al. (2009) Cancer Immunol Immunother, 58(9): 1387-1396; a mouse monoclonal antibody directed against a peptide corresponding to Lys 158 to Asn 171 of human heparanase, which comprises the substrate binding domain as described in Weissman et al. (2016) PNAS, 113(3): 704-709; a mouse monoclonal antibody (IgG) directed against full-length human heparanase, mAB H1023, as described in Weissman et al. (2016) PNAS, 113(3): 704-709; a rabbit polyclonal antibody raised against the 65 kDa heparanase precursor, Ab 1453, as described in Zester et al. (2004) Journal of Cell Science, 117: 2249-2258; a monoclonal anti-heparanase antibody described in WO 00/25817 A1; an antibody or portion thereof described in WO 2004/108065 A2; a monoclonal antibody described in WO 2004/043989 A2; and combinations thereof. In some embodiments, the antibody is a mouse monoclonal antibody directed against a peptide corresponding to Lys 158 to Asn 171 of human heparanase, which comprises the substrate binding domain as described in Weissman et al. (2016) PNAS, 113(3): 704-709; a mouse monoclonal antibody (IgG) directed against full-length human heparanase, mAB H1023, as described in Weissman et al. (2016) PNAS, 113(3): 704-709; or a combination thereof. The entire contents of the publications listed above is herein incorporated by reference.

In some embodiments, the heparanase inhibitor is an antibody or portion thereof described in WO 2004/108065 A2, the entire content of which is herein incorporated by reference. Accordingly, in some embodiments, the heparanase inhibitor is an isolated antibody or portion thereof capable of specifically binding to at least one epitope of a heparanase protein, said heparanase protein being at least 60% homologous to the amino acid sequence of any one of SEQ ID NOs: 1-5 and 11. In some embodiments, the heparanase protein is at least 70%, 80% or 90% homologous to the amino acid sequence of any one of SEQ ID NOs: 1-5 and 11. In some embodiments, the heparanase protein comprises the amino acid sequence of any one of SEQ ID NOs: 1-5 and 11. In some embodiments, the at least one epitope comprises a sequence being at least 70%, 80% or 90% homologous to the amino acid sequence of any one of SEQ ID NOs: 6-10. In particular embodiments, the at least one epitope comprises an amino acid sequence of any one of SEQ ID NOs: 6-10. The antibody or portion thereof may be a polyclonal, chimeric, humanized, single chain, immobilized, labeled, or monoclonal antibody, or may be a Fab fragment.

SEQ ID NO: 1
KKFKNSTYSRSSVDVLYTFANCSGLDLIFGLNALLRTADLQWNSSNAQLLLDYCSSKGYNISWELGNEPNSFLKK
ADIFINGSQLGEDFIQLHKLLRKSTFKNAKLYGPDVGQPRRKTAKMLKSFLKAGGEVIDSVTWHHYYLNGRTATR
EDFLNPDVLDIFISSVQKVFQVVESTRPGKKVWLGETSSAYGGGAPLLSDTFAAGFMWLDKLGLSARMGIEVVM
RQVFFGAGNYHLVDENFDPLPDYWLSLLFKKLVGTKVLMASVQGSKRRKLRVYLHCTNTDNPRYKEGDLTLYAIN
LHNVTKYLRLPYPFSNKQVDKYLLRPLGPHGLLSKSVQLNGLTLKMVDDQTLPPLMEKPLRPGSSLGLPAFSYSFF
VIRNAKVAACI
SEQ ID NO: 2
MLRLLLLWLWGPLGALAQGAPAGTAPTDDVVDLEFYTKRPLRSVSPSFLSITIDASLATDPRFLTFLGSPRLRALA
RGLSPAYLRFGGTKTDFLIFDPDKEPTSEERSYWKSQVNHDICRSEPVSAAVLRKLQVEWPFQELLLLREQYQKE
FKNSTYSRSSVDMLYSFAKCSGLDLIFGLNALLRTPDLRWNSSNAQLLLDYCSSKGYNISWELGNEPNSFWKKA
HILIDGLQLGEDFVELHKLLQRSAFQNAKLYGPDIPIyQPRGKTVKLLRSFLKAGGEVIDSLTWHHYYLNGRIATKE
DFLSSDALDTFILSVQKILKVTKEITPGKKVWLGETSSAYGGGAPLLSNTFAAGFMWLDKLGLSAQMGIEVVMR
QVFFGAGNYHLVDENFEPLPDYWLSLLFKKLVGPRVLLSRVKGPDRSKLRVYLHCTNVYHPRYQEGDLTLYVLNL
HNVTKHLKVPPPLFRKPVDTYLLKPSGPDGLLSKSVQLNGQILKMVDEQTLPALTEKPLPAGSALSLPAFSYGFFVI
RNAKIAACI
SEQ ID NO: 3
MLRPLLLLWLWGRLRALTQGTPAGTAPTKDVVDLEFYTKRLFQSVSPSFLSITIDASLATDPRFLTFLGSPRLRALA
RGLSPAYLRFGGTKTDFLIFDPNKEPTSEERSYWQSQDNNDICGSERVSADVLRKLQMEWPFQELLLLREQYQR
EFKNSTYSRSSVDMLYSFAKCSRLDLIFGLNALLRTPDLRWNSSNAQLLLNYCSSKGYNISWELGNEPNSFWKK
AQISIDGLQLGEDFVELHKLLQKSAFQNAKLYGPDIGQPRGKTVKLLRSFLKAGGEVIDSLTWHHYYLNGRVATK
EDFLSSDVLDTFILSVQKILKVTKEMTPGKKVWLGETSSAYGGGAPLLSNTFAAGFMWLDKLGLSAQLGIEVVMR
QVFFGAGNYHLVDENFEPLPDYWLSLLFKKLVGPKVLMSRVKGPDRSKLRVYLHCTNVYHPRYREGDLTLYVLNL
HNVTKHLKLPPPMFSRPVDKYLLKPFGSDGLLSKSVQLNGQTLKMVDEQTLPALTEKPLPAGSSLSVPAFSYGFFV
IRNAKIAACI
SEQ ID NO: 4
MLLRSKPALPPPLMLLLLGPLGPLSPGALPRPAQAQDVVDLDFFTQEPLHLVSPSFLSVTIDANLATDPRFLILLGSP
KLRTLARGLSPAYLRFGGTKTDFLIFDPKKESTFEERSYWQSQVNQDICKYGSIPPDVEEKLRLEWPYQEQLLLRE
HYQKKFKNSTYSRSSVDVLYTFANCSGLDLIFGLNALLRTADLQWNSSNAQLLLDYCSSKGYNISWELGNEPNS
FLKKADIFINGSQLGEDFIQLHKLLRKSTFKNAKLYGPDVGQPRRKTAKMLKSFLKAGGEVIDSVTWHHYYLNGR
TATREDFLNPDVLDIFISSVQKVFQVVESTRPGKKVWLGETSSAYGGGAPLLSDTFAAGFMWLDKLGLSARMGI
EVVMRQVFFGAGNYHLVDENFDPLPDYWLSLLFKKLVGTKVLMASVQGSKRRKLRVYLHCTNTDNPRYKEGDLT
LYAINLHNVTKYLRLPYPFSNKQVDKYLLRPLGPHGLLSKSVQLNGLTLKMVDDQTLPPLMEKPLRPGSSLGLPAF
SYSFFVIRNAK
VAACI
SEQ ID NO: 5
MLVLLLLVLLLAVPPRRTAELQLGLREPIGAVSPAFLSLTLDASLARDPRFVALLRHPKLHTLASGLSPGFLRFGGTS
TDFLIFNPNKDSTWEEKVLSEFQAKDVCEAWPSFAVVPKLLLTQWPLQEKLLLAEHSWKKHKNTTITRSTLDILH
TFASSSGFRLVFGLNALLRRAGLQWDSSNAKQLLGYCAQRSYNISWELGNEPNSFRKKSGICIDGFQLGRDFVH
LRQLLSQHPLYRHAELYGLDVGQPRKHTQHLLRSFMKSGGKAIDSVTWHHYYVNGRSATREDFLSPEVLDSFAT
AIHDVLGIVEATVPGKKVWLGETGSAYGGGAPQLSNTYVAGFMWLDKLGLAARRGIDVVMRQVSFGAGSYHLV
DAGFKPLPDYWLSLLYKRLVGTRVLQASVEQADARRPRVYLHCTNPRHPKYREGDVTLFALNLSNVTQSLQLPKQ
LWSKSVDQYLLLPHGKDSILSREVQLNGRLLQMVDDETLPALHEMALAPGSTLGLPAFSYGFYVIRNAKAIACI
SEQ ID NO: 6
CTNTDNPRYK
SEQ ID NO: 7
PAYLRFGGTKTDFLIFDPK
SEQ ID NO: 8
SWELGNEPNSFLKKA
SEQ ID NO: 9
RPGKKVWLGETSSAY
SEQ ID NO: 10
TWHHYYLNGRTATR
SEQ ID NO: 11
QDVVDLDFFTQEPLHLVSPSFLSVTIDANLATDPRFLILLGSPKLRTLARGLSPAYLRFGGTKTDFLIFDPKKE

In some embodiments, the heparanase inhibitor is a monoclonal antibody described in WO 2004/043989 A2, the entire content of which is herein incorporated by reference. Accordingly, in some embodiments, the heparanase inhibitor is an isolated human monoclonal antibody which binds to and inhibits activity of human heparanase. In some embodiments, the antibody is encoded by a human heavy chain nucleic acid comprising a nucleotide sequence in the variable region selected from the group consisting of the nucleotide sequences as set forth in SEQ ID NOs: 12, 13, 14, 15 and 16, and a human kappa light chain nucleic acid comprising a nucleotide sequence in the variable region selected from the group consisting of the nucleotide sequences as set forth in SEQ ID NOs: 17, 18, 19, 20 and 21, and conservative sequence modifications thereof. In some embodiments, the antibody comprises a heavy chain variable region comprising an amino acid sequence selected from the group consisting of the amino acid sequences as set forth in SEQ ID NOs: 22, 23, 24, 25 and 26, and a kappa light chain variable region comprising an amino acid sequence selected from the group consisting of the amino acid sequences as set forth in SEQ ID NOs: 22, 23, 24, 25 and 26, and conservative sequence modifications thereof. In some embodiments, the antibody comprises a CDR domain having a human heavy and light chain CDR1 region, a human heavy and light chain CDR2 region, and a human heavy and light chain CDR3 region, wherein (a) the CDR1 human heavy and light chain region comprises the amino acid sequence CDR1 of SEQ ID NO: 27 and SEQ ID NO: 28, respectively, and conservative sequence modifications thereof, and (b) the CDR2 human heavy and light chain region comprises the amino acid sequence CDR2 of SEQ ID NO: 29 and SEQ ID NO: 30, respectively, and conservative sequence modifications thereof, and (c) the CDR3 human heavy and light chain region comprises the amino acid sequence CDR3 of SEQ ID NO: 31 and SEQ ID NO: 32, respectively, and conservative sequence modifications thereof.

SEQ ID NO: 12
cag gtc cag ctg gta cag tct ggg gct gag gtg aag aag cct ggg gcc tca gtg aag gtc tcc tgc aag gtt tcc
gga tac acc ctc act gaa tta tcc atg cac tgg gtg cga cag gct cct gga aaa ggg ctt gag tgg atg gga ggt
ttt gat cct gaa gat ggt gaa aca atc tac gca cag aag ttc cag gac aga gtc acc atg acc gag gac aca tct
aca gac aca gcc tac atg gag ctg agc agc ctg aga tct gag gac acg gcc gta tat tac tgt aca aca gag agc
ttg gta cga tat ttt gac tgg tta tcc cac ttt gac tac tgg ggc cag gga acc ctg gtc acc gtc tcc tca
SEQ ID NO: 13
cag gtc cag ctg gta cag tct ggg gct gag gtg aag aag cct ggg gcc tca gtg aag gtc tcc tgc aag gtt tcc
gga tac acc ctc act gaa tta tcc atg cac tgg gtg cga cag gct cct gga aaa ggg ctt gag tgg atg gga ggt
ttt gat cct gaa gat ggt gaa aca atc tac gca cag aag ttc cag ggc aga gtt acc atg acc gag gac aca tct
aca gac aca gcc tac atg gag ctg agc agc ctg aga tct gac gac acg gcc gtg tat tac tgt gca aca gag agc
ttg gta cga tat ttt gac tgg tta tcc cac ttt gac tac tgg ggc cag gga acc ctg gtc acc gtc tcc tca
SEQ ID NO: 14
gag gtg cag ctg gtg gag tct ggg gga ggc ttg gtc cag cct ggg ggg tcc ctg aga ctc tcc tgt gca gcc tct
gga ttc acc ttt agt agc tat tgg atg agc tgg gtc cgc cag gct cca ggg aag ggg ctg gag tgg gtg gcc agc
ata tac caa gat gga agt gag aaa tac tat gtg gac tct gtg aag ggc cga ttc acc atc tcc aga gac aac gcc
aag aac tca ctg tat ctg caa atg aac agc ctg aga gcc gag gac acg gct atg tat tac tgt gcg aga gaa tta
gac tgg gga tgg gac tac tgg ggc cag gga acc ctg gtc acc gtc tcc tca
SEQ ID NO: 15
cag gtc cag ctg gta cag tct ggg gct gag gtg aag aag cct ggg gcc tca gtg aag gtc tcc tgc aag gtt tcc
gga tac acc ctc act gaa tta tcc atg cac tgg gtg cga cag gct cct gga aaa ggg ctt gag tgg atg gga ggt
ttt gat cct gaa gat ggt gaa aca atc tac gca cag aag ttc cag ggc aga gtc acc atg acc gag gac aca tct
aca gac aca gcc tac atg gag ctg agc agc ctg aga tct gag gac acg gcc gtg tat tac tgt gca aca gag agc
ttg gta cga tat ttt gac tgg tta tcc cac ttt gac tac tgg ggc cag gga acc ctg gtc acc gtc tcc tca
SEQ ID NO: 16
gag gtg cag ctg gtg gag tct ggg gga ggc ttg gtc cag cct ggg ggg tcc ctg aga ctc tcc tgt gca gcc tct
gga ttc acc ttt agt agc tat tgg atg agc tgg gtc cgc cag gct cca ggg aag ggg ctg gag tgg gtg gcc agc
ata tac caa gat gga agt gag aaa tac tat gtg gac tct gtg aag ggc cga ttc acc atc tcc aga gac aac gcc
aag aac tca ctg tat ctg caa atg aac agc ctg aga gcc gag gac acg gct gtg tat tac tgt gcg aga gaa tta
gac tgg gga tgg gac tac tgg ggc cag gga acc ctg gtc acc gtc tcc tca
SEQ ID NO: 17
gac atc cag atg acc cag tct cca tcc tca ctg tct gca tct gta gga gac aga gtc acc atc act tgt cgg gcg
agt cag ggt att agc agc tgg tta gcc tgg tat cag cag aaa cca gag aaa gcc cct aag tcc ctg atc tat gct
gca tcc agt ttg caa agt ggg gtc cca tca agg ttc agc ggc agt gga tct ggg aca gat ttc act ctc acc atc
agc agc ctg cag cct gaa gat ttt gca act tat tac tgc caa cag tat aat agt tac ccg tac act ttt ggc cag ggg
acc aag ctg gag atc aaa
SEQ ID NO: 18
gac atc cag atg acc cag tct cca tcc tca ctg tct gca tct gta gga gac aga gtc acc atc act tgt cgg gcg
agt cag ggt att agc agc tgg tta gcc tgg tat cag cag aaa cca gag aaa gcc cct aag tcc ctg atc tat gct
gca tcc agt ttg caa agt ggg gtc cca tca agg ttc agc ggc agt gga tct ggg aca gat ttc act ctc acc atc
agc agc ctg cag cct gaa gat ttt gca act tat tac tgc caa cag tat aat agt tac ccg tac act ttt ggc cag ggg
acc aag ctg gag atc aaa
SEQ ID NO: 19
gcc atc cag ttg acc cag tct cca tcc tcc ctg tct gca tct gta gga gac aga gtc acc atc act tgc cgg gca agt
cag ggc att agc agt gct tta gcc tgg tat cag cag aaa cca ggg aaa gct cct aag ctc ctg atc tat gat gcc
tcc agt ttg gaa agt ggg gtc cca tca agg ttc agc ggc agt gga tct ggg aca gat ttc act ctc acc atc agc
agc ctg cag cct gaa gat ttt gca act tat tac tgt caa cag ttt aat agt tac ccg atc acc ttc ggc caa ggg aca
cga ctg gag att aaa
SEQ ID NO: 20
gac atc cag atg acc cag tct cca tcc tca ctg tct gca tct gta gga gac aga gtc acc atc act tgt cgg gcg
agt cag ggt att agc agc tgg tta gcc tgg tat cag cag aaa cca gag aaa gcc cct aag tcc ctg atc tat gct
gca tcc agt ttg caa agt ggg gtc cca tca agg ttc agc ggc agt gga tct ggg aca gat ttc act ctc acc atc
agc agc ctg cag cct gaa gat ttt gca act tat tac tgc caa cag tat aat agt tac ccg tac act ttt ggc cag ggg
acc aag ctg gag atc aaa
SEQ ID NO: 21
gcc atc cag ttg acc cag tct cca tcc tcc ctg tct gca tct gta gga gac aga gtc acc atc act tgc cgg gca agt
cag ggc att agc agt gct tta gcc tgg tat cag cag aaa cca ggg aaa gct cct aag ctc ctg atc tat gat gcc
tcc agt ttg gaa agt ggg gtc cca tca agg ttc agc ggc agt gga tct ggg aca gat ttc act ctc acc atc agc
agc ctg cag cct gaa gat ttt gca act tat tac tgt caa cag ttt aat agt tac ccg atc acc ttc ggc caa ggg aca
cga ctg gag att aaa
SEQ ID NO: 22
QVQLVQSGAEVKKPGASVKVSCKVSGYTLTELSMHWVRQAPGKGLEWMGGFDPEDGETIYAQKFQDRVTMTE
DTSTDTAYMELSSLRSEDTAVYYCTTESLVRYFDWLSHFDYWGQGTLVTVSS
SEQ ID NO: 23
QVQLVQSGAEVKKPGASVKVSCKVSGYTLTELSMHWVRQAPGKGLEWMGGFDPEDGETIYAQKFQGRVTMTE
DTSTDTAYMELSSLRSDDTAVYYCATESLVRYFDWLSHFDYWGQGTLVTVSS
SEQ ID NO: 24
EVQLVESGGGLVQPGGSLRLSCAASGFTFSSYWMSWVRQAPGKGLEWVASIYQDGSEKYYVDSVKGRFTISR
DNAKNSLYLQMNSLRAEDTAMYYCARELDWGWDYWGQGTLVTVSS
SEQ ID NO: 25
QVQLVQSGAEVKKPGASVKVSCKVSGYTLTELSMHWVRQAPGKGLEWMGGFDPEDGETIYAQKFQGRVTMTE
DTSTDTAYMELSSLRSEDTAVYYCATESLVRYFDWLSHFDYWGQGTLVTVSS
SEQ ID NO: 26
EVQLVESGGGLVQPGGSLRLSCAASGFTFSSYWMSWVRQAPGKGLEWVASIYQDGSEKYYVDSVKGRFTISR
DNAKNSLYLQMNSLRAEDTAVYYCARELDWGWDYWGQGTLVTVSS
SEQ ID NO: 27
ELSMH
SEQ ID NO: 28
RASQGISSWLA
SEQ ID NO: 29
GFDPEDGETIYAQKFQG
SEQ ID NO: 30
AASSLQS
SEQ ID NO: 31
ESLVRYFDWLSHFDY
SEQ ID NO: 32
QQYNSYPYT

2.6 Protein Inhibitors

The heparanase inhibitor may also be a protein, peptide or polypeptide. Suitable protein, peptide or polypeptide heparanase inhibitors include, but are not limited to, histidine-rich glycoprotein described in Freeman and Parish (1997) Biochem J, 325: 229-237; major basic protein described in Temkin et al. (2004) J Allergy Clin Immunol, 113(4): 703-709; and a peptide of heparan sulfate interacting protein (CRPKAKAKAKAKDQTK) described in Marchetti et al. (1997) J Biol Chem, 272(25): 15891-15897; and pharmaceutically acceptable salts and combinations thereof. The entire contents of the publications listed above is herein incorporated by reference.

2.7 Polymeric Inhibitors

The present invention also contemplates the use of polymeric heparanse inhibitors.

Suitable polymeric inhibitors include, but are not limited to, polymers of carboxylated phenols including a polymer of (4-hydroxyphenoxy)acetic acid with a molecular weight of approximately 5800 Da (RG-13577) described in Benezra et al. (2002) J Cell Physiol, 192(3): 245-358; poly(N-acryl amino acids) including poly(N-acrylleucine) described in Bentolila et al. (2000) J Med Chem, 43(13): 2591-2600; and pharmaceutically acceptable salts and combinations thereof. In particular embodiments, the polymeric inhibitor is a poly(N-acryl amino acids) including poly(N-acrylleucine) described in Bentolila et al. (2000) J Med Chem, 43(13): 2591-2600, or a pharmaceutically acceptable salt or combination thereof. The entire contents of the publications listed above is herein incorporated by reference.

2.8 Screening Assay to Identify Heparanase Inhibitors

The present invention not only encompasses known heparanase inhibitors but heparanase inhibitors identified by any suitable screening assay. Accordingly, the present invention also extends to methods of screening for agents that are useful for inhibiting heparanase and, in turn, for treating, or inhibiting the progression or development of, an ocular inflammatory disorder, such as diabetic retinopathy or AMD.

In some embodiments, the screening methods comprise: (1) contacting a preparation with a test agent, wherein the preparation comprises a polypeptide comprising an amino acid sequence corresponding to at least a biologically active fragment of heparanase or to a variant or derivative thereof; and (2) detecting a change in the functional activity of the polypeptide relative to a reference functional activity in the absence of the test agent. A detected reduction in the functional activity of the polypeptide relative to a normal or reference functional activity in the absence of the test agent indicates that the test agent is useful for treating, or inhibiting the development or progression of, an ocular inflammatory disorder. Suitably this is confirmed by analyzing or detecting whether the test agent treats, or inhibits the development or progression of, an ocular inflammatory disorder.

Screening for heparanase inhibitors according to the invention may be achieved by any suitable method. Suitable assays for identifying heparanase inhibitors are known in the art, see, for example, the in vitro assays described in Rivara et al. (2016) Future Med Chem, 8(6): 647-680. For example, the method may include contacting a preparation comprising heparanase and a heparanase substrate (e.g. heparan sulfate or fondaparinux) with a test agent and detecting the amount of the intact substrate in comparison to a reference level of intact substrate in the absence of the test agent, or detecting the modulation of the activity of a downstream target of the intact heparanase substrate. Detecting the amount of intact substrate or modulation may be achieved using techniques including, but not limited to, ELISA, cell-based ELISA, inhibition ELISA, western blots, RIA, immunoprecipitation, immunostaining, a solid-phase labeled substrate assay such as a solid phase radio- or fluorescently-labeled or biotinylated substrate, an ultrafiltration assay, proximity assays such as HTRF and scintillation proximity assays, fluorescent assays using e.g. fluorescent substrate-heparanase substrate conjugates such as fluorescein or rhoda mine, colorimetric assays and fluorescent immunoassays.

These methods provide a mechanism for performing high throughput screening of putative heparanase inhibitors such as compounds contained in synthetic, combinatorial, chemical and natural libraries.

In some embodiments, test agents may be screened using commercially available assays, illustrative examples of which include Cisbio heparanse assay toolbox (Biotin-Heparan sulfate-Eu cryptate; Catalogue No. 61BHSKAA; Cisbio Bioassays, Codolet France), Amsbio heparanase assay kit (Catalogue No. Ra001-BE-K; AMS Biotechnology Ltd, Abington UK) and InSight heparanase activity kit (Catalogue No. INS-26-4-0000-10; InSight Biopharmaceuticals, Rehovot, Israel).

The present invention also contemplates methods of screening for agents that are useful for inhibiting macrophage, preferably microglial, activation. In some embodiments, the screening methods comprise: (1) contacting a preparation with a test agent, wherein the preparation comprises an ocular macrophage; and (2) detecting a change in the activation of the macrophage relative to a reference level of activation in the absence of the test agent. A detected reduction in the activation of the macrophage relative to a reference level of activation in the absence of the test agent indicates that the test agent is useful for treating, or inhibiting the development or progression of, an ocular inflammatory disorder. This may be confirmed by analysing or detecting whether the test agent treats, or inhibits the development or progression of, an ocular inflammatory disorder.

A skilled person will be well aware of suitable methods for screening for agents that inhibit ocular macrophage activation. For example, the ability of test agents to inhibit ocular macrophage activation may be assessed using immunofluorescence, ELISA, cell-based ELISA, inhibition ELISA, western blots, RIA, immunoprecipitation, immunostaining, an ultrafiltration assay, fluorescent assays, colorimetric assays and fluorescent immunoassays, including fluorescence microscopy. The above tests may directly or indirectly detect an activated macrophage, such as detecting a marker for an activated macrophage, e.g. IBA1, or cytokine production, e.g. production of IL-1β, TNF-α, IL-6, CXCL8, or IL-12.

The present invention also provides methods of screening for agents that are useful for inhibiting complement fixation. In some embodiments, the screening methods comprise: (1) contacting a preparation with a test agent, wherein the preparation comprises complement proteins, an antigen, antibody and serum; and (2) detecting a change in the levels of a particular complement protein relative to a reference level of the complement protein in the absence of the test agent. A detected reduction in the level of the particular complement protein relative to a reference level of the complement protein in the absence of the test agent indicates that the test agent is useful for treating, or inhibiting the development or progression of, an ocular inflammatory disorder. This may be confirmed by analysing or detecting whether the test agent treats, or inhibits the development or progression of, an ocular inflammatory disorder.

Suitable methods for screening for agents that inhibit complement fixation are known in the art. For example, the inhibition of complement fixation may be assessed using immunofluorescence, a complement fixation test, ELISA, cell-based ELISA, inhibition ELISA, western blots, RIA, immunoprecipitation, immunostaining, a haemolytic assay, fluorescent assays, colorimetric assays and fluorescent immunoassays, including fluorescence microscopy. The above tests may directly or indirectly detect complement fixation, e.g. the assays may detect a particular complement protein such as C3d, or C3b. Suitable assays are also described in Kirschfink and Mollnes (2003) Clin Vaccine Immunol, 10(6): 982-989.

Candidate agents encompass numerous chemical classes, although typically they are organic molecules, preferably small molecules; peptides; polypeptides; proteins; peptidomimetics; carbohydrates such as oligosaccharides and polysaccharides; oligosaccharide-aglycone conjugates; antibodies; lipopolysaccharides; lipids; or polymers. In particular embodiments, the candidate agent is an anionic molecule.

Screening may also be directed to known pharmaceutically active compounds and chemical analogues thereof.

The activity of test agents of interest in in vitro or ex vivo models of ocular inflammatory disorders may then be determined. For example, the activity of test agents may be ascertained using assays involving retinal pigment epithelium (RPE) cells, such as human fetal RPE cells, described in Forest et al. (2015) Disease Models and Mechanisms, 8: 421-427; a retinal angiogenesis assay described in Mi et al. (2014) Drug Des Devel Ther, 8: 2311-2319, and Rezzola et al. (2014) Angiogenesis, 17(3): 429-442; assays assessing macrophage activation; a 3D model using human keratoconus stromal cells described in Karamichos et al. (2012) J Funct Biomater, 3(4): 760-775; assays assessing anti-oxidant activity and protection of cells from reactive oxygen species based on islet beta cell surivival in Ziolkowski et al. (2012) J Clin Invest, 122(1): 132-141, and Choong (2015) Am J Transplant, 15: 2851-2864; and assays determining growth factor activity such as SPR-Biacore, a luciferase assay, a calcium mobilization assay, or a modified Boyden chamber assay such as those described in Papadopoulos et al. (2012) Angiogenesis, 15(2): 171-185.

Compounds may be further tested in animal models to identify those agents having the most potent in vivo effects. These agents may serve as lead compounds for the further development of pharmaceuticals by, for example, subjecting the compounds to sequential modification, molecular modeling and other procedures routine in the art for rational drug design. Suitable animal models include, but are not limited to, those described in Pennisi et al. (2012) Mol Aspects Med, 33(4): 487-509; Jiang et al. (2015) Curr Eye Res, 40(8): 761-771; Mi et al. (2014) Drug Des Devel Ther, 8: 2311-2319; Caspi (2006) Drug Discovery Today: Disease Models, 3(1): 3-9; Rutar et al. (2010) Curr Eye Res, 35(7): 631-643; and Rutar et al. (2015) Journal of Neuroinflammation, 12: 8.

A person skilled in the art will be familiar with heparanase inhibitors and, accordingly, would readily be able to synthesize and/or source the heparanase inhibitors, for example, from Sigma Aldrich Co. LLC. For example, pentosan polysulfate is commercially available under various brand names including, Elmiron, Fibrase, Fibrezym, Hemoclar, Pentosanpolysulfat SP 54, Polyanion SP54, SP 54, Tavan-SP, Thrombocid, Cartorphen Vet and Pentosan equine. Pentosan polysulfate is prepared by the sulfation of a xylan polysaccharide extracted from plants, typically beechwood, as described in U.S. Pat. No. 4,717,373, the content of which is hereby incorporated by reference in its entirety. Muparfostat (PI-88) may be synthesized using the procedure described in WO 96/33726 A1, the entire content of which is herein incorporated by reference.

### 3. Compositions

The present invention also provides compositions comprising a heparanase inhibitor which may be used for treating, or inhibiting the progression of development of, an ocular inflammatory disorder in a subject. Thus, in some embodiments, the heparanase inhibitor may be in the form of a pharmaceutical composition, wherein the pharmaceutical composition comprises a heparanase inhibitor and a pharmaceutically acceptable carrier or diluent.

The heparanase inhibitor may be formulated into the pharmaceutical composition as a neutral or salt form.

As will be appreciated by those skilled in the art, the choice of pharmaceutically acceptable carrier or diluent will be dependent on the route of administration and on the nature of the condition and subject to be treated. The particular carrier or delivery system and route of administration may be readily determined by a person skilled in the art. The carrier or delivery system and route of administration should be carefully selected to ensure that the activity of the heparanase inhibitor is not depleted during preparation of the formulation and the heparanase inhibitor is able to reach the site of action intact.

Suitable pharmaceutically acceptable carriers or diluents include, but are not limited to, an aqueous carrier such as water, saline, aqueous buffer and aqueous solution comprising a water soluble or water miscible additive such as glucose or glycerol; an oil such as almond oil, mineral oil, olive oil, peanut oil, coconut oil, soybean oil, corn oil, anise oil, clove oil, cassia oil, silicone oil, cinnamon oil, arachis oil, maize oil, caraway oil, rosemary oil, peppermint oil, eucalyptus oil, or a seed oil such as canola oil, cottonseed oil, linseed oil, safflower oil, sesame oil or sunflower oil; a fatty acid carrier; or combinations thereof. In some embodiments, the carrier is in the form of an emulsion, especially an oil in water emulsion where the oil is present in an amount in the range of from, for example, 0.2% to 20% w/v.

When saline is used in the carrier, it is preferably isotonic for the point of administration in the eye. For example, in some embodiments, the saline comprises 0.15 to 8% w/v sodium chloride, especially 0.18% to 7% w/v, 0.22% to 5% w/v, 0.45% to 3% w/v sodium chloride, more especially 0.5 to 2% w/v sodium chloride, more especially 0.65% to 1.5% w/v sodium chloride, most especially about 0.9% w/v sodium chloride.

In some embodiments, where the carrier is not isotonic, for example, water, the composition may contain a tonicity agent. Any pharmaceutically acceptable tonicity agent well known in the art may be used. Suitable tonicity agents include, but are not limited to, boric acid, sodium acid phosphate buffer, sodium chloride, glucose, trehalose, potassium chloride, calcium chloride, magnesium chloride, polypropylene glycol, glycerol, mannitol, or salts or combinations thereof. The tonicity agent may be present in the composition in an amount that provides isotonicity with the point of administration in the eye, for example, in the range of from 0.02 to 15% w/v.

In some embodiments, the carrier is a buffer, wherein the buffer maintains a pH in the range of from 3 to 8.5, especially in the range of from 5 to 8.5, more especially in the range of from 6.8 to 8.2, most especially about 7.4. Suitable buffering agents include, but are not limited to, acetic acid, citric acid, sodium metabisulfite, histidine, sodium bicarbonate, sodium hydroxide, boric acid, borax, alkali metal phosphates, phosphate such as sodium phosphate, sulfate or citrate buffers or combinations thereof. The buffering agent may be present in the composition in an amount suitable to maintain the desired pH.

In some embodiments, the pH of the composition is in the range of from 3 to 8.5, 5 to 8.5 or 6.8 to 8.2. In particular embodiments, the pH of the composition is about 7.4.

In particular embodiments, the composition comprises a permeation enhancing agent. Suitable permeation enhancing agents include, but are not limited to, dimethyl sulfoxide; a cyclodextrin such as alpha-, beta- or gamma-cyclodextrin; ethylene diamine tetraacetic acid (EDTA); decamethonium; glycocholate; cholate; a saponin; fusidate; a taurocholate; a polyethylene glycol ether; a polysorbate; a nanoparticle; a liposome; a micelle; or salts, derivatives or combinations thereof. The permeation enhancing agent should be present in an amount that facilitates permeation of the heparanase inhibitor to the site of action such as, for example, the retina. In some embodiments, the permeation enhancing agent is present in an amount in the range of from 0.1% to 30% w/v of the composition.

The composition may further comprise a surfactant. Suitable pharmaceutically acceptable surfactants are known in the art. Exemplary surfactants include, but are not limited to, surfactants of the following classes: alcohols; amine oxides; block polymers; carboxylated alcohol or alkylphenol ethoxylates; carboxylic acids/fatty acids; ethoxylated arylphenols; ethoxylated fatty esters, oils, fatty amines or fatty alcohols such as cetyl alcohol; fatty esters; fatty acid methyl ester ethoxylates; glycerol esters such as glycerol monostearate; glycol esters; lanolin-based derivatives; lecithin or derivatives thereof; lignin or derivatives thereof; methyl esters; monoglycerides or derivatives thereof; polyethylene glycols; polypropylene glycols; alkylphenol polyethylene glycols; alkyl mercaptan polyethylene glycols; polypropylene glycol ethoxylates; polyethylene glycol ethers such as Cetomacrogol 1000; polymeric surfactants; propoxylated and/or ethoxylated fatty acids, alcohols or alkylphenols; protein-based surfactants; sarcosine derivatives; sorbitan derivatives such as polysorbates; sorbitol esters; esters of sorbitol polyglycol ethers; fatty acid alkylolamides; N-alkylpolyhydroxy fatty acid amide; N-alkoxypolyhydroxy fatty acid amide; alkyl polyglycosides; quaternary ammonium compounds such as benzalkonium chloride; cyclodextrins such as alpha-, beta- or gamma-cyclodextrin; sucrose or glucose esters or derivatives thereof; sulfosuccinates such as dioctyl sodium sulfosuccinate; or salts or combinations thereof. The surfactant may be present in an amount in the range of from about 0.1% to 30% w/v of the composition.

In some embodiments, the composition further comprises a rheology modifier. The rheology modifier may be used to alter the surface tension and flow of the composition and may also contribute to the composition's residence time on the surface of or in the eye and the absorption of the composition. Suitable rheology modifiers are well known in the art. For example, the rheology modifier may be selected from, but is not limited to, hyaluronic acid; chitosan; polyvinyl alcohol; polyacrylic acid; polyethylene glycol; polyvinyl pyrrolidone; dextran; cellulose derivatives such as methylcellulose, hydroxypropylmethylcellulose, carboxymethylcellulose, hydroxyethylcellulose and hydroxypropylcellulose; hydroxypropyl guar; acrylates such as Carbopol polymers; poloxamers; gum arabic; xanthan gum; guar gum; locust bean gum; alginate; starch (from rice, corn, potato or wheat); carrageenan; konjac; aloe vera gel; agarose; pectin; tragacanth; curdlan gum; gellan gum; scleroglucan; and derivatives and combinations thereof. The rheology modifier should be present in an amount sufficient to obtain the desired viscosity of the composition, such as an amount in the range of from about 0.5% to 5% w/v of the composition.

The composition of the invention may further comprise a preservative. Suitable preservatives may include, but are not limited to, sodium perborate, stabilized oxychloro complex, polyquaternium compounds such as polyquaternium-1, phenylmercuric acid, benzalkonium chloride, chlorbutanol, phenylmercuric acetate, phenylmercuric nitrate, chlorhexidine, benzododecinium bromide, cetrimonium chloride, thiomersal, methyl parahydroxybenzoate, propyl parahydroxybenzoate, polyquaternium ammonium chloride, polyaminopropyl biguanide, polyhexamethylene biguanide, hydrogen peroxide, benzoic acid, phenolic acids, sodium chlorite, sorbic acid, benzyl alcohol, EDTA, a borate-polyol complex such as Sofzia® (a combination of borate, sorbitol, propylene glycol and zinc), or salts or combinations thereof. The preservative may be particularly useful for preventing microbial contamination in a composition which is subject to multiple uses from the same container, for example, when formulated for topical administration in a multiple unit dosage form. The preservative should be present in an amount that provides sufficient antimicrobial activity, for example in an amount in the range of from 0.001% to 1% w/v of the composition.

In some embodiments, the composition may comprise a chelating agent. Suitable chelating agents include, but are not limited to amino carboxylic acids or salts thereof such as EDTA, nitrilotriacetic acid, nitrilotripropionic acid, diethylenetriamine pentacetic acid, 2-hydroxyethyl-ethylenediamine-triacetic acid, 1,6-diamino-hexamethylene-tetraacetic acid, 1,2-diamino-cyclohexane tetraacetic acid, O,O′-bis(2-aminoethyl)-ethyleneglycol-tetraacetic acid, 1,3-diaminopropane-tetraacetic acid, N,N-bis(2-hydroxybenzyl)ethylenediamine-N,N-diacetic acid, ethylenediamine-N,N′-diacetic acid, ethylenediamine-N,N′-dipropionic acid, triethylenetetraamine hexaacetic acid, 7,19,30-trioxa-1,4,10,13,16,22,27,33-octaazabicyclo[11,11,11]pentatriacontane (O-bis-tren), ethylenediamine-N,N′-bis(methylenephosphonic acid), iminodiacetic acid, N,N-bis(2-hydroxyethyl)glycine, 1,3-diamino-2-hydroxypropane-tetraacetic acid, 1,2-diaminopropane-tetraacetic acid, ethylenediamine-tetrakis(methylenephosphonic acid), N-(2-hydroxyethyl)iminodiacetic acid, or salts or combinations thereof. In some embodiments, the chelating agent is present in an amount in the range of from 0.01% to 1% of the composition.

The composition of the invention may further comprise any other pharmaceutically acceptable excipient commonly present in ocular formulations. For example, the compositions may further comprise an alcohol such as isopropanol, benzyl alcohol, cetearyl alcohol and/or ethanol; a lubricant such as glucose, glycerol, polyethylene glycol, polypropylene glycol and/or derivatives thereof; an antioxidant such as ascorbic acid or vitamin C, phenolic acids, sorbic acid, sodium bisulfite, sodium metabisulfite, sodium thiosulfate, acetyl cysteine, sodium thiosulfate, EDTA, sodium nitrite, ascorbyl stearate, ascorbyl palmitate, alpha-thioglycerol, erythorbic acid, cysteine hydrochloride, citric acid, tocopherol or vitamin E, tocopherol acetate, dibutylhydroxytoluene, soybean lecithin, sodium thioglycolate, butylhydroxyanisole, propyl gallate, uric acid, melatonin, thiourea, or pharmaceutically acceptable salts or derivatives thereof; a stabilizer; or combinations thereof.

The pharmaceutical compositions of the invention may be administered locally to an eye using a variety of routes including, but not limited to, topical, through an ocular implant or direct injection into the eye. In particular embodiments, the pharmaceutical composition of the invention is administered locally to the eye using intravitreal injection, subconjunctival injection, sub-tenon injection, retrobulbar injection, suprachoroidal injection, intrascleral injection, intracorneal injection, subretinal injection or intracameral injection; especially intravitreal injection. In some embodiments, the composition is administered using a microneedle, for example, through intrascleral or intracorneal injection. In some embodiments, the composition is administered using an ocular implant, for example, a biodegradable implant such as those made from, for example, polylactic acid (PLA), polyglycolic acid, poly(lactide-co-glycolide) (PLGA), cross-linked gelatin derivatives, hypromellose, polyesters and/or polycaprolactones; or a non-biodegradable implant such as those made from, for example, polyvinyl alcohol, ethylene vinyl acetate, silicon and/or polysulfone capillary fiber.

Thus, in some embodiments, the composition of the invention is formulated in a sustained release formulation or depot. Exemplary sustained release formulations or depots include a microsphere; matrix; emulsion; lipid-based; polymer-based; nanomicelle; micelle; nanovesicle such as a liposome, noisome, transfersome, discome, pharmacosome, emulsome or spanlastic, especially a liposome; microparticle; nanoparticle such as a nanocapsule or nanosphere composed of e.g. lipids, proteins, natural or synthetic polymers such as albumin, sodium alginate, chitosan, PLGA, PLA and/or polycaprolactone; or in situ gel such as an in situ hydrogel drug delivery system.

In some embodiments, the composition of the invention is formulated for topical administration to the eye. Thus, the composition may be in the form of an eye drop, gel or ointment; especially an eye drop. The composition may be in a single unit dose or multiple unit dose form.

In some embodiments, the composition of the invention is formulated for injection into the eye. In particular embodiments, the composition of the invention is formulated for intravitreal injection, subconjunctival injection, sub-tenon injection, retrobulbar injection, suprachoroidal injection, intrascleral injection, intracorneal injection, subretinal injection or intracameral injection; especially intravitreal injection.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

Other excipients and components of the composition may be readily determined by a person skilled in the art. Techniques for formulation and administration may be found in, for example, Remington (1980) Remington's Pharmaceutical Sciences, Mack Publishing Co., Easton, Pa., latest edition; and suitable excipients may be found in, for example, Katdare and Chaubel (2006) Excipient Development for Pharmaceutical, Biotechnology and Drug Delivery Systems (CRC Press).

In some embodiments, the composition of the invention further comprises one or more further pharmaceutically active agents as described in Section 4 below. The one or more further pharmaceutically active agents may be administered simultaneously, separately or sequentially to the heparanase inhibitor and, thus, the heparanse inhibitor and one or more further pharmaceutically active agents may be provided in kit form.

A person skilled in the art would be familiar with the components of the compositions of the invention and, accordingly, would readily be able to synthesize or source the components, such as from, for example, Sigma Aldrich Co. LLC.

### 4. Methods of Treating, or Inhibiting the Development or Progression of, an Ocular Inflammatory Disorder

The present inventors have found that heparanase inhibitors also block ocular macrophage activation and complement fixation when locally administered to an eye. Accordingly, the inventors have conceived that heparanase inhibitors are useful for treating, or inhibiting the progression or development of, an ocular inflammatory disorder. Heparanase inhibitors may also be used in the manufacture of a medicament for the uses described herein.

Without wishing to be bound by theory, it is thought that local administration of heparanase inhibitors will avoid adverse effects associated with systemic administration and will also enable a lower dose of the inhibitor to be administered. Furthermore, local administration enables ocular macrophages, or microglia, to be specifically targeted.

In one aspect of the invention, there is provided a method for inhibiting complement fixation comprising contacting an ocular macrophage cell with a heparanase inhibitor or a pharmaceutically acceptable salt thereof. The invention also provides a use of a heparanase inhibitor or a pharmaceutically acceptable salt thereof for inhibiting complement fixation, wherein an ocular macrophage cell is contacted with the heparanase inhibitor. The heparanase inhibitor, in one or more of these aspects further inhibits macrophage activation.

While the inhibition of activation of all ocular macrophages is contemplated by the present invention, in some embodiments, the macrophage is a macrophage which expresses heparanase. In preferred embodiments, the ocular macrophage is a microglia cell.

In some embodiments, the complement fixation preferably arises from, or is otherwise associated with, microglial activation.

In some embodiments, the complement fixation arises from, or is otherwise associated with, retinal inflammation.

In a further aspect of the invention, there is provided a method of treating, or inhibiting the progression or development of, an ocular inflammatory disorder in a subject comprising locally administering a heparanase inhibitor or a pharmaceutically acceptable salt thereof to the subject.

The present invention also provides the use of a heparanase inhibitor or a pharmaceutically acceptable salt thereof for treating, or inhibiting the progression or development of, an ocular inflammatory disorder in a subject, wherein the heparanase inhibitor is formulated for local administration to an eye of the subject; and in the manufacture of a medicament for treating, or inhibiting the progression or development of, an ocular inflammatory disorder in a subject, wherein the heparanase inhibitor is formulated for local administration to an eye of the subject.

In another aspect, the present invention provides a heparanase inhibitor or a pharmaceutically acceptable salt thereof for use for treating, or inhibiting the progression or development of, an ocular inflammatory disorder in a subject, wherein the heparanase inhibitor is formulated for local administration to an eye of the subject.

The ocular inflammatory disorder may be any disorder of the eye which has an inflammatory component. Suitable ocular inflammatory disorders include, but are not limited to, age-related macular degeneration (AMD) including the exudative or ‘wet’ and ‘dry’ form of AMD, diabetic retinopathy, retinitis pigmentosa, retinal vein occlusion, retinoblastoma, uveitis, macular edema, dry eye, ocular inflammation associated with an infection and/or keratoconus; especially AMD, diabetic retinopathy, ocular inflammation associated with an infection and retinitis pigmentosa; more especially AMD and diabetic retinopathy; most especially AMD. Although acute ocular inflammatory disorders are contemplated by the invention, in particular embodiments, the ocular inflammatory disorder is a chronic disorder.

In some embodiments, the ocular inflammatory disorder is a disorder associated with heparanase activity, especially heparanase catalytic activity.

In some embodiments, the ocular inflammatory disorder is a disorder of the anterior or posterior segment of the eye; especially the posterior segment of the eye.

Suitable ocular inflammatory disorders of the anterior segment of the eye include, but are not limited to, dry eye, keratoconus and anterior segment uveitis. Suitable ocular inflammatory disorders of the posterior segment of the eye include, but are not limited to, AMD, diabetic retinopathy, retinitis pigmentosa, retinoblastoma, macular edema, retinal vein occlusion and posterior segment uveitis.

In some embodiments, the ocular inflammatory disorder is ocular inflammation associated with an infection. In preferred embodiments, the infection is a pathogenic infection. The ocular inflammation may be associated with a viral, bacterial, protozoan, nematode, cestode or fungal infection, especially a viral infection.

Suitable viral infections include, but are not limited to, an infection caused by herpes simplex virus (HSV), especially type 1 or type 2 HSV; human herpesvirus 6; adenovirus; molluscum contagiosum virus; varicella-zoster virus; Epstein-Barr virus; cytomegalovirus; picornavirus; hepatitis B virus; mumps virus; measles virus; and influenza virus; especially type 1 or type 2 HSV; most especially type 1 HSV.

Suitable bacterial infections include, but are not limited to, an infection caused by Neisseria species, such as N. gonorrhoeae and N. meningitides; Staphylococcus spp. including S. aureus, S. epidermidis and S. pyogenes; Streptococcus spp. including S. pneumoniae; Haemophilus influenza; Moraxella species including M. lacunata, M. nonliquefaciens, M. liquefaciens and M. catarrhalis; Chlamydia trachomatis; Pneumococcus spp.; Bacteroides spp.; Peptostreptococcus spp.; Propionibacterium acnes; Bacillus cereus; Pseudomonas aeruginosa; Treponema pallidum; Mycobacterium tuberculosis; Mycobacterium leprae; and Borrelia burgdorferi.

Suitable protozoan infections include, but are not limited to, an infection caused by Acanthamoeba spp. including A. castellanii, A. polyphaga, A. culbertsoni, A. hatchetti, A. rhysodes, A. lugdunensis, A. quina and A. griffin; Toxoplasma gondii; Trypanosoma cruzi; Plasmodium spp. including P. vivax, P. ovale, P. malariae and P. falciparum; Leishmania spp.; Encephalitozoon spp.; Nosema spp.; Microsporidium spp.; Septata spp.; Giardia lamblia; and Rhinosporidium seeberi.

Suitable nematode infections include, but are not limited to, an infection caused by Onchocerca volvulus; Loa loa; Dirofilaria immitis; and Gnathostoma spp. including G. spinigerum. Suitable cestode infections including, but are not limited to, an infection caused by Taenia spp. including T. solium; Toxocara spp. including T. canis; and Echinococcus spp. including E. granulosus.

Suitable fungal infections include, but are not limited to, infections caused by Candida spp. including C. albicans, C. famata, C. parapsilosis, C. lipolytica, C. humicola, C. guilliermondii and C. glabrata; Aspergillus spp. including A. flavus, A. niger, A. fumigatus, A. terreus, A. glaucus, and A. nidulans; Fusarium spp. including F. solani and F. moniliforme; Cryptococcus spp. including C. neoformans; Pneumocystis spp. including P. carinii; Histoplasma spp. including H. capsulatum; Bipolaris spp.; Zygomycetes spp.; Coccidioides immitis; Blastomyces dermatitidis; Lasiodiplodia theobromae; Alternaria spp.; Sporothrix schenckii; Paecilomyces lilacinus; Acremonium kiliense; Exophiala jeanselmei; Pseudallescheria boydii; Scytalidium dimidiatum; Helminthosporium spp.; Penicillium chrysogenum; Absydia spp.; Rhizopus spp.;

Curvularia spp.; Phialophora spp.; Paracoccidioides brasiliensis; Malassezia spp. including M. furfur and M. pachydermatis; Conidiobolus coronatus; Rhodotorula spp.; Drechslera spp.; Curvularia spp.; Mucor spp.; and Absidia spp.

In some embodiments, the ocular inflammatory disorder is, but is not limited to, inflammation associated with conjunctivitis, keratitis, cellulitis, endophthalmitis, episcleritis, uveitis, retinitis, dacryocystitis, hordeolum, chalazion, endotheliitis, blepharitis, vitritis, chorioretinitis, sarcoidosis and onchocerciasis.

In particular embodiments, the ocular inflammatory disorder is AMD or diabetic retinopathy; especially AMD. In some embodiments, the ocular inflammatory disorder is wet AMD or dry AMD.

The ocular inflammatory disorder may be associated with heparanase activity, macrophage activation and/or complement fixation. In some embodiments, the ocular inflammatory disorder is a disorder in respect of which inhibition of macrophage activation is associated with effective treatment. The ocular inflammatory disorder, in some embodiments, is a disorder which is associated with macrophage activation. In some embodiments, the ocular inflammatory disorder is a disorder in respect of which inhibition of complement fixation is associated with effective treatment. The ocular inflammatory disorder, in some embodiments, is a disorder which is associated with complement fixation. The invention also contemplates ocular inflammatory disorders which are associated with macrophage activation and complement fixation. In some embodiments, the ocular inflammatory disorder is a disorder in respect of which heparanase inhibition is associated with effective treatment, and/or is a disorder which is associated with heparanase activity, particularly heparanase catalytic activity.

In some embodiments, the ocular inflammatory disorder preferably arises from, or is otherwise associated with, microglial activation.

In some embodiments, the ocular inflammatory disorder is a retinopathy, including AMD, diabetic retinopathy, retinitis pigmentosa, retinal vein occlusion or retinoblastoma.

In some embodiments, the ocular inflammatory disorder arises from, or is otherwise associated with, retinal inflammation. Suitable conditions include, but are not limited to, AMD, diabetic retinopathy, macular edema, retinitis pigmentosa, retinal vein occlusion or retinoblastoma; especially AMD, diabetic retinopathy, retinitis pigmentosa, retinal vein occlusion or retinoblastoma; more especially AMD or diabetic retinopathy; most especially AMD.

Suitable embodiments of the heparanase inhibitor and compositions thereof are as described in Sections 2 and 3 supra.

In some embodiments, the heparanase inhibitor has one or more activities selected from the group consisting of inhibition of macrophage activation, complement fixation, growth factor activity, such as VEGF, and oxidative damage. In some embodiments, the heparanase inhibitor is an inhibitor of VEGF activity. Without wishing to be bound by theory, it is thought that heparanase inhibitors which inhibit heparanase, macrophage activation, complement fixation, growth factor activity e.g. VEGF activity, and oxidative damage may have improved efficacy for the treatment, and/or inhibition of the progression or development of, an ocular inflammatory disorder.

Although the heparanase inhibitor may be the sole pharmaceutically active agent administered to the subject, the administration of other pharmaceutically active agents is within the scope of the invention. For example, the heparanase inhibitor may be administered with one or more further pharmaceutically active agents, such as an agent that inhibits macrophage activation, an agent that inhibits complement fixation, a growth factor inhibitor, an antioxidant, an anti-inflammatory agent, an antiviral, an antibacterial, an antifungal, an anthelmitic, an antiprotozoal or an agent that is otherwise useful for treating, or inhibiting the progression or development of, an ocular inflammatory disorder described herein. The further pharmaceutically active agent may be administered simultaneously, sequentially or separately with the heparanase inhibitor. In some embodiments, the composition of the invention comprises a heparanase inhibitor and one or more further pharmaceutically active agents.

In some embodiments, the one or more further pharmaceutically active agent is a growth factor inhibitor. Suitable growth factor inhibitors include, but are not limited to, a vascular endothelial growth factor (VEGF) inhibitor, such as ranibizumab, aflibercept, bevacizumab, pegaptanib, conbercept, abicipar pegol (MP0112) and MP0250; a platelet derived growth factor (PDGF) inhibitor, such as E10030 (anti-PDGF PEGylated aptamer), trapidil and pegpleranib; and pharmaceutically acceptable salts and combinations thereof. In some embodiments, the one or more pharmaceutically active agent is a VEGF inhibitor selected from the group consisting of ranibizumab, aflibercept, bevacizumab, pegaptanib, conbercept and pharmaceutically acceptable salts and combinations thereof.

The methods involve local administration of the heparanase inhibitor to an eye of a subject. The heparanase inhibitor may be administered locally through topical administration to the surface of the eye, may be administered using an ocular implant or may be administered via direct injection into the eye.

In some embodiments, the heparanase inhibitor is administered via injection into an eye. In particular embodiments, the heparanase inhibitor is administered locally to the eye using an intravitreal injection, subconjunctival injection, sub-tenon injection, retrobulbar injection, suprachoroidal injection, intrascleral injection, intracorneal injection, subretinal injection or intracameral injection; especially intravitreal injection. In some embodiments, the heparanase inhibitor is administered using a microneedle, for example, through intrascleral or intracorneal injection.

In some embodiments, the heparanase inhibitor is administered using an ocular implant, for example, a biodegradable implant such as those made from, for example, polylactic acid, polyglycolic acid, poly(lactide-co-glycolide), cross-linked gelatin derivatives, hypromellose, polyesters and/or polycaprolactones; or a non-biodegradable implant such as those made from, for example, polyvinyl alcohol, ethylene vinyl acetate, silicon and/or polysulfone capillary fiber.

The heparanase inhibitor may be administered in the form of a composition as described in Section 3 supra. Thus, in some embodiments, the heparanase inhibitor may be administered in a sustained release formulation or depot including, but not limited to, a microsphere; matrix; emulsion; lipid-based; polymer-based; nanomicelle; micelle; nanovesicle such as a liposome, noisome, transfersome, discome, pharmacosome, emulsome or spanlastic, especially a liposome; microparticle; nanoparticle such as a nanocapsule or nanosphere composed of e.g. lipids, proteins, natural or synthetic polymers such as albumin, sodium alginate, chitosan, poly(lactide-co-glycolide) (PLGA), polylactic acid (PLA) and/or polycaprolactone; or in situ gel such as an in situ hydrogel drug delivery system.

In some embodiments, the heparanase inhibitor is administered via topical administration. When topically administered, the heparanase inhibitor may be administered in the form of an eye drop, gel or ointment; especially an eye drop.

In some embodiments, administration of the heparanase inhibitor may be accompanied or followed by application of an active force to increase penetration of the heparanase inhibitor through ocular tissue. Suitable active forces include, but are not limited to, sound waves, such as ultrasound waves, and iontophoresis. In this regard, the heparanase inhibitor may be applied topically as described herein, followed or accompanied by sound wave application, especially ultrasound wave application, or the application of an iontophoretic current. In another embodiment, the heparanase inhibitor may be injected into the eye as described herein, followed or accompanied by sound wave application, especially ultrasound wave application, or the application of an iontophoretic current. In a further embodiments, the heparanase inhibitor may be administered using an ocular implant, as described herein, followed or accompanied by sound wave application, especially ultrasound wave application, or the application of an iontophoretic current. Suitable methods for ocular drug delivery using sound waves or an iontophoretic current are described in, for example, US 2011/0066101 A1, WO 2007/050645 A2, WO 2008/013913 A2, WO 2006/047788 A2, WO 2003/043689 A1, WO 2003/030989 A2, Zderic et al. (2004) J Ultrasound Med, 23: 1349-1359, Mitragotri (2005) Nat Rev Drug Discov, 4: 255-260, Lafond et al. (2017) Expert Opinion on Drug Delivery, 14(4): 539-550, WO 2016/118933 A1, WO 2010/078246 A1, WO 2010/009087 A1, WO 2007/081750 A2, and WO 2007/099406 A2, the entire contents of which are hereby incorporated by reference in their entirety.

Dosage regimes may be established for different indications in accordance with methodologies well known to a person skilled in the art. The dosage of the composition and frequency of dosing will depend on the condition to be treated, the severity of the condition, the age of the subject and the route of administration.

The heparanase inhibitor may be administered at a frequency of about twice a day, or once a day, week, 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months or greater than 12 months (and all integers therebetween). In some embodiments, the heparanase inhibitor may be administered at a higher frequency for a specified period of time, followed by a less frequent dosing administration, for example, the heparanase inhibitor may be administered at a high frequency, such as once a week, for a period of one month, followed by a lower frequency, such as once every six months.

When topically administered in the form of an eye drop, the heparanase inhibitor may be administered in an amount in the range of from 1 to 6 drops per eye (and all integers therebetween), which may equate to, for example, an amount in the range of from 0.04 mL to 0.24 mL per eye (and all integers therebetween). Drops may be applied to each eye from 1 to 4 times daily. When the heparanase inhibitor is formulated as a gel or ointment, an equivalent dose is provided. A skilled person will be well aware of suitable dispensers for topical application of the heparanase inhibitor.

When administered by injection, the heparanase inhibitor may be administered in an amount in the range of from 0.001 mL to 0.5 mL per eye (and all integers therebetween); especially about 0.05 mL.

When administered as an ocular implant, the heparanase inhibitor may be administered in an amount in the range of from 0.01 to 10 mg (and all integers therebetween), which is released over an extended period of time, e.g. from about 1 week to greater than 12 months (and all integers therebetween).

The heparanase inhibitor may be administered in an amount suitable to provide a dose of the heparanase inhibitor in the range of from 0.0001 to 20 mg/kg/day (and all integers therebetween).

In order that the invention may be readily understood and put into practical effect, particular preferred embodiments will now be described by way of the following non-limiting examples.

### EXAMPLES

All materials used in the following examples are commercially available from, for example, Sigma-Aldrich Co. LLC unless otherwise indicated.

### Example 1—Retinal Complement Deposition by Macrophages Following Retinal Damage

### Materials and Methods

### Animal Experimentation

All experiments were conducted in accordance with the ARVO Statement for Use of Animals in Ophthalmic and Vision Research. The study was approved by the Australian National University Animal Experimentation Ethics Committee. C57BL63 mice were born and raised in 12:12 hrs light:dark cycle of 5 lux in individually vented cages, with free access to food and water. Age-matched adult mice (8-10 weeks) were randomly assigned to light damage and dim-reared control (non-light damage) groups. Animals of the light damage group were continuously exposed to 100 k lux white LED light for 1, 3, 5, and 7 days. Pupils were dilated twice daily at 10 am and bpm with a single drop of 1% atropine sulfate (8.3 mg of atropine). Dim-reared control animals were also pupil dilated twice each day, but were returned to dim cyclic light (12:12 hrs light:dark, 5 lux light). Even distribution between males and females was performed where appropriate to allow for differences in sex.

### Light Exposure Device

Retinal damage was induced by bright-light exposure. During bright-light exposure, animals were housed in plastic boxes (two per box), with free access to food and water. The floors of the cages were coated with a reflective Perspex surface and illuminated by a 100-W 65000 k natural white LED (high CRI LED, Yuji, Beijing) mounted 18 cm above the plastic boxes. The LED light has an emission spectrum which more closely resembles daylight than halogen or incandescent bulbs. Temperature in the cages was maintained at ˜23±2° C. with a dual exhaust system to alleviate any heat generated from the LED, with one exhaust fan mounted next to the LED light source, and another one on the side of the cage. In order to regulate accurate illumination each box was equipped with a dimmer and adjusted to 100 k lux using a light meter data logging device (Extech HD450). Animals were provided with bedding, food and water during the time course of light exposure, and their behaviour was monitored daily.

### In Situ Hybridization on Human and Rodent Retinas

To localize C3 mRNA transcripts in retinal cryosections, C3 was cloned from PCR products derived from human (460 bp amplicon) and rat (483 bp amplicon) retinal cDNA. These cloned templates were then synthesized into a digoxigenin (DIG)-labelled riboprobe that was specific to human or rat C3 mRNA, according to previously published methodology (Rutar et al. (2011) Invest Ophthalmol Vis Sci, 52: 5347-5358). In situ hybridization was performed on cryosections from either human AMD donor tissue or rat retinas subjected to photo-oxidative damage. AMD tissues had been extensively categorized and processed for cryosectioning in an earlier investigation (Shelley et al. (2009) Arch Ophthalmol, 127: 483-492). In brief, human eyes were collected with informed consent through the Lions NSW Eye Bank, Sydney, Australia, with ethical approval from the Human Research Ethics Committee of the University of Sydney and The Australian National University. Grading for the eyes ranged from normal to early- or late-AMD, and was assigned by a team of experienced graders according to published pathological criteria (Curcio et al. (1998) Invest Ophthalmol Vis Sci, 39: 1085-1096). In situ hybridization was conducted using a previously established protocol (Cornish et al. (2005) Vis Neurosci, 22: 447-459). Both rat and human C3 riboprobes were hybridized overnight at 57° C. and then washed in saline sodium citrate (pH 7.4) at 60° C. The bound probe was visualized with NBT/BCIP, and sections were double-labelled using IBA1 immunohistochemistry.

### Results

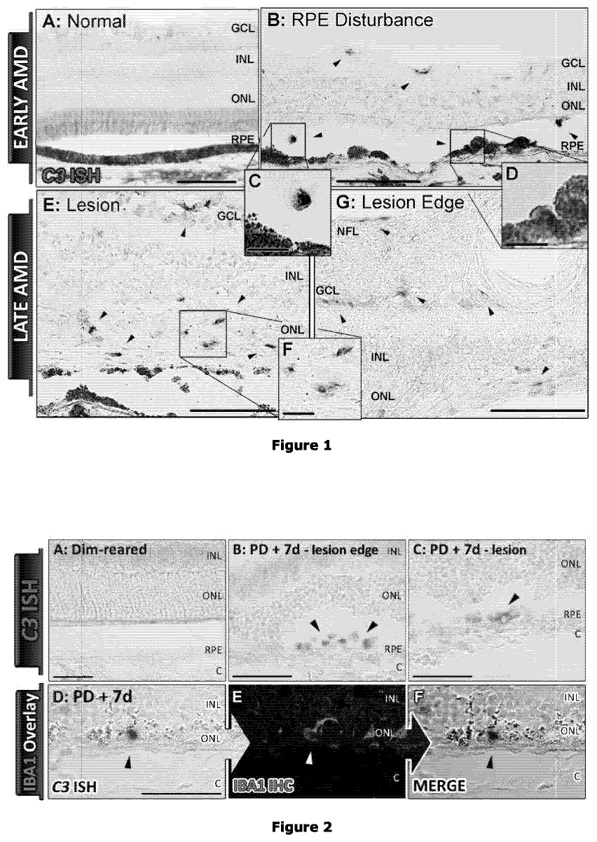

Following retinal damage, complement is deposited in the retina by macrophages in both human and rodents (FIGS. 1 and 2).

In normal aged human donor retinas and age-matched AMD-affected retinas, expression and localization of C3 mRNA was examined using in situ hybridization (FIG. 1). No or minimal C3 mRNA expression was detected in normal retinas, and little-to-none within RPE cells or choroid (FIG. 1A). In contrast, in early AMD retinas, C3-expressing cells were detected in the nerve fibre layer (NFL), superficial retinal vasculature, and subretinal space in regions adjacent to RPE disturbance (FIGS. 1B-D). Numerous C3-expressing cells were detected in regions of advanced scarrings (FIG. 1E-F), at lesion edges (FIG. 1G). Counter immunolabelling indicated that most of the C3-expressing cells were immunopositive for the macrophage/microglia marker IBA1.

Expression and localization of C3 in light-damaged retinas was also examined in rodents using in situ hybridization (ISH) to localize expression of C3 mRNA at 7 days following bright-light induced retinal damage. C3 mRNA was expressed in the neural retina and subretinal space in light induced lesions (FIGS. 2A-C). Accumulations of C3-expressing macrophages predominantly clustered amongst the remnants of the ONL and at the lesion edges, and near the inner retinal vasculature (FIGS. 2A-C). Co-localization with IBA1+ immunolabelling (FIGS. 2D-F) suggests the identity of these C3-expressing cells as macrophages. C3 expression was not detected in other cell types in the retina, including RPE cells.

Microglia activation and activated macrophage infiltration of the subretinal space is a key feature of retinopathy and AMD histopathology, and the pathogenic processes induced by macrophages that is demonstrated to include C3 expression and complement deposition lead to progression of retinopathy and AMD. These results show that C3 is expressed specifically and at a significant level by retinal and subretinal macrophages in AMD-affected retinas and light-induced retinal lesions. In contrast, C3 is not expressed by RPE or other retinal cells that are not microglia or macrophage cells.

### Example 2—Heparanase and Complement Expression in Activated Macrophages Following Retinal Photoreceptor Cell Death

### Materials and Methods

### Gene Expression Analysis

Quantitative real-time polymerase chain reaction (qPCR) RNA extraction and purification was performed on IBA1+ microglia/macrophage cells from retinas following light-induced retinal photoreceptor death using a combination of TRIzol reagent (Thermo Fisher Scientific) and an RNAqueous Total RNA Isolation Kit (Thermo Fisher Scientific) as described previously (Natoli et al. (2008) Mol Vis, 14: 1983-1994). cDNA was prepared from 500 ng of each RNA sample using a Tetro cDNA Synthesis Kit (Bioline Reagents, London, UK) according to the manufacturer's protocol. Gene expression changes were measured via qPCR using Taqman hydrolysis probes and Taqman Gene Expression Master Mix (Thermo Fisher Scientific). Each qPCR was run using a QuantStudio 12K Flex instrument (Applied Biosystems). Analysis was performed using the comparative cycle threshold method (ΔΔCt) which was normalized to the expression of Gapdh and Actb reference genes, as established previously (Rutar et al. (2011) Invest Ophthalmol Vis Sci, 52: 2379-2388; Rutar et al. (2014) PLoS One, 9: e93343).

### Results

Following retinal photoreceptor cell death, IBA1-positive retinal microglia/macrophages activated by local tissue damage expressed significantly elevated levels of heparanase directly (6.50 fold, 0 days; Table 1). Increased expression of heparanase was observed for at least 7 days post-damage (3.48 fold; Table 1). Complement component C3 expression dropped initially but was also significantly upregulated at 7 days post-damage (1.93 fold; Table 1). The elevated C3 expression by activated retinal macrophages is consistent with the in situ expression data from in human AMD and rodent light-induced retina lesions (Example 1). In contrast to heparanase and C3, expression of the regulator Complement Factor H was decreased initially (−2.84 fold, 0 days; Table 1) but did not show statistically significant elevation at 7 days (Table 1). Thus, elevated heparanase and C3 expression in activated IBA1+ retinal microglia/macrophages is a coordinated regulatory feature of these genes that is not shared by Complement Factor H.

TABLE 1
Heparanase and Complement Expression in Activated Macrophages
Following Retinal Damage

| | 0 days after damage | | 7 days after damage | |
|---|---|---|---|---|
| Gene | Fold change | P-value | Fold change | P-value |
| Heparanase | 6.50 | 0.000 | 3.48 | 0.012 |
| Complement | −2.84 | 0.000 | −1010 | 0.766 |
| Factor H | | | | |
| Complement | −1.94 | 0.020 | 1.93 | 0.014 |
| Component 3 | | | | |
| (C3) | | | | |

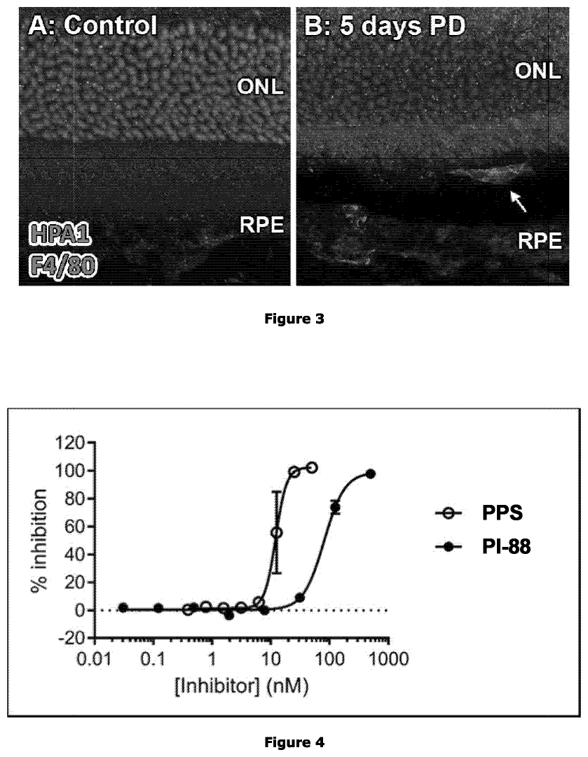

Elevated expression of heparanase protein in activated retinal macrophages in light-induced retinal lesions was further demonstrated through immunohistochemical staining for heparanase protein following photo-oxidative damage (FIG. 3). In control animals (FIG. 3A), no microglia or macrophages were detected by the microglia/macrophage marker F4/80+ immunohistochemical labelling in the outer retina, between the outer nuclear layer and the retinal pigment epithelium. In contrast, after 5 days of photo-oxidative damage, F4/80+ macrophages were recruited into the outer retina and these macrophages were found to express heparanase (FIG. 3B). These results indicate that high levels of heparanase are expressed by activated macrophages that migrate to areas of photo-oxidative damage associated with the initiation and progression of AMD and retinopathy.

### Example 3—Effect of Heparanase Inhibitors on Macrophage Activation and Complement Deposition

### Materials and Methods

### Heparanase Enzyme Inhibition Assay

Heparanase assays were conducted as described previously (Hammond et al. (2010) Anal Biochem, 396(1): 112-116). Recombinant human active heparanase derived from Chinese hamster ovary cells was from R&D Systems. Bovine serum albumin-coated 96 well microplates (96F Maxisorp NNC #456537, Thermo Scientific) were used for the assays and were prepared by incubation of the plates with 1% (w/v) BSA dissolved in phosphate-buffered saline containing 0.05% (v/v) Tween-20 (PBST) at 37° C. for 1 h. The plates were washed three times with PBST, shaken dry, and stored for up to two weeks at 4° C. before use. Assay mixtures typically contained 42.5 mM sodium acetate buffer (pH 5.0), 0.8 nM heparanase, 100 μM fondaparinux (Arixtra™, Aspen Pharmacare), 5% (v/v) dimethyl sulfoxide (DMSO), and varying concentrations of inhibitor in a total volume of 100 μL. Following initiation of the reaction by addition of fondaparinux, the plate was sealed with adhesive film and incubated at 37° C. for 20-24 h. 100 μL of 1.69 mM WST-1 (Dojindo) solution in 0.1 M NaOH was added to the assay mixture. The plate was resealed and developed by incubation at 60° C. for 1 h, and the absorbance was measured at 584 nm. For the enzymatic assays, test compounds (pentosan polysulfate and PI-88) were dissolved in water and added to the assay at varying concentrations to calculate the level of inhibition. Pharmaceutical grade pentosan polysulfate was derived from 10 capsules (2.29 g) of Elmiron (Arthropharm Pty. Ltd.) that were suspended in water (20 mL) and the mixture filtered through cotton wool to remove magnesium stearate and cellulose. The cloudy mixture was then syringe filtered (ChromTech, Nylon, 0.45 μm) to afford a clear solution which was concentrated by a gentle stream of nitrogen. The resulting glass was then dried under high vacuum (1 mmHg, 18° C., 3 hr), ground into a fine powder with a mortar and pestle and further dried (1 mmHg, 18° C., 2 hr) to afford pentosan polysulfate (878 mg) as a cream-coloured powder. PI-88 was prepared as described previously (Karoli et al. (2005) J Med Chem, 48(26): 8229-8236).

### Pentosan Polysulfate Formulation

Purified and desiccated pentosan polysulfate (average MW=4,000-6,000) was reconstituted in phosphate-buffered saline (PBS) prior to intravitreal injection. Pentosan polysulfate was formulated for injection by suspending in water and syringe filtering (ChromTech, Nylon, 0.45 μm) to afford a clear solution which was concentrated by a gentle stream of nitrogen. The resulting glass was then dried under high vacuum (1 mmHg, 18° C., 3 hr), ground into a fine powder with a mortar and pestle and further dried (1 mmHg, 18° C., 2 hr) to afford pentosan polysulfate (878 mg) as a cream-coloured powder. This was resuspended in PBS at the appropriate concentration for delivery at 2 μg per eye by intravitreal injection.

### TUNEL Staining and Quantification of TUNEL+ Cells

TUNEL was used to quantify photoreceptor apoptosis during bright light exposure, and performed on retinal cryosections using a protocol published previously (Rutar et al. (2010) Curr Eye Res, 35(7): 631-643; and Natoli et al. (2016) Exp Eye Res, 147: 114-127). For negative control experiments, the terminal deoxynucleotidyl transferase (TdT) enzyme was omitted. A retinal histological section containing the optic nerve (ON) head was taken to compare all regions of the retina in the superior and inferior regions. In each region, the number of TUNEL+ cells was quantified in increments of 500 μm along the full length of the retina, starting at the optic nerve (ON) head and extending toward the periphery in the superior and inferior regions. In addition, the average number of TUNEL+ cells was calculated for the superior and inferior regions of each retina. To investigate the extent of cell death, the process of quantification was performed on two retinal sections, and calculated as the average for each animal. The values for the dim-reared control and each light damage group were compared by one-way ANOVA followed by Tukey's multiple comparison post-test (TUNEL counts across time points) and two-way ANOVA (TUNEL counts for eccentricity and superior-inferior analysis) followed by Sidak's multiple comparison post-test, with a n=4 per experimental group.

### Immunohistochemistry

Adjacent sections to those used for the TUNEL assay were selected for immunohistochemistry. Sections were incubated in 10% normal goat serum (Sigma Aldrich, Australia) for 1 h at room temperature (RT), followed by overnight incubation in primary antibody at 4° C. Antigen retrieval was performed for IBA1 (rabbit IBA1, Wako Osaka, JP) and F4/80 (Abcam, UK). With this antibody, sections were incubated in Revealit-Ag Antigen Recovery Solution (ImmunoSolutions, QLD, Australia) for 1 h at 37° C. before the overnight incubation of the antibody. The sections were then washed in 0.1 M PBS, and incubated with appropriate secondary antibody-AlexaFluor 488 or 594 (ThermoFisher Scientific) for 4 h at room temperature. For F4/80, biotinylated secondary antibody was used for 2 h incubation followed by 1.5 h incubation of streptavidin-AlexaFluor 488 or 594 conjugates (ThermoFisher Scientific) at room temperature. Sections were then stained with bisbenzimide (Sigma Aldrich) to identify cellular layers, and coverslipped with Aqua-Poly/Mount (Polysciences, PA, USA). To control for non-specific binding the primary antibody was omitted from some sections. Visualization of immunofluorescence and image acquisition was performed using the Nikon A1 Confocal Microscope.

### Measurement of Retinal Function Using Electroretinography (ERG)

Full-field scotopic ERGs were performed to assess the retinal function of dim-reared control and 7 days light damaged animals. Briefly, mice were dark adapted overnight, anesthetized by intraperitoneal injection of xylazine (10 mg/kg) and ketamine (100 mg/kg) and the pupils dilated with a single drop of 1% atropine sulfate (8.3 mg of atropine). A single- or twin-flash paradigm was used to elicit mixed (rod and cone) or isolated cone responses, respectively. Flash stimuli for mixed responses were provided by an LED-based system (FS-250A Enhanced Ganzfeld, Photometric Solutions International, Melbourne), over a stimulus intensity range of 6.3 log cd s m⁻² (range −4.4-1.9 log cd s m⁻²). Interstimulus interval was increased from 2 s for the lowest intensities to 5 min for the highest intensities to allow complete recovery of the b-wave between stimuli. Isolated cone responses were obtained at 1.6 log cd s m⁻² following a rod-saturating stimulus of 1.9 log cd s m⁻² given 400 ms before the test stimulus. This short interval after a rod-saturating flash does not allow recovery of rod function, thereby revealing cone-only responses. The a-wave amplitude was measured from the baseline to the trough of the a-wave response and the b-wave amplitude was measured from the trough of the a-wave to the peak of the b-wave. Data are expressed as the mean wave amplitude ±SEM (pV). Two-way ANOVA, with Tukey's multiple comparisons post-hoc test, was performed to compare the responses from control and light damaged mice over the flash stimulus range. The a-wave and b-wave data were fitted with a Naka-Rushton equation [R/Rₘₐₓ=I/(I+K)] using the Solver function in Excel (Microsoft Version 2013) to determine Rₘₐₓ (maximum amplitude) and K (semisaturation constant) from the response amplitude (R) and the flash intensity (I) over the range of −4.4 to 1.9 log cd s m⁻². Statistics were performed using Prism (GraphPad Software V5; GraphPad Software, Inc., La Jolla, Calif., USA) and either a 2-way ANOVA for mixed a-wave and b-wave and students t-test for cone b-wave. For each analysis, differences with a p<0.05 were considered statistically significant.

### Intravitreal Injections

Intravitreal injections were performed as described in detail previously (Rutar et al. (2012) J Neuroinflammation, 9: 221) wherein animals were anesthetized using an intraperitoneal injection of ketamine (100 mg/kg; Troy Laboratories, NSW, Australia) and xylazil (12 mg/kg; Troy Laboratories). Injections into individual animals consisted of a 1 μL solution containing PBS (control), Eylea (2 μg/μL) or pentosan polysulfate heparanase inhibitor (2 μg/μL) formulated as per the procedure above. Animals were allowed to wake from anesthetic, during which corneal hydration was maintained though application of a synthetic tear gel (GenTeal Gel; Novartis, NSW, Australia). Animals were exposed to photo-oxidative damage for 5 days as previously described in Example 1.

### Results

Pentosan polysulfate (PPS) and PI-88 were found to potently inhibit heparanase, with IC₅₀ values (inhibitor concentration leading to a 50% reduction in heparanase activity) of 12 nM and 81 nM, respectively (Table 2). The IC₅₀ values were calculated from the heparanase enzyme inhibition curve shown in FIG. 4.

TABLE 2
Heparanase inhibition by PPS and PI-88

| | | | Hill |
|---|---|---|---|
| Inhibitor | IC₅₀ (nM) | Max. Inhibition (%) | coefficient |
| PPS | 12 | 102 | 4.7 |
| PI-88 | 81 | 99 | 2.5 |

Notably, the calculated Hill coefficients for both PPS and PI-88 are greater than 1, suggesting these inhibitors bind to heparanase cooperatively. Further, the calculated Hill coefficient of PPS (4.7) is significantly greater than PI-88 (2.5), suggesting a greater level of cooperative binding by PPS may contribute to PPS being a more potent heparanase inhibitor as reflected in the lower IC₅₀ value for PPS (12 nM) compared to PI-88 (81 nM).

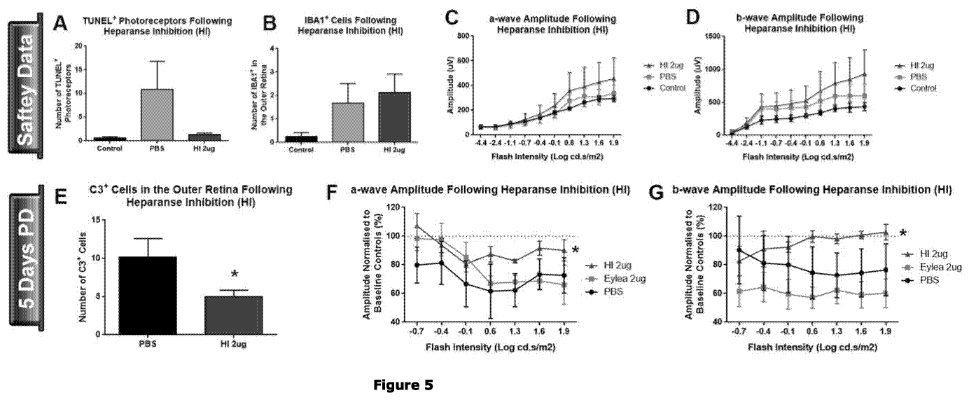

Pentosan polysulfate was also found to inhibit macrophage activation and associated C3 expression and deposition in IBA1+ microglia/macrophages in the retina. Treatment with pentosan polysulfate did not cause any detrimental effects to the retina when administered via intravitreal injection in a mouse eye (FIG. 5A-D). Specifically, pentosan polysulfate administration to wild-type control mice did not increase photoreceptor cell death (FIG. 5A) or the number of IBA1 inflammatory cells (FIG. 5B), and did not modulate retinal function (FIGS. 5C and D). In mice with induced ocular photo-oxidative damage, pentosan polysulfate (HI 2 μg) treatment significantly decreased the number of IBA1+ and C3+ cells in the outer retina compared to PBS-treated control (PBS) mice (FIG. 5E).

### Example 4—In Vivo Efficacy of Heparanase Inhibitors Following Photo-Oxidative Damage

### Materials and Methods

### Animal Experimentation

All experiments were conducted in accordance with the ARVO Statement for Use of Animals in Ophthalmic and Vision Research. The study was approved by the Australian National University Animal Experimentation Ethics Committee. C57BL63 mice were born and raised in 12:12 hrs light:dark cycle of 5 lux in individually vented cages, with free access to food and water. Age-matched adult mice (8-10 weeks) were randomly assigned to light damage and dim-reared control (non-light damage) groups. Animals of the light damage group were continuously exposed to 100 k lux white LED light for 5 days. Pupils were dilated twice daily at 10 am and bpm with a single drop of 1% atropine sulfate (8.3 mg of atropine). Dim-reared control animals were also pupil dilated twice each day, but were returned to dim cyclic light (12:12 hrs light:dark, 5 lux light).

### Light Exposure Device

During bright-light exposure, animals were housed in plastic boxes (two per box), with free access to food and water. The floors of the cages were coated with a reflective Perspex surface and illuminated by a 100-W 65000 k natural white LED (high CRI LED, Yuji, Beijing) mounted 18 cm above the plastic boxes. The LED light has an emission spectrum which more closely resembles daylight than halogen or incandescent bulbs. Temperature in the cages was maintained at ˜23±2° C. with a dual exhaust system to alleviate any heat generated from the LED, with one exhaust fan mounted next to the LED light source, and another one on the side of the cage. In order to regulate accurate illumination, each box was equipped with a dimmer and adjusted to 100 k lux using a light meter data logging device (Extech HD450). Animals were provided with bedding, food and water during the time course of light exposure, and their behaviour was monitored daily. All graphing and statistical analysis was performed using Prism 6 (GraphPad Software, CA, USA). Significant trends in time-course datasets were ascertained using the one-way or two-way analysis of variance (ANOVA) to determine statistical significance (p<0.05).

### Measurement of Retinal Function Using Electroretinography (ERG)

Full-field scotopic ERGs were performed to assess the retinal function of dim-reared control and 7 days light damaged animals. Briefly, mice were dark adapted overnight, anesthetized by intraperitoneal injection of xylazine (10 mg/kg) and ketamine (100 mg/kg) and the pupils dilated with a single drop of 1% atropine sulfate (8.3 mg of atropine). A single- or twin-flash paradigm was used to elicit mixed (rod and cone) or isolated cone responses, respectively. Flash stimuli for mixed responses were provided by an LED-based system (FS-250A Enhanced Ganzfeld, Photometric Solutions International, Melbourne), over a stimulus intensity range of 6.3 log cd s m⁻² (range −4.4-1.9 log cd s m⁻²). Interstimulus interval was increased from 2 s for the lowest intensities to 5 min for the highest intensities to allow complete recovery of the b-wave between stimuli. Isolated cone responses were obtained at 1.6 log cd s m⁻² following a rod-saturating stimulus of 1.9 log cd s m⁻² given 400 ms before the test stimulus. This short interval after a rod-saturating flash does not allow recovery of rod function, thereby revealing cone-only responses. The a-wave amplitude was measured from the baseline to the trough of the a-wave response and the b-wave amplitude was measured from the trough of the a-wave to the peak of the b-wave. Data are expressed as the mean wave amplitude ±SEM (pV). Two-way ANOVA, with Tukey's multiple comparisons post-hoc test, was performed to compare the responses from control and light damaged mice over the flash stimulus range. The a-wave and b-wave data were fitted with a Naka-Rushton equation [R/Rₘₐₓ=I/(I+K)] using the Solver function in Excel (Microsoft Version 2013) to determine Rₘₐₓ (maximum amplitude) and K (semisaturation constant) from the response amplitude (R) and the flash intensity (I) over the range of −4.4 to 1.9 log cd s m⁻². Statistics were performed using Prism (GraphPad Software V5; GraphPad Software, Inc., La Jolla, Calif., USA) and either a 2-way ANOVA for mixed a-wave and b-wave and students t-test for cone b-wave. For each analysis, differences with a p<0.05 were considered statistically significant.

### Intravitreal Injections

Intravitreal injections were performed as described in detail previously (Rutar M V et al (2012) J Neuroinflammation 9: 221) wherein animals were anesthetized using an intraperitoneal injection of ketamine (100 mg/kg; Troy Laboratories, NSW, Australia) and xylazil (12 mg/kg; Troy Laboratories). Injections into individual animals consisted of a 1 μL solution containing PBS (control), Eylea (2 μg/μL) or pentosan polysulfate (HI; 2 μg/μL) as formulated in Example 3. Animals were allowed to wake from anesthetic, during which corneal hydration was maintained though application of a synthetic tear gel (GenTeal Gel; Novartis, NSW, Australia). Animals were exposed to photo-oxidative damage for 5 days as described in Example 1.

### Results

Pentosan polysulfate (HI 2 μg) delivered by intravitreal injection maintained normal or near normal retinal function (FIGS. 5F and G) in comparison to treatment with vehicle alone (PBS) or aflibercept (Eylea 2 μg) in mice exposed to photo-oxidative damage. The a-wave and b-wave responses of the ERG (FIGS. 5F and G) reflects the differences in retinal morphology of untreated and treated animals described above. ERG a-wave and b-wave intensity response characteristics between pentosan polysulfate treated (HI 2 μg) mice was significantly different (p<0.05) compared to control mice (PBS) and aflibercept-treated mice (Eylea 2 μg). The pentosan polysulfate treated group (HI 2 μg) had higher a- and b-wave responses that were near normal and significantly higher than both control (PBS) and aflibercept-treated (Eylea 2 μg) groups. The benefit of pentosan polysulfate treatment was demonstrated across multiple flash intensities and was most pronounced at the highest flash intensity (p<0.05, FIGS. 5F-G).

### Example 5—Synthesis of Compounds of Formula 1

Unless otherwise specified, proton (¹H) and carbon (¹³C) NMR spectra were recorded at room temperature in base-filtered CDCl₃ with a Bruker spectrometer operating at 400 MHz for proton and 100 MHz for carbon nuclei. For ¹H NMR spectra, signals arising from the residual protio forms of the solvent were used as the internal standards. ¹H NMR spectroscopic data are recorded as follows: chemical shift (δ) [multiplicity, coupling constant(s) J (Hz), relative integral] where multiplicity is defined as: s=singlet; d=doublet; t=triplet; q=quartet; m=multiplet; br=broad or combinations of thereof. The signal due to residual CHCl₃ appearing at δH=7.26 ppm and the central resonance of the CDCl₃ “triplet” appearing at δC=77.0 or 77.16 ppm were used to reference ¹H and ¹³C NMR spectra, respectively. The quintet due to residual DMSO-d₅ appearing at δH=2.50 ppm and the central resonance of the DMSO-d₆ “multiplet” appearing at δC=39.52 ppm were used to reference ¹H and ¹³C NMR spectra, respectively. Infrared spectra (IR: max) were recorded with a Perkin-Elmer 1800 series FTIR spectrometer or a Perkin-Elmer UATR Spectrum Two FTIR spectrometer. Samples were analyzed as thin films on KBr plates or compressed and flattened on a diamond window. Low-resolution ESI mass spectra were recorded on a single quadrupole liquid chromatograph-mass spectrometer, while high-resolution measurements were conducted on a time-of-flight instrument. Low- and high-resolution EI mass spectra were recorded with a magnetic-sector machine. Melting points were measured with an Optimelt automated melting point system and are uncorrected. Analytical thin layer chromatography (TLC) was performed on aluminium-backed 0.2 mm thick silica gel 60 F254 plates. Eluted plates were visualized with a 254 nm UV lamp and/or by treatment with a suitable dip followed by heating. These dips included phosphomolybdic acid/ceric sulfate/sulfuric acid (concd.)/water (37.5 g:7.5 g:37.5 g:720 mL) or potassium permanganate/potassium carbonate/5% sodium hydroxide aqueous solution/water (3 g:20 g:5 mL:300 mL). Flash chromatographic separations were carried out according to protocols defined by Still et al. (1978) J. Org. Chem., 43: 2923 with silica gel 60 (40-63 μm) as the stationary phase and with the AR- or HPLC-grade solvents indicated. Starting materials and reagents were generally available from the Sigma-Aldrich, Merck, TCI, Strem, AK Scientific or Lancaster chemical companies and were used as supplied. Drying agents and other inorganic salts were purchased from the AJAX, BDH or Unilab chemical companies. Tetrahydrofuran (THF), diethyl ether, methanol and dichloromethane (DCM) were dried by using a Glass Contour solvent purification system that is based upon a technology originally described by Grubbs et al. (1996) Organometallics, 15: 1518. Where necessary or desirable, reactions were performed under nitrogen. Microwave reactions were conducted with a CEM Explorer microwave reactor. Microwave vessels were sealed with a snap-cap and irradiated for the time and at the temperatures specified, typically with a ramp time of 1 minute to the specified temperature at a maximum power of 200 W.

### General Procedure A—Preparation of 2,4-dichloroquinazolines

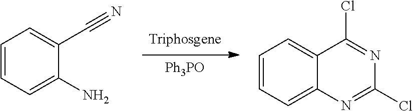

Using a procedure analogous to that reported by Zhong et al. (2012) Heterocycles, 85: 1417-1426, a magnetically stirred solution of triphenylphosphine oxide (170 mg, 0.6 mmol) in chlorobenzene (5 mL) at 0° C. was treated with triethylamine (400 μL). The solution was then treated dropwise with a solution of triphosgene (630 mg, 2.1 mmol) in chlorobenzene (6 mL) and stirring was continued at rt for 0.5 h. The mixture was then treated with 2-aminobenzonitrile (354 mg, 3 mmol) in one portion and heated at 120° C. for 5 hr. The mixture was cooled and stirred for 18 h at rt then water was added and the mixture extracted with ethyl acetate (EtOAc) (3×15 mL). The combined organic layers were dried (Na₂SO₄) and concentrated in vacuo to afford a yellow solid which was subjected to flash column chromatography [silica, 1:10 v/v EtOAc/Pet spirit elution] to give, after concentration of the appropriate fractions the title compound (268 mg, 45%) as a white solid. Spectral data were consistent with those reported by Zhong et al (2012). ¹H NMR (CDCl₃, 400 MHz) δ 8.27 (d, J=8.4 Hz, 1H), 8.04-7.97 (m, 2H), 7.79-7.71 (m, 1H); vₘₐₓ 1670, 1668, 1616, 1434, 1404, 1290, 1140, 753, 683 cm⁻¹.

### General Procedure B—Addition of Amines to 2,4-dichloroquinazolines

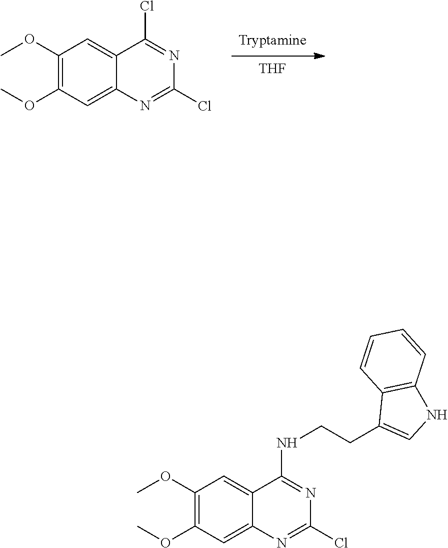 [SMILES: COc1cc2nc(Cl)nc(Cl)c2cc1OC ; COc1cc2nc(Cl)nc(NCCc3c[nH]c4ccccc34)c2cc1OC ; "THF"]

A magnetically stirred suspension of tryptamine (2.47 g, 15.4 mmol) in THF (100 mL) was treated with 2,4-dichloro-6,7-dimethoxyquinazoline (2.00 g, 7.72 mmol) followed by dropwise addition of triethylamine (1.08 mL, 7.72 mmol). The mixture was stirred for 18 h at 18° C., concentrated in vacuo and the residue diluted with DCM (80 mL) and washed with a saturated solution of sodium hydrogen carbonate (20 mL) and brine (20 mL) then dried (Na₂SO₄) and concentrated in vacuo to afford N-(2-(1H-indol-3-yl)ethyl)-2-chloro-6,7-dimethoxyquinazolin-4-amine (1.65 g; 56%). ¹H NMR (DMSO-d₆, 400 MHz) δ 10.84 (s, 1H), 8.53 (t, J=5.6 Hz, 1H), 7.71 (d, J=7.8 Hz, 1H), 7.61 (s, 1H), 7.35 (d, J=8.1 Hz, 1H), 7.20 (d, J=2.1 Hz, 1H), 7.10-7.05 (m, 1H), 7.09 (s, 1H), 6.99 (t, J=7.8 Hz, 1H), 3.89 (s, 3H), 3.87 (s, 3H) 3.80-3.71 (m, 2H), 3.09-3.04 (m, 2H); ¹³C NMR (DMSO-d₆, 100 MHz) δ 160.0, 155.3, 154.4, 148.4, 147.2, 136.3, 127.3, 122.7, 121.0, 118.5, 118.3, 111.7, 111.4, 107.0, 106.5, 102.3, 56.1, 55.8, 41.8, 24.6; (+)-LRESIMS m/z (rel. int.) 383 (100); (+)-HRESIMS calcd. for C₂₀H₂₀ClN₄O₂ [M+H]⁺ 383.1269, found 383.1271. vₘₐₓ 3408, 1587, 1499, 1425, 1336, 1249, 1220, 1148, 850, 743, 499 cm⁻¹.

### General Procedure C—Suzuki-Miyaura Cross-Coupling Procedure

The general procedure is illustrated with respect to the synthesis of BT-1060.

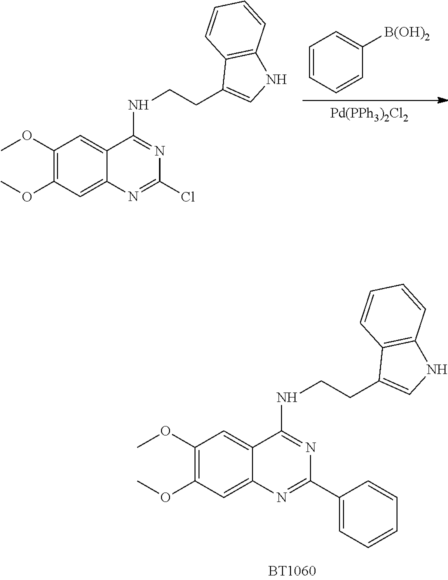

A 10 mL snap-cap microwave vessel fitted with a magnetic stirring bar was charged with a mixture of phenylboronic acid (9.4 mg, 77.2 μmol), N-(2-(1H-indol-3-yl)ethyl)-2-chloro-6,7-dimethoxyquinazolin-4-amine (20.0 mg, 52.3 μmol) and potassium carbonate (38.0 mg, 274 μmol) then treated with a degassed mixture of dimethoxyethane, water and ethanol (7:3:2, 1 mL). Bis(triphenylphosphine)palladium(II) dichloride (1.8 mg, 5 mol %) was added and the mixture was sparged with nitrogen for 0.05 hr, sealed then subjected to microwave irradiation (120° C./0.33 h, ramp time 1 minute, maximum power 200 W). The mixture was treated with water (1 mL) and extracted with EtOAc (3×2 mL) and the combined organic layers washed with brine and concentrated under a gentle stream of nitrogen. The resulting residue was subjected to flash column chromatography [silica, 1:1 v/v EtOAc/petroleum spirit elution] to give, after concentration of the appropriate fractions the compound BT-1060 as a white solid (14.4 mg, 65%). ¹H NMR (CDCl₃, 400 MHz) δ 8.58-8.54 (m, 2H), 8.15 (s, 1H), 7.73 (d, J=7.9 Hz, 1H), 7.52-7.43 (m, 3H), 7.41 (d, J=7.9 Hz, 1H), 7.30 (s, 1H), 7.23 (app. t, J=7.5 Hz, 1H), 7.14 (app. t, J=7.5 Hz, 1H), 7.10 (d, J=2.0 Hz, 1H), 6.60 (s, 1H), 5.54 (s, 1H), 4.15-4.09 (m, 2H, EtOAc obscured), 4.00 (s, 3H), 3.81 (s, 3H), 3.27 (app. t, J=6.6 Hz, 2H); ¹³C NMR (CDCl₃, 100 MHz) δ 159.5, 158.4, 154.2, 148.5, 147.3, 139.1, 136.4, 129.7, 128.2 (2C), 128.1 (2C), 127.7, 122.2, 119.7, 118.8, 115.3, 113.5, 111.4, 108.0, 107.3, 99.5, 56.2, 56.1, 42.2, 24.9; (+)-LRESIMS m/z (rel. int.) 425 (100) [M+H]⁺, 447 (8) [M+Na]⁺; (+)-HRESIMS calcd. for C₂₆H₂₅N₄O₂ [M+H]⁺ 425.1972, found 425.1978; vₘₐₓ 3347, 1624, 1594, 1524, 1501, 1458, 1422, 1368, 1254, 1214, 1128, 1027, 854 cm⁻¹.

### General Procedure D—Addition of Amines to 2-chloroquinazolines

The general procedure is illustrated with respect to the synthesis of the compound BT-2029.

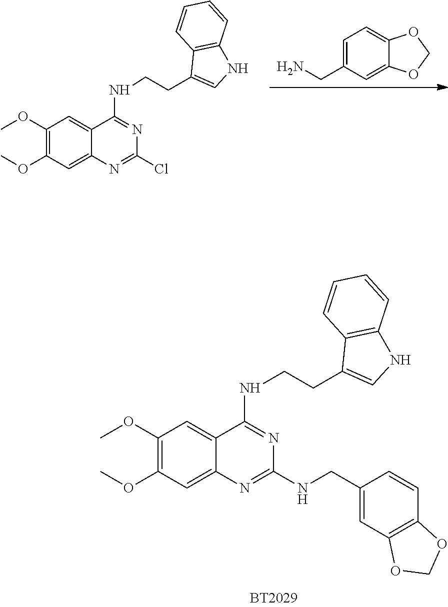 [SMILES: COc1cc2nc(Cl)nc(NCCc3c[nH]c4ccccc34)c2cc1OC ; NCc1ccc2c(c1)OCO2 ; COc1cc2nc(NCc3ccc4c(c3)OCO4)nc(NCCc3c[nH]c4ccccc34)c2cc1OC]

A 10 mL snap-cap microwave vessel was charged with a mixture of piperonyl amine (103 mg, 0.68 mmol), N-(2-(1H-indol-3-yl)ethyl)-2-chloro-6,7-dimethoxyquinazolin-4-amine (65 mg, 0.17 mmol), N,N-diisopropylethylamine (89 μL, 0.51 mmol) and n-butanol (1.5 mL) sealed then subjected to microwave irradiation (160° C./0.5 h, ramp time 2 minutes, maximum power 200 W). The mixture was cooled and concentrated in vacuo and the resulting residue was subjected to flash column chromatography [silica, 1:10 v/v ammoniacal methanol/DCM elution] to give, after concentration of the appropriate fractions the compound BT-2029 as a white solid (67 mg, 79%). ¹H NMR (DMSO-d₆, 400 MHz) δ 10.81 (s, 1H), 7.76-7.69 (m, 1H), 7.57 (d, J=7.9 Hz, 1H), 7.40 (s, 1H), 7.34 (d, J=8.1 Hz, 1H), 7.16 (d, J=2.3 Hz, 1H), 7.06 (t, J=7.5 Hz, 1H), 6.96 (t, J=7.4 Hz, 1H), 6.92 (s, 1H), 6.81 (dd, J=8.0, 0.9 Hz, 1H), 6.78 (d, J=8.0 Hz, 1H), 6.68 (s, 1H), 6.68-6.65 (m, 1H), 5.93 (s, 2H), 4.46 (d, J=6.3 Hz, 2H), 3.81 (s, 3H), 3.79 (s, 3H), 3.76-3.69 (m, 2H), 3.03 (app. t, J=7.6 Hz, 2H); (+)-LRESIMS m/z (rel. int.) 498 (100) [M+H]⁺; (+)-HRESIMS calcd. for C₂₈H₂₈N₅O₄ [M+H]⁺ 498.2136, found 498.2143; vmax 1626, 1498, 1488, 1456, 1435, 1359, 1231, 1209, 1035, 740 cm⁻¹.

### General Procedure E—Amide Preparation

The general procedure is illustrated with respect to the synthesis of the compound BT-2161.

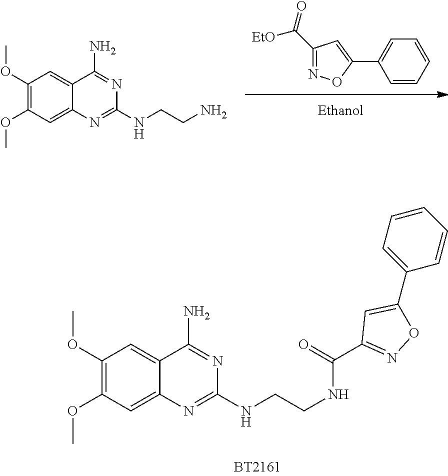 [SMILES: COc1cc2nc(NCCN)nc(N)c2cc1OC ; CCOC(=O)c1cc(-c2ccccc2)on1 ; COc1cc2nc(NCCNC(=O)c3cc(-c4ccccc4)on3)nc(N)c2cc1OC]

A 10 mL snap-cap microwave vessel was charged with N²-(2-aminoethyl)-6,7-dimethoxyquinazoline-2,4-diamine (121.2 mg, 0.46 mmol), ethyl 5-phenylisoxazole-3-carboxylate (50.0 mg, 0.23 mmol) prepared according to the procedure of Watterson et al. (J. Med. Chem. 2016, 59, 2820) and ethanol (1 mL). The tube was sealed and irradiated at 80° C. for 1 h using a CEM Explorer microwave reactor (ramp time 1 minute, maximum power 200 W), before being stirred at 18° C. for 48 h. Solvent was evaporated and the resultant residue subjected to column chromatography [silica, 10:90 v/v methanol/dichloromethane elution] to afford, after concentration of the appropriate fractions (R_{f}=0.24) the compound BT-2161 (75.0 mg, 75%) as white solid. ¹H NMR (400 MHz, DMSO-d₆) δ 9.46 (brs, 1H), 7.88 (m, 2H), 7.53 (m, 3H), 7.45 (s, 1H), 7.34 (s, 1H), 7.15 (brs, 2H), 6.91 (s, 1H), 6.50 (brs, 1H), 3.88 (s, 3H), 3.79 (s, 3H), 3.51 (brs, 4H); ¹³C NMR (100 MHz, DMSO-d₆) δ 170.3, 161.2, 159.7, 159.2, 158.5, 154.1, 148.0, 144.8, 130.8, 129.3 (2C), 126.3, 125.7 (2C), 105.0, 104.0, 103.3, 99.9, 55.9, 55.4, 41.1, 40.2; (+)-LRESIMS m/z (rel. int.) 435 (100) [M+H]⁺; HRMS (ESI, +ve) Found: (M+H)⁺ 435.1766, C₂₂H₂₃N₆O₄ requires 435.1781; vₘₐₓ 3345, 3226, 2938, 1653, 1610, 1575, 1504, 1475, 1444, 1386, 1334, 1212, 1180, 1109, 1003, 853, 765 cm⁻¹.

### General Procedure F—Boc Deprotection with Trifluoroacetic Acid in Dichloromethane

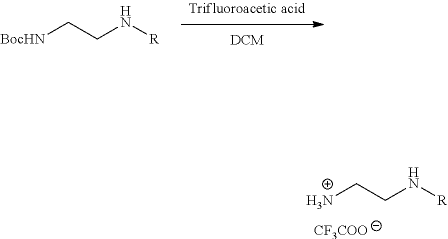

A magnetically stirred suspension of Boc-protected compound (1.80 mmol) in DCM (4 mL) maintained at 0° C. was treated with trifluoroacetic acid (1 mL) and magnetically stirred for 2 h. The cold bath was removed and the mixture was then stirred for a further 1 h at room temperature (rt). The reaction was checked for completion by TLC analysis and then the solvent was removed with a gentle stream of nitrogen and the remaining gum was triturated with diethyl ether (3×10 mL) then placed under high vacuum for 1 h to afford the amine trifluoroacetate salt as a powder and used directly without further purification.

### Preparation (i). 2,4-dichloro-6-methoxyquinazoline

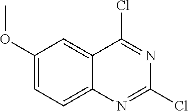

Prepared according to General Procedure A, from reaction of 2-amino-5-methoxybenzonitrile (444 mg, 3.00 mmol) and triphosgene (630 mg, 2.10 mmol) which afforded 2,4-dichloro-6-methoxyquinazoline (406 mg, 59%) as a white powder and used without further purification. ¹H NMR (CDCl₃, 400 MHz) δ 7.91 (d, J=9.2 Hz, 1H), 7.62 (dd, J=9.2, 2.7 Hz, 1H), 7.42 (d, J=2.8 Hz, 1H), 4.00 (s, 3H); ¹³C NMR (CDCl₃, 100 MHz) δ 161.7, 159.6, 152.7, 148.5, 129.4, 129.1, 123.4, 102.8, 56.0; vmax 1619, 1541, 1489, 1418, 1390, 1291, 1221, 1109, 1019, 854, 837 cm⁻¹.

### Preparation (ii). 2,4-dichloro-7-bromoquinazoline

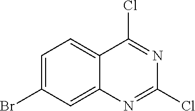 [SMILES: Clc1nc(Cl)c2ccc(Br)cc2n1]

Prepared according to General Procedure A, from reaction of 2-amino-4-bromobenzonitrile (591 mg, 3.00 mmol) and triphosgene (630 mg, 2.1 mmol) which afforded 2,4-dichloro-7-bromoquinazoline (527 mg, 64%) as a beige powder and used without further purification. ¹H NMR (CDCl₃, 400 MHz) δ 8.19 (d, J=1.3 Hz, 2H), 8.12 (d, J=8.8 Hz, 2H), 7.83 (dd, J=8.8, 1.3 Hz, 1H); LRMS (EI, 70 eV) m/z (rel. int.) 278 (100, M+.), 276 (63, M+.), 243 (75), 241 (58), 178 (50); HREIMS calcd. for C₈H₃⁷⁹BrCl₂N₂ [M+.] 275.8851, found 275.8860; vmax 3291, 3033, 2839, 1730, 1676, 1609, 1592, 1424, 1282, 1015, 858 cm⁻¹.

### Preparation (iii). N-(2-(1H-indol-3-yl)ethyl)-2-chloroquinazolin-4-amine

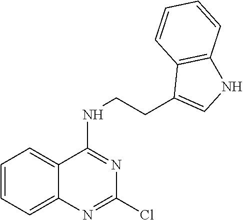 [SMILES: Clc1nc(NCCc2c[nH]c3ccccc23)c2ccccc2n1]

Prepared according to General Procedure B, from reaction of 2,4-dichloroquinazoline (153 mg, 0.77 mmol), tryptamine (308 mg, 1.54 mmol) and triethylamine (200 μL, 1.44 mmol) to afford a residue that was subjected to flash column chromatography [silica, 1:20 v/v MeOH/DCM elution] to give, after concentration of the appropriate fractions N-(2-(1H-indol-3-yl)ethyl)-2-chloroquinazolin-4-amine as a white solid (217 mg, 88%). ¹H NMR (CDCl₃, 400 MHz) δ 7.74 (d, J=8.0 Hz, 1H), 7.73-7.63 (m, 2H), 7.44-7.36 (m, 2H), 7.39-7.30 (m, 2H), 7.23 (d, J=8.0 Hz, 1H), 7.15 (t, J=7.5 Hz, 1H), 7.13-7.07 (m, 1H), 6.10-6.01 (m, 1H), 4.01 (app. q, J=6.2 Hz, 2H), 3.19 (t, J=6.6 Hz, 2H); ¹³C NMR (CDCl₃, 100 MHz) δ 160.8, 157.8, 150.7, 136.5, 133.3, 127.7, 127.3, 126.0, 122.5, 122.2, 120.7, 119.7, 118.7, 113.3, 112.6, 111.4, 41.8, 24.7; (+)-LRESIMS m/z (rel. int.) 323 (100) [M+H]⁺; (+)-HRESIMS calcd. for C₁₈H₁₆ClN₄ [M+H]⁺323.1058, found 323.1065; vₘₐₓ 3407, 1606, 1574, 1532, 1426, 1334, 1275, 1191, 945, 738 cm⁻¹.

### Preparation (iv). N-(2-(1H-indol-3-yl)ethyl)-7-bromo-2-chloroquinazolin-4-amine

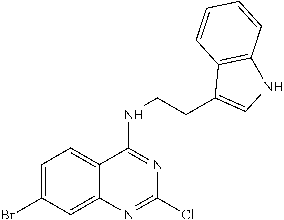 [SMILES: Clc1nc(NCCc2c[nH]c3ccccc23)c2ccc(Br)cc2n1]

Prepared according to General Procedure B, from reaction of 7-bromo-2,4-dichloroquinazoline (100 mg, 0.36 mmol), tryptamine (115 mg, 0.72 mmol) and triethylamine (200 μL, 1.44 mmol) to afford a residue that was subjected to flash column chromatography [silica, 1:20 v/v EtOAc/DCM elution] to give, after concentration of the appropriate fractions N-(2-(1H-indol-3-yl)ethyl)-7-bromo-2-chloroquinazolin-4-amine as a cream powder (115 mg, 80%). ¹H NMR (CDCl₃, 400 MHz) δ 8.12 (s, 1H), 7.91 (d, J=2.0 Hz, 1H), 7.67 (d, J=7.9 Hz, 1H), 7.46-7.36 (m, 2H), 7.26-7.09 (m, 4H), 5.96 (s, 1H), 4.00 (app. q, J=6.2 Hz, 2H), 3.20 (t, J=6.2 Hz, 2H); (+)-LRESIMS m/z (rel. int.) 401 (100), 403 (100) [M+H]⁺; (+)-HRESIMS calcd. for C₁₈H₁₅⁷⁹BrClN₄ [M+H]⁺ 401.0163, found 401.0179; vₘₐₓ 3407, 1606, 1574, 1532, 1426, 1334, 1275, 1191, 945, 738 cm⁻¹.

### Preparation (v). N-(2-(1H-indol-3-yl)ethyl)-2-chloro-6-methoxyquinazolin-4-amine

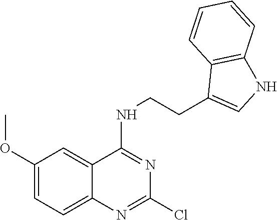 [SMILES: COc1ccc2nc(Cl)nc(NCCc3c[nH]c4ccccc34)c2c1]

Prepared according to General Procedure B, from reaction of 2,4-dichloro-6-methoxyquinazoline (200 mg, 0.87 mmol), tryptamine (280 mg, 1.75 mmol) and triethylamine (200 μL) to afford a residue that was subjected to flash column chromatography [silica, 1:20 v/v MeOH/DCM elution] to give, after concentration of the appropriate fractions N-(2-(1H-indol-3-yl)ethyl)-2-chloro-6-methoxyquinazolin-4-amine (211 mg, 69%) as a white powder. ¹H NMR

(CDCl₃, 400 MHz) δ 8.13 (s, 1H), 7.71 (d, J=8.1 Hz, 1H), 7.68 (d, J=9.2 Hz, 1H), 7.42 (d, J=8.1 Hz, 1H), 7.33 (dd, J=9.2, 2.7 Hz, 1H), 7.23 (dd, J=8.0, 0.8 Hz, 1H), 7.21-7.12 (m, 1H), 7.12 (d, J=2.3 Hz, 1H), 6.52 (d, J=2.7 Hz, 1H), 5.82-5.72 (m, 1H), 4.00 (app. q, J=6.2 Hz, 2H), 3.72 (s, 3H), 3.21 (app. t, J=6.2 Hz, 2H); ¹H NMR (DMSO-d₆, 400 MHz) δ 10.85 (s, 1H), 8.74 (t, J=5.5 Hz, 1H), 7.73 (d, J=7.8 Hz, 1H), 7.67 (d, J=2.8 Hz, 1H), 7.57 (d, J=9.0 Hz, 1H), 7.41 (dd, J=9.0, 2.8 Hz, 1H), 7.37 (d, J=7.8 Hz, 1H), 7.22 (d, J=2.0 Hz, 1H), 7.09 (t, J=7.2 Hz, 1H), 7.00 (t, J=7.2 Hz, 1H), 3.87 (s, 3H), 3.85-3.75 (m, 2H), 3.15-3.04 (m, 2H); ¹³C NMR (DMSO-d₆, 100 MHz) δ 160.4, 157.1, 155.0, 145.4, 136.3, 128.1, 127.3, 124.2, 122.8, 121.0, 118.5, 118.3, 114.2, 111.6, 111.4, 102.8, 55.9, 41.9, 24.4; (+)-LRESIMS m/z (rel. int.) 353 (100) [M+H]⁺, [M+H]⁺; (+)-HRESIMS calcd. for C₁₉H₁₈C1N₄O [M+H]⁺ 353.1164, found 353.1171. vₘₐₓ 3476, 1629, 1587, 1568, 1535, 1514, 1450, 1331, 1254, 1240, 1169, 1041, 940, 904, 824, 745, 578 cm⁻¹.

### Preparation (vi). N²-(2-aminoethyl)-6,7-dimethoxyquinazoline-2,4-diamine

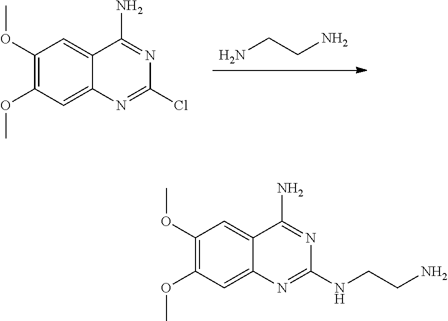

A solution of 2-chloro-6,7-dimethoxyquinazolin-4-amine (2.00 g, 8.34 mmol) and ethylenediamine (5.57 mL, 83.45 mmol) in water (25 mL) was heated under reflux for 16 h. The reaction mixture was then cooled down and solvent was evaporated under high vacuum to remove as much as possible the excess of ethylenediamine. The resulting residue was subjected to flash column chromatography [silica, 5:15:80 v/v 35% aqueous ammonia/methanol/chloroform elution] to give, after concentration of the appropriate fractions (R_{f}=0.19), N²-(2-aminoethyl)-6,7-dimethoxyquinazoline-2,4-diamine (1.80 g, 82%) as a light yellow solid. ¹H NMR (400 MHz, methanol-d₄) δ 7.27 (s, 1H), 6.79 (s, 1H), 3.88 (s, 3H), 3.84 (s, 3H), 3.45 (t, J=6.2 Hz, 2H), 2.83 (t, J=6.2 Hz, 2H); ¹³C NMR (100 MHz, methanol-d₄) δ 163.3, 160.8, 156.3, 149.8, 146.8, 105.1, 104.6, 104.4, 56.6, 56.2, 44.6, 42.5; (+)-LRESIMS m/z (rel. int.) 264 (100) [M+H]⁺; vₘₐₓ 3168, 2934, 1671, 1603, 1576, 1503, 1480, 1454, 1435, 1380, 1312, 1232, 1209, 1111, 1031, 1004, 841, 829, 784 cm⁻¹.

### Preparation (vii). 2-(3-Aminoazetidin-1-yl)-6,7-dimethoxyquinazolin-4-amine

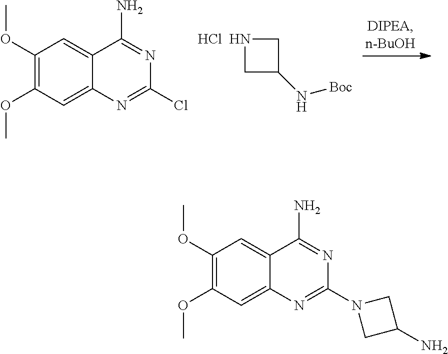

A 10 mL snap-cap microwave vessel was charged with a mixture of 2-chloro-6,7-dimethoxyquinazolin-4-amine (100.0 mg, 0.42 mmol), tert-butyl azetidin-3-ylcarbamate hydrochloride (130.6 mg, 0.63 mmol), N,N-diisopropylethylamine (0.18 mL, 1.04 mmol) and n-butanol (2 mL). The tube was sealed then subjected to microwave irradiation (120° C./1 h, ramp time 5 minutes, maximum power 250 W). The mixture was cooled and concentrated in vacuo and the resulting residue was subjected to flash column chromatography [silica, 5:95 to 20:80 v/v methanol/ethyl acetate elution] to give, after concentration of the appropriate fractions tert-butyl (1-(4-amino-6,7-dimethoxyquinazolin-2-yl)azetidin-3-yl)carbamate (R_{f}=0.40, 20:80 v/v methanol/ethyl acetate elution). ¹H NMR (400 MHz, methanol-d₄) δ 7.26 (s, 1H), 6.79 (s, 1H), 4.45 (brs, 1H), 4.33 (t, J=7.9 Hz, 2H), 3.92 (dd, J=8.5 and 5.6 Hz, 2H), 3.89 (s, 3H), 3.86 (s, 3H), 1.45 (s, 9H); (+)-LRESIMS m/z (rel. int.) 376 (100) [M+H]⁺; vₘₐₓ 3329, 3215, 2974, 1685, 1640, 1574, 1497, 1465, 1454, 1441, 1383, 1345, 1241, 1210, 1158, 1103, 1000, 842, 783 cm⁻¹. A solution of tert-butyl (1-(4-amino-6,7-dimethoxyquinazolin-2-yl)azetidin-3-yl)carbannate obtained above in DCM (2 mL) was treated dropwise with trifluoroacetic acid (0.5 mL) at 0° C. The resulting mixture was stirred at 20° C. until the completion conversion (observed by TLC). Solvent was then evaporated to obtain the TFA salt of 2-(3-aminoazetidin-1-yl)-6,7-dimethoxyquinazolin-4-amine. The product was then dissolved in pyridine (2 mL) and the solution was stirred for 15 min before evaporated and subjected to a short pad silica gel to afford the title compound (R_{f}=0.24, 10:90 v/v methanol saturated ammonia/dichloromethane elution) (100.0 mg, 87%). ¹H NMR (400 MHz, acetone-d₆) δ 9.11 (brs, 1H), 7.39 (s, 1H), 6.87 (s, 1H), 6.72 (s, 2H), 4.80 (m, 1H), 4.37 (t, J=8.3 Hz, 2H), 4.05 (dd, J=9.0 and 5.4 Hz, 2H), 3.89 (s, 3H); (+)-LRESIMS m/z (rel. int.) 276 (100) [M+H]⁺; vₘₐₓ 3343, 3195, 2957, 1640, 1606, 1558, 1492, 1438, 1411, 1376, 1345, 1277, 1237, 1210, 1128, 1097, 1031, 997, 839, 785 cm⁻¹.

### Preparation (viii). 2-Chloro-6,7-dimethoxy-N-(2-(pyridin-2-yl)ethyl)quinazolin-4-amine

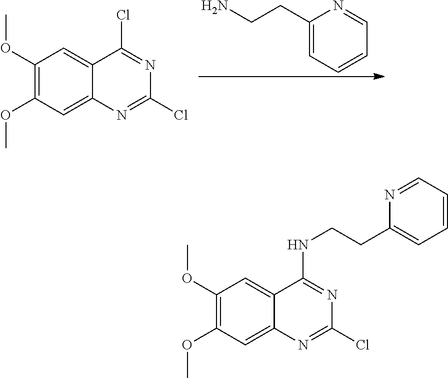 [SMILES: COc1cc2nc(Cl)nc(Cl)c2cc1OC ; NCCc1ccccn1 ; COc1cc2nc(Cl)nc(NCCc3ccccn3)c2cc1OC]

According to General Procedure B, a solution of 2,4-dichloro-6,7-dimethoxyquinazoline (400.0 mg, 1.54 mmol), 2-pyridylethylamine (370 μL, 3.08 mmol), triethylamine (215 μL, 1.54 mmol) in tetrahydrofuran (20 mL) was stirred at 18° C. for 18 h. 2-Chloro-6,7-dimethoxy-N-(2-(pyridin-2-yl)ethyl)quinazolin-4-amine (510.0 mg, 96%) (R_{f}=0.14, 97.5:2.5 v/v methanol/dicloromethane) was obtained as a white solid. ¹H NMR (400 MHz, chloroform-d) δ 8.52 (d, J=4.7 Hz, 1H), 8.03 (brs, NH), 7.65 (t, J=7.6 Hz, 1H), 7.24 (d, J=7.8 Hz, 1H), 7.22-7.14 (m, 1H), 7.07 (s, 1H), 7.00 (s, 1H), 4.00 (s, 3H), 3.97 (m, overlapped, 2H), 3.93 (s, 3H), 3.15 (t, J=6.0 Hz, 2H); ¹³C NMR (101 MHz, chloroform-d) δ 160.3, 159.9, 156.4, 154.8, 148.9, 148.8, 147.7, 137.3, 124.0, 122.1, 107.2, 107.2, 100.3, 56.3, 56.1, 40.8, 35.7; MS (ESI, +ve) m/z 367 and 369 [(M+Na), 33 and 100%]; vₘₐₓ 3266, 2935, 1619, 1587, 1522, 1499, 1474, 1432, 1405, 1355, 1253, 1219, 1174, 1144, 1042, 1000, 854, 801, 788, 772, 757, 638 cm⁻¹.

### Synthesis of BT-2162 and BT-2172

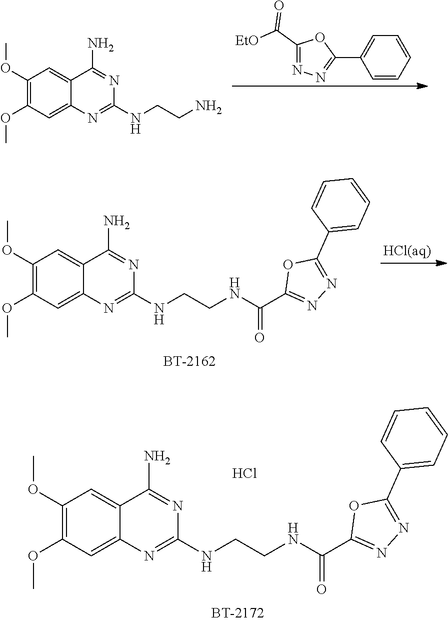 [SMILES: COc1cc2nc(NCCN)nc(N)c2cc1OC ; CCOC(=O)c1nnc(-c2ccccc2)o1 ; COc1cc2nc(NCCNC(=O)c3nnc(-c4ccccc4)o3)nc(N)c2cc1OC ; COc1cc2nc(NCCNC(=O)c3nnc(-c4ccccc4)o3)nc(N)c2cc1OC ; Cl]

According to general procedure E, a solution of N²-(2-aminoethyl)-6,7-dimethoxyquinazoline-2,4-diamine (120.7 mg, 0.46 mmol), ethyl 5-phenyl-1,3,4-oxadiazole-2-carboxylate (50.0 mg, 0.23 mmol) prepared according to the procedure of Dost et al. (1985) J. Prakt. Chem., 327: 109, in ethanol (1 mL) was irradiated at 80° C. for 3 h (ramp time 1 minute, maximum power 200 W). BT-2162 (91.0 mg, 91%) (R_{f}=0.24, 10:90 v/v methanol/dichloromethane) was obtained as a white solid. ¹H NMR (400 MHz, DMSO-d₆) δ 9.93 (brs, 1H), 8.04 (d, J=7.1 Hz, 2H), 7.67 (m, 1H), 7.61 (m, 2H), 7.43 (s, 1H), 7.16 (brs, 2H), 6.91 (s, 1H), 6.52 (brs, 1H), 3.86 (s, 3H), 3.78 (s, 3H), 3.51 (s, 4H); ¹³C NMR (100 MHz, DMSO-d₆) δ 164.9, 161.2, 159.1, 158.6, 154.1, 153.1, 147.8, 144.8, 132.6, 129.5 (2C), 127.0 (2C), 122.8, 104.8, 103.9, 103.3, 55.9, 55.4, 41.2, 39.9; (+)-LRESIMS m/z (rel. int.) 436 (100) [M+H]⁺; HRMS (ESI, +ve) Found: (M+H) 436.1719, C₂₁H₂₂N₇O₄ requires 436.1733; vₘₐₓ 3246, 2969, 1675, 1648, 1601, 1578, 1547, 1503, 1451, 1384, 1366, 1318, 1276, 1235, 1210, 1112, 1006, 829, 709 cm⁻¹.

A magnetically stirred suspension of BT-2162 (22.5 mg, 52 μmol) in dioxane (3 mL) maintained at 0° C. (ice water bath) was treated dropwise with a solution of HCl (100 μL, 4 M in dioxane). The mixture was stirred for 5 min then concentrated by a gentle stream of nitrogen then the solid triturated with ether (2 mL) and the residue held under high vacuum for 1 h to afford BT-2172 (20 mg, 82%), the hydrochloride salt of BT-2162 as a white powder. ¹H NMR (400 MHz, DMSO-d₆) δ 12.28 (br s, 1H), 9.47 (t, J=5.7 Hz, 1H), 8.81 (br s, 1H), 8.60 (br s, 1H), 8.07 (d, J=7.6 Hz, 2H), 7.86 (br s, 1H), 7.74-7.56 (m, 4H), 7.00 (br s, 1H), 3.88 (s, 3H), 3.82 (s, 3H), 3.65-3.61 (m, 2H), 3.59-3.55 (m, 2H); (+)-LRESIMS m/z (rel. int.) 436 [(M+H)⁺, 100%].

### Synthesis of BT-2005

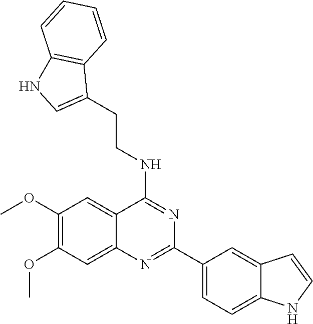 [SMILES: COc1cc2nc(-c3ccc4[nH]ccc4c3)nc(NCCc3c[nH]c4ccccc34)c2cc1OC]

Prepared according to General Procedure C from reaction of indole-5-boronic acid pinacol ester (19 mg, 79 μmol) and N-(2-(1H-indol-3-yl)ethyl)-2-chloro-6,7-dimethoxyquinazolin-4-amine (20 mg, 52.3 μmol) to afford a residue that was subjected to flash column chromatography [silica, 1:10 v/v MeOH/DCM elution] to give, after concentration of the appropriate fractions the compound BT-2005 as a white powder (6 mg, 26%). ¹H NMR N-Hs not observed (CD₃OD, 400 MHz) δ 8.51 (s, 1H), 8.01 (d, J=8.6 Hz, 1H), 7.63 (d, J=7.7 Hz, 1H), 7.44 (d, J=8.6 Hz, 1H), 7.35-7.30 (m, 3H), 7.13 (s, 1H), 7.10-7.05 (m, 2H), 6.96 (app. t, 3=7.7 Hz, 1H), 6.57-6.56 (m, 1H), 4.00 (app. t, J=7.7 Hz, 2H), 3.92 (s, 3H), 3.87 (s, 3H), 3.18 (t, J=7.5 Hz, 2H); (+)-LRESIMS m/z (rel. int.) 464 (100) [M+H]⁺; (+)-HRESIMS calcd. for C₂₈H₂₆N₅O₂ [M+H]⁺ 464.2081, found 464.2087; vₘₐₓ 1628, 1564, 1515, 1506, 1456, 1425, 1370, 1345, 1277, 1235, 1213, 1129, 854 cm⁻¹.

### Synthesis of BT-2007

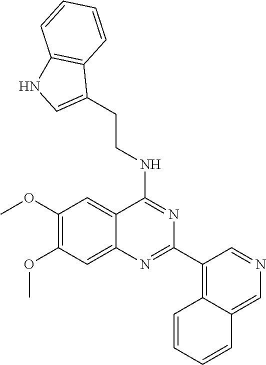 [SMILES: COc1cc2nc(-c3cncc4ccccc34)nc(NCCc3c[nH]c4ccccc34)c2cc1OC]

Prepared according to General Procedure C from reaction of isoquinoline-4-boronic acid pinacol ester (20 mg, 79 μmol) and N-(2-(1H-indol-3-yl)ethyl)-2-chloro-6,7-dimethoxyquinazolin-4-amine (20 mg, 52.3 μmol) to afford a residue that was subjected to flash column chromatography [silica, 1:10 v/v MeOH/DCM elution] to give, after concentration of the appropriate fractions the compound BT-2007 as a white powder (16 mg, 64%). ¹H NMR (CDCl₃, 400 MHz) δ 9.27 (s, 1H), 9.20 (s, 1H), 8.94 (d, J=8.6 Hz, 1H), 8.47 (s, 1H), 8.01 (d, J=8.2 Hz, 1H), 7.75-7.71 (m, 1H), 7.64-7.60 (m, 2H), 7.35 (d, J=8.2 Hz, 1H), 7.34 (s, 1H), 7.21-7.12 (m, 1H), 7.05 (s, 1H), 7.03 (t, J=7.3 Hz, 1H) 6.75 (s, 1H), 6.03 (s, NH), 4.06-4.02 (m, 2H), 4.00 (s, 3H), 3.82 (s, 3H), 3.21 (app. t, J=6.6 Hz, 2H); ¹³C NMR (CDCl₃, 100 MHz) δ 159.3, 158.5, 154.4, 153.2, 149.1, 146.5, 144.7, 136.4, 134.1, 130.9, 130.5, 128.7, 127.9, 127.6, 127.1, 125.8, 122.3, 122.1, 119.5, 118.6, 113.1, 111.4, 107.6, 107.1, 99.7, 56.2, 56.2, 42.4, 24.9; (+)-LRESIMS m/z (rel. int.) 476 (100) [M+H]⁺; (+)-HRESIMS calcd. for C₂₉H₂₆N₅O₂ [M+H]⁺ 476.2081, found 476.2087; vₘₐₓ 1622, 1584, 1525, 1500, 1424, 1359, 797, 742 cm⁻¹.

### Synthesis of BT-2057

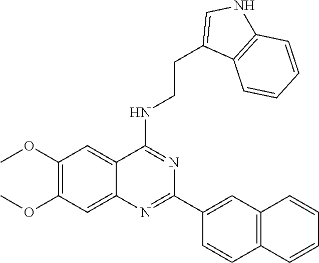 [SMILES: COc1cc2nc(-c3ccc4ccccc4c3)nc(NCCc3c[nH]c4ccccc34)c2cc1OC]

Prepared according to General Procedure C from reaction of 2-naphthalene boronic acid (44 mg, 0.25 mmol) and N-(2-(1H-indol-3-yl)ethyl)-2-chloro-6,7-dimethoxyquinazolin-4-amine (65 mg, 0.17 mmol) to afford a residue that was subjected to flash column chromatography [silica, 1:5 v/v MeOH/DCM elution] to give, after concentration of the appropriate fractions the compound BT-2057 as colourless needles (60 mg, 74%). Compound BT-2057 was recrystallized from dichloromethane/methanol. ¹H NMR (DMSO-d₆, 400 MHz) δ 10.89 (s, 1H), 9.05 (s, 1H), 8.66 (dd, J=8.6, 1.4 Hz, 1H), 8.22 (t, J=5.5 Hz, 1H), 8.05-7.96 (m, 3H), 7.74 (d, J=7.8 Hz, 1H), 7.66 (s, 1H), 7.59-7.53 (m, 2H), 7.38 (d, J=8.1 Hz, 1H), 7.30 (d, J=2.0 Hz, 1H), 7.26 (s, 1H), 7.11 (app. t, J=7.4 Hz, 1H), 7.01 (app. t, J=7.4 Hz, 1H), 4.06-3.98 (m, 2H), 3.96 (s, 3H), 3.92 (s, 3H), 3.22 (app. t, J=7.8 Hz, 2H); ¹³C NMR (DMSO-d₆, 100 MHz) one peak overlapping δ 158.6, 157.9, 153.9, 148.3, 146.8, 136.7, 136.3, 133.7, 132.8, 128.7, 127.6, 127.5, 127.4, 127.0, 126.6, 126.2, 125.3, 122.8, 121.0, 118.4, 118.3, 112.1, 111.5, 107.5, 102.2, 56.0, 55.7, 41.8, 25.1; (+)-LRESIMS m/z (rel. int.) 475 (100) [M+H]⁺; (+)-HRESIMS calcd. for C₃₀H₂₇N₄O₂ [M+H]⁺ 475.2129, found 475.2134; vₘₐₓ 3448, 1620, 1583, 1524, 1495, 1422, 1368, 1215, 856, 787, 743, 725 cm⁻¹.

### Synthesis of BT-2062

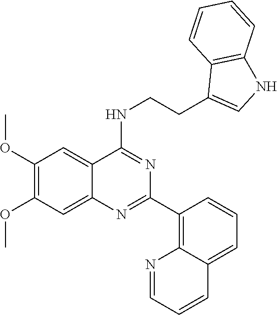 [SMILES: COc1cc2nc(-c3cccc4cccnc34)nc(NCCc3c[nH]c4ccccc34)c2cc1OC]

Prepared according to General Procedure C from reaction of 8-quinoline boronic acid (44 mg, 0.25 mmol) and N-(2-(1H-indol-3-yl)ethyl)-2-chloro-6,7-dimethoxyquinazolin-4-amine (65 mg, 0.17 mmol) to afford a residue that was subjected to flash column chromatography [silica, 1:30 v/v MeOH/DCM elution] to give, after concentration of the appropriate fractions the compound BT-2062 as colourless needles (55 mg, 68%). ¹H NMR (DMSO-d₆, 400 MHz) δ 10.72 (s, 1H), 8.82 (dd, J=4.1, 1.5 Hz, 1H), 8.43 (dd, J=8.3, 1.5 Hz, 1H), 8.17 (t, J=5.4 Hz, 1H), 8.06 (d, J=8.1 Hz, 1H), 7.87 (d, J=6.9 Hz, 1H), 7.70 (d, J=7.7 Hz, 1H), 7.67 (s, 1H), 7.51 (dd, 3=8.3, 4.1 Hz, 1H), 7.37 (d, J=7.7 Hz, 1H), 7.25 (d, J=8.1 Hz, 1H), 7.16 (s, 1H), 7.11 (d, J=1.7 Hz, 1H), 6.94 (t, J=7.5 Hz, 1H), 6.54 (t, J=7.5 Hz, 1H), 3.93 (s, 3H), 3.90 (s, 3H), 3.76-3.66 (m, 2H), 3.07 (app. t, J=7.7 Hz, 2H); ¹³C NMR (DMSO-d₆, 100 MHz) δ 161.5, 158.1, 153.8, 150.1, 148.2, 146.2, 145.9, 140.5, 136.2, 136.1, 129.5, 128.2, 128.0, 127.3, 125.9, 122.5, 121.2, 120.7, 118.6, 117.8, 112.1, 111.1, 107.2, 107.1, 102.0, 56.0, 55.7, 41.7, 24.9; (+)-LRESIMS m/z (rel. int.) 476 (100) [M+H]⁺; (+)-HRESIMS calcd. for C₂₉H₂₆N₅O₂ [M+H]⁺ 476.2081, found 476.2087; vₘₐₓ 1622, 1585, 1524, 1502, 1419, 1356, 1219, 851, 797, 745, 639 cm⁻¹.

### Synthesis of BT-2090

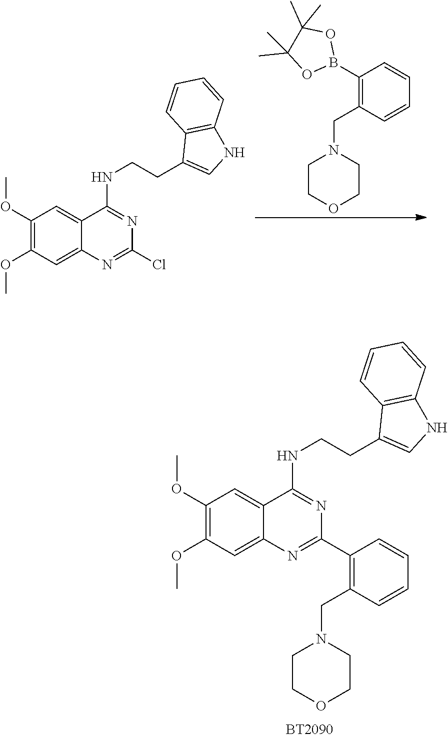 [SMILES: COc1cc2nc(Cl)nc(NCCc3c[nH]c4ccccc34)c2cc1OC ; CC1(C)OB(c2ccccc2CN2CCOCC2)OC1(C)C ; COc1cc2nc(-c3ccccc3CN3CCOCC3)nc(NCCc3c[nH]c4ccccc34)c2cc1OC]

Prepared according to General Procedure C from reaction of 4-(2-(4,4,5,5-tetramethyl-1,3,2-dioxaborolan-2-yl)benzyl)morpholine (119 mg, 0.39 mmol) and N-(2-(1H-indol-3-yl)ethyl)-2-chloro-6,7-dimethoxyquinazolin-4-amine (100 mg, 0.26 mmol) to afford a residue that was subjected to flash column chromatography [silica, 1:10 ammoniacal MeOH/DCM elution] to give, after concentration of the appropriate fractions BT-2090 as a white powder (57 mg, 42%). ¹H NMR (400 MHz, DMSO-d₆) δ 10.81 (s, 1H), 8.15 (s, 1H), 7.74 (d, J=7.5 Hz, 1H), 7.65 (s, 1H), 7.59 (d, J=7.9 Hz, 1H), 7.55 (dd, J=7.5, 0.8 Hz, 1H), 7.43-7.29 (m, 3H), 7.18 (d, J=2.3 Hz, 1H), 7.15 (s, 1H), 7.04 (ddd, J=8.0, 6.9, 1.1 Hz, 1H), 6.84 (ddd, J=8.0, 6.9, 1.0 Hz, 1H), 3.98-3.88 (m, 8H), 3.35-3.27 (m, 4H), 3.87-3.80 (m, 2H), 3.14-3.06 (m, 2H), 2.21-2.10 (m, 4H); (+)-LRESIMS m/z (rel. int.) 524 (100) [M+H]⁺; (+)-HRESIMS calcd. for C₃₁H₃₄N₅O₃ [M+H]⁺ 524.2656, found 524.2662.

### Synthesis of BT-2120

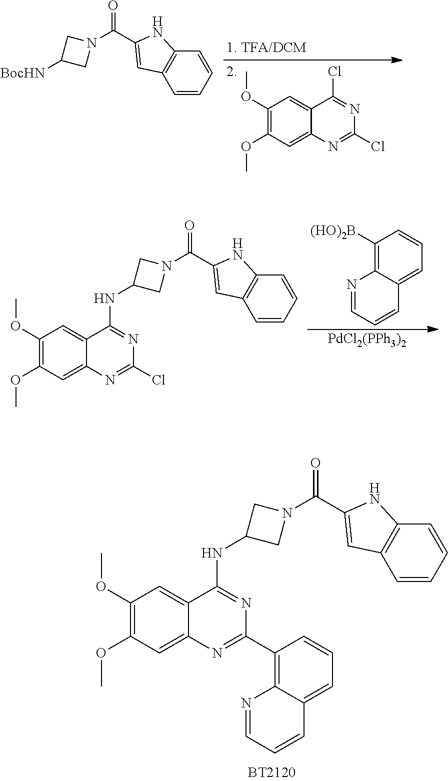

tert-Butyl (1-(1H-indole-2-carbonyl)azetidin-3-yl)carbamate (383 mg, 1.22 mmol) was deprotected following General Procedure F with TFA (2.0 mL) and DCM (8.0 mL) to afford after trituration with ether (10 mL) the TFA salt of (3-aminoazetidin-1-yl)(1H-indol-2-yl)methanone (348 mg, 87%) as a gum that was used directly in the next step without further purification. A magnetically stirred solution of the TFA-salt in DMF (5 mL), cooled to 0° C. was treated with 2,4-dichloro-6,7-dimethoxyquinazoline (274 mg, 1.06 mmol) followed by dropwise addition of triethylamine (595 μL, 4.24 mmol). The mixture was stirred at 0° C. for 5 h then a further 18 h at 18° C. The precipitate was collected by vacuum filtration and washed with DMF (5 mL) then ether (15 mL) to afford (3-((2-chloro-6,7-dimethoxyquinazolin-4-yl)amino)azetidin-1-yl)(1H-indol-2-yl)methanone (329 mg, 71%) as a white powder and used without further purification. ¹H NMR (400 MHz, DMSO-d₆) δ 11.66 (s, 1H), 8.89 (d, J=6.4 Hz, 1H), 7.72 (s, 1H), 7.63 (d, J=8.0 Hz, 1H), 7.45 (d, J=8.3 Hz, 1H), 7.20 (t, J=8.0 Hz, 1H), 7.12 (s, 1H), 7.05 (t, J=7.3 Hz, 1H), 6.90-6.86 (m, 1H), 5.14-5.01 (m, 1H), 5.00-4.92 (m, 1H), 4.62-4.46 (m, 2H), 4.32-4.18 (m, 1H), 3.91 (s, 3H), 3.90 (s, 3H). (+)-LRESIMS m/z (rel. int.) 438 (80) [M+H]⁺, 460 (100) [M+Na]⁺; vₘₐₓ 3303, 1617, 1577, 1541, 1516, 1452, 1432, 1240, 1149, 960, 736 cm⁻¹. The product formed directly above (100 mg, 0.23 mmol) was subjected to a palladium catalysed Suzuki-Miyaura reaction and reacted with 8-quinoline boronic acid (59 mg, 0.35 mmol) according to General Procedure C to afford a residue that was subjected to flash column chromatography [silica, 1:10 MeOH:DCM] to give, after concentration of the appropriate fractions BT-2120 (75 mg, 61%) as white crystals. ¹H NMR (400 MHz, DMSO-d₆) δ 11.63 (s, 1H), 8.94 (dd, J=4.4, 1.7 Hz, 1H), 8.70 (s, 1H), 8.49 (d, J=8.5 Hz, 1H), 8.15-8.04 (m, 2H), 7.78 (s, 1H), 7.72 (t, J=7.6 Hz, 1H), 7.66-7.59 (m, 2H), 7.44 (d, J=8.3 Hz, 1H), 7.30 (s, 1H), 7.20 (t, J=7.6 Hz, 1H), 7.05 (t, J=7.5 Hz, 1H), 6.83 (d, J=1.7 Hz, 1H), 5.14-5.02 (m, 1H), 4.90-4.80 (m, 1H), 4.64-4.55 (m, 1H), 4.50-4.41 (m, 1H), 4.32-4.24 (m, 1H), 3.96 (s, 3H), 3.93 (s, 3H). (+)-LRESIMS m/z (rel. int.) 531 (100) [M+hl]⁺; vₘₐₓ 3248, 1602, 1586, 1519, 1499, 1447, 1416, 1218, 794, 743 639 cm⁻¹.

### Synthesis of BT-2148

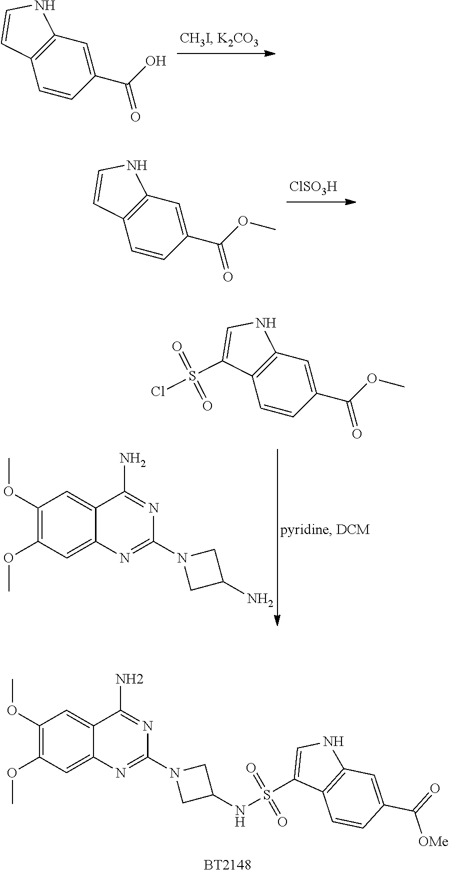 [SMILES: O=C(O)c1ccc2cc[nH]c2c1 ; COC(=O)c1ccc2cc[nH]c2c1 ; "ClSO3H" ; COC(=O)c1ccc2c(S(=O)(=O)Cl)c[nH]c2c1 ; COc1cc2nc(N3CC(N)C3)nc(N)c2cc1OC ; COC(=O)c1ccc2c(S(=O)(=O)NC3CN(c4nc(N)c5cc(OC)c(OC)cc5n4)C3)c[nH]c2c1]

### Methyl 1H-indole-6-carboxylate

A magnetically stirred solution of 1H-indole-6-carboxylic acid (5.00 g, 31.02 mmol) in DMF (40 mL) was treated with potassium carbonate (4.29 g, 31.02 mmol) and dropwise with methyl iodide (1.93 mL, 31.02 mmol). After stirring at room temperature for 3 h, the resulting mixture was diluted with diethyl ether (150 mL) then washed with water (2×100 mL) before being dried over magnesium sulphate, filtered and concentrated under reduced pressure. The ensuing residue was subjected to flash chromatography [silica, 20:80 v/v diethyl ether/hexane] and concentration of the appropriate fractions (R_{f}=0.42) afforded methyl 1H-indole-6-carboxylate (4.29 g, 83%) as a light yellow crystalline solid. ¹H NMR (400 MHz, chloroform-d) δ 8.71 (brs, 1H), 8.19 (s, 1H), 7.84 (dd, J=8.4 and 1.3 Hz, 1H), 7.67 (d, J=8.4 Hz, 1H), 7.37 (t, J=2.8 Hz, 1H), 6.60 (s, 1H), 3.95 (s, 3H); ¹³C NMR (100 MHz, chloroform-d) δ 168.5, 135.3, 131.7, 127.8, 123.6, 120.9, 120.4, 113.7, 103.0, 52.1; (+)-LRESIMS m/z (rel. int.) 198 (100) [M+Na]⁺; vₘₐₓ 3337, 1680, 1617, 1569, 1508, 1438, 1335, 1290, 1262, 1220, 1205, 1128, 1115, 1084, 982, 911, 828, 775, 736, 659 cm⁻¹.

Methyl 3-(chlorosulfonyl)-1H-indole-6-carboxylate:

Methyl 1H-indole-6-carboxylate (1.10 g, 6.28 mmol) was added in small portions to chlorosulfonic acid (2 mL) with intensive stirring. After 15 min, the mixture was carefully pipetted out into a flask placed in an ice bath containing ethyl acetate (30 mL). Then the resulting mixture was slowly poured into ice and the separated aqueous layer was extracted with ethyl acetate (3×30 mL). The combined organic phase was washed with NaHCO₃ (a saturated aqueous solution, 1×50 mL), brine (1×50 mL) before being dried over magnesium sulphate, filtered and concentrated under reduced pressure. The ensuing residue was subjected to flash chromatography (silica, 80:20 v/v diethyl ether/hexane) and concentration of the appropriate fractions (R_{f}=0.36, 80:20 v/v diethyl ether/hexane) afforded methyl 3-(chlorosulfonyl)-1H-indole-6-carboxylate (1.10 g, 64%) as a yellow crystalline solid. ¹H NMR (400 MHz, acetone-d₆) δ 12.08 (s, 1H), 8.55 (d, J=3.4 Hz, 1H), 8.35 (s, 1H), 8.05 (dd, J=8.5 and 1.3 Hz, 1H), 8.00 (d, J=8.5 Hz, 1H), 3.92 (s, 3H); ¹³C NMR (100 MHz, acetone-d₆) δ 167.1, 136.6, 136.2, 127.4, 126.7, 124.8, 120.0, 119.6, 116.1, 52.5; (+)-LRESIMS m/z (rel. int.) 270 (70) [M-Cl+MeOH]⁺; vₘₐₓ 3287, 1710, 1625, 1495, 1438, 1367, 1311, 1294, 1215, 1159, 1116, 1087, 1018, 766 cm⁻¹.

BT-2148:

A solution of methyl 3-(chlorosulfonyl)-1H-indole-6-carboxylate (149.1 mg, 0.54 mmol) in dichloromethane (3 mL) was treated with a solution of 2-(3-aminoazetidin-1-yl)-6,7-dimethoxyquinazolin-4-amine (50 mg, 0.18 mmol) in pyridine (0.5 mL) at 0° C. After 30 min, the resulting mixture was allowed to warm up to 20° C. for 2 h. Solvent was evaporated and the residue thus obtained was subjected to flash column chromatography [silica, 10:90 v/v methanol/dichloromethane elution] to give, after concentration of the appropriate fractions (R_{f}=0.22), BT-2148 as a white solid (75 mg, 75%). ¹H NMR (400 MHz, acetone-d₆) δ 11.57 (s, 1H), 8.33 (s, 1H), 8.15 (s, 1H), 8.03 (d, J=8.5 Hz, 1H), 7.91 (dd, J=8.5 and 1.4 Hz, 1H), 7.32 (s, 1H), 7.17 (d, J=9.5 Hz, 1H), 6.74 (s, 1H), 6.57 (s, 1H), 4.31 (m, 1H), 4.02 (t, J=8.2 Hz, 2H), 3.91 (s, 3H), 3.83 (s, 3H), 3.79 (s, 3H), 3.64 (dd, J=8.9 and 5.7 Hz, 2H); (+)-LRESIMS m/z (rel. int.) 513 (100) [M+H]⁺; HRMS (ESI, +ve) Found: [M+H]⁺ 513.1555, C₂₃H₂₅N₆O₆S requires 513.1556; vₘₐₓ 3180, 1703, 1649, 1625, 1565, 1486, 1455, 1438, 1380, 1311, 1241, 1210, 1140, 1105, 1019, 999, 844, 768 cm⁻¹.

### Synthesis of BT-2161

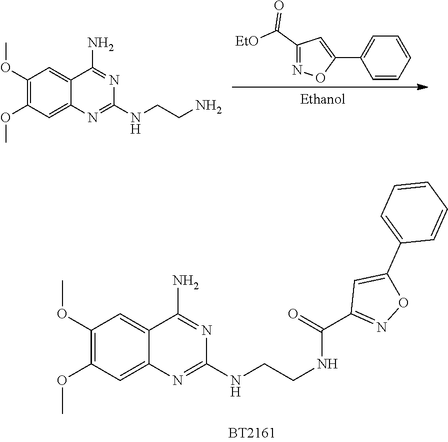 [SMILES: COc1cc2nc(NCCN)nc(N)c2cc1OC ; CCOC(=O)c1cc(-c2ccccc2)on1 ; COc1cc2nc(NCCNC(=O)c3cc(-c4ccccc4)on3)nc(N)c2cc1OC]

A 10 mL snap-cap microwave vessel was charged with N²-(2-aminoethyl)-6,7-dimethoxyquinazoline-2,4-diamine (121.2 mg, 0.46 mmol), ethyl 5-phenylisoxazole-3-carboxylate (50.0 mg, 0.23 mmol) prepared according to the procedure of Watterson et al. (J. Med. Chem. 2016, 59, 2820) and ethanol (1 mL). The tube was sealed and irradiated at 80° C. for 1 h before being stirred at 18° C. for 48 h. Solvent was evaporated and the resultant residue subjected to column chromatography [silica, 10:90 v/v methanol/dichloromethane elution] to afford, after concentration of the appropriate fractions (R_{f}=0.24) the compound BT-2161 (75.0 mg, 75%) as white solid. ¹H NMR (400 MHz, DMSO-d₆) δ 9.46 (brs, 1H), 7.88 (m, 2H), 7.53 (m, 3H), 7.45 (s, 1H), 7.34 (s, 1H), 7.15 (brs, 2H), 6.91 (s, 1H), 6.50 (brs, 1H), 3.88 (s, 3H), 3.79 (s, 3H), 3.51 (brs, 4H); ¹³C NMR (100 MHz, DMSO-d₆) δ 170.3, 161.2, 159.7, 159.2, 158.5, 154.1, 148.0, 144.8, 130.8, 129.3 (2C), 126.3, 125.7 (2C), 105.0, 104.0, 103.3, 99.9, 55.9, 55.4, 41.1, 40.2; (+)-LRESIMS m/z (rel. int.) 435 (100) [M+H]⁺; HRMS (ESI, +ve) Found: (M+H)⁺435.1766, C₂₂H₂₃N₆O₄ requires 435.1781; vₘₐₓ 3345, 3226, 2938, 1653, 1610, 1575, 1504, 1475, 1444, 1386, 1334, 1212, 1180, 1109, 1003, 853, 765 cm⁻¹.

### Synthesis of BT-2164

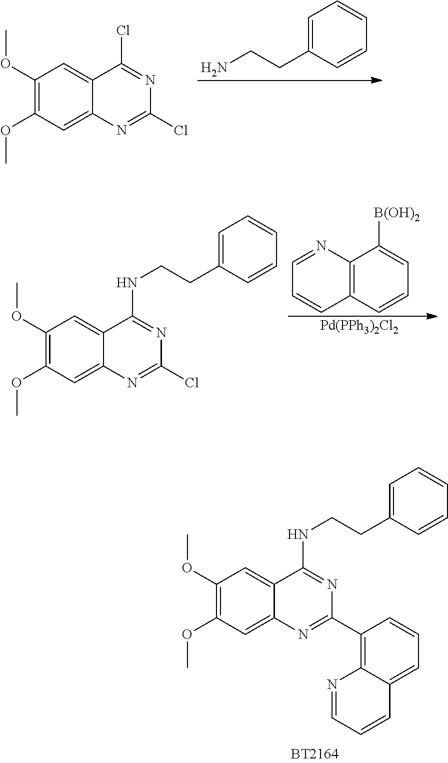

According to General Procedure B a solution of 2,4-dichloro-6,7-dimethoxyquinazoline (390.0 mg, 1.52 mmol), phenethylamine (382 μL, 3.04 mmol), triethylamine (212 μL, 1.52 mmol) in tetrahydrofuran (10 mL) was stirred at 18° C. for 18 h. 2-Chloro-6,7-dimethoxy-N-phenethylquinazolin-4-amine (500.0 mg, 96%) (R_{f}=0.21, 50:50 v/v ethyl acetate/hexane) was obtained as a white solid. ¹H NMR (400 MHz, chloroform-d) δ 7.30-7.27 (m, 2H), 7.20-7.23 (m, 3H), 7.03 (s, 1H), 6.88 (s, 1H), 6.26 (t, J=5.7 Hz, 1H), 3.87-3.91 (m, overlapped, 2H), 3.88 (s, 3H), 3.83 (s, 3H), 3.00 (t, J=7.1 Hz, 2H); ¹³C NMR (101 MHz, chloroform-d) δ 159.9, 156.2, 154.9, 149.0, 147.8, 138.7, 128.9 (2C), 128.7 (2C), 126.7, 106.9, 106.9, 100.0, 56.2, 56.1, 42.6, 35.2; MS (ESI, +ve) m/z 366.1 [(M+Na), 100%]; vₘₐₓ 3399, 3279, 2936, 1621, 1582, 1530, 1508, 1454, 1425, 1340, 1240, 1221, 1146, 942, 851, 750, 700 cm⁻¹. According to General Procedure C, a solution of 2-chloro-6,7-dimethoxy-N-phenethylquinazolin-4-amine (50.0 mg, 0.14 mmol), quinolin-8-ylboronic acid (37.7 mg, 0.21 mmol), K₂CO₃ (100.5 mg, 0.72 mmol) and Pd(PPh₃)₂Cl₂ (5.1 mg, 0.007 mmol) in dimethoxyethane-water-ethanol (2 mL) was irradiated at 120° C. for 25 min. BT-2164 (60.0 mg, 94%) (R_{f}=0.33, 10:90 v/v methanol/dichloromethane) was obtained as a yellow green solid. ¹H NMR (400 MHz, chloroform-d) δ 9.74 (brs, NH), 9.07 (s, 1H), 8.67 (d, J=7.4 Hz, 1H), 8.31 (d, J=8.0 Hz, 1H), 8.03 (d, J=8.0 Hz, 1H), 8.00 (s, 1H), 7.69 (t, J=7.7 Hz, 1H), 7.52 (dd, J=8.3, 4.0 Hz, 1H), 7.32 (s, 1H), 7.16-7.24 (m, 5H), 4.05 (s, 3H), 3.98 (s, 3H), 3.81 (t, J=7.5 Hz, 2H), 3.07 (t, J=7.5 Hz, 2H); ¹³C NMR (101 MHz, chloroform-d) δ 158.5, 157.9, 154.9, 150.4, 149.4, 146.0, 139.7, 137.7, 132.3, 132.1, 131.1, 129.1 (2C), 128.8, 128.5 (2C), 126.7, 126.3, 121.6, 107.4, 104.1, 103.1, 56.9, 56.6, 43.1, 35.3; MS (ESI, +ve) m/z 437.1 [(M+H), 100%]; HRMS (ESI, +ve) Found: (M+H)⁺437.1978, C₂₇H₂₅N₄O₂ requires 437.1978; vₘₐₓ 3393, 3235, 3027, 1625, 1597, 1549, 1514, 1479, 1401, 1279, 1232, 1126, 1036, 794 cm⁻¹.

### Synthesis of BT-2167

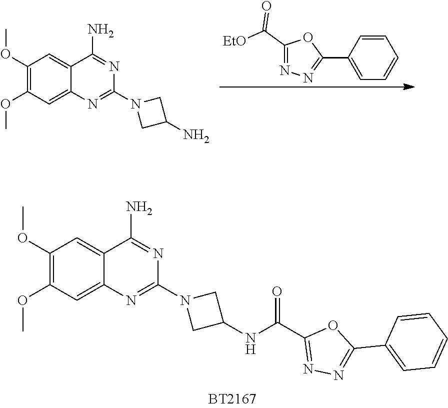 [SMILES: COc1cc2nc(N3CC(N)C3)nc(N)c2cc1OC ; CCOC(=O)c1nnc(-c2ccccc2)o1 ; COc1cc2nc(N3CC(NC(=O)c4nnc(-c5ccccc5)o4)C3)nc(N)c2cc1OC]

According to General Procedure E, a solution of 2-(3-aminoazetidin-1-yl)-6,7-dimethoxyquinazolin-4-amine (100.0 mg, 0.36 mmol), ethyl 5-phenyl-1,3,4-oxadiazole-2-carboxylate (52.8 mg, 0.24 mmol) in ethanol (1 mL) was irradiated at 80° C. for 4 h. BT-2167 (40.0 mg, 25%) (R_{f}=0.28, 5:95 v/v methanol/dichloromethane) was obtained as white solid. ¹H NMR (400 MHz, DMSO-d₆) δ 9.98 (d, J=7.2 Hz, 1H), 8.11 (m, 2H), 7.66 (m, 3H), 7.44 (s, ¹H), 7.26 (brs, 2H), 6.79 (s, 1H), 4.81 (h, J=7.3 Hz, 1H), 4.29 (t, J=8.2 Hz, 2H), 4.08 (dd, J=8.6 and 5.7 Hz, 2H), 3.83 (s, 3H), 3.79 (s, 3H); ¹³C NMR (100 MHz, DMSO-d₆) δ 164.9, 161.3, 160.3, 158.3, 154.2, 152.8, 148.5, 145.0, 132.7, 129.5 (2C), 127.1 (2C), 122.7, 105.0, 103.7, 103.1, 56.4 (2C), 55.8, 55.5, 39.9; (+)-LRESIMS m/z (rel. int.) 448 [(M+H)⁺, 100%]; HRMS (ESI, +ve) Found: (M+H)⁺448.1732, C₂₂H₂₂N₇O₄ requires 448.1733; vₘₐₓ 3341, 2871, 2393, 1672, 1651, 1624, 1556, 1479, 1452, 1434, 1414, 1377, 1336, 1240, 1214, 1005, 851 cm⁻¹.

### Synthesis of BT-2169 and BT-2173

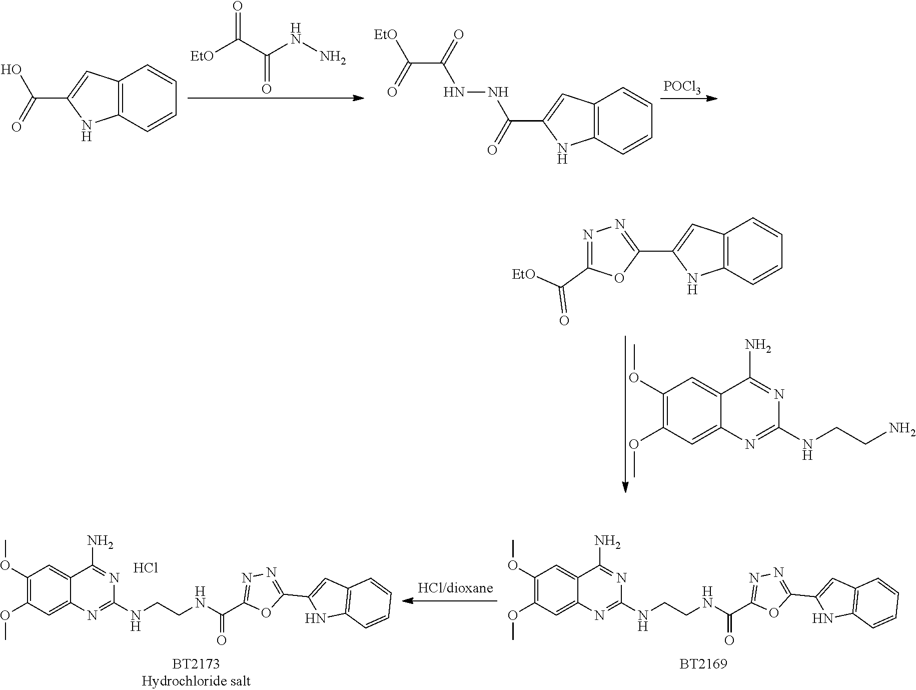

A magnetically stirred suspension of 1H-indole-2-carboxylic acid (3.56 g, 22.1 mmol) in DCM (80 mL) at 0° C. was treated with oxalyl chloride (1.99 mL, 23.2 mmol) dropwise, followed by DMF (1 drop). The mixture was stirred at 0° C. for 1 h and then the cold-bath was removed and stirring was continued at rt for 1 h. The clear solution was then concentrated with a gentle stream of nitrogen with heating, in a water bath at 40° C. The resulting tan coloured powder was then placed under high vacuum (1 mm Hg) for 1 h and redissolved in dioxane (60 mL) and ethyl 2-hydrazineyl-2-oxoacetate (2.92 g, 22.1 mmol) was added dropwise followed by sodium hydrogen carbonate (1.86, 22.1 mmol) and then magnetically stirred at 45-50° C. for 18 h. The mixture was then filtered and the filtrate concentrated in vacuo to afford a tan coloured solid which was washed with cold ether (15 mL) to afford ethyl 2-(2-(1H-indole-2-carbonyl)hydrazinyl)-2-oxoacetate (2.08 g) and used directly without further purification. MS (ESI, +ve) m/z 298 [(M+Na), 100%]. A portion of the above formed product (1.00 g, 3.64 mmol) was suspended in phosphoryl chloride (5 mL) and magnetically stirred under an atmosphere of nitrogen at 65° C. for 4 h. The phosphoryl chloride was removed by short-path distillation under high vacuum, and water (100 mL, ice-cold) was added and stirred for 0.2 h. The orange-coloured precipitate was collected and purified by flash chromatography (1:10 v/v diethyl ether/dichloromethane) to provide ethyl 5-(1H-indol-2-yl)-1,3,4-oxadiazole-2-carboxylate (351 mg, 38%) as a yellow powder. ¹H NMR (400 MHz, CDCl₃) δ 9.71 (s, 1H), 7.72 (d, J=8.1 Hz, 1H), 7.60 (d, J=8.3 Hz, 1H), 7.42-7.35 (m, 2H), 7.20 (app. t, J=7.6 Hz, 1H), 4.58 (q, J=7.1 Hz, 2H), 1.50 (t, J=7.1 Hz, 3H); 13C NMR (100 MHz, CDCl₃) δ 161.4, 155.9, 154.2, 137.9, 127.6, 126.0, 122.2, 121.4, 119.7, 112.2, 108.4, 63.6, 14.1; MS (ESI, +ve) m/z 280 [(M+Na), 100%].

According to General Procedure E, a solution of N²-(2-aminoethyl)-6,7-dimethoxyquinazoline-2,4-diamine (102.4 mg, 0.38 mmol), ethyl 5-(1H-indol-2-yl)-1,3,4-oxadiazole-2-carboxylate (50.0 mg, 0.19 mmol) in ethanol (1 mL) was irradiated at 80° C. for 3 h. BT2169 (80.0 mg, 87%) (Rf=0.22, 15:85 v/v methanol/dichloromethane) was obtained as a white solid. ¹H NMR (400 MHz, DMSO-d₆) δ 12.37 (s, 1H), 9.93 (s, 1H), 7.69 (d, J=8.0 Hz, 1H), 7.49 (d, J=8.3 Hz, 1H), 7.45 (s, 1H), 7.29 (t, overlapped, J=7.2 Hz, 1H), 7.26 (s, overlapped, 1H), 7.21 (brs, overlapped, NH₂), 7.11 (t, J=7.5 Hz, 1H), 6.93 (s, 1H), 6.57 (brs, 1H), 3.88 (s, 3H), 3.79 (s, 3H), 3.52 (s, 4H); ¹³C NMR (100 MHz, DMSO-d₆) δ 161.3, 160.3, 158.8, 157.9, 154.2, 153.1, 147.6, 144.9, 138.0, 127.2, 124.6, 121.7, 120.5, 120.4, 112.4, 106.1, 104.8, 104.0, 103.3, 55.9, 55.5, 41.4, 39.9; (+)-LRESIMS m/z (rel. int.) 475 [(M+H)⁺, 100%]; HRMS (ESI, +ve) Found: (M+H)⁺448.1732, C₂₂H₂₂N₇O₄ requires 448.1733. vₘₐₓ 3465, 3331, 3216, 3158, 2966, 1681, 1636, 1609, 1572, 1505, 1484, 1460, 1397, 1346, 1320, 1277, 1238, 1228, 1212, 1183, 1109, 1007, 831, 814, 736 cm⁻¹.

BT-2173: A magnetically stirred suspension of BT-2169 (21.7 mg, 52 μmol) in dioxane (3 mL) maintained at 0° C. (ice water bath) was treated dropwise with a solution of HCl (100 μL, 4 M in dioxane). The mixture was stirred for 5 min then concentrated by a gentle stream of nitrogen then the solid triturated with ether (2 mL) and the residue held under high vacuum for 1 h to afford BT-2173 (19.5 mg, 82%), the hydrochloride salt of BT-2169 as a white powder. ¹H NMR (400 MHz, DMSO-d₆) One NH not observed δ 12.42 (s, 1H), 9.47 (t, J=5.7 Hz, 1H), 8.84 (br s, 1H), 8.62 (br s, 1H), 7.89 (s, 1H), 7.72 (s, 1H), 7.69 (d, J=8.2 Hz, 1H), 7.49 (d, J=8.3 Hz, 1H), 7.32-7.26 (m, 2H), 7.12 (t, J=7.5 Hz, 1H), 6.99 (br s, 1H), 3.89 (s, 3H), 3.82 (s, 3H), 3.71-3.61 (m, 2H), 3.64-3.53 (m, 2H).

### Synthesis of BT-2170

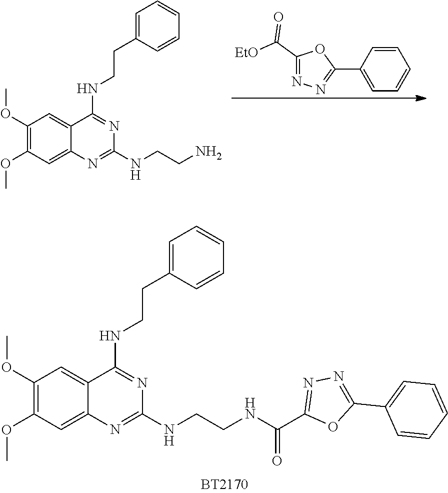 [SMILES: COc1cc2nc(NCCN)nc(NCCc3ccccc3)c2cc1OC ; CCOC(=O)c1nnc(-c2ccccc2)o1 ; COc1cc2nc(NCCNC(=O)c3nnc(-c4ccccc4)o3)nc(NCCc3ccccc3)c2cc1OC]

According to General Procedure E, a solution of N²-(2-aminoethyl)-6,7-dimethoxy-N⁴-phenethylquinazoline-2,4-diamine (101 mg, 0.24 mmol), ethyl 5-(1H-indol-2-yl)-1,3,4-oxadiazole-2-carboxylate (30.0 mg, 0.14 mmol) in ethanol (1 mL) was irradiated at 80° C. for 3 h. BT-2170 (70.0 mg, 94%) (R_{f}=0.14, 3.5:96.5 v/v methanol saturated ammonia/dichloromethane) was obtained as a yellow solid. ¹H NMR (400 MHz, chloroform-d) δ 10.45 (s, NH), 8.03 (d, J=7.5 Hz, 2H), 7.58-7.37 (m, 4H), 7.31-7.14 (m, 5H), 6.59 (s, 1H), 5.40 (brs, overlapped, NH), (brs, overlapped, NH), 4.11 (s, 3H), 3.81 (s, 3H), 3.77 (q, overlapped, 3=6.6 Hz, 2H), 3.70 (s, 4H), 2.94 (t, J=7.0 Hz, 2H); ¹³C NMR (100 MHz, chloroform-d) δ 166.0, 159.9, 159.3, 159.0, 154.80, 153.8, 148.1, 146.0, 139.3, 132.4, 129.2 (2C), 129.0 (2C), 128.8 (2C), 127.5 (2C), 126.7, 123.2, 106.9, 104.1, 100.4, 56.6, 56.2, 44.2, 42.3, 41.1, 35.6; (+)-LRESIMS m/z (rel. int.) 540.3 (100) [M+H]⁺; HRMS (ESI, +ve) Found: [M+H]⁺ 540.2356, C₂₉H₃₀N₇O₄ requires 540.2359. [M+Na]⁺ 562.2182, C₂₉H₂₉N₇O₄Na requires 562.2179; vₘₐₓ 3402, 2934, 1680, 1626, 1582, 1547, 1504, 1474, 1451, 1354, 1316, 1250, 1233, 1213, 1014, 855, 710 cm⁻¹.

### Synthesis of BT-2171

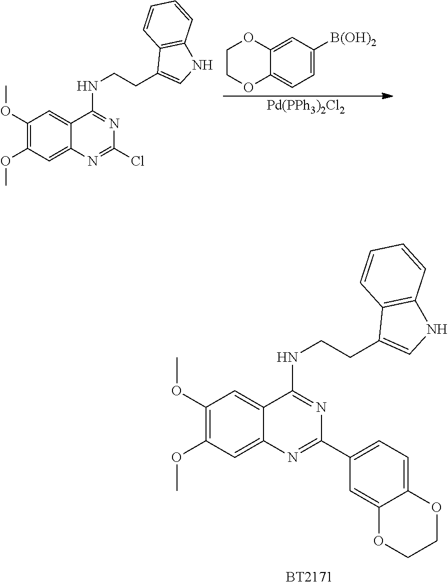

According to General Procedure C, a solution of N-(2-(1H-indol-3-yl)ethyl)-2-chloro-6,7-dimethoxyquinazolin-4-amine (50.0 mg, 0.13 mmol), 1,4-benzodioxane-6-boronic acid (35.5 mg, 0.19 mmol), K₂CO₃ (90.2 mg, 0.65 mmol) and Pd(PPh₃)₂Cl₂ (4.6 mg, 0.006 (5) mmol) in dimethoxyethane-water-ethanol (2 mL) was irradiated at 120° C. for 25 min. BT-2171 (40.0 mg, 63%) (R_{f}=0.25, 70:30 v/v ethyl acetate/hexane) was obtained as a white solid. ¹H NMR (400 MHz, DMSO-d₆) δ 10.86 (s, NH), 8.11 (t, J=5.5 Hz, NH), 8.03-7.98 (m, 2H), 7.67 (d, J=7.8 Hz, 1H), 7.61 (s, 1H), 7.37 (d, J=8.0 Hz, 1H), 7.24 (d, J=2.2 Hz, 1H), 7.15 (s, 1H), 7.09 (t, J=8.0 Hz, 1H), 7.01 (t, J=7.8 Hz, 1H), 6.94 (d, J=8.4 Hz, 1H), 4.30 (s, 4H), 3.93 (s, 3H), 3.91 (m, overlapped, 2H), 3.89 (s, 3H), 3.16 (m, overlapped, 2H); ¹³C NMR (101 MHz, DMSO-d₆) δ 158.4, 157.5, 153.8, 147.9, 146.7, 144.9, 143.0, 136.3, 132.6, 127.3, 122.7, 121.0, 120.8, 118.3, 118.3, 116.7, 116.3, 112.1, 111.4, 107.34, 107.2, 102.1, 64.3, 64.1, 56.0, 55.7, 41.7, 25.0; MS (ESI, +ve) m/z 483.2 [(M+H), 100%]; HRMS (ESI, +ve) Found: (M+H)⁺483.2035, C₂₈H₂₇N₄O₄ requires 483.2032; vₘₐₓ 3307, 2932, 1622, 1577, 1528, 1502, 1457, 1433, 1420, 1359, 1313, 1283, 1259, 1237, 1222, 1210, 1171, 1127, 1065, 1049, 1025, 894, 740 cm⁻¹.

### Synthesis of BT-2177

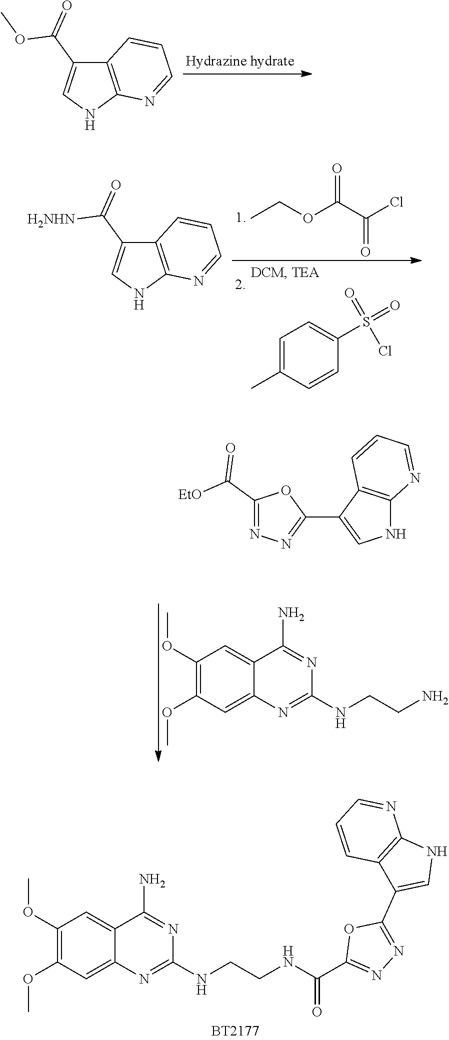

1H-pyrrolo[2,3-b]pyridine-3-carbohydrazide:

A solution of methyl 1H-pyrrolo[2,3-b]pyridine-3-carboxylate (882 mg, 5.20 mmol) in dioxane (5 mL) was treated with hydrazine monohydrate (1.51 mL, 31 mmol) and refluxed for 18 h. The reaction mixture was cooled and the solid collected by vacuum filtration and the crystals washed with ether (10 mL) to afford 1H-pyrrolo[2,3-b]pyridine-3-carbohydrazide (553 mg, 60%), as fine white crystals, which was used without further purification. ¹H NMR (400 MHz, DMSO-d₆) δ 12.03 (s, 1H), 9.27 (s, 1H), 8.43 (d, J=7.8 Hz, 1H), 8.26 (d, J=4.6 Hz, 1H), 8.08 (s, 1H), 7.16 (dd, J=7.9, 4.7 Hz, 1H), 4.34 (s, 2H).

Ethyl 5-(1H-pyrrolo[2,3-b]pyridin-3-yl)-1,3,4-oxadiazole-2-carboxylate:

A solution of 1H-pyrrolo[2,3-b]pyridine-3-carbohydrazide (500.0 mg, 2.84 mmol) in DCM (20 mL) at 0° C. was treated with triethylamine (1.19 mL, 8.51 mmol) then dropwise ethyl chlorooxoacetate (0.33 mL, 2.98 mmol). The mixture was stirred at the same temperature for 30 min then warmed up to 18° C. for 30 min before being added p-toluenesulfonyl chloride (541.0 mg, 2.84 mmol). Ethyl 5-(1H-pyrrolo[2,3-b]pyridin-3-yl)-1,3,4-oxadiazole-2-carboxylate (120.0 mg, 16%) (R_{f}=0.31, 2:98 v/v methanol/diethyl ether) was obtained as white solid. ¹H NMR (400 MHz, DMSO-d₆) δ 12.79 (s, 1H), 8.48 (s, 1H), 8.47-8.36 (m, 2H), 7.34 (dd, J=7.8, 4.8 Hz, 1H), 4.45 (q, 3=7.1 Hz, 2H), 1.37 (t, J=7.1 Hz, 3H); ¹³C NMR (100 MHz, DMSO-d₆) δ 162.9, 154.8, 154.2, 148.7, 144.7, 130.2, 128.6, 117.9, 116.6, 97.5, 62.8, 13.9; (+)-LRESIMS m/z (rel. int.) 281.1 (100) [M+Na]⁺; vₘₐₓ 3140, 2993, 2817, 1732, 1624, 1578, 1533, 1495, 1470, 1411, 1330, 1265, 1167, 1037, 1014, 839, 808, 780, 730 cm⁻¹.

BT-2177:

According to General Procedure E, a solution of N²-(2-aminoethyl)-6,7-dimethoxy-N⁴-phenethylquinazoline-2,4-diamine (80.0 mg, 0.30 mmol), ethyl 5-(1H-pyrrolo[2,3-b]pyridin-3-yl)-1,3,4-oxadiazole-2-carboxylate (46.1 mg, 0.18 mmol) in ethanol (1 mL) was irradiated at 80° C. for 3 h. BT-2177 (60.0 mg, 71%) (R_{f}=0.47, 2.5:17.5:80 v/v/v methanol saturated ammonia/methanol/dichloromethane) was obtained as a cream solid. ¹H NMR (400 MHz, DMSO-d₆) δ 12.40 (brs, NH), 9.98 (brs, NH), 8.50-8.32 (m, 3H), 7.41 (s, 1H), 7.34 (m, 1H), 7.05 (brs, NH₂), 6.96 (s, 1H), 6.42 (brs, NH), 3.90 (s, 3H), 3.78 (s, 3H), 3.51 (s, 4H); ¹³C NMR (100 MHz, DMSO-d₆) δ 162.8, 161.6, 160.0, 157.4, 154.5, 153.7, 149.2, 149.0, 145.1 (CH+Cq), 130.2, 129.0, 118.2, 117.0, 105.9, 104.2, 103.8, 98.2, 56.3, 55.8, 41.9, 40.5; (+)-LRESIMS m/z (rel. int.) 476.3 (100) [M+H]⁺; HRMS (ESI, +ve) Found: (M+H)⁺476.1797, C₂₂H₂₂N₉O₄ requires 476.1795; vₘₐₓ 3556, 3398, 3279, 1686, 1651, 1630, 1563, 1502, 1446, 1313, 1271, 1231, 1211, 1184, 1143, 1103, 997, 852, 807, 791, 766 cm⁻¹.

### Synthesis of BT-2178

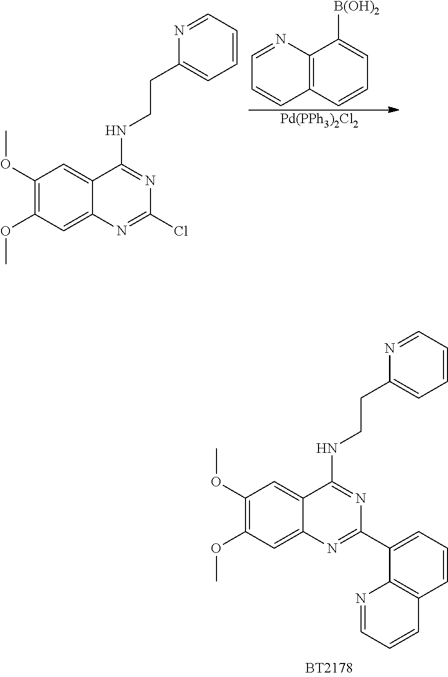

According to General Procedure C, a solution 2-chloro-6,7-dimethoxy-N-(2-(pyridin-2-yl)ethyl)quinazolin-4-amine (50.0 mg, 0.14 mmol), quinolin-8-ylboronic acid (37.6 mg, 0.21 mmol), K₂CO₃ (100.2 mg, 0.72 mmol) and Pd(PPh₃)₂Cl₂ (5.1 mg, 0.007 (3) mmol) in dimethoxyethane-water-ethanol (1.5 mL) was irradiated at 120° C. for 25 min. BT-2178 (55.0 mg, 87%) (R_{f}=0.18, 5:95 v/v methanol saturated ammonia/dichloromethane) was obtained as a white solid. ¹H NMR (400 MHz, DMSO-d₆) δ 8.83 (d, J=4.0 Hz, 1H), 8.46 (d, J=4.6 Hz, 1H), 8.41 (d, J=8.3 Hz, 1H), 8.08 (t, J=5.1 Hz, NH), 8.03 (d, J=8.2 Hz, 1H), 7.85 (d, J=7.0 Hz, 1H), 7.68 (t, J=7.7 Hz, 1H), 7.64 (s, 1H), 7.61 (t, J=7.7 Hz, 1H), 7.52 (dd, J=8.2, 4.0 Hz, 1H), 7.20-7.13 (m, 3H), 3.90 (s, 3H), 3.89 (s, 3H), 3.80 (q, J=6.5 Hz, 2H), 3.17 (m, overlapped with MeOH, 2H); ¹³C NMR (101 MHz, DMSO-d₆) δ 161.5, 159.5, 158.1, 153.7, 150.0, 149.0, 148.2, 146.5, 145.9, 140.9, 136.3, 136.0, 129.4, 128.1, 128.0, 125.9, 123.1, 121.4, 121.2, 107.2, 107.12, 101.9, 56.0, 55.6, 40.6, 36.9; MS (ESI, +ve) m/z 438.3 [(M+H), 100%]; HRMS (ESI, +ve) Found: (M+H)⁺438.1929, C₂₆H₂₄N₅O₂ requires 438.1930; vₘₐₓ 3266, 2935, 1619, 1586, 1522, 1499, 1474, 1423, 1355, 1253, 1219, 1175, 1145, 1042, 1000, 855, 836, 801, 788 cm⁻¹.

### Synthesis of BT-2179

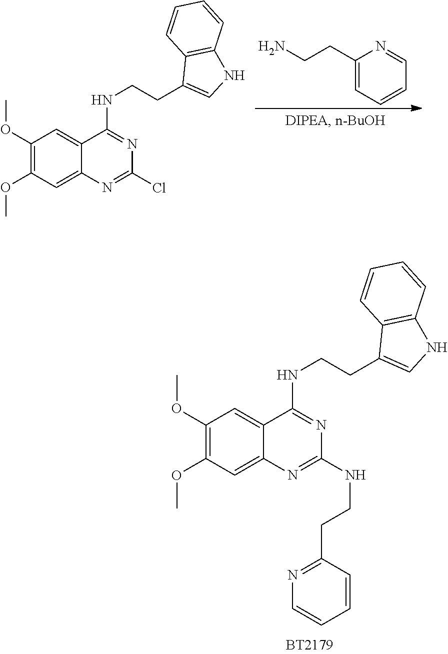 [SMILES: COc1cc2nc(Cl)nc(NCCc3c[nH]c4ccccc34)c2cc1OC ; NCCc1ccccn1 ; COc1cc2nc(NCCc3ccccn3)nc(NCCc3c[nH]c4ccccc34)c2cc1OC]

A 10 mL snap-cap microwave vessel was charged with a mixture of N-(2-(1H-indol-3-yl)ethyl)-2-chloro-6,7-dimethoxyquinazolin-4-amine (75.0 mg, 0.19 mmol), 2-pyridylethylamine (117 μL, 0.98 mmol), N,N-diisopropylethylamine (63 μL, 0.49 mmol) and n-butanol (2 mL). The tube was sealed then subjected to microwave irradiation (120° C./2 h, ramp time 5 minutes, maximum power 250 W). The mixture was cooled and concentrated in vacuo and the resulting residue was subjected to flash column chromatography [silica, 5:95 v/v methanol saturated ammonia/ethyl acetate elution] and concentration of the appropriate fractions (R_{f}=0.39, 5:95 v/v methanol saturated ammonia/ethyl acetate elution) to give the compound BT-2179 (60.0 mg, 65%) as a white solid. ¹H NMR (400 MHz, methanol-d₄) δ 8.38 (d, J=4.7 Hz, 1H), 7.54 (m, 2H), 7.32 (d, J=8.1 Hz, 1H), 7.20 (s, 1H), 7.15 (m, 2H), 7.06 (t, overlapped, J=7.4 Hz, 1H), 7.04 (s, 1H), 6.92 (t, J=7.4 Hz, 1H), 6.76 (s, 1H), 3.85 (s, 3H), 3.84 (m, overlapped, 2H), 3.80 (s, 3H), 3.76 (m, overlapped, 2H), 3.12 (t, J=7.4 Hz, 2H), 3.07 (t, J=7.0 Hz, 2H); ¹³C NMR (101 MHz, methanol-d) δ 169.1, 168.9, 168.6, 163.7, 157.5, 157.0, 154.7, 146.4, 146.1, 136.9, 133.1, 131.3, 130.8, 130.2, 127.5, 127.4, 121.9, 120.2, 113.2, 112.9, 111.9, 64.6, 64.1, 51.1, 50.4, 47.2, 34.2; MS (ESI, +ve) m/z 469.2 [(M+H), 100%]; HRMS (ESI, +ve) Found: (M+H)⁺469.2352, C₂₇H₂₉N₆O₂ requires 469.2352; vₘₐₓ 3408, 2933, 1626, 1583, 1505, 1457, 1435, 1359, 1303, 1234, 1212, 1008, 853, 744 cm⁻¹.

### Synthesis of BT-2181

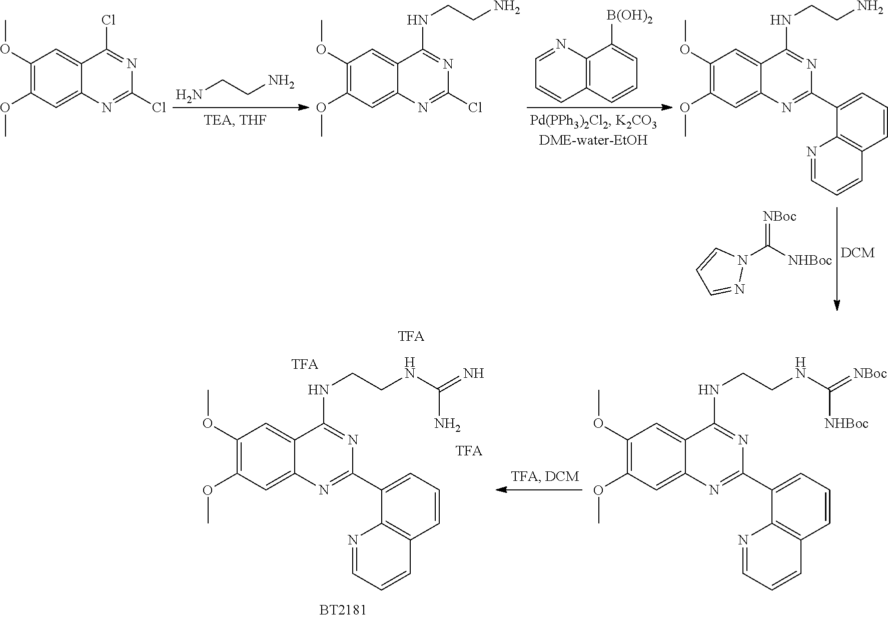

In accordance with General Procedure B, a solution of 2,4-dichloro-6,7-dimethoxyquinazoline (200.0 mg, 0.78 mmol), ethylenediamine (104 μL, 1.56 mmol), triethylamine (108 μL, 0.78 mmol) in tetrahydrofuran (10 mL) was stirred at 18° C. for 18 h. Flash chromatography (R_{f}=0.19, 12.5:87.5 v/v methanol saturated ammonia/dicloromethane) provided N¹-(2-chloro-6,7-dimethoxyquinazolin-4-yl)ethane-1,2-diamine (200.0 mg, 91%) as an ivory solid. ¹H NMR (400 MHz, DMSO-d₆) δ 8.31 (s, NH), 7.63 (s, 1H), 7.05 (s, 1H), 3.88 (s, 6H), 3.49 (t, J=6.6 Hz, 2H), 2.97 (overlapped, brs, NH₂), 2.81 (t, J=6.6 Hz, 3H); ¹³C NMR (101 MHz, DMSO-d₆) δ 160.2, 155.2, 154.3, 148.4, 147.1, 107.0, 106.5, 102.4, 56.1, 55.8, 44.3, 40.6; MS (ESI, +ve) m/z 283 [(M+H)⁺, 100%].

According to General Procedure C, a solution of N¹-(2-chloro-6,7-dimethoxyquinazolin-4-yl)ethane-1,2-diamine (126.0 mg, 0.73 mmol), quinolin-8-ylboronic acid (189.0 mg, 1.09 mmol), K₂CO₃ (503.3 mg, 3.64 mmol) and Pd(PPh₃)₂Cl₂ (25.6 mg, 0.03 (6) mmol) in dimethoxyethane-water-ethanol (4 mL) was irradiated at 120° C. for 25 min. Flash chromatography (R_{f}=0.14, 15:15:95 v/v/v methanol saturated ammonia/methanol/ethylacetate) provided N¹-(6,7-dimethoxy-2-(quinolin-8-yl)quinazolin-4-yl)ethane-1,2-diamine (200.0 mg, 73%) as a yellow solid contaminated with quinolin-8-ylboronic acid. MS (ESI, +ve) m/z 376 [(M+H)⁺, 100%].

A solution of the above N¹-(6,7-dimethoxy-2-(quinolin-8-yl)quinazolin-4-yl)ethane-1,2-amine (100.0 mg, 0.27 mmol) in dicloromethane (2 mL) was treated with tert-butyl (Z)-(((tert-butoxycarbonyl)amino)(1H-pyrazol-1-yl)methylene)carbamate (82.7 mg, 0.27 mmol) and the resulting reaction mixture was stirred at 18° C. for 18 h. Solvent was evaporated and the residue was subjected to flash column chromatography (silica, 5:95 v/v methanol saturated ammonia/diethyl ether elution) and concentration of the appropriate fractions (R_{f}=0.19, 5:95 v/v methanol saturated ammonia/diethylether elution) to give the di-Boc-protected guanidine derivate (128.0 mg, 77.8%) as an ivory solid. ¹H NMR (400 MHz, Chloroform-d) δ 11.45 (s, 1H), 9.14 (d, J=4.1 Hz, 1H), 8.46 (t, J=5.3 Hz, 1H), 8.28 (d, J=8.3 Hz, 1H), 7.96 (d, J=7.6 Hz, 1H), 7.93 (d, J=7.6 Hz, 1H), 7.67 (t, J=7.6 Hz, 1H), 7.49 (dd, J=8.3, 4.1 Hz, 1H), 7.31-7.29 (m, 1H), 7.05 (s, 1H), 7.04 (s, 1H), 3.92 (s, 3H), 3.66 (s, 3H), 3.57 (m, 2H), 3.51 (m, 2H), 1.49 (s, 9H), 1.45 (s, 9H); ¹³C NMR (101 MHz, Chloroform-d) δ 163.6, 161.5, 159.0, 156.6, 153.9, 153.1, 150.2, 148.3, 146.7, 146.5, 140.8, 136.9, 130.5, 128.9, 128.3, 126.7, 121.1, 107.9, 107.2, 101.0, 83.1, 79.3, 56.1, 56.0, 40.3, 39.9, 28.4 (3C), 28.1 (3C); MS (ESI, +ve) m/z 618 [(M+H)+, 100%]; IR (ATR) vₘₐₓ 3285, 2980, 2254, 1720, 1621, 1582, 1528, 1501, 1415, 1367, 1344, 1252, 1218, 1134, 1045, 1021, 905, 724 cm⁻¹.

A solution of Boc-protected guanidine compound formed above, in dichloromethane (5 mL) was treated dropwise with trifluoroacetic acid (2.0 mL) at 0° C. The resulting mixture was stirred at 18° C. until the completion conversion (observed by TLC). Solvent was then evaporated to obtain the TFA salt of BT-2181 (100.0 mg) as a yellow solid. ¹H NMR (400 MHz, DMSO-d₆) δ 9.88 (t, J=5.8 Hz, NH), 9.31-9.19 (m, 1H), 9.07 (d, J=7.5 Hz, 1H), 8.75 (d, J=8.2 Hz, 1H), 8.41 (d, J=8.2 Hz, 1H), 8.13 (t, J=6.3 Hz, NH), 7.92-7.87 (m, 2H), 7.81 (s, 1H), 7.74 (s, 1H), 7.38 (brs, 3NH), 4.04 (s, 3H), 3.99-3.97 (m, 2H), 3.94 (s, 3H), 3.62-3.57 (m, 2H); ¹³C NMR (101 MHz, DMSO-d₆) δ 159.5, 157.6, 156.5, 155.1, 151.2, 150.5, 145.0, 139.8, 134.9, 134.7, 134.1, 128.9, 127.5, 124.3, 123.0, 106.6, 103.6, 101.9, 57.2, 56.8, 41.0, 40.3; MS (ESI, +ve) m/z 418 [(M+H)⁺, 100%]; HRMS (ESI, +ve) Found: (M+H)+418.1993, C₂₂H₂₄N₇O₂ requires 418.1991; IR (ATR) vₘₐₓ 3333, 3095, 1710, 1673, 1621, 1598, 1549, 1510, 1471, 1443, 1424, 1401, 1274, 1198, 1176, 1119, 1105, 1053, 1042, 1027, 835, 796, 719 cm⁻¹.

### Synthesis of BT-2182

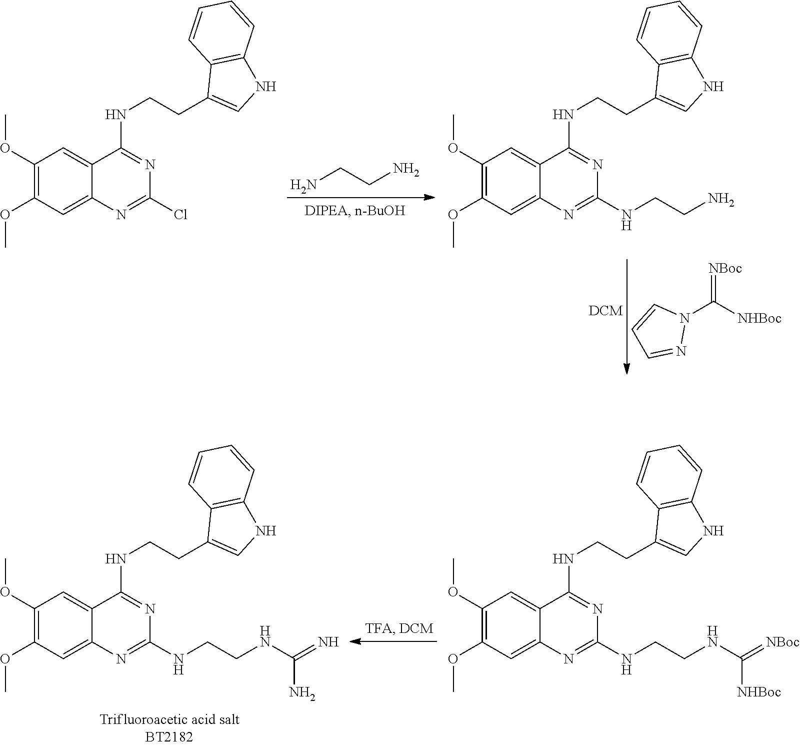

A 10 mL snap-cap microwave vessel was charged with a mixture of N-(2-(1H-indol-3-yl)ethyl)-2-chloro-6,7-dimethoxyquinazolin-4-amine (100.0 mg, 0.26 mmol), ethylenediamine (261 μL, 3.91 mmol), N,N-diisopropylethylamine (159 μL, 0.91 mmol) and n-butanol (2 mL). The tube was sealed then subjected to microwave irradiation (120° C./2 h, ramp time 5 minutes, maximum power 250 W). The mixture was cooled and concentrated in vacuo and the resulting residue was subjected to flash column chromatography (silica, 12.5:87.5 v/v methanol saturated ammonia/dichloromethane elution) and concentration of the appropriate fractions (R_{f}=0.24, 12.5:87.5 v/v methanol saturated ammonia/dichloromethane elution) afforded N⁴-(2-(1H-indol-3-yl)ethyl)-N²-(2-aminoethyl)-6,7-dimethoxyquinazoline-2,4-diamine (95.0 mg, 89%) as a yellow oil. ¹H NMR (400 MHz, Methanol-d₄) δ 7.57 (d, J=7.9 Hz, 1H), 7.33 (d, J=8.1 Hz, 1H), 7.22 (s, 1H), 7.10-7.06 (m, 2H), 6.97 (t, J=7.5 Hz, 1H), 6.80 (s, 1H), 3.89 (s, 3H), 3.85 (overlapped t, J=7.3 Hz, 2H), 3.82 (s, 3H), 3.49 (t, J=6.2 Hz, 3H), 3.14 (t, J=7.3 Hz, 2H), 2.85 (t, J=6.2 Hz, 2H); ¹³C NMR (101 MHz, Methanol-d₄) δ 161.2, 161.0, 155.8, 149.0, 146.8, 138.2, 129.0, 123.4, 122.3, 119.5, 119.4, 113.9, 112.2, 105.3, 105.1, 104.0, 56.7, 56.2, 44.8, 43.1, 42.6, 26.3; MS (ESI, +ve) m/z 407.3 [(M+H)+, 100%].

A solution of the above N⁴-(2-(1H-indol-3-yl)ethyl)-N²-(2-aminoethyl)-6,7-dimethoxyquinazoline-2,4-diamine (84.0 mg, 0.21 mmol) in dicloromethane (2 mL) was treated with tert-butyl (Z)-(((tert-butoxycarbonyl)amino)(1H-pyrazol-1-yl)methylene)carbamate (70.5 mg, 0.23 mmol) and the resulting reaction mixture was stirred at 18° C. for 18 h. The solvent was evaporated and the residue was subjected to flash column chromatography (silica, 5:95 v/v methanol saturated ammonia/diethylether elution) and concentration of the appropriate fractions (R_{f}=0.19, 5:95 v/v methanol saturated ammonia/diethyl ether elution) to give the Boc-protected guanidine derivative (110.0 mg, 82.0%) as an ivory solid. ¹H NMR (400 MHz, Chloroform-d) δ 11.50 (s, 1H), 8.85 (s, 1H), 8.57 (m, 1H), 7.60 (d, J=8.0 Hz, 1H), 7.34 (d, J=8.0 Hz, 1H), 7.15 (t, J=7.5 Hz, 1H), 7.06 (t, J=7.5 Hz, 1H), 7.02 (s, 1H), 6.82 (s, 1H), 6.59 (s, 1H), 5.95 (brs, 1H), 4.86 (brs, 1H), 3.94-3.83 (overlapped, m, 2H), 3.83 (s, 3H), 3.62 (m, 7H), 3.12 (t, J=6.3 Hz, 2H), 1.47 (s, 9H), 1.40 (s, 9H). ¹³C NMR (101 MHz, Chloroform-d) δ 163.5, 159.4, 159.3, 156.7, 154.2, 153.0, 148.7, 145.2, 136.4, 127.7, 122.3, 122.0, 119.4, 118.6, 113.3, 111.5, 105.6, 104.1, 101.2, 83.0, 79.4, 55.9 (3), 55.8 (7), 41.9, 41.4, 40.7, 28.3 (3C), 28.0 (3C), 24.9; MS (ESI, +ve) m/z 649.5 [(M+H)+, 100%]; IR (ATR) vₘₐₓ 3328, 2977, 1722, 1615, 1580, 1499, 1455, 1413, 1323, 1294, 1228, 1211, 1132, 1050, 1024, 732 cm⁻¹.

A solution of Boc-protected guanidine compound (70 mg, 0.11 mmol) obtained above in dichloromethane (5 mL) was treated dropwise with trifluoroacetic acid (1.0 mL) at 0° C. The resulting mixture was stirred at 20° C. until the completion conversion (observed by TLC). Solvent was then evaporated to obtain the TFA salt of BT-2182 (70.0 mg) as an ivory solid. ¹H NMR (400 MHz, DMSO-d₆) δ 12.87 (brs, 1H), 10.90 (s, 1H), 9.40 (brs, 1H), 8.36 (brs, 1H), 7.93 (brs, 1H), 7.70 (s, 1H), 7.56 (d, J=7.9 Hz, 1H), 7.36 (d, J=8.2 Hz, 1H), 7.20 (s, 1H), 7.07 (t, J=7.7 Hz, 1H), 7.00-6.95 (m, 2H), 3.89-3.84 (overlapped, m, 8H), 3.58 (brs, 2H), 3.40 (brs, 2H), 3.11 (brs, 2H); ¹³C NMR (101 MHz, DMSO-d₆) δ 159.6, 157.6, 155.6, 153.2, 147.1, 136.7, 135.8, 127.6, 123.4, 121.4, 118.8, 118.6, 111.9, 111.9, 104.9, 102.6, 98.7, 65.4, 56.6, 56.5, 42.6, 24.8, 15.6; MS (ESI, +ve) m/z 449.3 [(M+H)+, 100%]; HRMS (ESI, +ve) Found: (M+H)⁺449.2413, C₂₃H₂₉N₈O₂ requires 449.2413; IR (ATR) vₘₐₓ 3310, 3145, 1673, 1611, 1580, 1515, 1432, 1395, 1278, 1199, 1176, 1127, 835, 743, 720 cm⁻¹.

### Synthesis of BT-2184

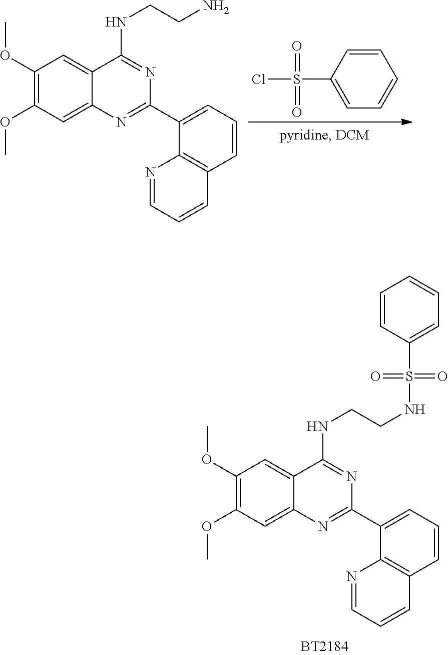 [SMILES: COc1cc2nc(-c3cccc4cccnc34)nc(NCCN)c2cc1OC ; O=S(=O)(Cl)c1ccccc1 ; COc1cc2nc(-c3cccc4cccnc34)nc(NCCNS(=O)(=O)c3ccccc3)c2cc1OC]

A solution of benzene sulfonyl chloride (45.5 mg, 0.40 mmol) in dichloromethane (2 mL) and pyridine (1 mL) was treated with N¹-(6,7-dimethoxy-2-(quinolin-8-yl)quinazolin-4-yl)ethane-1,2-diamine (50.0 mg, 0.13 mmol) at 0° C. The resulting mixture was stirred at same temperature for 1 h then warmed up to 18° C. until the conversion was complete (observed by TLC). Solvent was then evaporated and the residue was subjected to flash column chromatography (silica, 5:95 v/v methanol saturated ammonia/ethyl acetate elution) and concentration of the appropriate fractions (R_{f}=0.30, 5:95 v/v methanol saturated ammonia/ethyl acetate elution) to give BT-2184 (50.0 mg, 44%) as an off white solid. ¹H NMR (400 MHz, Chloroform-d) δ 9.00 (d, J=3.8 Hz, 1H), 8.23 (d, J=8.2 Hz, 1H), 8.08 (d, J=7.0 Hz, 1H), 7.91 (d, J=8.2 Hz, 1H), 7.66 (t, J=7.6 Hz, 1H), 7.66 (overlapped, brs, NH), 7.50 (d, J=7.6 Hz, 2H), 7.42 (dd, J=8.1, 4.1 Hz, 1H), 7.15 (t, J=7.4 Hz, 1H), 7.07 (s, 1H), 7.04-7.01 (m, 2H), 6.72 (s, 1H), 6.63 (brs, NH), 3.89 (s, 3H), 3.67 (s, 3H), 3.52 (m, 2H), 3.22 (m, 2H); ¹³C NMR (101 MHz, Chloroform-d) δ 161.0, 158.8, 154.0, 150.6, 148.4, 146.7, 146.0, 140.6, 139.2, 136.9, 131.5, 131.2, 129.0, 128.8 (2C), 126.5, 126.3 (2C), 121.2, 107.2, 107.0, 100.4, 77.4, 56.1, 56.0, 44.6, 39.9; MS (ESI, +ve) m/z 516 [(M+H), 100%]; HRMS (ESI, +ve) Found: (M+H)+516.1695, C₂₇H₂₆N₅O₄S requires 516.1706; IR (ATR) vₘₐₓ 3273, 3002, 2936, 2833, 1623, 1589, 1526, 1501, 1420, 1361, 1321, 1305, 1258, 1244, 1217, 1156, 1092, 999, 796, 754, 689 cm⁻¹.

### Synthesis of BT-2185

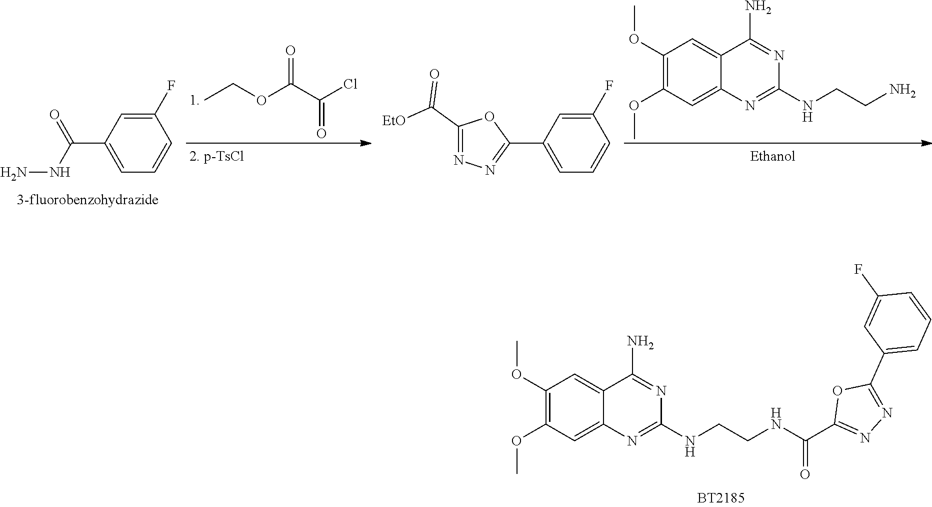

A magnetically stirred solution of 3-fluorobenzohydrazide (502 mg, 3.26 mmol) in DCM (20 mL) maintained at 0° C. was treated with triethylamine (1.20 mL, 8.60 mmol) followed by dropwise addition of ethyl chlorooxoacetate (0.33 mL, 2.98 mmol). The mixture was stirred for 0.5 h then warmed and held at 18° C. for 0.5 h before p-toluenesulfonyl chloride (543 mg, 2.85 mmol) was added in one portion. The mixture was stirred for 18 h then a saturated aqueous solution of NaHCO₃ (10 mL) was added and the solution extracted with DCM (2×15 mL). The combined organic layers were then washed with brine (15 mL), dried (Na₂SO₄) and concentrated in vacuo to afford a residue which was purified by flash chromatography (1:20 v/v diethyl ether/DCM elution) to afford ethyl 5-(3-fluorophenyl)-1,3,4-oxadiazole-2-carboxylate (442 mg, 66%) as white crystals. ¹H NMR (400 MHz, Chloroform-d) δ 7.99-7.95 (m, 1H), 7.89-7.85 (m, 1H), 7.57-7.51 (m, 1H), 7.34-7.28 (m, 1H), 4.56 (q, J=7.1 Hz, 2H), 1.49 (t, J=7.2 Hz, 3H); ¹³C NMR (101 MHz, Chloroform-d) δ 165.5 (d, JC-F=3.3 Hz), 164.2, 161.7, 155.6 (d, J=242.7 Hz), 131.3 (d, JC-F=8.1 Hz), 124.7 (d, JC-F=8.6 Hz), 123.5 (d, JC-F=3.2 Hz), 120.1 (d, JC-F=21.1 Hz), 114.7 (d, JC-F=24.5 Hz), 63.8, 14.2.

According to General Procedure E, a solution of N²-(2-aminoethyl)-6,7-dimethoxyquinazoline-2,4-diamine (83.6 mg, 0.32 mmol), ethyl 5-(3-fluorophenyl)-1,3,4-oxadiazole-2-carboxylate (50.0 mg, 0.21 mmol) described directly above, and ethanol (1 mL) was irradiated at 80° C. for 3 h. Flash chromatography (R_{f}=0.32, 8:92 v/v methanol saturated ammonia/dichloromethane) provided BT-2185 (85.0 mg, 88%) as an off white solid. ¹H NMR (400 MHz, DMSO-d₆) δ 9.94 (brs, 1H), 7.87 (d, J=7.7 Hz, 1H), 7.79 (d, J=9.0 Hz, 1H), 7.66 (q, 3=7.6 Hz, 1H), 7.52 (t, J=8.2 Hz, 1H), 7.41 (s, 1H), 7.08 (brs, 2H), 6.88 (s, 1H), 6.44 (brs, 1H), 3.86 (s, 3H), 3.78 (s, 3H), 3.52 (brs, 4H); ¹³C NMR (101 MHz, DMSO-d₆) δ 163.8 (JC-F=3.2 Hz), 162.2 (JC-F=245.6 Hz), 161.2, 159.4, 158.8, 154.1, 152.9, 148.2, 144.7, 131.8 (JC-F=8.3 Hz), 124.8 (JC-F=8.9 Hz), 123.3 (JC-F=3.0 Hz), 119.5 (JC-F=21.1 Hz), 113.8 (JC-F=21.4 Hz), 105.1, 103.8, 103.3, 55.8, 55.4, 41.3, 39.9; MS (ESI, +ve) m/z 454.1 [(M+H), 100%]; HRMS (ESI, +ve) Found: (M+H)+454.1639, C₂₁H₂₁N₇O₄F requires 454.1639; IR (ATR) vₘₐₓ 3542, 3412, 3345, 3231, 3045, 1681, 1652, 1605, 1578, 1554, 1504, 1455, 1434, 1364, 1321, 1227, 1208, 1112, 998, 852, 830, 789, 727 cm⁻¹.

### Synthesis of BT-2187

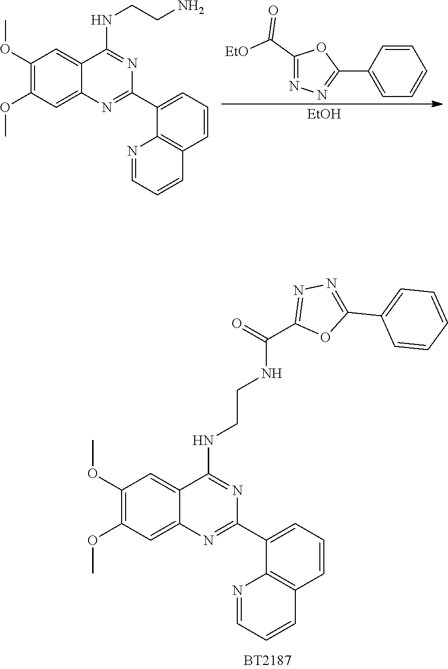 [SMILES: "EtOH" ; COc1cc2nc(-c3cccc4cccnc34)nc(NCCN)c2cc1OC ; CCOC(=O)c1nnc(-c2ccccc2)o1 ; COc1cc2nc(-c3cccc4cccnc34)nc(NCCNC(=O)c3nnc(-c4ccccc4)o3)c2cc1OC]

According to General Procedure E, a solution of N¹-(6,7-dimethoxy-2-(quinolin-8-yl)quinazolin-4-yl)ethane-1,2-diamine (103.2 mg, 0.27 mmol), ethyl 5-phenyl-1,3,4-oxadiazole-2-carboxylate (40.0 mg, 0.18 mmol) and ethanol (1 mL) was irradiated at 80° C. for 3 h. Flash chromatography (R_{f}=0.19, 10:90 v/v methanol saturated ammonia/diethyl ether) provided BT-2187 (79.5 mg, 79%) as an ivory solid. ¹H NMR (400 MHz, Chloroform-d) δ 9.03-9.02 (m, 1H), 8.64 (brs, 1H), 8.24 (d, J=8.3 Hz, 1H), 8.04 (d, J=7.0 Hz, 1H), 7.99-7.92 (m, 2H), 7.87 (d, J=8.3 Hz, 1H), 7.66-7.58 (m, 1H), 7.58-7.47 (m, 2H), 7.47-7.38 (m, 3H), 7.11 (s, 1H), 6.95 (s, 1H), 3.80 (s, 3H), 3.65 (m, 5H), 3.59 (s, 2H); ¹³C NMR (101 MHz, Chloroform-d) δ 166.0, 161.3, 159.3, 158.3, 154.4, 154.1, 149.9, 148.5, 146.5, 146.3, 140.0, 137.2, 132.5, 130.9, 129.2 (2C), 128.8, 128.6, 127.4 (2C), 126.8, 122.9, 121.2, 107.5, 107.0, 100.8, 56.1, 55.9, 40.8, 40.2. MS (ESI, +ve) m/z 548 [(M+H), 100%]; HRMS (ESI, +ve) Found: (M+H)⁺548.2044, C₃₀H₂₆N₇O₄ requires 548.2046; IR (ATR) vₘₐₓ 3279, 3060, 3006, 2937, 2833, 2251, 1683, 1622, 1578, 1548, 1525, 1499, 1449, 1421, 1352, 1245, 1217, 1174, 1143, 911, 798, 727, 711 cm⁻¹.

### Synthesis of BT-2229

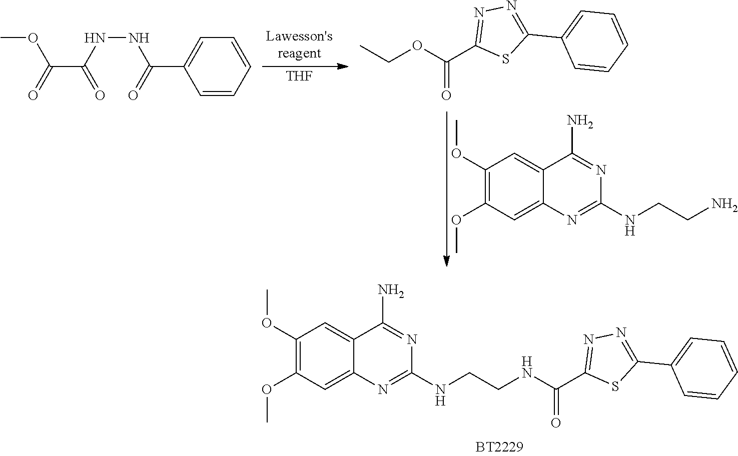

Following a procedure reported by Banville, J et al in WO 2013/163279, a magnetically stirred mixture of ethyl 2-(2-benzoylhydrazineyl)-2-oxoacetate (790 mg, 3.35 mmol) in anhydrous THF (5 mL) was treated at rt with Lawesson's Reagent (1.35 g, 3.35 mmol) and then heated at reflux for 24 h. The solvent was removed in vacuo and the residue purified by flash column chromatography (silica, 1:20 v/v EtoAc/DCM elution) to afford ethyl 5-phenyl-1,3,4-thiadiazole-2-carboxylate (239 mg, 30%) as a white powder. ¹H NMR (400 MHz, CDCl₃) δ 8.06-7.99 (m, 2H), 7.59-7.46 (m, 3H), 4.54 (q, J=7.1 Hz, 2H), 1.48 (t, J=7.1 Hz, 3H); ¹³C NMR (101 MHz, CDCl₃) δ 172.6, 159.6, 158.8, 132.1, 129.4 (3C), 128.3 (2C), 63.3, 14.2; MS (ESI, +ve) m/z 257 [(M+Na), 100%].

According to General Procedure E, a solution of N²-(2-aminoethyl)-6,7-dimethoxyquinazoline-2,4-diamine (247 mg, 0.94 mmol), 5-phenyl-1,3,4-thiadiazole-2-carboxylate (110 mg, 0.47 mmol) and ethanol (4 mL) was irradiated at 80° C. for 3 h. Flash chromatography (1:20 v/v methanol saturated ammonia/dichloromethane) provided BT-2229 (177 mg, 83%) as a yellow solid. ¹H NMR (400 MHz, DMSO-d₆) δ 10.04 (br s, 1H), 8.08-7.99 (m, 2H), 7.65-7.55 (m, 3H), 7.42 (s, 1H), 7.07 (br s, 2H), 6.99 (br s, 1H), 6.45 (s, 1H), 3.94 (s, 3H), 3.78 (s, 3H), 3.56-3.47 (m, 4H); ¹³C NMR (101 MHz, DMSO-d₆) one peak obscured or overlapping δ 171.3, 165.5, 161.2, 159.5, 157.4, 154.0, 148.4, 144.7, 132.0, 129.6 (2C), 129.1, 127.9 (2C), 103.8, 103.3, 64.9, 55.8, 55.4, 41.9, 40.1; MS (ESI, +ve) m/z 452 [(M+H), 100%]; Single Crystal X-Ray Data collection: CrysA/is PRO 1.171.38.46 (Rigaku OD, 2015); cell refinement: CrysA/is PRO 1.171.38.46 (Rigaku OD, 2015); data reduction: CrysAlis PRO 1.171.38.46 (Rigaku OD, 2015); program(s) used to solve structure: SHELXT (Sheldrick (2015) Acta Cryst., A 71: 3-8); program(s) used to refine structure: SHELXL (Sheldrick (2015) Acta Cryst., A 71: 3-8); molecular graphics: Olex2 (Dolomanov et al. (2009) J. Appl. Cryst., 42: 339-341); software used to prepare material for publication: Olex2 (Dolomanov et al. (2009) J. Appl. Cryst., 42: 339-341). Crystal data: C₂₁H₂₁N₇O₃S Mr=451.51 Monoclinic, P21/n a=9.3352 (10) Å b=17.4633 (9) Å c=13.1223 (11) Å β=97.834 (9°) V=2119.3 (3) Å3 Z=4 F(000)=944 Dx=0.415 Mg m⁻³ Cu Kα radiation, λ=1.54184 Å Cell parameters from 2892 reflections θ=5.1-71.8° μ=1.70 mm⁻¹.

### Example 6—Inhibition of Heparanase Activity by Compounds of formula 1

### Materials and Methods

Heparanase inhibition assays were conducted according to the protocol of Example 3. Test compounds were dissolved and added to the assay at varying concentrations to calculate the level of inhibition.

### Results

Heparanase inhibition activity of various compounds of the invention was determined using standard methods. The results, expressed as the half maximal inhibitory concentration (IC₅₀), being the concentration of the compound of the invention required to achieve 50% inhibition of heparanase, are shown in Table 3.

TABLE 3
Heparanase inhibition by compounds of Formula 1

| Compound | Structure | IC₅₀ |
|---|---|---|
| BT-2005 | 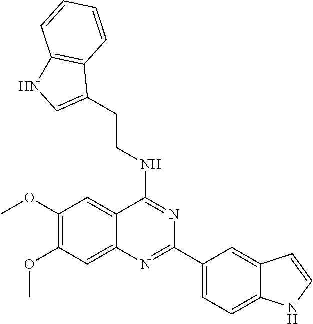 [SMILES: COc1cc2nc(-c3ccc4[nH]ccc4c3)nc(NCCc3c[nH]c4ccccc34)c2cc1OC] | 41 μM |
| BT-2007 | 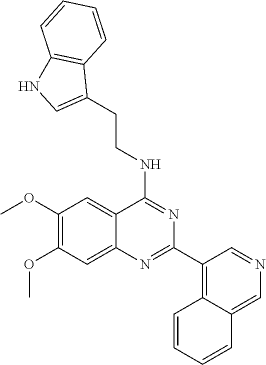 [SMILES: COc1cc2nc(-c3cncc4ccccc34)nc(NCCc3c[nH]c4ccccc34)c2cc1OC] | 92.7 μM |
| BT-2013 (doxazosin mesylate) | 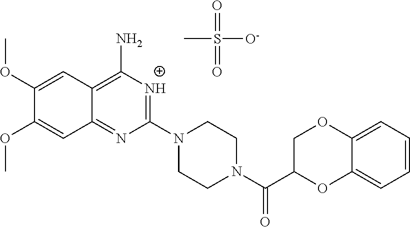 | 57.5 μM |
| BT-2057 | 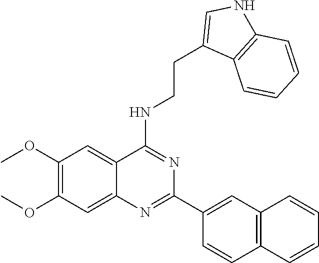 [SMILES: COc1cc2nc(-c3ccc4ccccc4c3)nc(NCCc3c[nH]c4ccccc34)c2cc1OC] | 56 μM |
| BT-2062 | 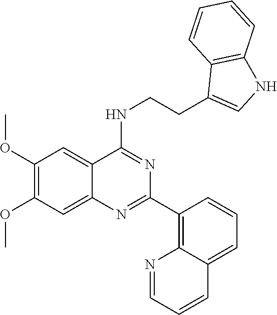 [SMILES: COc1cc2nc(-c3cccc4cccnc34)nc(NCCc3c[nH]c4ccccc34)c2cc1OC] | 1 μM |
| BT-2090 | 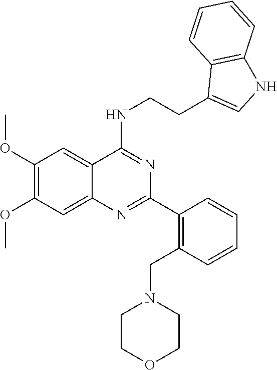 [SMILES: COc1cc2nc(-c3ccccc3CN3CCOCC3)nc(NCCc3c[nH]c4ccccc34)c2cc1OC] | 53.6 μM |
| BT-2120 | 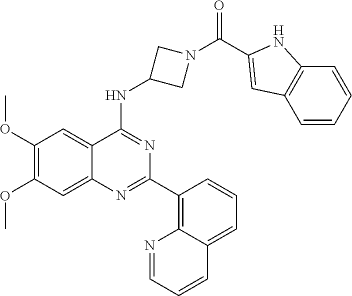 [SMILES: COc1cc2nc(-c3cccc4cccnc34)nc(NC3CN(C(=O)c4cc5ccccc5[nH]4)C3)c2cc1OC] | 0.7 μM |
| BT-2148 | 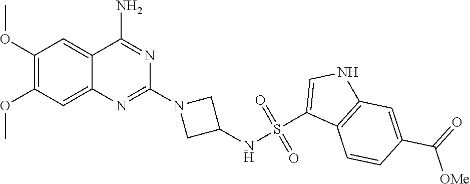 [SMILES: COC(=O)c1ccc2c(S(=O)(=O)NC3CN(c4nc(N)c5cc(OC)c(OC)cc5n4)C3)c[nH]c2c1] | 20.5 μM |
| BT-2161 | 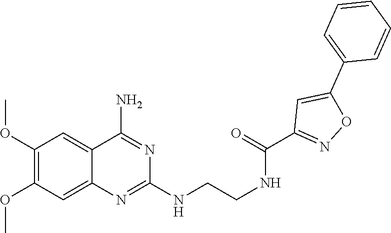 [SMILES: COc1cc2nc(NCCNC(=O)c3cc(-c4ccccc4)on3)nc(N)c2cc1OC] | 18.9 μM |
| BT-2162 | 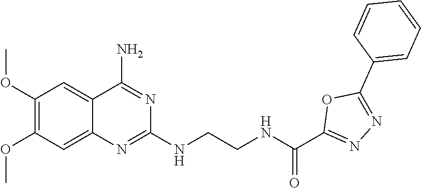 [SMILES: COc1cc2nc(NCCNC(=O)c3nnc(-c4ccccc4)o3)nc(N)c2cc1OC] | 22.6 μM |
| BT-2164 | 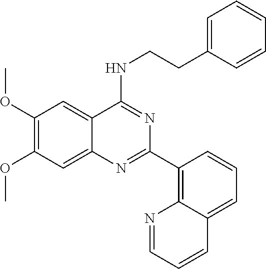 [SMILES: COc1cc2nc(-c3cccc4cccnc34)nc(NCCc3ccccc3)c2cc1OC] | 6.1 μM |
| BT-2167 | 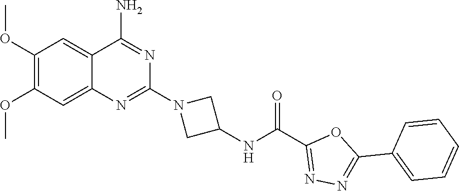 [SMILES: COc1cc2nc(N3CC(NC(=O)c4nnc(-c5ccccc5)o4)C3)nc(N)c2cc1OC] | 33 μM |
| BT-2169 | 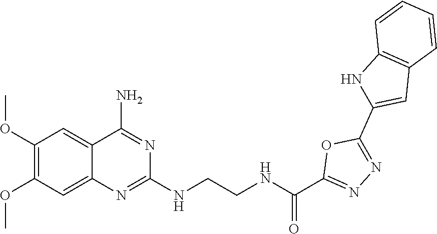 [SMILES: COc1cc2nc(NCCNC(=O)c3nnc(-c4cc5ccccc5[nH]4)o3)nc(N)c2cc1OC] | 3.3 μM |
| BT-2170 | 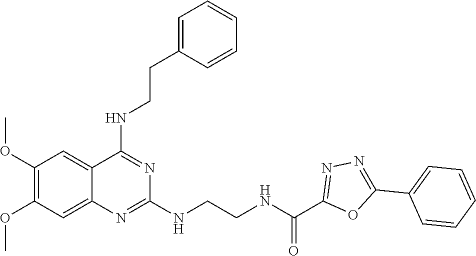 [SMILES: COc1cc2nc(NCCNC(=O)c3nnc(-c4ccccc4)o3)nc(NCCc3ccccc3)c2cc1OC] | 55 μM |
| BT-2171 | 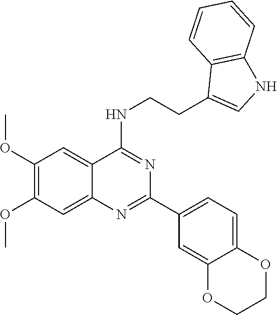 [SMILES: COc1cc2nc(-c3ccc4c(c3)OCCO4)nc(NCCc3c[nH]c4ccccc34)c2cc1OC] | 14 μM |
| BT-2172 (BT-2162 HCl) | 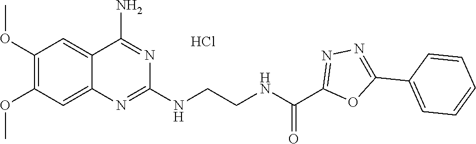 [SMILES: COc1cc2nc(NCCNC(=O)c3nnc(-c4ccccc4)o3)nc(N)c2cc1OC ; Cl] | 6.7 μM |
| BT-2173 (BT-2169 HCl) | 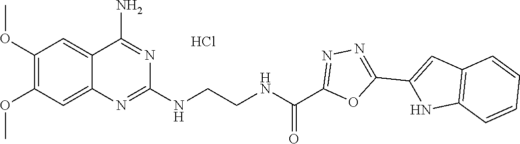 [SMILES: COc1cc2nc(NCCNC(=O)c3nnc(-c4cc5ccccc5[nH]4)o3)nc(N)c2cc1OC ; Cl] | 4.3 μM |
| BT-2177 | 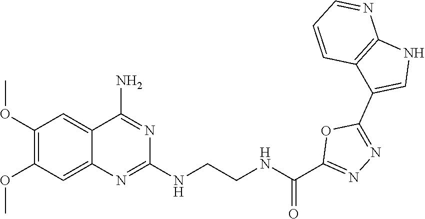 [SMILES: COc1cc2nc(NCCNC(=O)c3nnc(-c4c[nH]c5ncccc45)o3)nc(N)c2cc1OC] | 21 μM |
| BT-2178 | 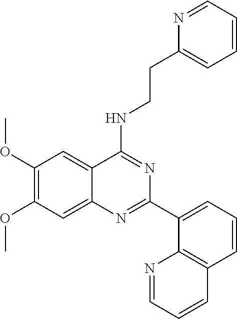 [SMILES: COc1cc2nc(-c3cccc4cccnc34)nc(NCCc3ccccn3)c2cc1OC] | 11 μM |
| BT-2179 | 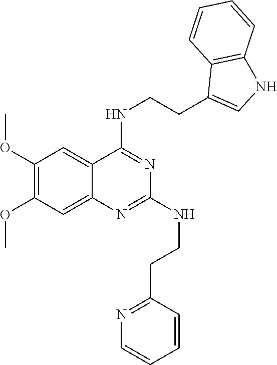 [SMILES: COc1cc2nc(NCCc3ccccn3)nc(NCCc3c[nH]c4ccccc34)c2cc1OC] | 18 μM |
| BT-2181 | 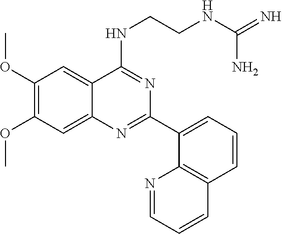 [SMILES: COc1cc2nc(-c3cccc4cccnc34)nc(NCCNC(=N)N)c2cc1OC] | 11 μM |
| BT-2182 | 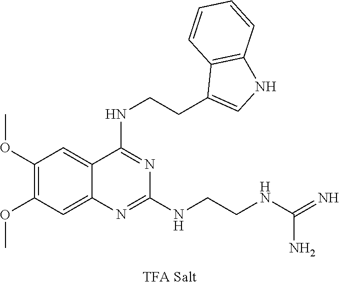 [SMILES: COc1cc2nc(NCCNC(=N)N)nc(NCCc3c[nH]c4ccccc34)c2cc1OC] | 18 μM |
| BT-2184 | 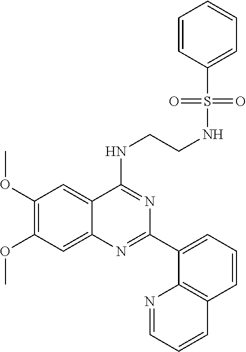 [SMILES: COc1cc2nc(-c3cccc4cccnc34)nc(NCCNS(=O)(=O)c3ccccc3)c2cc1OC] | 3.39 μM |
| BT-2185 | 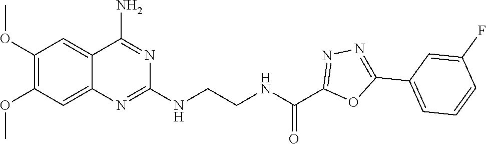 [SMILES: COc1cc2nc(NCCNC(=O)c3nnc(-c4cccc(F)c4)o3)nc(N)c2cc1OC] | 22 μM |
| BT-2187 | 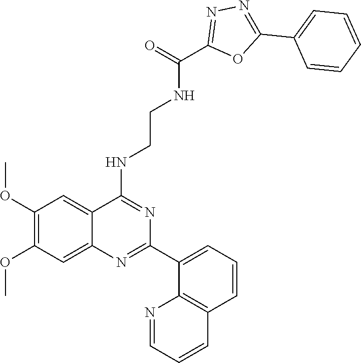 [SMILES: COc1cc2nc(-c3cccc4cccnc34)nc(NCCNC(=O)c3nnc(-c4ccccc4)o3)c2cc1OC] | 4.1 μM |
| BT-2229 | 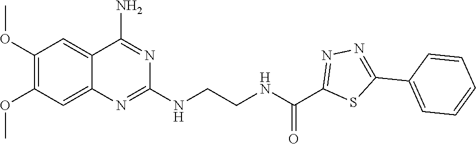 [SMILES: COc1cc2nc(NCCNC(=O)c3nnc(-c4ccccc4)s3)nc(N)c2cc1OC] | 13.4 μM |

### Example 7—In Vivo Efficacy of Bt-2172 and Pentosan Polysulfate Compared to C3 Knock Out Following Photo-Oxidative Damage in a Mouse Model of Dry Amd

### Materials and Methods

Animal experimentation, light exposure device and measurement of retinal function was performed in accordance with the procedure of Example 4.

### C3 Complement Factor Gene Knockout Mice

C57BL/63 mice and C3-knockout mice (C3−/−, 12954-C3tm1Crr/J), both aged 60-80 days, were obtained from the Australian Phenomics Facility (Canberra, ACT, Australia). For the C3-knockout mice experiments, the knockout mice were compared to isogenic C57BL/63 littermates using the photooxidative damage protocols as described in Example 4. In brief, C57BL/63 and C3−/− mice were housed in Perspex boxes coated with a reflective interior, and exposed to 100 k lux of natural white LED for 5 days, with free access to food and water. Each animal was administered with pupil dilator eye-drops twice daily during photooxidative damage (Atropine Sulfate 1% w/v eye-drops; Bausch and Lomb, NSW, Australia). Following photooxidative damage, electroretinography (ERG) was used to measure mouse retinal function in response to full-field flash stimuli under scotopic conditions in dim-reared control and 7-day damaged mice as described in Example 4.

### Intravitreal Injections

Intravitreal injections were performed as described in detail previously (Rutar M V et al (2012) J Neuroinflammation 9: 221) wherein animals were anesthetized using an intraperitoneal injection of ketamine (100 mg/kg; Troy Laboratories, NSW, Australia) and xylazil (12 mg/kg; Troy Laboratories).

The BT-2172 formulation was prepared as follows. BT-2172 (prepared in accordance with Example 5) was suspended in ultrapure PBS to a concentration of 200 μM and sonicated until dissolved. The solution was then filtered using a 0.22 μm syringe filtration system.

Injections into individual animals consisted of a 1 μL solution containing PBS (control), pentosan polysulfate (PPS; 2 μg/μL) as formulated in Example 3 or BT-2172 (200 μM) as formulated above (n=7-11 for each group). Animals were allowed to wake from anesthetic, during which corneal hydration was maintained though application of a synthetic tear gel (GenTeal Gel; Novartis, NSW, Australia). Animals were exposed to photo-oxidative damage for 5 days as described in Example 1.

### Results

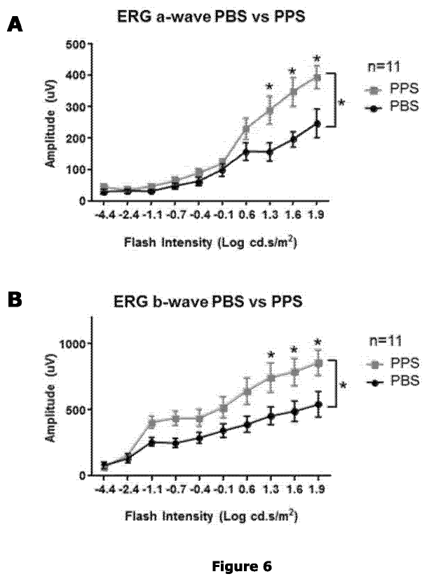

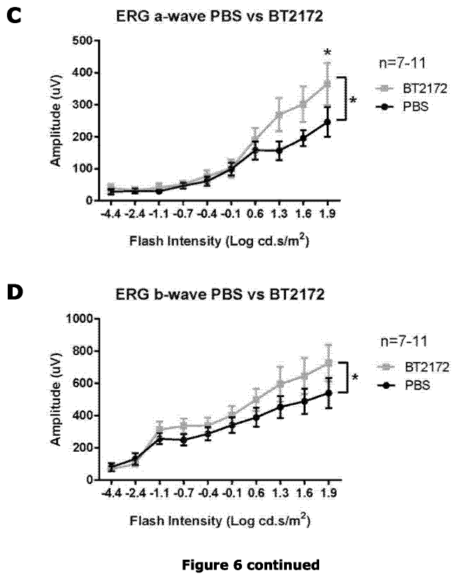

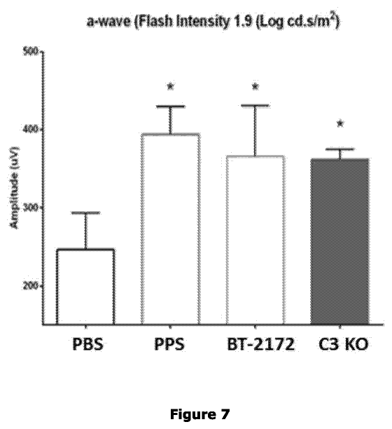

BT-2172 and pentosan polysulfate delivered by intravitreal injection maintained retinal function in mice exposed to photo-oxidative damage, a model related to dry age-related macular degeneration (FIGS. 6A-D). The a-wave and b-wave responses of the ERG (FIG. 6) reflects the differences in retinal morphology of untreated and treated animals described above. ERG a-wave and b-wave intensity response characteristics for BT-2172 treated mice (FIGS. 6C and D) and pentosan polysulfate treated mice (FIGS. 6A and B) were significantly different (p<0.05 for both treatments) compared to control mice (PBS). The benefit of BT-2172 and pentosan polysulfate treatment was demonstrated across multiple flash intensities and was most pronounced at the highest flash intensity (p<0.05 for both treatments, FIGS. 6A-D). The BT-2172 and pentosan polysulfate treated mice had higher a- and b-wave responses than the control (PBS) group and were similar to the C3 complement factor gene knockout mice at high flash intensities (FIG. 7).

### Example 8—Synthesis of Compounds of Formula 2, 3, and 4

Analytical techniques, starting materials and general synthetic techniques are as described in Example 5.

### General Procedure A—Preparation of 2-isothiocyanatobenzonitriles

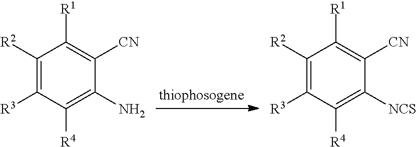

Following the procedure of Calestani et al. (2001) Tetrahedron, 57(33): 7221, a magnetically stirred mixture of thiophosgene (1.35 mL, 17.5 mmol) in DCM (2 mL) and water (4 mL) was treated dropwise with a solution of the substituted amino-benzonitrile (14.3 mmol) in DCM (15 mL). The mixture was stirred for two hours and was then transferred to a separatory funnel and the aqueous layer extracted with DCM (20 mL). The organic layers were combined, washed with water (2×20 mL) then dried over Na₂SO₄. The DCM was removed with a gentle stream of nitrogen with heating at 40° C. and the residue obtained was then held at rt under high vacuum (1 mmHg) for 4 h to afford the desired 2-isothiocyanatobenzonitrile which may be used without further purification.

### General Procedure B—Reaction of Primary Amines with 2-isothiocyanatobenzonitriles

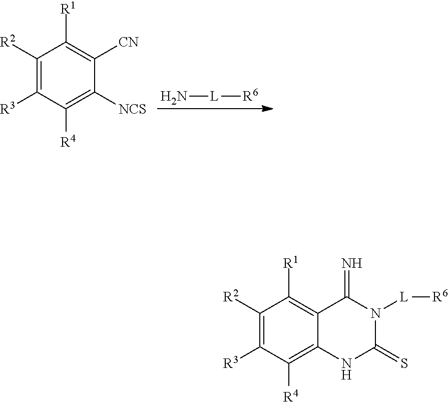

Following a modified procedure analogous to that reported by Pazdera (1989) Chem. Papers, 43(3): 465, a magnetically stirred solution of substituted 2-isothiocyanatobenzonitrile (1.36 mmol) in a mixture of DCM and petroleum spirit (2.5 mL:2.5 mL) was treated with a solution of primary amine in DCM (7 mL). The mixture was stirred for 0.5 h and then petroleum spirit (15 mL) was added. The solid was collected by vacuum filtration and then magnetically stirred in a solution of ethanol (30 mL) and heated at 70° C. for 2 h. The mixture was cooled to 0° C. and the solid was collected by vacuum filtration, washed with ethanol (10 mL) and dried at the pump (1 mmHg).

### General Procedure C—Reaction of Primary Amines with 2-isothiocyanatobenzonitriles—Alternative Procedure

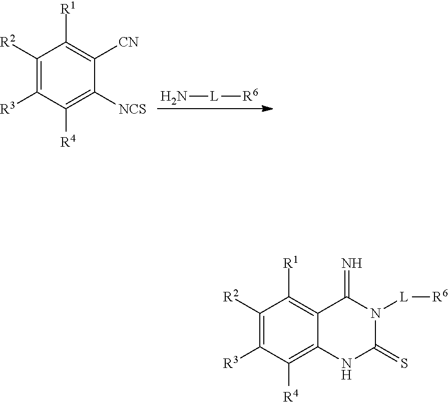

Crude amine hydrochloride (0.27 mmol) suspended in ethanol (6 mL), was treated with triethylamine (96 μL, 0.68 mmol) and then a solution of 2-isothiocyanato-4,5-dimethoxybenzonitrile (50 mg, 0.27 mmol) in DCM (3 mL) was added. The mixture was stirred at rt for 1 h then concentrated by a gentle stream of nitrogen and re-suspended in ethanol (5 mL). The mixture was heated at reflux for 4 h and the precipitate that formed was collected and washed with ethanol (15 mL) then ether (10 mL) and dried at the pump.

### General Procedure D—Suzuki Cross-Coupling Procedure

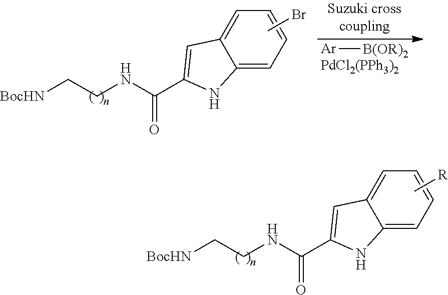

A 10 mL snap-cap microwave vessel, fitted with a magnetic stirring bar, was charged with a mixture of boronic acid or ester (77.2 μmol), aryl bromide or triflate (52.3 μmol) and potassium carbonate (38 mg, 274 μmol) then treated with a degassed mixture of dimethoxyethane, water and ethanol (7:3:2, 1 mL). Bis(triphenylphosphine)palladium(II) dichloride (1.8 mg, 5 mol %) was added and the mixture was sparged with nitrogen for 0.05 hr, sealed then subjected to microwave irradiation (120° C./0.33 h, ramp time 1 minute, maximum power 200 W). The mixture was treated with water (1 mL) and extracted with EtOAc (3×2 mL) and the combined organic layers washed with brine and concentrated under a gentle stream of nitrogen. The residue obtained was subjected to flash column chromatography [silica, 1:10 v/v MeOH/DCM elution] (unless otherwise specified) to give, after concentration of the appropriate fractions the desired compound.

### General Procedure E—Boc Deprotection with Hydrogen Chloride in Dioxane

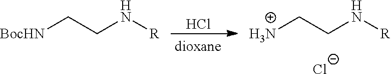

A solution of hydrogen chloride (2 mL, 4M in dioxane) at 4° C. was added to the Boc-protected compound and magnetically stirred for 1 h at rt. Diethyl ether (5 mL) was added and the mixture vigorously stirred for 0.05 h then magnetic stirring was ceased and the precipitate or gum allowed to settle. The solvent was decanted off the precipitate and this procedure was repeated a further 3 times and the resulting compound placed under high vacuum for 3 hours and used without further purification.

### General Procedure F—Boc Deprotection with Trifluoroacetic Acid in Dichloromethane

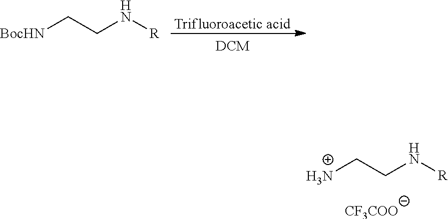

A magnetically stirred suspension of Boc-protected compound (1.80 mmol) in DCM (4 mL) maintained at 0° C. was treated with trifluoroacetic acid (1 mL) and magnetically stirred for 2 h. The cold bath was removed and the mixture was then stirred for a further 1 h at rt. The solvent was removed with a gentle stream of nitrogen and the remaining gum was triturated with diethyl ether (3×10 mL) then placed under high vacuum for 1 h to afford the amine trifluoroacetate salt as a powder and used directly without further purification.

### General Procedure G—Acid Chloride Formation and Amine Addition

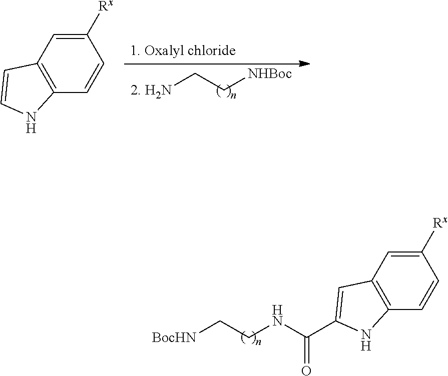

A magnetically stirred suspension of carboxylic acid (6.25 mmol) in DCM (30 mL) at 0° C. was treated with oxalyl chloride (563 μL, 6.57 mmol) dropwise, followed by DMF (1 drop). The mixture was stirred at 0° C. for 1 h and then the cold-bath was removed and stirring was continued at rt for 2.5 hr. The clear solution was then concentrated with a gentle stream of nitrogen with heating, in a water bath at 40° C. The resulting cream coloured powder was then placed under high vacuum (1 mm Hg) for 1 hr and redissolved in THF (20 mL) and added transferred dropwise by syringe to a magnetically stirred, ice-cold solution of amine (6.25 mmol) and N,N-diisopropylethylamine (1.75 mL, 12.5 mmol) in DCM (20 mL). After 1 hr at 0° C. the cold bath was removed and stirred a further one hr at 18° C. then a solution of sodium hydrogen carbonate (50 mL, half saturated) was added and the DCM and THF were removed in vacuo. The precipitate was collected by vacuum filtration and washed with water (200 mL) and was dried under high vacuum for 18 hours.

### General Procedure H

The general procedure is illustrated with respect to the synthesis of the compound BT-2152.

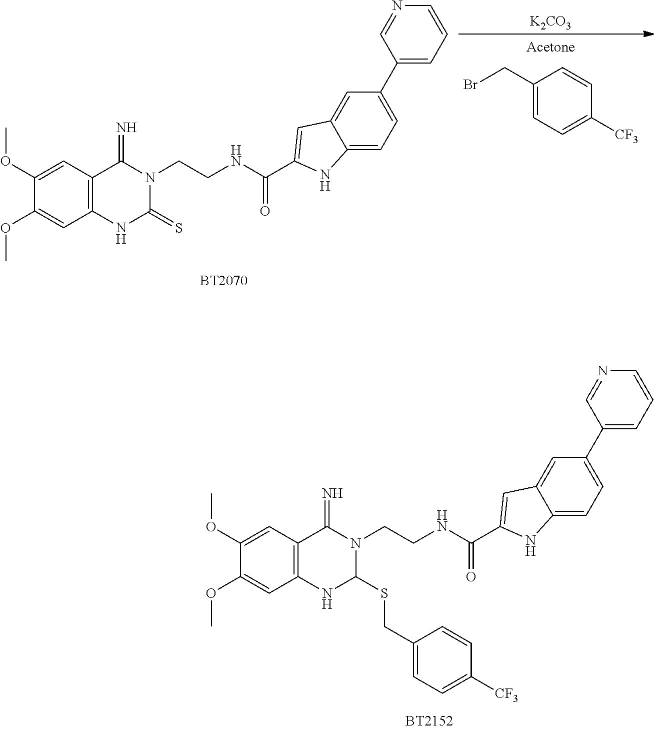

A mixture of BT-2070 (50 mg, 0.10 mmol), potassium carbonate (28 mg, 0.20 mmol) in acetone (1.5 mL) at rt was treated with 1-(bromomethyl)-4-(trifluoromethyl)benzene (24 μL, 0.10 mmol) in one portion. The mixture was stirred for 24 h and then the mixture was purified by flash column chromatography [silica, 1:20 v/v ammoniacal MeOH/DCM elution] to give, after concentration of the appropriate fractions, BT-2152 as a white powder (11 mg, 17%). ¹H NMR (DMSO-d₆, 400 MHz) δ 11.73 (s, 1H), 8.91 (d, J=2.0 Hz, 1H), 8.82 (app. t, J=5.9 Hz, 1H), 8.52 (dd, J=4.7, 1.6 Hz, 1H), 8.48 (br s, 1H), 8.07 (ddd, J=8.0, 2.4, 1.6 Hz, 1H), 7.98 (s, 1H), 7.65 (s, 1H), 7.58 (dd, J=8.8, 1.7 Hz, 1H), 7.54 (d, J=8.8 Hz, 1H), 7.47 (dd, J=8.0, 4.7 Hz, 1H), 7.39 (d, J=8.0 Hz, 2H), 7.28 (d, J=8.0 Hz, 2H), 7.12 (d, J=2.0 Hz, 1H), 6.86 (s, 1H), 4.42 (s, 2H), 4.34-4.27 (s, 2H), 3.86 (s, 3H), 3.85 (s, 3H), 3.71-3.64 (m, 2H). (+)-LRESIMS m/z (rel. int.) 659 (100) [M+H]⁺; vₘₐₓ 1623, 1601, 1547, 1510, 1328, 1233, 1106, 1018, 799 cm⁻¹.

### Preparation (i). 2-Isothiocyanato-4,5-dimethoxybenzonitrile

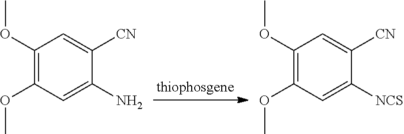

Prepared according to General Procedure A, from reaction of 2-amino-4,5-dimethoxybenzonitrile (2.50 g, 14.3 mmol) and thiophosgene (1.35 mL, 17.5 mmol) to afford 2-isothiocyanato-4,5-dimethoxybenzonitrile as a pale-orange powder (2.60 g, 84%). ¹H NMR (CDCl₃, 400 MHz) δ 6.96 (s, 1H), 6.75 (s, 1H), 3.93 (s, 3H), 3.90 (s, 3H); ¹³C NMR (CDCl₃, 100 MHz) δ 153.4, 148.1, 139.8, 128.9, 115.8, 113.6, 109.4, 101.0, 56.5, 56.4; LRMS (EI, 70 eV) m/z (rel. int.) 220 (100, M+), 205 (65), 177 (39), 119 (28); HREIMS calcd. for C₁₀H₁₈N₂O₂S [M+.220.0301, found 220.0312; vₘₐₓ 3311, 2221, 1713, 1598, 1527, 1406, 1239, 1213, 1191, 1118, 1000, 688 cm⁻¹.

### Preparation (ii): Tert-Butyl (2-(5-bromo-1H-indole-2-carboxamido)ethyl)carbamate

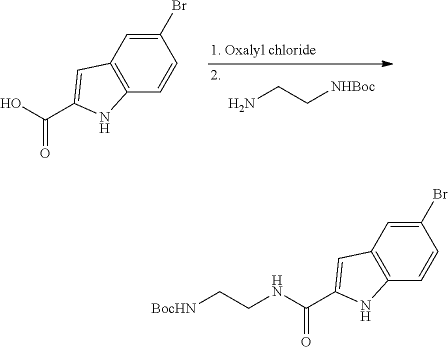 [SMILES: O=C(O)c1cc2cc(Br)ccc2[nH]1 ; CC(C)(C)OC(=O)NCCN ; CC(C)(C)OC(=O)NCCNC(=O)c1cc2cc(Br)ccc2[nH]1]

Following General Procedure G, 5-bromo-1H-indole-2-carboxylic acid (1.50 g, 6.25 mmol) was converted to 5-bromo-1H-indole-2-carbonyl chloride and reacted with tert-butyl (2-aminoethyl)carbamate (1.00 g, 6.25 mmol) to afford tert-butyl (2-(5-bromo-1H-indole-2-carboxamido)ethyl)carbamate (2.17 g, 91%) as a white powder. ¹H NMR (DMSO-d₆, 400 MHz) δ 11.78 (s br, 1H), 8.57 (t, J=5.7 Hz, 1H), 7.84 (d, J=2.0 Hz, 1H), 7.39 (d, J=8.7 Hz, 1H), 7.28 (dd, J=8.7, 2.0 Hz, 1H), 7.08 (s, 1H), 6.92 (t, J=5.7 Hz, 1H), 3.32 (app. q, J=6.3 Hz, 2H), 3.13 (app. q, J=6.3 Hz, 2H), 1.37 (s, 9H); ¹³C NMR (DMSO-d₆, 100 MHz) 2 signals obscured by DMSO-d₆ δ 160.9, 155.7, 135.0, 133.1, 128.9, 125.8, 123.6, 114.3, 112.1, 101.9, 77.7, 28.2 (3C); (+)-LRESIMS m/z (rel. int.) 404 (100) [M+H], 406 (100) [M+H]⁺; (+)-HRESIMS calcd. for C₁₆H₂₀N₃O₃Na⁸¹Br [M+Na]⁺ 406.0560, found 406.0561; vₘₐₓ 3357, 3269, 1684, 1629, 1546, 1531, 1367, 1275, 1252, 1167, 880, 797, 768 cm⁻¹.

### Synthesis of BT-2058

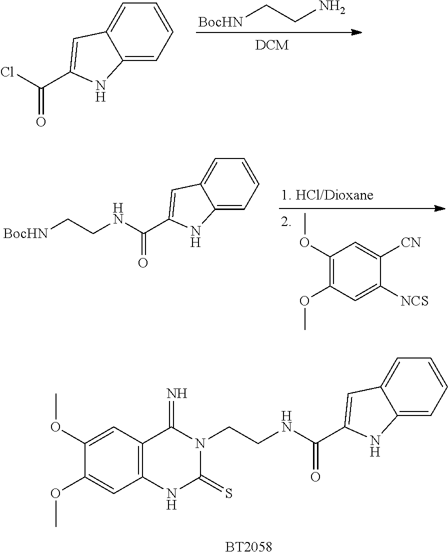

A magnetically stirred solution of 1H-indole-2-carbonyl chloride, prepared according to Kumarb et. al. (2012) Eur. J. Chem., 3(2), 214, (350 mg, 1.95 mmol) in DCM (5 mL) at −10° C. was treated dropwise with a solution of pyridine (1 mL), diisopropylethylamine (272 μL, 1.95 mmol) and tert-butyl (2-aminoethyl)carbamate (312 mg, 1.95 mmol) in DCM (20 mL). The mixture was allowed to warm to rt over 18 h and then the mixture was diluted with DCM (30 mL) and washed with aqueous HCl (2×10 mL, 0.2M) followed by a half saturated aqueous solution of sodium bicarbonate (2×15 mL) then brine (10 mL). The organic layer was dried (Na₂SO₄) and concentrated in vacuo to afford a solid that was subjected to flash column chromatography [silica, 1:20 v/v MeOH/DCM elution] to give, after concentration of the appropriate fractions, tert-butyl (2-(1H-indole-2-carboxamido)ethyl)carbamate (296 mg, 50%) as a tan coloured solid. ¹H NMR (CDCl₃, 400 MHz) one NH not observed δ 9.19 (s, 1H), 7.65 (d, J=8.0 Hz, 1H), 7.43 (d, J=8.2 Hz, 1H), 7.29 (d, J=7.2 Hz, 1H), 7.14 (t, J=7.5 Hz, 1H), 6.90 (s, 1H), 5.02-4.93 (m, 1H), 3.63-3.54 (m, 2H), 3.50-3.39 (m, 2H), 1.45 (s, 9H); (+) LRESIMS m/z (rel. int.) 326 (100) [M+Na]⁺; (+)-HRESIMS calcd. for C₁₆H₂₁N₃NaO₃ [M+Na]⁺ 326.1475, found 326.1481; vₘₐₓ 3308, 3226, 1694, 1609, 1541, 1422, 1280, 1254, 1152, 1018, 825, 745 cm⁻¹. tert-Butyl (2-(1H-indole-2-carboxamido)ethyl)carbamate (100 mg, 0.33 mmol) was treated with a solution of hydrogen chloride (2 mL, 4M in dioxane) and magnetically stirred for 1 h at 4° C. then a further 0.5 h at rt. Ether (5 mL) was added to precipitate the product and the remaining gum was triturated with ether (3×5 mL) then placed under high vacuum for 1 h to afford N-(2-aminoethyl)-1H-indole-2-carboxamide hydrochloride as a cream coloured gum and used directly without further purification. A mixture of triethylamine (66 μL, 0.47 mmol), N-(2-aminoethyl)-1H-indole-2-carboxamide hydrochloride (57 mg, 0.24 mmol, crude from above) and DCM (2 mL) was added to a magnetically stirred solution of 2-isothiocyanato-4,5-dimethoxybenzonitrile (50 mg, 0.27 mmol) in a mixture of DCM and petroleum spirit (1 mL:1 mL). The mixture was stirred for 1 h and then petroleum spirit (5 mL) was added. The solid was collected by vacuum filtration then suspended in ethanol (5 mL) and the mixture heated at 70° C. for 1 h. The mixture was cooled to rt and the solid was collected by vacuum filtration, washed with ethanol (5 mL) and dried at the pump (1 mmHg) to afford BT-2058 as a cream powder (32 mg, 33%). ¹H NMR (DMSO-d₆, 400 MHz) δ 11.93 (s br, 1H), 11.56 (s, 1H), 9.78 (s br, 1H), 8.80 (s, 1H), 7.73 (s, 1H), 7.60 (d, J=7.9 Hz, 1H), 7.42 (d, J=8.2 Hz, 1H), 7.17 (t, J=7.6 Hz, 1H), 7.07 (s, 1H), 7.03 (t, J=7.5 Hz, 1H), 6.89 (s, 1H), 5.01-4.73 (m, 2H), 3.84 (s, 6H), 3.76-3.65 (m, 2H); (+)-LRESIMS m/z (rel. int.) 424 (100) [M+H]⁺; (+)-HRESIMS calcd. for C₂₁H₂₂N₅O₃S [M+H]⁺ 424.1438, found 424.1442. vₘₐₓ 3243, 1623, 1557, 1545, 1512, 1282, 1189, 1020, 766, 738 cm⁻¹.

### Synthesis of BT-2134

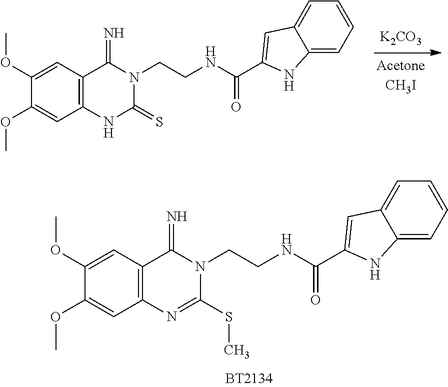

A mixture of BT-2058 (50 mg, 0.12 mmol), potassium carbonate (32 mg, 0.23 mmol) in acetone at rt was treated with methyl iodide (29 μL, 0.47 mmol) in one portion. The mixture was stirred for 48 h and then water (4 mL) was added and the mixture stirred for 1 min. The mixture was filtered by vacuum filtration to afford a solid that was washed with acetone (3 mL) to afford BT-2134 as a white powder (23 mg, 45%). ¹H NMR (DMSO-d₆, 400 MHz) δ 11.56 (s, 1H), 8.81 (app. t, J=5.6 Hz, 1H), 8.46 (s, 1H), 7.64 (s, 1H), 7.60 (d, J=8.0 Hz, 1H), 7.41 (d, J=8.0 Hz, 1H), 7.17 (t, J=7.6 Hz, 1H), 7.04 (s, 1H), 7.01 (d, J=7.6 Hz, 1H), 6.83 (s, 1H), 4.39-4.32 (m, 2H), 3.85 (s, 3H), 3.84 (5, 3H), 3.72-3.64 (m, 2H), 2.48 (s, 3H). ¹³C NMR (DMSO-d₆, 100 MHz) δ 161.4, 155.2, 152.8, 147.5, 139.3, 136.4, 131.8, 127.1, 123.2, 121.4, 119.6, 112.3, 111.0 (br), 107.5, 106.1, 102.3, 56.1, 55.7, 44.7, 39.5, 36.6, 14.6. (+)-LRESIMS m/z (rel. int.) 438 (100) [M+H]⁺; vₘₐₓ 3409, 3250, 1708, 1636, 1607, 1549, 1503, 1230, 1015, 744 cm⁻¹.

### Synthesis of BT-2066

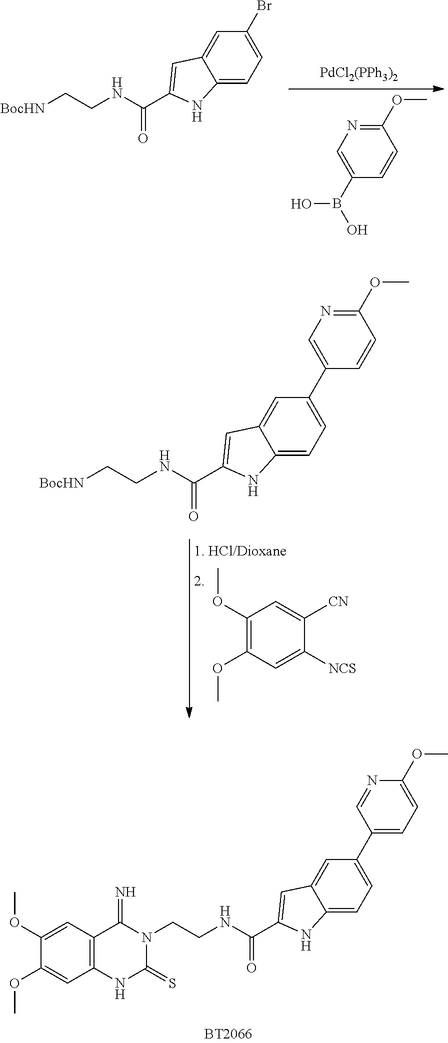

tert-Butyl (2-(5-(6-methoxypyridin-3-yl)-1H-indole-2-carboxamido)ethyl)carbamate was prepared and purified according to General Procedure D from reaction of 2-methoxy-5-pyridine boronic acid (60 mg, 0.39 mmol) and tert-butyl (2-(5-bromo-1H-indole-2-carboxamido)ethyl)carbamate (100 mg, 0.26 mmol) to afford the desired compound (75 mg, 70%) as a white powder. ¹H NMR (DMSO-d₆, 400 MHz) δ 11.64 (s, 1H), 8.53 (t, J=5.5 Hz, 1H), 8.47 (s, 1H), 8.06-7.98 (m, 1H), 7.87 (s, 1H), 7.50 (d, J=8.5 Hz, 1H), 7.46 (d, J=8.5 Hz, 1H), 7.15 (s, 1H), 6.97-6.89 (m, 1H), 6.89 (d, J=8.6 Hz, 1H), 3.89 (s, 3H), 3.38-3.27 (m, 2H), 3.13 (app. q, J=6.4 Hz, 2H), 1.38 (s, 9H); ¹³C NMR (DMSO-d₆, 100 MHz) 2 signals obscured by DMSO-d₆ δ 162.4, 161.1, 155.7, 144.4, 137.6, 135.9, 132.5, 130.5, 128.9, 127.7, 122.4, 119.1, 112.9, 110.3, 102.8, 77.7, 53.1, 28.2 (3C); (+)-LRESIMS m/z (rel. int.) 411 (100) [M+H]⁺; (+)-HRESIMS calcd. for C₂₂H₂₇N₄O₄ [M+H]⁺ 411.2027, found 411.2029; vₘₐₓ 3314, 3227, 1696, 1613, 1554, 1497, 1367, 1291, 1279, 1151, 1040, 836, 812, 625 cm⁻¹. tert-Butyl (2-(5-(6-methoxypyridin-3-yl)-1H-indole-2-carboxamido)ethyl)carbamate (100 mg, 0.41 mmol) was deprotected according to General Procedure E to afford N-(2-aminoethyl)-5-(6-methoxypyridin-3-yl)-1H-indole-2-carboxamide dihydrochloride as a white powder, and used directly without further purification. The crude dihydrochloride formed above (87 mg, 0.27 mmol) in a solution of ethanol (6 mL) was treated with triethylamine (96 μL, 0.68 mmol) and then a solution of 2-isothiocyanato-4,5-dimethoxybenzonitrile (50 mg, 0.27 mmol) in DCM (3 mL) was added. The mixture was stirred at rt for 1 h then concentrated by a gentle stream of nitrogen and suspended in ethanol (5 mL) and refluxed for 4 h. The precipitate formed was collected and washed with ethanol (15 mL) then diethyl ether (10 mL) and dried at the pump to afford BT-2066 (33 mg, 27%) as a cream coloured powder. ¹H NMR (DMSO-d₆, 400 MHz) δ 11.94 (s, 1H), 11.61 (s, 1H), 9.14 (s, 1H), 8.65 (s, 1H), 8.50-8.44 (m, 1H), 8.03-7.99 (m, 1H), 7.85 (s, 1H), 7.62 (s, 1H), 7.49 (d, J=8.5 Hz, 1H), 7.46 (d, J=8.0 Hz, 1H), 7.11 (s, 1H), 6.89 (d, J=8.6 Hz, 1H), 6.84 (s, 1H), 4.94-4.84 (s, 2H), 3.89 (s, 3H), 3.82 (s, 3H), 3.79 (s, 3H), 3.79-3.66 (m, 2H); (+)-LRESIMS m/z (rel. int.) 531 (100) [M+H]⁺; (+)-HRESIMS calcd. for C₂₇H₂₇N₆O₄S [M+H]⁺ 531.1809, found 542.1816. vₘₐₓ 3209, 1625, 1551, 1513, 1438, 1280, 1238, 1020, 804, 765, 738 cm⁻¹.

### Synthesis of BT-2068

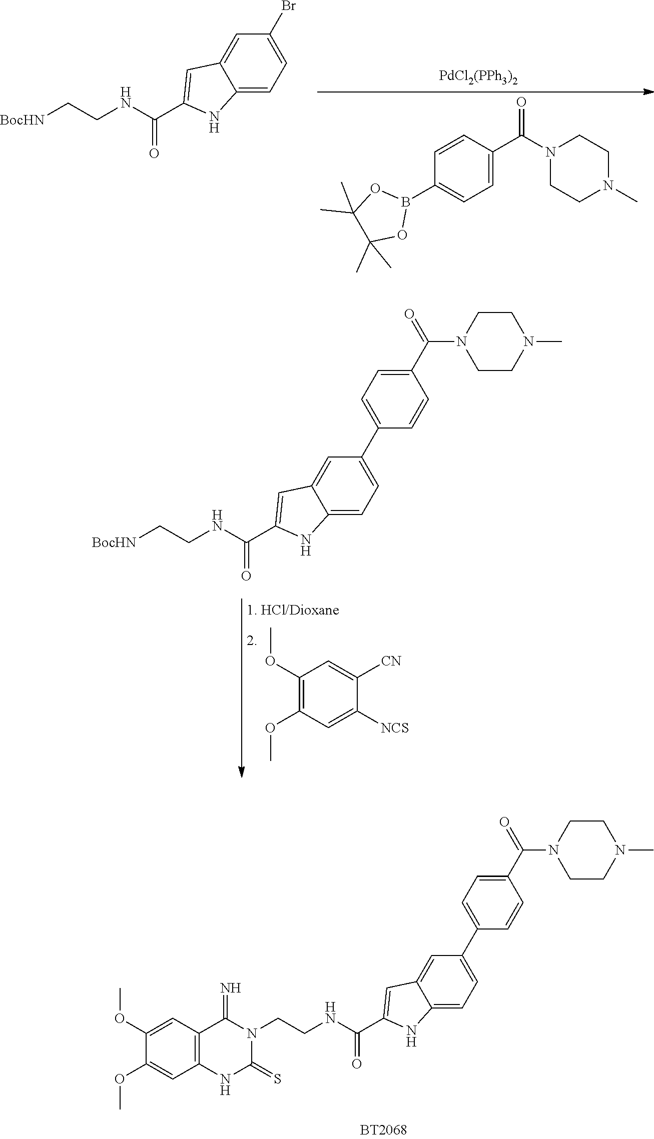

tert-Butyl (2-(5-(4-(4-methylpiperazine-1-carbonyl)phenyl)-1H-indole-2-carboxamido)ethyl)carbamate was prepared and purified according to General Procedure D from reaction of [4-(4-methylpiperazine-1-carbonyl)phenyl]boronic acid pinacol ester (130 mg, 0.39 mmol) and tert-butyl (2-(5-bromo-1H-indole-2-carboxamido)ethyl)carbamate (100 mg, 0.26 mmol) to afford the desired compound (72 mg, 54%) as a white powder. ¹H NMR (DMSO-d₆, 400 MHz) δ 11.68 (s, 1H), 8.55 (t, J=5.5 Hz, 1H), 7.94 (s, 1H), 8.05 (d, J=8.0 Hz, 2H), 7.55-7.50 (m, 2H), 7.45 (d, J=8.0 Hz, 2H), 7.17 (s, 1H), 6.93 (t, J=5.5 Hz, 1H), 3.65-3.33 (m, 6H), 3.14 (app. q, J=5.7 Hz, 2H), 2.38-2.28 (m, 4H), 2.20 (s, 3H), 1.38 (s, 9H); ¹³C NMR (DMSO-d₆, 100 MHz) 2 signals obscured by DMSO-d₆ δ 168.9, 161.1, 155.7, 142.5, 136.2, 133.7, 132.6, 131.2, 127.7, 127.6 (2C), 127.6, 126.5 (2C), 122.7, 119.6, 112.8, 102.9, 77.7, 54.6 (br, 2C), 45.6, 28.2 (3C); (+)-LRESIMS m/z (rel. int.) 506 (100) [M+H]⁺; (+)-HRESIMS calcd. for C₂₈H₃₆N₅O₄ [M+H]⁺ 506.2762, found 506.2759; vₘₐₓ 3257, 1691, 1625, 1605, 1557, 1522, 1423, 1293, 1246, 1170, 1128, 1004, 808, 764 cm⁻¹. The Boc protected compound (100 mg, 0.20 mmol) was deprotected according to General Procedure E to afford the corresponding amine dihydrochloride (87 mg, 91%) as a white powder, and used directly without further purification. The crude dihydrochloride formed above (87 mg, 0.18 mmol) was reacted with triethylamine (76 μL, 0.55 mmol) and 2-isothiocyanato-4,5-dimethoxybenzonitrile (40 mg, 0.18 mmol) according to General Procedure C to afford BT-2068 (25 mg, 22%) as a white powder. ¹H NMR (DMSO-d₆, 400 MHz) 1H obscured or overlapping δ 11.95 (s br, 1H), 11.66 (s br, 1H), 9.14 (s br, 1H), 8.68 (s br, 1H), 7.93 (s, 1H), 7.74 (d, J=7.6 Hz, 2H), 7.61 (s br, 1H), 7.56-7.49 (m, 2H), 7.45 (d, J=7.6 Hz, 2H), 7.15 (s br, 1H), 4.97-4.84 (m, 2H), 3.82 (s, 3H), 3.79 (s, 3H), 3.78-3.71 (m, 2H), 3.66-3.48 (m, 4H), 2.38-2.27 (s br, 4H), 2.21 (s, 3H); ¹³C NMR (DMSO-d₆, 100 MHz) complex spectrum due to slow rotation δ 173.8 (br), 168.9, 161.0 (br), 154.1 (br), 153.1 (br), 146.4, 142.5, 136.2, 133.7 (br), 132.9, 131.3, 130.7 (br), 127.7, 127.6 (br), 126.5 (br), 122.7 (br), 119.6, 112.9, 107.4 (br), 102.9 (br), 98.2 (br), 56.2, 55.7, 54.6 (br), 46.1 (br), 45.6, 36.6 (br). (+)-LRESIMS m/z (rel. int.) 626 (100) [M+H]⁺; (+)-HRESIMS calcd. for C₃₃H₃₆N₇O₄S [M+H]⁺ 626.2544, found 626.2552; vₘₐₓ 3216, 1624, 1545, 1513, 1436, 1278, 1235, 1188, 1021, 851, 805, 762 cm⁻¹.

### Synthesis of BT-2070

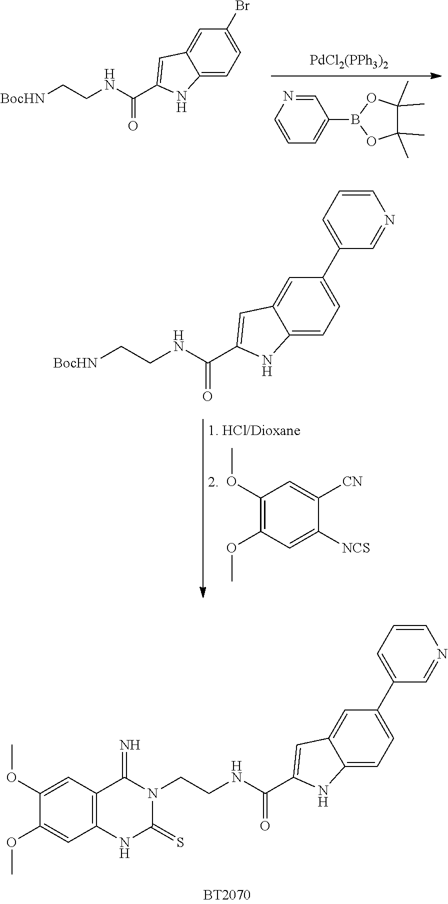

tert-Butyl (2-(5-(pyridin-3-yl)-1H-indole-2-carboxamido)ethyl)carbamate was prepared and purified according to General Procedure D from reaction of 3-pyridylboronic acid pinacol ester (80 mg, 0.39 mmol) and tert-butyl (2-(5-bromo-1H-indole-2-carboxamido)ethyl)carbamate (100 mg, 0.26 mmol) to afford after flash chromatography, the desired compound (62 mg, 62%) as a white powder. ¹H NMR (DMSO-d₆, 400 MHz) δ 11.71 (s, 1H), 8.92 (d, J=2.2 Hz, 1H), 8.56 (t, J=5.5 Hz, 1H), 8.51 (dd, J=4.7, 1.4 Hz, 1H), 8.08 (dt, J=8.0, 2.2 Hz, 1H), 7.98 (s, 1H), 7.54 (s, 2H), 7.45 (dd, J=8.0, 4.7 Hz, 1H), 7.18 (d, J=1.4 Hz, 1H), 6.94 (t, J=5.5 Hz, 1H), 3.33 (app. q, J=6.7 Hz, 2H), 3.14 (app. q, J=6.7 Hz, 2H), 1.38 (s, 9H); ¹³C NMR (DMSO-d₆, 100 MHz) 2 signals obscured by DMSO-d₆ δ 161.1, 155.7, 147.7, 147.5, 136.7, 136.2, 133.9, 132.7, 128.9, 127.8, 123.7, 122.6, 119.9, 113.0, 102.9, 77.7, 28.2 (3C); (+)-LRESIMS m/z (rel. int.) 381 (100) [M+H]⁺; (+)-HRESIMS calcd. for C₂₁H₂₅N₄O₃ [M+H]⁺ 381.1921, found 381.1926; vₘₐₓ 3371, 3222, 2974, 1686, 1634, 1564, 1528, 1330, 1267, 1168, 795 cm⁻¹. The Boc protected compound (97 mg, 0.25 mmol) was deprotected according to General Procedure E to afford the corresponding amine dihydrochloride (75 mg, 85%) as a white powder, and used directly without further purification. The crude dihydrochloride formed above (75 mg, 0.21 mmol) was reacted with triethylamine (118 μL, 0.85 mmol) and 2-isothiocyanato-4,5-dimethoxybenzonitrile (47 mg, 0.21 mmol) according to General Procedure C to afford BT-2070 (18 mg, 17%) as a white powder. ¹H NMR (DMSO-d₆, 400 MHz) δ 11.96 (s br, 1H), 11.68 (s, 1H), 9.17 (s br, 1H), 8.91 (s, 1H), 8.72 (s br, 1H), 8.52 (d, J=4.0 Hz, 1H), 8.09 (dt, J=7.9, 4.0 Hz, 1H), 7.96 (s, 1H), 7.62 (s, 1H), 7.55-7.53 (m, 2H), 7.46 (dd, J=7.9, 4.7 Hz, 1H), 7.15 (s, 1H), 6.85 (s, 1H), 4.89 (s br, 2H), 3.82 (s, 3H), 3.80 (s, 3H), 3.76-3.69 (m, 2H); (+)-LRESIMS m/z (rel. int.) 501 (100) [M+H]⁺; (+)-HRESIMS calcd. for C₂₆H₂₅N₆O₃S [M+H]⁺ 501.1703, found 501.1709. vₘₐₓ 3202, 1623, 1546, 1512, 1437, 1396, 1279, 1233, 1186, 1018, 796, 762 cm⁻¹.

### Synthesis of BT-2071

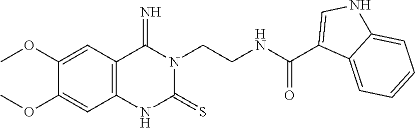 [SMILES: COc1cc2[nH]c(=S)n(CCNC(=O)c3c[nH]c4ccccc34)c(=N)c2cc1OC]

Following a procedure analogous to that used in General Procedure G, 1H-indole-3-carboxylic acid (400 mg, 2.48 mmol) was converted to 1H-indole-3-carbonyl chloride and reacted with tert-butyl (2-aminoethyl)carbamate (397 mg, 2.48 mmol) to afford tert-butyl (2-(1H-indole-3-carboxamido)ethyl)carbamate (352 mg, 47%) as a white solid. ¹H NMR (CD₃OD, 400 MHz) NH protons not observed δ 8.13-8.05 (m, 1H), 7.85 (s, 1H), 7.45-7.38 (m, 1H), 7.21-7.11 (m, 2H), 3.47 (app. t, J=6.1 Hz, 2H), 3.29 (app. t, J=6.1 Hz, 2H), 1.41 (s, 9H); ¹³C NMR (CD₃OD, 100 MHz) δ 168.8, 158.9, 138.1, 129.2, 126.9, 123.4, 121.9, 121.7, 112.8, 111.8, 80.2, 41.3, 40.8, 28.7; (+)-LRESIMS m/z (rel. int.) 326 (100) [M+Na]⁺; (+)-HRESIMS calcd. for C₁₆H₂₁N₃O₃Na [M+Na]⁺ 326.1475, found 326.1481; vₘₐₓ 3357, 3222, 1684, 1611, 1547, 1495, 1441, 1321, 1234, 1210, 1151, 1105, 777, 739 cm⁻¹. A portion of the Boc protected compound (136 mg, 0.45 mmol) was deprotected according to General Procedure E to afford (100 mg, 93%) as a grey gum, and used directly without further purification. The crude hydrochloride formed above (65 mg, 0.27 mmol) was reacted with triethylamine (118 μL, 0.85 mmol) and 2-isothiocyanato-4,5-dimethoxybenzonitrile (47 mg, 0.21 mmol) according to General Procedure C to afford BT-2071 (41 mg, 36%) as a pale yellow solid. ¹H NMR (DMSO-d₆, 400 MHz) 1H obscured or overlapping δ 11.97 (s br, 1H), 11.56 (s br, 1H), 9.23 (s br, 1H), 8.12 (d, J=7.7 Hz, 1H), 7.99 (s, 1H), 7.60 (s, 1H), 7.43 (d, J=7.7 Hz, 1H), 7.19-7.07 (m, 2H), 6.83 (s, 1H), 4.86 (s br, 2H), 3.83 (s, 3H), 3.81 (s, 3H), 3.70-3.62 (m, 2H); (+)-LRESIMS m/z (rel. int.) 424 (100) [M+H]⁺; (+)-HRESIMS calcd. for C₂₁H₂₂N₅O₃S [M+H]⁺ 424.1438, found 424.1445; vₘₐₓ 1704, 1620, 1503, 1440, 1207, 1064, 743 cm⁻¹.

### Synthesis of BT-2072

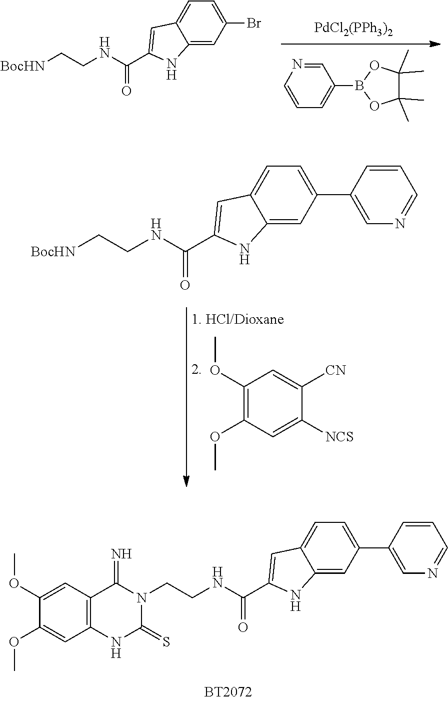

tert-Butyl (2-(6-(pyridin-3-yl)-1H-indole-2-carboxamido)ethyl)carbamate was prepared and purified according to General Procedure D from reaction of 3-pyridylboronic acid pinacol ester (80 mg, 0.39 mmol) and tert-butyl (2-(5-bromo-1H-indole-2-carboxamido)ethyl)carbamate (100 mg, 0.26 mmol) to afford after flash chromatography the desired compound (53 mg, 53%) as a white powder. ¹H NMR (DMSO-d₆, 400 MHz) δ 11.73 (s, 1H), 8.87 (d, J=1.7 Hz, 1H), 8.62-8.49 (m, 2H), 8.09-8.00 (m, 1H), 7.74 (d, J=8.3 Hz, 1H), 7.68 (s, 1H), 7.48 (dd, J=8.0, 4.7 Hz, 1H), 7.39 (d, J=8.4 Hz, 1H), 7.14 (s, 1H), 6.94 (t, J=5.2 Hz, 1H), 3.36-3.29 (m, 2H), 3.13 (q, J=6.5 Hz, 2H), 1.38 (s, 9H); ¹³C NMR (DMSO-d₆, 100 MHz) two signals obscured by DMSO-d₆ δ 161.0, 155.7, 147.9, 147.7, 136.9, 136.7, 134.1, 132.8, 132.3, 127.0, 123.8, 122.2, 119.2, 110.4, 102.4, 77.7, 28.2 (3C); (+)-LRESIMS m/z (rel. int.) 381 (100) [M+H]⁺, 403 (80) [M+Na]⁺; (+)-HRESIMS calcd. for C₂₁H₂₅N₄O₃ [M+H]⁺ 381.1921, found 381.1927; vₘₐₓ 3355, 3310, 1684, 1640, 1533, 1270, 1239, 1165, 991, 791 cm⁻¹. Boc-protected compound (96 mg, 0.25 mmol) was deprotected according to General Procedure E to afford (78 mg, 88%) as a white powder, and used directly without further purification. (+)-LRESIMS m/z (rel. int.) 281 (100) [M+H]⁺; (+)-HRESIMS calcd. for C₁₆H₁₇N₄O [M+H]⁺ 281.1397, found 281.1404. A suspension of the crude hydrochloride formed above (72 mg, 0.20 mmol) was suspended in ethanol (4 mL) and treated with triethylamine (113 μL, 0.82 mmol) and sonicated until the mixture became clear. 2-Isothiocyanato-4,5-dimethoxybenzonitrile (45 mg, 0.20 mmol) was added in portion and stirred for 0.5 h at rt and then heated at 70° C. for 4 h. The precipitate was collected by vacuum filtration and was subjected to flash column chromatography [silica, 1:10 v/v MeOH/DCM elution] to give, after concentration of the appropriate fractions BT-2072 (15 mg, 15%) as a cream powder. ¹H NMR (DMSO-d₆, 400 MHz) δ 11.96 (s br, 1H), 11.71 (s br, 1H), 9.15 (s, 1H), 8.87 (s br, 1H), 8.68 (s, 1H), 8.55 (dd, J=4.7, 1.2 Hz, 1H), 8.09-8.01 (m, 1H), 7.73 (d, J=8.3 Hz, 1H), 7.68 (s, 1H), 7.62 (s, 1H), 7.49 (dd, J=8.3, 4.7 Hz, 1H), 7.39 (d, J=8.5 Hz, 1H), 7.10 (s, 1H), 6.84 (s, 1H), 4.89 (s br, 2H), 3.82 (s, 3H), 3.79 (s, 3H), 3.78-3.67 (m, 2H); (+)-LRESIMS m/z (rel. int.) 501 (100) [M+H]⁺; (+)-HRESIMS calcd. for C₂₆H₂₅N₆O₃S [M+H]⁺ 501.1703, found 501.1707; vₘₐₓ 3215, 1625, 1550, 1514, 1459, 1436, 1282, 1237, 1022, 809, 633 cm⁻¹.

### Synthesis of BT-2100

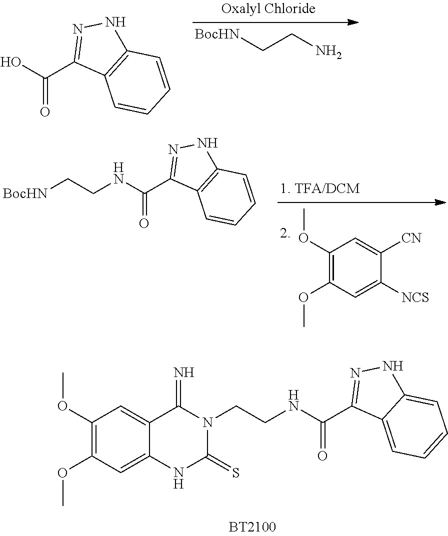

A magnetically stirred mixture of 1H-indazole-3-carboxylic acid (100 mg, 0.62 mmol) in DMF (5 mL) was treated sequentially with N-(3-dimethylaminopropyl)-N′-ethylcarbodiimide hydrochloride (EDC) (142 mg, 0.74 mmol), 1-hydroxybenzotriazole hydrate (100 mg, 0.74 mmol) and triethylamine (103 μL, 0.74 mmol) and stirred at rt 0.5 h. tert-Butyl (2-aminoethyl)carbamate (118 mg, 0.74 mmol) was added and the mixture stirred for 60 h then concentrated in vacuo and purified by flash column chromatography [silica, 1:20 v/v MeOH/DCM elution] to give, after concentration of the appropriate fractions, tert-butyl (2-(1H-indazole-3-carboxamido)ethyl)carbamate (32 mg, 14%) as a white powder. ¹H NMR (400 MHz, Chloroform-d) δ 10.65 (s, 1H), 8.40 (dt, J=8.2, 1.1 Hz, 1H), 7.56 (br s, 1H), 7.53 (d, J=8.4 Hz, 1H), 7.44 (ddd, J=8.4, 6.9, 1.0 Hz, 1H), 7.30 (ddd, J=7.9, 6.9, 1.0 Hz, 1H), 5.06-4.94 (m, 1H), 3.64 (app. q, J=6.1 Hz, 2H), 3.44 (app. q, J=6.0 Hz, 2H), 1.42 (s, 9H); (+)-LRESIMS m/z (rel. int.) 305 (30) [M+H]′, 327 (100) [M+Na]⁺ 631 (40) [2M+Na]⁺. The Boc-protected compound formed directly above (32 mg, 0.11 mmol) was deprotected following General Procedure F with TFA (1.0 mL) and DCM (4.0 mL) to afford after trituration with ether (10 mL) TFA salt of N-(2-aminoethyl)-1H-indazole-3-carboxamide as a gum that was used directly in the next step without further purification. The amine TFA-salt (0.11 mmol) was then suspended in ethanol (5 mL) and treated with triethylamine (175 μL, 1.26 mmol) and magnetically stirred for 5 min. The reaction mixture was then treated with a solution of 2-isothiocyanato-4,5-dimethoxybenzonitrile (23 mg, 0.11 mmol) in DCM (3 mL). The mixture was then stirred for 1 h followed by stirring at 75° C. for 1 h. The reaction mixture was cooled and then the precipitate was collected by vacuum filtration and washed with ether (5 mL) to afford BT-2100 (24 mg, 51%) as a pale-yellow solid. ¹H NMR (400 MHz, DMSO-d₆) δ 13.48 (s, 1H), 11.95 (s, 1H), 9.12 (s, 1H), 8.51 (s, 1H), 8.13 (d, J=8.6 Hz, 1H), 7.67-7.54 (m, 2H), 7.45-7.33 (m, 1H), 7.28-7.18 (m, 1H), 6.83 (s, 1H), 4.98-4.84 (m, 2H), 3.81 (s, 3H), 3.78 (s, 3H), 3.73-3.78 (m, 2H); (+)-LRESIMS m/z (rel. int.) 425 (100) [M+H]⁺, vₘₐₓ 2134, 1684, 1647, 1560, 1532, 1500, 1438, 1401, 1233, 1211, 1159, 1211, 1159, 1063 cm⁻¹.

### Synthesis of BT-2103

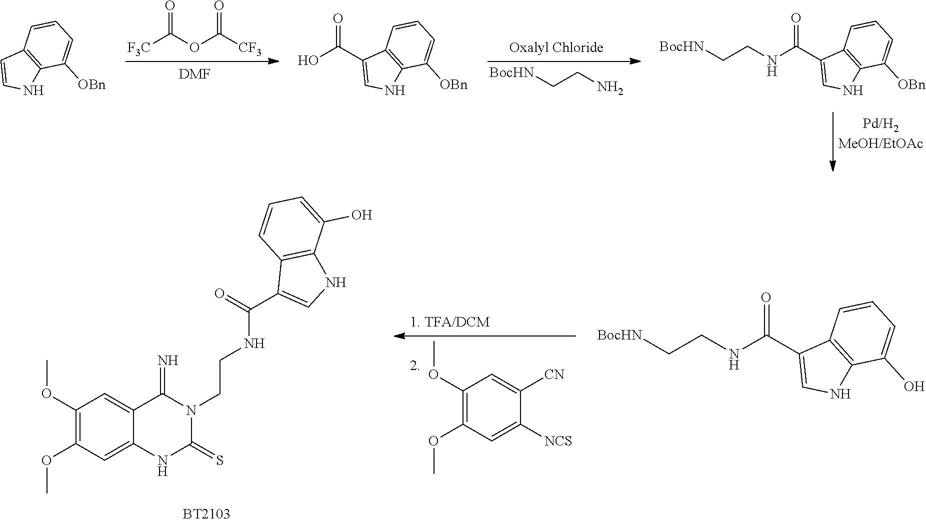

7-(benzyloxy)-1H-indole-3-carboxylic Acid:

A magnetically stirred solution of 7-(benzyloxy)-1H-indole (2.0 g, 8.9 mmol) in anhydrous DMF (8 mL) maintained at 0° C. under an atmosphere of nitrogen was treated with trifluoroacetic acid anhydride (1.8 mL, 12.7 mmol) dropwise over 0.17 h. The mixture was then stirred for 2 h and poured into water (150 mL) and stirred vigorously for 0.17 h. The solid was collected by vacuum filtration and added to flask containing an aqueous solution of sodium hydroxide (50 mL, 4M) and refluxed for 2 h. The mixture was cooled over 18 h then diluted with water (150 mL) and extracted with ether (2×50 mL). The aqueous layer was then acidified to pH 0-1 with aqueous HCl (6M) and the precipitate collected by vacuum filtration and held under high vacuum for several hours to afford 7-(benzyloxy)-1H-indole-3-carboxylic acid as a brown powder (1.43 g, 60%) and used without further purification. ¹H NMR (400 MHz, DMSO-d₆) δ 12.00 (s, 1H), 11.92 (s, 1H), 7.85 (d, J=3.1 Hz, 1H), 7.61-7.55 (m, 3H), 7.44-7.38 (m, 2H), 7.36-7.31 (m, 1H), 7.05 (t, J=7.8 Hz, 1H), 6.84 (d, J=7.8 Hz, 1H), 5.28 (s, 2H); ¹³C NMR (100 MHz, DMSO-d₆) δ 165.9, 145.3, 137.1, 131.5, 128.4 (2C), 127.8, 127.7, 127.6 (2C), 126.7, 121.6, 113.4, 108.0, 104.1, 69.3; (+)-LRESIMS m/z (rel. int.) 290 (100) [M+Na]⁺, 557 (90) [2M+Na]⁺; vₘₐₓ 3427, 1652, 1629, 1529, 1506, 1276, 1251, 1194, 1090, 1004, 759 cm⁻¹.

tert-butyl (2-(7-(benzyloxy)-1H-indole-3-carboxamido)ethyl)carbamate:

Following a procedure analogous to General Procedure G, 7-(benzyloxy)-1H-indole-3-carboxylic acid (300 mg, 1.12 mmol) was converted to 7-(benzyloxy)-1H-indole-3-carbonyl chloride with oxalyl chloride (118 μL, 1.40 mmol) and reacted with tert-butyl (2-aminoethyl)carbamate (180 mg, 1.12 mmol) to afford tert-butyl (2-(7-(benzyloxy)-1H-indole-3-carboxamido)ethyl)carbamate (252 mg, 55%) as a tan solid. ¹H NMR (400 MHz, DMSO-d₆) δ 11.65 (s, 1H), 7.91 (d, J=2.7 Hz, 1H), 7.87 (app. t, J=5.3 Hz, 1H), 7.71 (d, J=8.0 Hz, 1H), 7.57 (d, J=7.3 Hz, 2H), 7.41 (t, J=7.3 Hz, 2H), 7.37-7.32 (m, 1H), 6.99 (t, J=8.0 Hz, 1H), 6.89 (t, J=5.4 Hz, 1H), 6.80 (d, J=8.0 Hz, 1H), 5.26 (s, 2H), 3.27 (app. q, J=6.2 Hz, 2H), 3.09 (app. q, J=6.2 Hz, 2H), 1.38 (s, 9H); ¹³C NMR (100 MHz, DMSO-d₆) one CH₂N obscured by DMSO-d₆ δ 164.7, 155.7, 145.1, 137.2, 128.4 (2C), 127.8, 127.8, 127.6 (2C), 127.3, 126.4, 120.8, 113.9, 111.1, 103.6, 77.6, 69.2, 38.8, 28.2 (3C). (+)-LRESIMS m/z (rel. int.) 432 (100) [M+Na]⁺; vₘₐₓ 3296, 1695, 1614, 1537, 1437, 1284, 1215, 1170, 785, 723, 689 cm⁻¹.

tert-butyl (2-(7-hydroxy-1H-indole-3-carboxamido)ethyl)carbamate:

A mixture of tert-butyl (2-(7-(benzyloxy)-1H-indole-3-carboxamido)ethyl)carbamate (200 mg, 0.49 mmol) and 10% palladium on carbon (10 mg), in a mixture of methanol (15 mL) and ethyl acetate (5 mL) was stirred under a balloon of hydrogen for 2 h. The mixture was treated with celite (5 mL) and then the reaction mixture filtered and the celite washed with EtOAc (10 mL). The filtrate was concentrated in vacuo to afford a residue that was purified by flash column chromatography [silica, 1:10 v/v MeOH/DCM elution] to give, after concentration of the appropriate fractions the debenzylated product as a tan powder (140 mg, 90%). ¹H NMR (400 MHz, DMSO-d₆) δ 11.41 (s, 1H), 9.70 (s, 1H), 7.89-7.82 (m, 1H), 7.80 (t, J=5.6 Hz, 1H), 7.55 (d, J=8.0 Hz, 1H), 6.91-6.87 (m, 1H), 6.86 (dd, J=9.3, 6.6 Hz, 1H), 6.54 (d, J=7.5 Hz, 1H), 3.26 (app. q, J=6.5 Hz, 2H), 3.09 (app. q, J=6.5 Hz, 2H), 1.38 (s, 9H); (+)-LRESIMS m/z (rel. int.) 342 (100) [M+Na]⁺; vₘₐₓ 3345, 3270, 1688, 1601, 1601, 1550, 1524, 1449, 1242, 1157, 630 cm⁻¹. The product formed directly above (140 mg, 0.44 mmol) was deprotected at 0° C. using a method analogous to General Procedure F to afford the deprotected, TFA-salt as a brown paste that was used without further purification. The TFA salt was then dissolved in ethanol (8 mL) and treated with triethylamine (244 μL, 1.76 mmol) and magnetically stirred for 5 min then treated with a solution of 2-isothiocyanato-4,5-dimethoxybenzonitrile (97 mg, 0.44 mmol) in DCM (3 mL). The mixture was then stirred for 1 h at rt and then heated to 72° C. and stirred for 1 h. The precipitate was collected by vacuum filtration which was washed with ether (5 mL) to afford BT-2103 (102 mg, 53%) as a tan powder. (+)-LRESIMS m/z (rel. int.) 440 (100) [M+H]⁺, 462 [M+Na]⁺; vₘₐₓ 1614, 1583, 1505, 1440, 1293, 1233, 1175, 1073, 1046, 989, 736 cm⁻¹. ¹H NMR analyses of BT-2103 resulted in severe broadening. To overcome this, the trifluoroacetic acid salt of BT-2103 was prepared. Accordingly, a portion of BT-2103 (20 mg) suspended in CDCl₃ (1 mL) was treated with TFA (100 μL) and then after 1 min the solution was concentrated under a gentle stream of N₂ to furnish the TFA salt as a tan powder. ¹H NMR (400 MHz, DMSO-d₆) δ 13.75 (s, 1H), 11.65 (d, J=3.1 Hz, 1H), 10.90 (s, 1H), 10.59 (s, 1H), 10.34-9.06 (br s, 1H), 8.74 (t, J=5.6 Hz, 1H), 7.94 (d, J=3.1 Hz, 1H), 7.77 (s, 1H), 7.51 (d, J=8.0 Hz, 1H), 6.96 (s, 1H), 6.90 (t, J=8.0 Hz, 1H), 6.58 (d, J=7.5 Hz, 1H), 5.74-4.95 (br m, 2H), 3.91 (s, 3H), 3.86 (s, 3H), 3.64 (app. q, J=7.1 Hz, 2H); ¹³C NMR (DMSO-d₆, 100 MHz) δ 171.3, 167.6, 158.4 (q, ²J_{C-F}=35.6 Hz), 157.2, 155.4, 147.7, 143.9, 135.2, 128.3, 127.7, 126.3, 121.6, 116.0 (q, ¹J_{C-F}=292.8 Hz), 111.5, 109.3, 106.5, 105.1, 102.4, 97.8, 56.4, 56.4, 47.8, 34.7.

### Synthesis of BT-2133

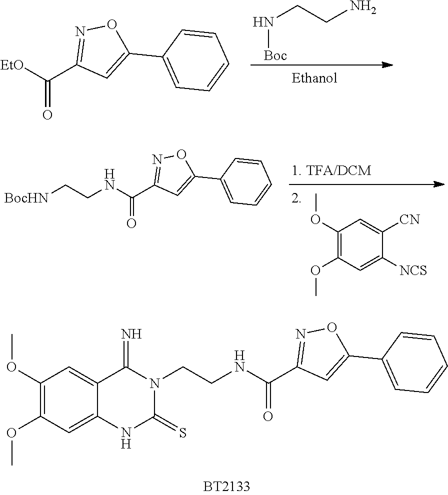

A mixture of ethyl 5-phenylisoxazole-3-carboxylate (100 mg, 0.46 mmol) prepared according to the procedure of Watterson et. al. (2016) J. Med. Chem., 59: 2820, and tert-butyl (2-aminoethyl)carbamate (100 mg, 0.62 mmol) in ethanol (2 mL) was stirred for 72 h at rt. The mixture was then concentrated in vacuo and purified by flash column chromatography [silica, 1:10 v/v ether/DCM elution] to give, after concentration of the appropriate fractions, tert-butyl (2-(5-phenylisoxazole-3-carboxamido)ethyl)carbamate as a white powder (32 mg, 21%). ¹H NMR (400 MHz, chloroform-d) δ 7.84-7.76 (m, 2H), 7.54-7.44 (m, 3H), 7.33-7.25 (m, 1H), 6.96 (s, 1H), 4.89 (s, 1H), 3.59 (app. q, J=5.8 Hz, 2H), 3.39 (app. q, J=5.9 Hz, 2H), 1.44 (s, 9H). ¹³C NMR (100 MHz, CDCl₃) δ 171.6, 159.5, 159.0, 156.4, 130.7, 129.1, 126.8, 125.9, 99.0, 79.8, 40.2, 40.2, 28.3; (+)-LRESIMS m/z (rel. int.) 354 (100) [M+Na]⁺; vₘₐₓ 3382, 3343, 1685, 1665, 1548, 1524, 1446, 1274, 1262, 1154, 1174, 969, 766 cm⁻¹. The boc-protected compound tert-butyl (2-(5-phenylisoxazole-3-carboxamido)ethyl)carbamate (62 mg, 0.19 mmol) was deprotected at 0° C. following General Procedure F with TFA (1.5 mL) and DCM (6.0 mL) to afford after trituration with ether (3×5 mL) to afford the TFA salt of N-(2-aminoethyl)-5-phenylisoxazole-3-carboxamide as a sticky gum that was used directly in the next step without further purification. The amine TFA-salt (0.19 mmol) was then suspended in ethanol (4 mL) and treated with triethylamine (156 μL, 1.12 mmol) and magnetically stirred for 5 min. The reaction mixture was then treated with a solution of 2-isothiocyanato-4,5-dimethoxybenzonitrile (41 mg, 0.19 mmol) in DCM (2 mL). The mixture was then maintained at rt with stirring for 2 h and further at 70° C. for 1 h. The mixture was cooled to rt and the precipitate collected by vacuum filtration and washed with ether (5 mL) to afford BT-2133 (38 mg, 47%) as a white powder. ¹H NMR (400 MHz, DMSO-d₆) δ 11.93 (s, 1H), 9.13 (s, 1H), 8.99 (s, 1H), 7.96-7.88 (m, 2H), 7.63 (s, 1H), 7.60-7.50 (m, 3H), 7.32 (s, 1H), 6.83 (s, 1H), 4.96-4.82 (m, 2H), 3.82 (s, 3H), 3.79 (s, 3H), 3.75-3.68 (m, 2H); (+)-LRESIMS m/z (rel. int.) 452 (100) [M+hl]⁺; vₘₐₓ 1653, 1553, 1535, 1512, 1434, 1228, 1193, 1020, 859, 761, 592 cm⁻¹.

### Synthesis of BT-2136

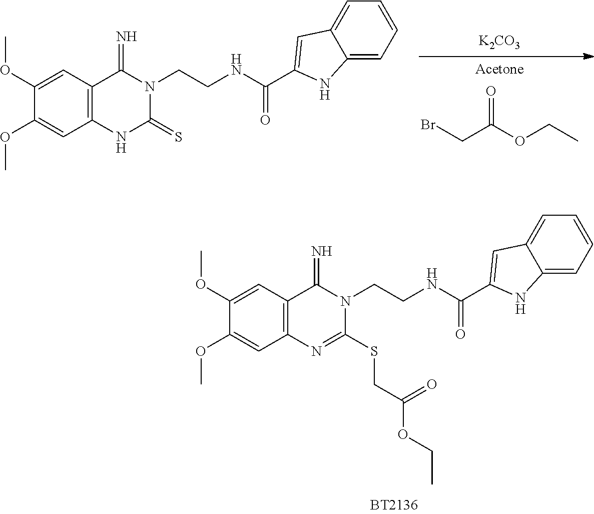

BT-2136 was prepared and purified using General Procedure H from BT-2058 (50 mg, 0.12 mmol), potassium carbonate (32 mg, 0.23 mmol) and ethyl 2-bromoacetate (53 μL, 0.47 mmol) in acetone (2.0 mL) to afford BT-2136 as a white powder (23 mg, 39%). ¹H NMR

(DMSO-d₆, 400 MHz) δ 11.55 (s, 1H), 8.81 (t, J=5.4 Hz, 1H), 8.53 (s, 1H), 7.65 (s, 1H), 7.60 (d, J=8.0 Hz, 1H), 7.41 (d, J=8.0 Hz, 1H), 7.17 (t, J=7.5 Hz, 1H), 7.06 (d, J=1.2 Hz, 1H), 7.02 (t, J=7.5 Hz, 1H), 6.67 (s, 1H), 4.35 (app. t, J=6.0 Hz, 2H), 4.09 (q, J=7.1 Hz, 2H), 3.96 (s, 2H), 3.85 (s, 3H), 3.82 (s, 3H), 3.74-3.65 (app. q, J=6.0 Hz, 2H), 1.17 (t, J=7.1 Hz, 3H). (+)-LRESIMS m/z (rel. int.) 510 (100) [M+H]⁺; vₘₐₓ 3295, 1739, 1631, 1600, 1543, 1501, 1232, 1152, 1017, 744 cm⁻¹.

### Synthesis of BT-2137

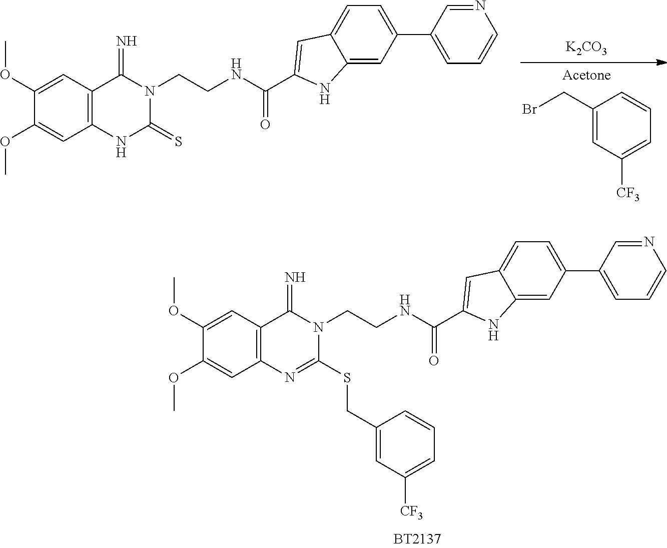

Prepared and purified using a method analogous to General Procedure H from BT-2072 (50 mg, 0.10 mmol), potassium carbonate (28 mg, 0.20 mmol) and 1-(bromomethyl)-3-(trifluoromethyl)benzene (48 mg, 0.20 mmol) in acetone (1.5 mL) to afford BT-2137 as a white powder (13 mg, 20%). ¹H NMR (400 MHz, DMSO-d₆) δ 11.74 (s, 1H), 8.89 (d, J=1.6 Hz, 1H), 8.83 (t, J=5.8 Hz, 1H), 8.55 (dd, J=4.6, 0.9 Hz, 1H), 8.49 (br s, 1H), 8.06 (dt, J=8.0, 2.0 Hz, 1H), 7.88 (s, 1H), 7.74 (d, J=8.3 Hz, 1H), 7.69 (s, 1H), 7.64 (s, 1H), 7.53-7.45 (m, 3H), 7.41 (dd, J=8.3, 1.6 Hz, 1H), 7.25 (t, J=7.8 Hz, 1H), 7.07 (d, J=0.8 Hz, 1H), 6.89 (s, 1H), 4.43 (s, 2H), 4.34-4.26 (m, 2H), 3.86 (s, 3H), 3.85 (s, 3H), 3.69-3.63 (m, 2H); (+)-LRESIMS m/z (rel. int.) 659 (100) [M+H]⁺; vₘₐₓ 1603, 1547, 1501, 1439, 1329 1231, 1161, 1114, 1070, 1017, 803, 698 cm⁻¹.

### Synthesis of BT-2138

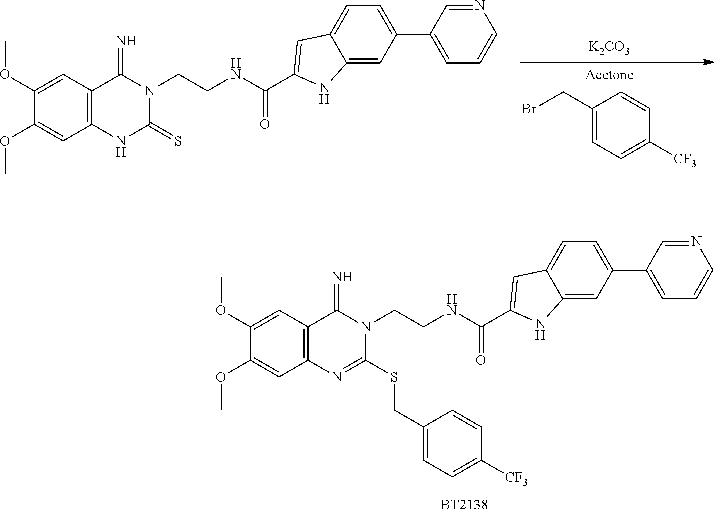

Prepared and purified using a method analogous to General Procedure H from BT-2072 (50 mg, 0.10 mmol), potassium carbonate (28 mg, 0.20 mmol) and 1-(bromomethyl)-4-(trifluoromethyl)benzene (48 mg, 0.20 mmol) in acetone (1.5 mL) to afford, after flash column chromatography [silica, 1:10 v/v ammoniacal MeOH/DCM elution] BT-2138 as a white powder (11 mg, 17%). ¹H NMR (DMSO-d₆, 400 MHz) δ 11.75 (s, 1H), 8.88 (d, J=1.5 Hz, 1H), 8.83 (app. t, J=5.8 Hz, 1H), 8.55 (d, J=4.7 Hz, 1H), 8.48 (br s, 1H), 8.05 (dt, J=8.0, 1.5 Hz, 1H), 7.75 (d, J=8.4 Hz, 1H), 7.69 (s, 1H), 7.65 (s, 1H), 7.50 (dd, J=7.8, 4.8 Hz, 1H), 7.42 (dd, J=8.4, 1.5 Hz, 1H), 7.38 (d, J=7.8 Hz, 2H), 7.28 (d, J=7.9 Hz, 2H), 7.10 (br s, 1H), 6.86 (s, 1H), 4.42 (s, 2H), 4.37-4.25 (m, 2H), 3.86 (s, 3H), 3.85 (s, 3H), 3.74-3.63 (m, 2H); vₘₐₓ 1644, 1601, 1545, 1500, 1438, 1325, 1229, 1156, 1110, 1066, 1017, 810 cm⁻¹.

### Synthesis of BT-2153

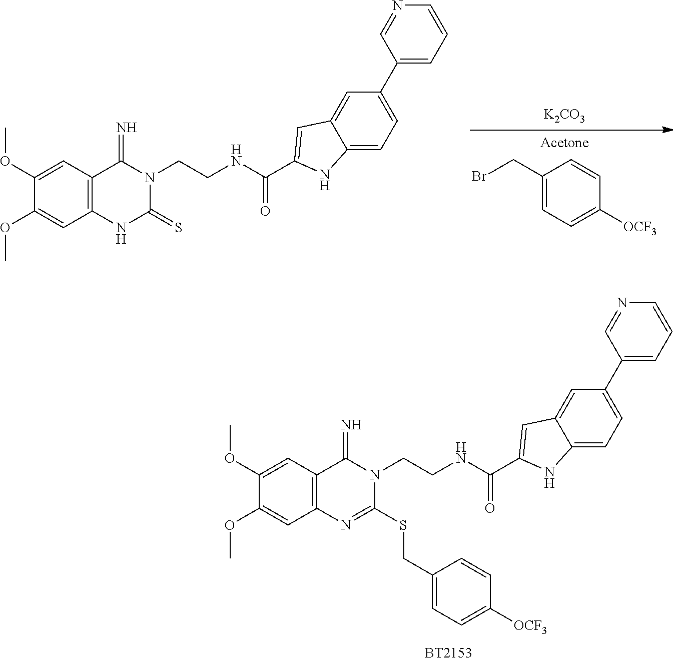 [SMILES: COc1cc2[nH]c(=S)n(CCNC(=O)c3cc4cc(-c5cccnc5)ccc4[nH]3)c(=N)c2cc1OC ; FC(F)(F)Oc1ccc(CBr)cc1 ; "K2CO3" ; COc1cc2nc(SCc3ccc(OC(F)(F)F)cc3)n(CCNC(=O)c3cc4cc(-c5cccnc5)ccc4[nH]3)c(=N)c2cc1OC]

Prepared and purified using a method analogous to General Procedure H from BT-2070 (50 mg, 0.10 mmol), potassium carbonate (28 mg, 0.20 mmol) and 1-(bromomethyl)-4-(trifluoromethoxy)benzene (26 mg, 0.10 mmol) in acetone (1.5 mL) to afford BT-2153 as a white powder (36 mg, 53%). ¹H NMR (DMSO-d₆, 400 MHz) δ 11.73 (s, 1H), 8.92 (d, J=1.9 Hz, 1H), 8.84 (t, J=5.8 Hz, 1H), 8.52 (dd, J=4.9, 1.9 Hz, 1H), 8.47 (s, 1H), 8.07 (ddd, J=7.9, 1.7 Hz, 1H), 7.98 (s, 1H), 7.65 (s, 1H), 7.57 (dd, J=8.7, 1.7 Hz, 1H), 7.54 (d, J=8.7 Hz, 1H), 7.46 (dd, J=7.9, 4.9, 1H), 7.30 (d, J=8.3 Hz, 2H), 7.13 (d, J=1.5 Hz, 1H), 6.96 (d, J=8.3 Hz, 2H), 6.87 (s, 1H), 4.36 (s, 2H), 4.34-4.29 (m, 2H), 3.86 (s, 3H), 3.85 (s, 3H), 3.72-3.65 (m, 2H). ¹³C NMR (100 MHz, DMSO-d₆) δ 161.4, 155.3, 154.1, 152.8, 147.6, 147.6, 147.5, 147.2, 139.1, 136.8, 136.5, 136.2, 133.9, 132.8, 130.9, 128.9, 127.8, 123.7 (2C), 122.6, 120.6 (2C), 119.9 (q, J_{C-F}=256.2 Hz), 119.9, 113.0, 111.2, 107.4, 106.1, 102.9, 56.2, 55.7, 45.2, 36.3, 34.5. (+)-LRESIMS m/z (rel. int.) 675 (100) [M+H]⁺; vₘₐₓ 1623, 1601, 1547, 1502, 1436, 1266, 1214, 1159, 1019, 794 cm⁻¹.

### Synthesis of BT-2113

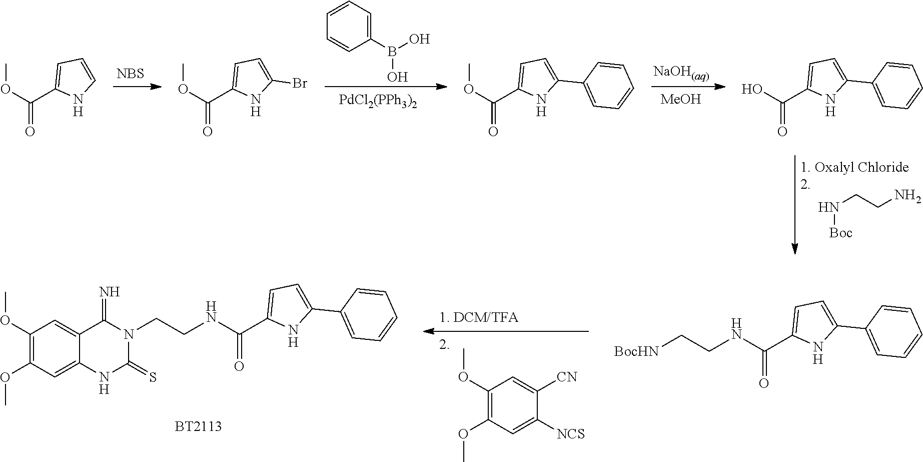

Following a procedure analogous to that used by Trost et. al. (2009) Chem. Eur. J., 15: 6910, a magnetically stirred solution of methyl 1H-pyrrole-2-carboxylate (1.00 g, 7.99 mmol) in THF (80 mL) and methanol (40 mL) maintained at 0° C. was treated with N-bromosuccinimide (245 mg, 1.38 mmol) and followed by addition of portions of (315 mg, 1.77 mmol), (255 mg, 1.43 mmol), (635 mg, 3.57 mmoL) 0.5 h apart. The mixture was then and held at 2° C. without stirring for 18 h and then concentrated in vacuo and purified by gradient flash column chromatography [silica, 1:10→1:5 v/v EtOAc/petroleum spirit elution] to give, after concentration of the appropriate fractions, methyl 5-bromo-1H-pyrrole-2-carboxylate (562 mg, 34%) as a white powder. Spectral data were consistent with those reported by Trost et. al. ¹H NMR (400 MHz, Chloroform-d) δ 9.20 (s, 1H), 6.82 (dd, J=3.9, 2.7 Hz, 1H), 6.22 (dd, J=3.9, 2.7 Hz, 1H), 3.86 (s, 3H).

Methyl 5-bromo-1H-pyrrole-2-carboxylate (147 mg, 0.72 mmol), was subjected to a palladium catalysed Suzuki-Miyaura reaction and reacted with phenylboronic acid (132 mg, 1.08 mmol) according to General Procedure D to afford a residue that was subjected to flash column chromatography [silica, 1:4 ether: petroleum spirit elution] to give, after concentration of the appropriate fractions methyl 5-phenyl-1H-pyrrole-2-carboxylate (88 mg, 75%) as a white solid. Obtained spectral data were consistent with those reported by Laha et. al. (2016) Chem. Commun., 52: 4329). ¹H NMR (400 MHz, Chloroform-d) δ 9.28 (s, 1H), 7.60-7.54 (m, 2H), 7.46-7.38 (m, 2H), 7.34-7.28 (m, 1H), 6.96 (dd, J=3.9, 2.4 Hz, 1H), 6.55 (dd, J=3.9, 2.7 Hz, 1H), 3.88 (s, 3H); (+)-LRESIMS m/z (rel. int.) 224 (100) [M+Na]⁺; vmax 3291, 1675, 1466, 1441, 1339, 1268, 1152, 1005, 799, 757 cm⁻¹. A portion of the above compound, methyl 5-phenyl-1H-pyrrole-2-carboxylate (80 mg, 0.4 mmol) was dissolved in a mixture of methanol (8 mL) and aqueous sodium hydroxide (4 mL, 3M) and held at reflux for 4 h. The clear solution was then neutralised to pH 1 with aqueous HCl (1M) and the mixture extracted with EtOAc (2×15 mL) and DCM (2×15 mL). The EtOAc and DCM extracts were separately washed with brine (10 mL) then combined and dried (Na2SO4). Concentration in vacuo afforded a purple residue (68 mg, 91%) that was used without further purification. ¹H NMR (400 MHz, DMSO-d₆) δ 12.29 (s, 1H), 11.96 (s, 1H), 7.84 (d, J=7.3 Hz, 2H), 7.37 (t, J=7.7 Hz, 2H), 7.27-7.22 (m, 1H), 6.81 (dd, J=3.8, 2.3 Hz, 1H), 6.62 (dd, J=3.8, 2.5 Hz, 1H); (+)-LRESIMS m/z (rel. int.) 186 (100) [M−H]⁻; vₘₐₓ 3420, 1657, 1561, 1514, 1469, 1435, 1333, 1268, 1040, 910, 765, 749, 691 cm⁻¹. A portion of the acid, 5-phenyl-1H-pyrrole-2-carboxylic acid (60 mg, 0.18 mmol) was then converted to its acid chloride with oxalyl chloride (34 μL, 0.40 mmol) and reacted with tert-butyl(2-aminoethyl)carbamate (56 mg, 0.35 mmol) according to General Procedure G. The residue obtained after workup, was subjected to flash column chromatography [silica, 1:5 ether: petroleum spirit elution] to give, after concentration of the appropriate fractions tert-butyl (2-(5-phenyl-1H-pyrrole-2-carboxamido)ethyl)carbamate (70 mg, 61%) as a white solid. ¹H NMR (400 MHz, DMSO-d₆) δ 11.69 (s, 1H), 8.13 (t, J=4.9 Hz, 1H), 7.79 (app. d, J=7.3 Hz, 2H), 7.41-7.30 (m, 2H), 7.26-7.16 (m, 1H), 6.96-6.88 (m, 1H), 6.80 (d, J=3.8 Hz, 1H), 6.56 (d, J=3.8 Hz, 1H), 3.27 (app. q, J=6.3 Hz, 2H), 3.09 (app. q, J=6.3 Hz, 2H), 1.38 (s, 9H); (+)-LRESIMS m/z (rel. int.) 352 (100) [M+Na]⁺, 681 (10) [2M+Na]; vₘₐₓ 3302, 3224, 1695, 1606, 1562, 1537, 1269, 1155, 808, 773 cm⁻¹.

A portion of the Boc-protected compound formed directly above (60 mg, 0.18 mmol) was deprotected at rt following General Procedure F with TFA (1.5 mL) and DCM (4.0 mL) to afford after trituration with ether (3×5 mL) to afford the TFA salt of N-(2-aminoethyl)-5-phenyl-1H-pyrrole-2-carboxamide as a sticky gum that was used directly in the next step without further purification. The amine TFA-salt (0.18 mmol) was then suspended in ethanol (4 mL) and treated with triethylamine (304 μL, 2.22 mmol) and magnetically stirred for 5 min. The reaction mixture was then treated with a solution of 2-isothiocyanato-4,5-dimethoxybenzonitrile (40 mg, 0.18 mmol) in DCM (3 mL). The mixture was then maintained with stirring at 70-72° C. for 1 h, cooled to rt and then the precipitate was collected by vacuum filtration and washed with ether (5 mL) to afford BT-2113 (62 mg, 77%) as a pale pink powder. ¹H NMR (400 MHz, DMSO-d₆) δ 11.77 (s, 1H), 11.62 (s, 1H), 9.15 (s, 1H), 8.20 (s, 1H), 7.78 (d, J=7.5 Hz, 2H), 7.62 (s, 1H), 7.36 (t, J=7.5 Hz, 2H), 7.21 (t, J=7.2 Hz, 1H), 6.91-6.75 (m, 2H), 6.56 (s, 1H), 4.91-4.76 (m, 2H), 3.82 (s, 3H), 3.79 (s, 3H), 3.71-3.62 (m, 2H); (+)-LRESIMS m/z (rel. int.) 450 (100) [M+Na]⁺; vₘₐₓ 1611, 1538, 1500, 1436, 1230, 1015, 857, 751 cm⁻¹.

### Example 9—Inhibition of Heparanase Activity by Compounds of Formula 2-4

### Materials and Methods

Heparanase inhibition assays were conducted according to the protocol of Example 3. Test compounds were dissolved and added to the assay at varying concentrations to calculate the level of inhibition.

### Results

Heparanase inhibition activity of various compounds of the invention was determined using standard methods. The results, expressed as the half maximal inhibitory concentration (IC₅₀), being the concentration of the compound of the invention required to achieve 50% inhibition of heparanase, are shown in Table 4.

TABLE 4
Heparanase inhibition by compounds of Formula 2-4

| Compound | Structure | IC₅₀ |
|---|---|---|
| BT-2058 | 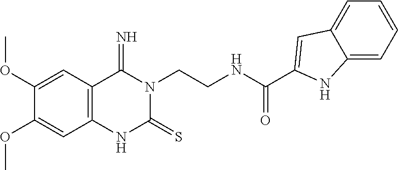 [SMILES: COc1cc2[nH]c(=S)n(CCNC(=O)c3cc4ccccc4[nH]3)c(=N)c2cc1OC] | 9 μM |
| BT-2066 | 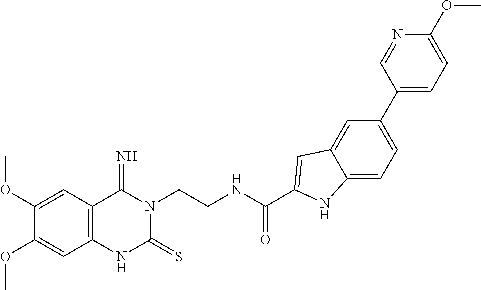 [SMILES: COc1ccc(-c2ccc3[nH]c(C(=O)NCCn4c(=S)[nH]c5cc(OC)c(OC)cc5c4=N)cc3c2)cn1] | 4.2 μM |
| BT-2068 | 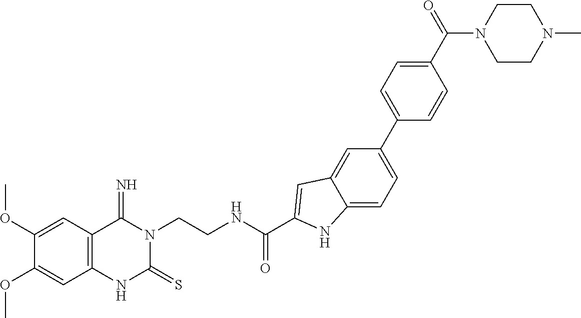 [SMILES: COc1cc2[nH]c(=S)n(CCNC(=O)c3cc4cc(-c5ccc(C(=O)N6CCN(C)CC6)cc5)ccc4[nH]3)c(=N)c2cc1OC] | 2.8 μM |
| BT-2070 | 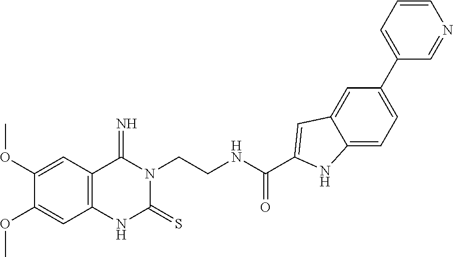 [SMILES: COc1cc2[nH]c(=S)n(CCNC(=O)c3cc4cc(-c5cccnc5)ccc4[nH]3)c(=N)c2cc1OC] | 1.0 μM |
| BT-2071 | 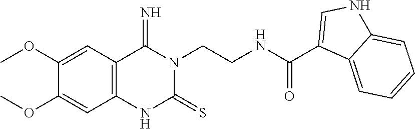 [SMILES: COc1cc2[nH]c(=S)n(CCNC(=O)c3c[nH]c4ccccc34)c(=N)c2cc1OC] | 7 μM |
| BT-2072 | 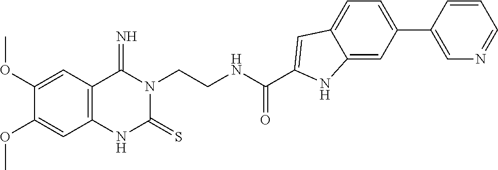 [SMILES: COc1cc2[nH]c(=S)n(CCNC(=O)c3cc4ccc(-c5cccnc5)cc4[nH]3)c(=N)c2cc1OC] | 3.1 μM |
| BT-2103 | 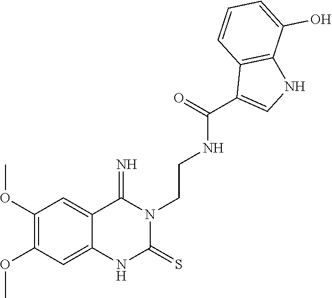 [SMILES: COc1cc2[nH]c(=S)n(CCNC(=O)c3c[nH]c4c(O)cccc34)c(=N)c2cc1OC] | 10.7 μM |
| BT-2113 | 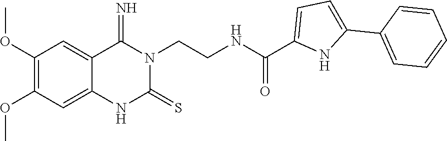 [SMILES: COc1cc2[nH]c(=S)n(CCNC(=O)c3ccc(-c4ccccc4)[nH]3)c(=N)c2cc1OC] | 10.6 μM |
| BT-2133 | 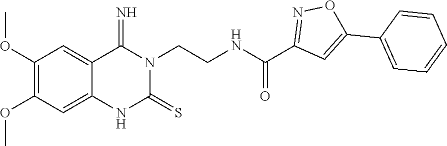 [SMILES: COc1cc2[nH]c(=S)n(CCNC(=O)c3cc(-c4ccccc4)on3)c(=N)c2cc1OC] | 31.7 μM |
| BT-2134 | 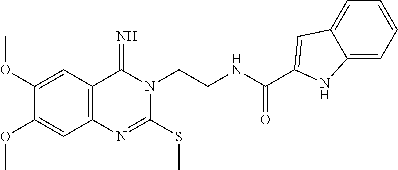 [SMILES: COc1cc2nc(SC)n(CCNC(=O)c3cc4ccccc4[nH]3)c(=N)c2cc1OC] | 24.9 μM |
| BT-2136 | 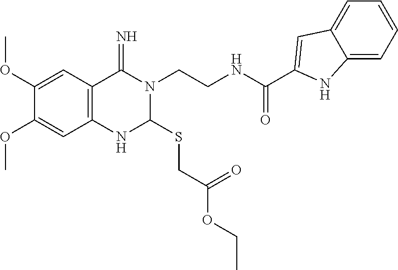 [SMILES: CCOC(=O)CSC1Nc2cc(OC)c(OC)cc2C(=N)N1CCNC(=O)c1cc2ccccc2[nH]1] | 26.8 μM |
| BT-2137 | 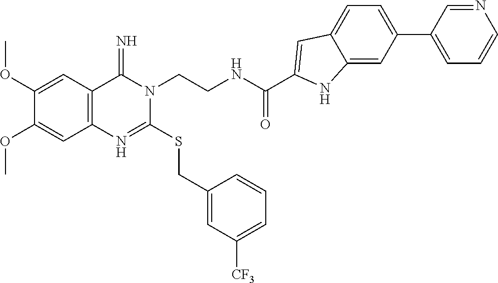 [SMILES: COc1cc2nc(SCc3cccc(C(F)(F)F)c3)n(CCNC(=O)c3cc4ccc(-c5cccnc5)cc4[nH]3)c(=N)c2cc1OC] | 28.0 μM |
| BT-2138 | 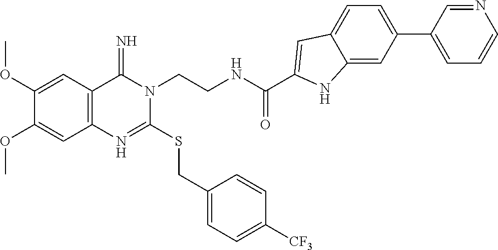 [SMILES: COc1cc2nc(SCc3ccc(C(F)(F)F)cc3)n(CCNC(=O)c3cc4ccc(-c5cccnc5)cc4[nH]3)c(=N)c2cc1OC] | 5.9 μM |
| BT-2153 | 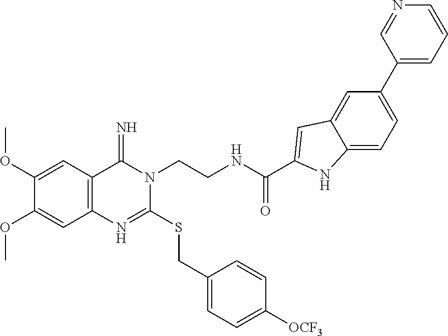 [SMILES: COc1cc2nc(SCc3ccc(OC(F)(F)F)cc3)n(CCNC(=O)c3cc4cc(-c5cccnc5)ccc4[nH]3)c(=N)c2cc1OC] | 5.7 μM |
| BT-2100 | 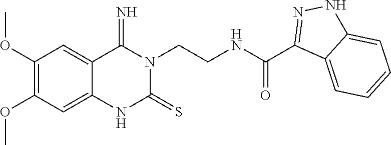 [SMILES: COc1cc2[nH]c(=S)n(CCNC(=O)c3n[nH]c4ccccc34)c(=N)c2cc1OC] | 37.0 μM |
| BT-2152 | 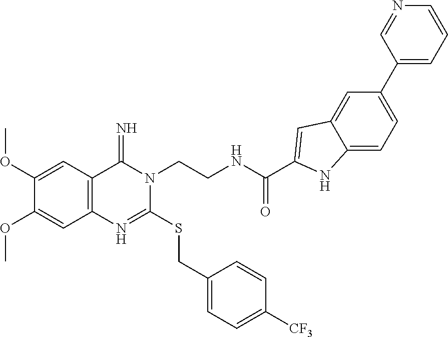 [SMILES: COc1cc2nc(SCc3ccc(C(F)(F)F)cc3)n(CCNC(=O)c3cc4cc(-c5cccnc5)ccc4[nH]3)c(=N)c2cc1OC] | 2.89 μM |

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1.-178. (canceled)

179. A method of treating, or inhibiting the progression or development of, an ocular inflammatory disorder in a subject comprising locally administering pentosan polysulfate or a pharmaceutically acceptable salt thereof to an eye of the subject.

180. The method according to claim 179, wherein the pentosan polysulfate is administered topically, using an ocular implant or directly injected into the eye.

181. The method according to claim 179, wherein the pentosan polysulfate is administered using an intravitreal injection.

182. The method according to claim 179, wherein the ocular inflammatory disorder is selected from the group consisting of age-related macular degeneration, diabetic retinopathy, retinitis pigmentosa, retinal vein occlusion, retinoblastoma, macular edema, uveitis, dry eye, keratoconus and ocular inflammation associated with an infection.

183. The method according to claim 182, wherein the ocular inflammatory disorder is selected from the group consisting of age-related macular degeneration, diabetic retinopathy and retinitis pigmentosa.

184. The method according to claim 183, wherein the ocular inflammatory disorder is age-related macular degeneration.

185. The method according to claim 184, wherein the ocular inflammatory disorder is wet age-related macular degeneration.

186. The method according to claim 184, wherein the ocular inflammatory disorder is dry age-related macular degeneration.

187. The method according to claim 182, wherein the ocular inflammatory disorder is ocular inflammation associated with an infection.

188. The method according to claim 187, wherein the infection is a viral infection.

189. The method according to claim 188, wherein the infection is a herpes simplex virus infection.

190. The method according to claim 179, wherein the pentosan polysulfate inhibits complement fixation.

191. The method according to claim 179, wherein the pentosan polysulfate inhibits macrophage activation.

192. The method according to claim 179, wherein the pentosan polysulfate inhibits growth factor activity.

193. The method according to claim 192, wherein the growth factor is vascular endothelial growth factor (VEGF).

194. The method according to claim 179, wherein the pentosan polysulfate further inhibits oxidative damage.

195. The method according to claim 179, further comprising administering one or more vascular endothelial growth factor (VEGF) inhibitors.

196. The method according to claim 195, wherein the VEGF inhibitor is selected from the group consisting of ranibizumab, aflibercept, bevacizumab, pegaptanib, conbercept and combinations thereof.
